(19) United States
(12) Patent Application Publication
(10) Pub. No.: US 2022/0213113 A1
(43) Pub. Date: Jul. 7, 2022

(54) NITROGEN-CONTAINING CONDENSED CYCLIC COMPOUNDS, FLUORESCENCE EMITTERS, ORGANIC ELECTROLUMINESCENT DEVICES, AND MATERIALS FOR ORGANIC ELECTROLUMINESCENT DEVICES

(71) Applicant: Samsung Electronics Co., Ltd., Suwon-si (KR)

(72) Inventors: Norifumi KISHI, Kanagawa (JP); Mitsunori ITO, Kanagawa (JP); Atsushi IMAMURA, Kanagawa (JP); Eigo MIYAZAKI, Kanagawa (JP); Joonghyuk KIM, Seoul (KR); Inkoo KIM, Suwon-si (KR); Masaru KINOSHITA, Kanagawa (JP); Soonok JEON, Suwon-si (KR); Wataru SOTOYAMA, Kanagawa (JP); Yongsik JUNG, Seoul (KR)

(21) Appl. No.: 17/562,257

(22) Filed: Dec. 27, 2021

(30) Foreign Application Priority Data
Dec. 25, 2020 (JP) 2020-217115
Apr. 28, 2021 (KR) 10-2021-0055000
Nov. 10, 2021 (JP) 2021-183715
Dec. 16, 2021 (KR) 10-2021-0181077

(51) Int. Cl. C07D 487/22; H01L 51/00

(52) U.S. Cl. CPC C07D 487/22; H01L 51/0072; H01L 51/5016; H01L 51/0067; H01L 51/0087

(57) ABSTRACT

The present disclosure relates to a nitrogen-containing condensed cyclic compound having a particular structure and satisfying a particular condition. The present disclosure also relates to a fluorescence emitter that includes the nitrogen-containing condensed cyclic compound having a particular structure and is used together with a phosphorescent complex. In addition, the present disclosure relates to a material for the organic electroluminescent device, including the nitrogen-containing condensed cyclic compound having a particular structure and a phosphorescent complex. Moreover, the present disclosure relates to an organic electroluminescent device including the nitrogen-containing condensed cyclic compound, the fluorescence emitter, and the material for an organic electroluminescent device.

### CROSS-REFERENCE TO RELATED APPLICATIONS

This application claims priority under 35 U.S.C. § 119 to Korean Patent Application No. 10-2021-0181077, filed on Dec. 16, 2021, and Korean Patent Application No. 10-2021-0055000, filed on Apr. 28, 2021, each in the Korean Intellectual Property Office, and Japanese Patent Application No. 2021-183715, filed on Nov. 10, 2021 and Japanese Patent Application No. 2020-217115, filed on Dec. 25, 2020, each in the Japanese Patent Office, the disclosures of which are incorporated by reference herein in their entirety.

### BACKGROUND

### 1. Field

One or more embodiments relate to a nitrogen-containing condensed cyclic compound, a fluorescence emitter, an organic electroluminescent device, and materials for the organic electroluminescent device.

### 2. Description of the Related Art

Recently, in order to improve the performance of organic electroluminescent devices, development of materials and devices that use thermally activated delayed fluorescence (TADF) mechanisms is actively underway. A TADF mechanism consists of a phenomenon wherein reverse intersystem crossing from a triplet exciton to a singlet exciton occurs in a compound, wherein the energy difference (ΔE_{ST}) between a singlet level and a triplet level is small. Details are described on pages 261-262 of Non-Patent Document 1 (Chihaya Adachi, Device properties of organic semiconductor, Kodansha, Mar. 22, 2012).

As a compound having TADF characteristics in the early stages of development, a D-A type fluorescent material having a donor site and an acceptor site has been used. However, because the full width at half maximum (FWHM) of a peak of an emission spectrum of such a compound is wide, the compound may not be sufficient in terms of the specifications used in a wide color gamut device. Therefore, in compounds having TADF characteristics, narrowing the FWHM has been considered a challenge. In this regard, recently there has been development including various studies on using compounds having TADF characteristics as sensitizers or hosts.

Non-Patent Document 2 (Hajime, Nakanotani et al., High-efficiency organic light-emitting diodes with fluorescent emitters, Nature Communications, 2014, 5, 4016 (DOI: 10.1038/ncomms5016)) discloses a technology known as a hyper fluoroluminescent device. This technology relates to a system that uses a compound having TADF characteristics as a sensitizer and transfers energy to a fluorescent dopant (light-emitting dopant) via the sensitizer to emit light. However, in this system, the spectrum of the light-emitting dopant becomes the emission spectrum of a device. For this reason, it has been a challenge for the emission spectrum of the device to become a spectrum wherein tetra t-buperylene (TBPe) used as a light-emitting dopant has a plurality of peaks.

To solve the above problem, Patent Document 1 (JP Publication no. 2020-053667) includes a particular fluorescent compound (nitrogen-containing condensed cyclic compound) having an emission spectrum with a narrow FWHM, and discloses a technology in which a compound having TADF characteristics is used as a sensitizer or a host. In this document, luminescence having an emission spectrum with a narrow FWHM may be obtained is assumed, but there is no description regarding the lifespan of a device. Therefore, it is understood that technology that can be put to practical use has not yet been developed.

In addition, in the field of organic semiconductors, nitrogen-containing condensed cyclic compounds other than compounds having skeleton structures disclosed in Patent Document 1 are also being studied. Such nitrogen-containing condensed cyclic compounds are disclosed in Patent Documents 2 to 4 and Non-Patent Documents 3 and 4.

Non-Patent Document 2 discloses that emission efficiency has been improved by a combination of a fluorescent material and a compound having TADF characteristics, but luminescence color purity of the organic electroluminescent device has not been sufficiently improved. This is because the dopant used to emit blue light has a plurality of peaks.

Through a combination of a particular fluorescent compound (the nitrogen-containing condensed cyclic compound) and a compound having TADF characteristics disclosed in Patent Document 1, high color purity caused by a narrow FWHM of the used nitrogen-containing condensed cyclic compound is achieved. However, emission efficiency remains a challenge as the nitrogen-containing condensed cyclic compound itself does not have TADF characteristics. In addition, because the lifespan of a device is not mentioned therein, it is assumed that lengthening of the lifespan also remains a challenge.

In Patent Document 2 (International Publication no. 2013/084805) and Patent Document 3 (International Publication no. 2013/084835), disclosed are an organic semiconductor material including a nitrogen-containing condensed cyclic compound having a particular structure and an organic electronic device, respectively. However, Patent Documents 2 and 3 mainly focus on electric field mobility of such nitrogen-containing condensed cyclic compounds, and the specially manufactured devices in Patent Documents 2 and 3 is are organic field-effect transistors. Therefore, there is no description or suggestion in Patent Documents 2 and 3 regarding using such nitrogen-containing condensed cyclic compounds as luminescent materials of the organic electroluminescent device or whether such nitrogen-containing condensed cyclic compounds have luminescence characteristics. In addition, there is no description or suggestion in Patent Documents 2 and 3 regarding the relationship between the structure and luminescence color purity of such nitrogen-containing condensed cyclic compounds.

Non-patent Documents 3 (Claude Niebel et al., Dibenzo [2,3:5,6] pyrrolizino [1,7-bc] indolo [1,2,3-Im] carbazole: a new electron donor┘, New Journal of Chemistry, 2010, 34, 1243-1246) and 4 (Morgane Rivoal et al., Substituted dibenzo [2,3:5,6]-pyrrolizino [1,7-bc] Indolo [1,2,3-Im carbazoles: a series of new electron donors, Tetrahedron, 69, (2013), 3302-3307) each disclose a synthesis method, structure, and basic properties of a nitrogen-containing condensed cyclic compound having a particular structure, and that such nitrogen-containing condensed cyclic compounds exhibit photoluminescence. However, there is no description or suggestion in Non-patent Documents 3 and 4 regarding color purity of such nitrogen-containing condensed cyclic compounds. In addition, there is no description or suggestion in Non-patent Documents 3 and 4 regarding combination with other materials when using such nitrogen-containing condensed cyclic compounds as luminescent materials of an organic electroluminescent device. In addition, there is no description or suggestion in Non-patent Documents 3 and 4 regarding the relationship between the structure and luminescence color purity of such nitrogen-containing condensed cyclic compounds.

Patent Document 4 (JP Publication no. 2020-107742) discloses the realization of improvement of emission efficiency through an organic electroluminescent device including a nitrogen-containing condensed cyclic compound having a particular structure. However, there is no description or suggestion in Patent Document 4 regarding color purity of such a nitrogen-containing condensed cyclic compound. In addition, there is no description regarding the TADF characteristics of the nitrogen-containing condensed cyclic compound itself, and emission efficiency remains a challenge. In addition, it is disclosed that using the nitrogen-containing condensed cyclic compound in combination with a phosphorescent complex (phosphorescent material) is not desirable. In Patent Document 4, there is no disclosure of the nitrogen-containing condensed cyclic compound being used in combination with a particular compound among compounds other than the host. In addition, there is no description or suggestion regarding the relationship between the structure and luminescence color purity of such a nitrogen-containing condensed cyclic compound. As described above, although several nitrogen-containing condensed cyclic compounds showing a narrow FWHM are known in the art, compatibility between high color purity and high emission efficiency of an organic electroluminescent device having the characteristics known in the art has not been achieved.

### SUMMARY

One or more embodiments provide a compound wherein a peak wavelength of an emission spectrum is within a blue wavelength region, thereby having high color purity and realizing high emission efficiency.

One or more embodiments provide a fluorescence emitter including the compound.

One or more embodiments provide a material for an organic electroluminescent device including the compound.

One or more embodiments provide a method wherein, in an organic electroluminescent device, a peak wavelength of an emission spectrum is within a blue wavelength region, thereby having high color purity and realizing a high emission efficiency.

Additional aspects will be set forth in part in the description, which follows and, in part, will be apparent from the description, or may be learned by practice of the presented embodiments of the disclosure.

According an aspect of an embodiment, a material for an organic electroluminescent device is provided, comprising a nitrogen-containing condensed cyclic compound having a structure of Formula (1) and a phosphorescent complex:

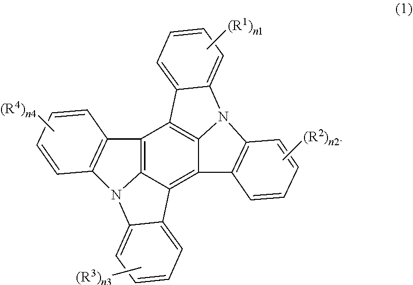

wherein, Formula (1) comprises a core portion, R¹ in the number of n1, R² in the number of n2, R³ in the number of n3, and R⁴ in the number of n4,

wherein, in Formula (1), R¹ to R⁴ are each independently a group of (a) to (g),

n1 to n4 are each independently 0, 1, 2, 3, or 4, and

not all of n1 to n4 are 0:

(a) is a halogen atom;

(b) is a cyano group;

(c) is a substituted or unsubstituted alkyl group having 1 to 20 carbon atoms;

(d) is a substituted or unsubstituted alkoxy group having 1 to 20 carbon atoms;

(e) is a substituted or unsubstituted aryl amino group having 6 to 20 carbon atoms;

(f) is a substituted or unsubstituted monovalent aromatic hydrocarbon group;

(g) is a substituted or unsubstituted monovalent heterocyclic group.

Here, when any one (one, a plurality of, or all) of n1 to n4 is 2 or more, each R¹, each R², each R³, or each R⁴ may be identical to or different from each other. Thus, when n1 is 2 or more, each R₁ may be identical to or different from each other, when n2 is 2 or more, each R₂ may be identical to or different from each other, when n3 is 2 or more, each R₃ may be identical to or different from each other, and when n4 is 2 or more, each R₄ may be identical to or different from each other, and each of R¹ to R⁴ are optionally linked to a ring-forming carbon of the core portion.

According an aspect of an embodiment, a fluorescence emitter includes the nitrogen-containing condensed cyclic compound having the structure represented by Formula (1) and is used together with a phosphorescent complex.

According an aspect of an embodiment, a nitrogen-containing condensed cyclic compound having the structure represented by Formula (1) and satisfying Conditions (i) to (iv).

ΔE_{ST}>ΔE_{ST2}+ΔE′_{TT} Condition (i)

0 eV<ΔE_{ST2}+ΔE′_{TT}≤1.0 eV Condition (ii)

0 eV<ΔE′_{TT}≤0.15 eV Condition (iii)

ΔE_{ST2}>0 eV Condition (iv)

wherein, in Conditions (i) to (iv),

ΔE_{ST}(eV) indicates a difference value obtained by subtracting the lowest triplet excitation energy (eV) calculated from a T₁ equilibrium structure from the lowest singlet excitation energy (eV) calculated from an S₁ equilibrium structure,

ΔE_{ST2}(eV) indicates a difference value obtained by subtracting the second lowest triplet excitation energy (eV) calculated from a T₂ equilibrium structure from the lowest singlet excitation energy (eV) calculated from the S₁ equilibrium structure, and

ΔE′_{TT}(eV) indicates a difference value obtained by subtracting the lowest triplet excitation energy (eV) calculated from the T₂ equilibrium structure from the second lowest triplet excitation energy (eV) calculated from the T₂ equilibrium structure.

According an aspect of an embodiment, a nitrogen-containing condensed cyclic compound has a structure of Formula (1A) or (1B):

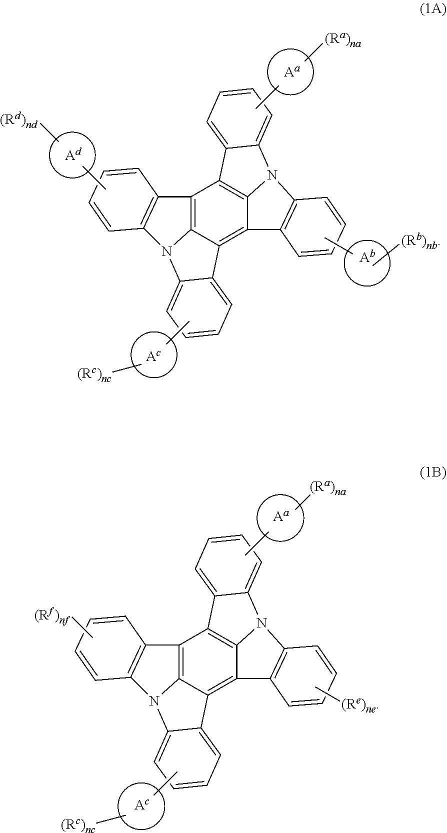

In Formulae (1A) and (1B),

A^{a} to A^{d} may each independently be a group derived from a benzene ring, a group derived from a carbazole ring, or a group represented by Formula (1C),

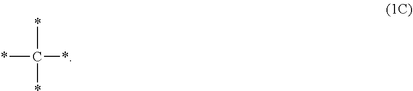

R^{a} to R^{d} may each independently be a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,

R^{e} and R^{f} may each independently be a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

when each of A^{a} to A^{d} is a group derived from a benzene ring, na to nd may each independently be 0, 1, 2, 3, 4, or 5,

when each of A^{a} to A^{d} is a group derived from a carbazole ring, na to nd may each independently be 0, 1, 2, 3, 4, 5, 6, 7, or 8,

when each of A^{a} to A^{d} is a group represented by Formula (1C), na to nd may each independently be 3,

ne and nf may each independently be 0, 1, 2, 3, or 4,

wherein, when na is 2 or more, each R^{a} may be identical to or different from each other,

when nb is 2 or more, each R^{b} may be identical to or different from each other,

when nc is 2 or more, each R^{c} may be identical to or different from each other,

when nd is 2 or more, each R^{d} may be identical to or different from each other,

when ne is 2 or more, each R^{e} may be identical to or different from each other,

when nf is 2 or more, each R^{f} may be identical to or different from each other,

each of R^{a} to R^{f} are optionally linked to a ring-forming carbon of the core portion, and

in Formula (1C), * indicates a binding site to a neighboring atom.

According an aspect of an embodiment, a nitrogen-containing condensed cyclic compound has a structure one of the groups represented by Formulae (2), (3), (7), (8), and (12) to (14):

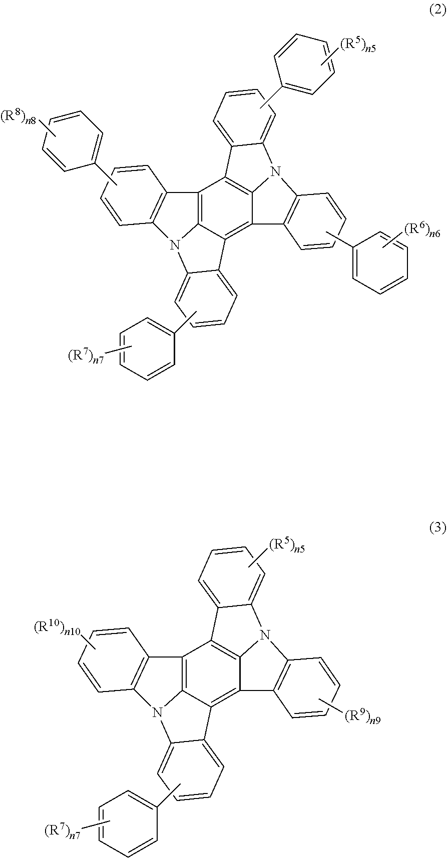

wherein, in Formulae (2) and (3),

R⁵ to R⁸ are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, or an unsubstituted aryl amino group having 6 to 20 carbon atoms,

R⁹ and R¹⁰ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n5 to n8 are each independently 0, 1, 2, 3, 4, or 5,

n9 and n10 are each independently 0, 1, 2, 3, or 4,

when one of n5 to n10 is 2 or more, each R⁵, each R⁶, each R⁷, each R⁸, each R⁹, or each R¹⁰ are identical to or different from each other,

in Formula (2), at least one of n5 to n8 is 3 or more or at least one of R⁵ to R⁸ is an unsubstituted branched alkyl group having 4 to 15 carbon atoms, and

in Formula (3), at least one of n5, n7, n9, and n10 is 3 or more, or at least one of R⁵, R⁷, R⁹, and R¹⁰ is an unsubstituted branched alkyl group having 4 to 15 carbon atoms,

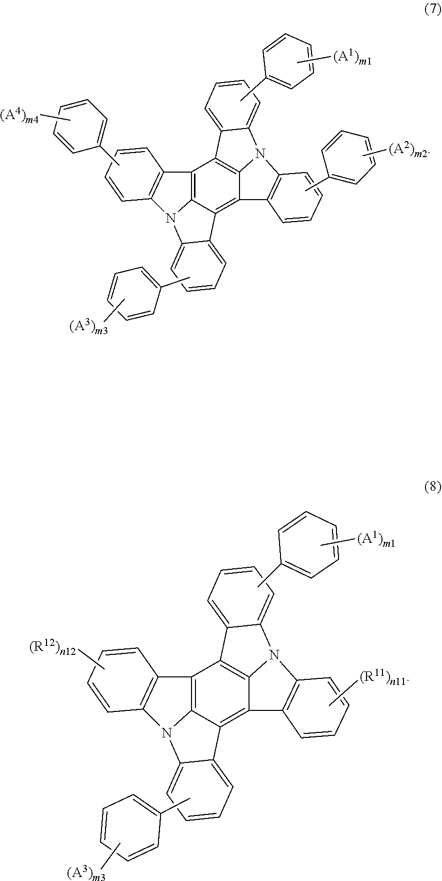

wherein, in Formulae (7) and (8),

A¹ to A⁴ may each independently be an unsubstituted alkyl group having 1 to 20 carbon atoms, a substituted or unsubstituted monovalent aromatic hydrocarbon group, or a substituted or unsubstituted monovalent aromatic heterocyclic group,

R¹¹ and R¹² are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n1 to n4 are each independently 0, 1, 2, 3, 4, or 5,

n11 and n12 are each independently 0, 1, 2, 3, or 4,

wherein, when m1 is 2 or more, each A¹ may be identical to or different from each other,

when m2 is 2 or more, each A² may be identical to or different from each other,

when m3 is 2 or more, each A³ may be identical to or different from each other,

when m4 is 2 or more, each A⁴ may be identical to or different from each other,

when n11 is 2 or more, each R¹¹ may be identical to or different from each other,

when n12 is 2 or more, each R¹² may be identical to or different from each other,

each of A¹ to A⁴ and R¹¹ to R¹² are optionally linked to a ring-forming carbon of the core portion,

wherein, in Formula (7),

not all of A¹ to A⁴ are alkyl groups having 1 to 20 carbon atoms, and not all m1 to m4 are 0,

wherein, in Formula (8),

not all of A¹ to A³ are alkyl groups having 1 to 20 carbon atoms, and not all of m1 and m3 are 0,

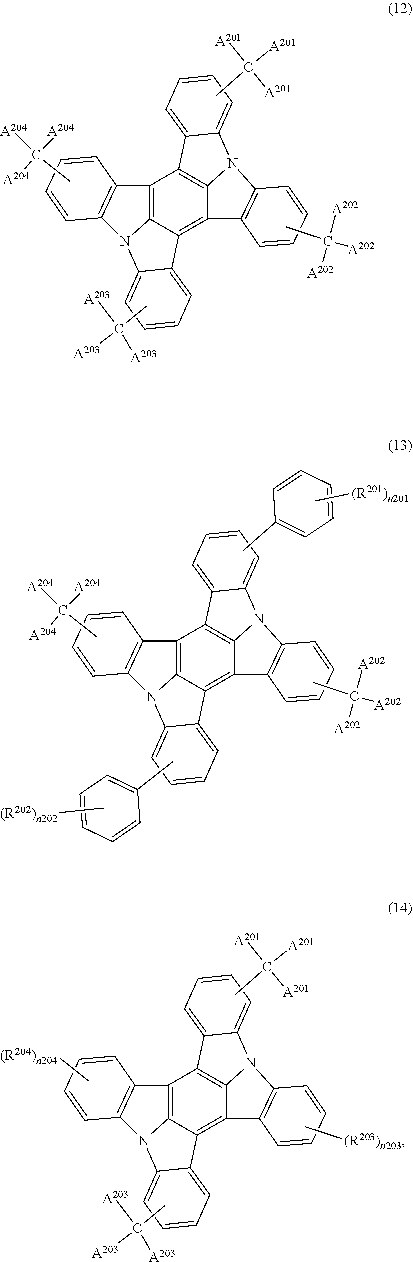

in Formulae (12) to (14),

A²⁰¹ to A²⁰⁴ may each independently be an unsubstituted alkyl group having 1 to 20 carbon atoms, a substituted or unsubstituted monovalent aromatic hydrocarbon group, or a substituted or unsubstituted monovalent aromatic heterocyclic group,

R²⁰¹ to R²⁰² are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,

R²⁰³ and R²⁰⁴ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n201 and n202 are each independently 0, 1, 2, 3, 4, or 5,

n203 and n204 are each independently 0, 1, 2, 3, or 4,

wherein, each A²⁰¹ may be identical to or different from each other, each A²⁰² may be identical to or different from each other, each A²⁰³ may be identical to or different from each other, and each A²⁰⁴ may be identical to or different from each other,

optionally, two or more of A²⁰¹, two or more of A²⁰², two or more of A²⁰³, and two or more of A²⁰⁴ may each form a ring,

when n201 is 2 or more, each R²⁰¹ may be identical to or different from each other, when n202 is 2 or more, each R²⁰² may be identical to or different from each other, when n203 is 2 or more, each R²⁰³ may be identical to or different from each other, and when n204 is 2 or more, each R²⁰⁴ may be identical to or different from each other,

each of A²⁰¹ to A²⁰⁴ are optionally linked to a ring-forming carbon of the core portion of Formula (12),

each of A²⁰², A²⁰⁴, R²⁰¹, and R²⁰² are optionally linked to a ring-forming carbon of the core portion of Formula (13),

each of A²⁰¹, A²⁰³, R²⁰³, and R²⁰⁴ are optionally linked to a ring-forming carbon of the core portion of Formula (14),

in Formula (12), not all of each A²⁰¹, each A²⁰², each A²⁰³, and each A²⁰⁴ are unsubstituted alkyl groups having 1 to 20 carbon atoms,

in Formula (13), not all of each A²⁰² and each A²⁰⁴ are unsubstituted alkyl groups having 1 to 20 carbon atoms, and

in Formula (14), not all of each A²⁰¹ and each A²⁰³ are unsubstituted alkyl groups having 1 to 20 carbon atoms.

### BRIEF DESCRIPTION OF THE DRAWINGS

FIG. 1 is a schematic cross-sectional view of an organic electroluminescent device according to an exemplary embodiment;

FIG. 2 is a schematic cross-sectional view of an organic electroluminescent device according to another exemplary embodiment;

FIG. 3 is a schematic cross-sectional view of an organic electroluminescent device according to another exemplary embodiment;

FIG. 4 is a diagram illustrating a qualitative relation of each energy;

FIG. 5 is a graph regarding known condensed cyclic compounds R1 to R3 showing a FWHM of luminescence in a measured photoluminescence (PL) relative to a reorganization energy (eV) calculated according to density functional theory.

FIG. 6 is a reorganization energy (eV) graph of a fluorescent luminescence of a measured PL calculated according to an FWHM-density functional theory regarding embodiment Compounds D1 to D8 according to an exemplary embodiment.

FIG. 7 is an emission wavelength graph of a measured PL calculated according to a fluorescent wavelength-density functional theory regarding embodiment Compounds D1 to D8 according to an exemplary embodiment.

### DETAILED DESCRIPTION

Hereinafter, embodiments of the present disclosure will be described. In addition, the present disclosure is not limited to the following embodiments. In addition, unless otherwise specified, measurements of operation and physical properties are measured at room temperature (20° C. or more and 25° C. or lower)/relative humidity of 40% RH or more and 50% RH or less.

In this specification, “X and Y are each independently” refers to X and Y being identical to or different from each other.

Further, in the present specification, “a group derived from a ring” refers to a group from which hydrogen atoms directly linked to ring-forming atoms of a ring structure are removed as much as a valence number and having free valence instead. Here, ring-forming atoms represent atoms that directly form a ring structure. For example, in the case of a benzene ring, the ring-forming atoms are carbon atoms, and hydrogen atoms are not included in the ring-forming atoms.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms These terms are only used to distinguish one element, component, region, layer or section from another element, component, region, layer or section Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures It will be understood that relative terms are intended to encompass different orientations of the device in addition to the orientation depicted in the Figures For example, if the device in one of the figures is turned over, elements described as being on the “lower” side of other elements would then be oriented on “upper” sides of the other elements The exemplary term “lower,” can therefore, encompasses both an orientation of “lower” and “upper,” depending on the particular orientation of the figure Similarly, if the device in one of the figures is turned over, elements described as “below” or “beneath” other elements would then be oriented “above” the other elements The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments As such, variations from the shapes of the illustrations as a result, for example, of manufacturing techniques and/or tolerances, are to be expected Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features Moreover, sharp angles that are illustrated may be rounded Thus, the regions illustrated in the figures are schematic in nature and their shapes are not intended to illustrate the precise shape of a region and are not intended to limit the scope of the present claims.

### Nitrogen-Containing Condensed Cyclic Compound

The present disclosure relates to a nitrogen-containing condensed cyclic compound represented by Formula (1):

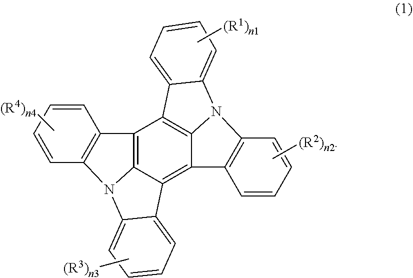

wherein, Formula (1) comprises a core portion, R¹ in the number of n1, R² in the number of n2, R³ in the number of n3, and R⁴ in the number of n4,

wherein, in Formula (1),

R¹ to R⁴ are each independently an atom or one group of (a) to (g),

n1 to n4 are each independently 0, 1, 2, 3, or 4, and

not all of n1 to n4 are 0:

(a) is a halogen atom;

(b) is a cyano group;

(c) is a substituted or unsubstituted alkyl group having 1 to 20 carbon atoms;

(d) is a substituted or unsubstituted alkoxy group having 1 to 20 carbon atoms;

(e) is a substituted or unsubstituted aryl amino group having 6 to 20 carbon atoms;

(f) is a substituted or unsubstituted monovalent aromatic hydrocarbon group;

(g) is a substituted or unsubstituted monovalent heterocyclic group,

Here, when any one (one, a plurality of, or all) of n1 to n4 is 2 or more, each R¹, each R², each R³, or each R⁴ may be identical to or different from each other. Thus, when n1 is 2 or more, each R₁ may be identical to or different from each other, when n2 is 2 or more, each R₂ may be identical to or different from each other, when n3 is 2 or more, each R₃ may be identical to or different from each other, and when n4 is 2 or more, each R₄ may be identical to or different from each other and each of R¹ to R⁴ are optionally linked to a ring-forming carbon of the core portion.

Hereinafter, the “nitrogen-containing condensed cyclic compound according to the present disclosure” may also be referred to as the nitrogen-containing condensed cyclic compound.

Without wishing to be bound by theory, an emission wavelength of a luminescence material may not only change by a skeletal structure but also by the type of substituent and bonding position. Because a particular substituent is introduced in a particular position in a nitrogen-containing condensed cyclic compound, a peak wavelength of the emission spectrum may lengthen to be within a blue wavelength region. As a result, the nitrogen-containing condensed cyclic compound may satisfy the characteristics of a luminescence material of an organic electroluminescent device, particularly, a blue luminescence material. Particularly, aggregation between molecules may be inhibited by the introduction of substituents, the solubility of the molecule itself may be improved, and the degree of purification may be improved, thereby improving luminescence color purity. In addition, in conventional materials, when the added amount of the compound is increased too much, aggregation between molecules occurs in a general luminescence material, luminescence due to the aggregation state occurs, and the width of the emission spectrum is increased, thereby degrading color purity. However, the nitrogen-containing condensed cyclic compound makes it difficult for aggregation between molecules to occur, and thus, color purity is not degraded despite the increase of the added amount of the compound, and high luminescence color purity may be realized. Emission efficiency may also be improved as a result. Also, when the nitrogen-containing condensed cyclic compound is used in combination with the phosphorescent complex, the lifespan of the organic electroluminescent device may be prolonged.

One aspect of the present disclosure relates to a nitrogen-containing condensed cyclic compound represented by Formula (1). Another aspect of the present disclosure relates to a fluorescence emitter including a combination of the nitrogen-containing condensed cyclic compound having the structure represented by Formula (1) and a phosphorescent complex to be described later. Another aspect of the present disclosure relates to a material for an organic electroluminescent device including the nitrogen-containing condensed cyclic compound having the structure represented by Formula (1) and a phosphorescent complex to be described later.

Hereinafter, a nitrogen-containing condensed cyclic compound according to an embodiment, a nitrogen-containing condensed cyclic compound included in a fluorescence emitter according to an embodiment, and a nitrogen-containing condensed cyclic compound included in a material for an organic electroluminescent device according to an embodiment will be described.

R₁ to R₄ in Formula (1) may be a group of (a) to (d), (f), or (g) among the atoms or groups of (a) to (g).

When the groups of (c) to (g) in Formula (1) are present, substituents substituting such groups are not particularly limited. However, in Formula (1), substituents substituting groups of (c) and (d) may each independently be at least one selected from a halogen atom, a cyano group, an unsubstituted alkoxy group having 1 to 20 carbon atoms, an unsubstituted alkyl amino group having 1 to 20 carbon atoms, an unsubstituted aryl amino group having 6 to 20 carbon atoms, and a substituted or unsubstituted monovalent aromatic hydrocarbon group having 6 to 30 carbon atoms. The substituted or unsubstituted monovalent aromatic hydrocarbon group having 6 to 30 carbon atoms may be an unsubstituted monovalent aromatic hydrocarbon group having 6 to 30 carbon atoms.

In Formula (1), substituents substituting groups of (e) may each independently be at least one selected from a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, and an unsubstituted aryl amino group having 6 to 20 carbon atoms. In Formula (1), substituents substituting groups of (f) may each independently be at least one selected from a halogen atom, a cyano group, a substituted or unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, and an unsubstituted monovalent heterocyclic group having 3 to 30 ring-forming atoms. A substituent of a substituted alkyl group having 1 to 20 carbon atoms may be, but not limited to, a monovalent aromatic hydrocarbon group having 6 to 30 carbon atoms unsubstituted or substituted with an unsubstituted alkyl group having 1 to 20 carbon atoms, or a halogen atom. In Formula (1), substituents substituting groups of (f) may each independently be at least one selected from a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, and an unsubstituted monovalent heterocyclic group having 3 to 30 ring-forming atoms. In Formula (1), substituents substituting groups of (g) may each independently be at least one selected from a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, and an unsubstituted monovalent aromatic hydrocarbon group having 6 to 30 carbon atoms.

In Formula (1), when n1, n2, n3, or n4 is 0, R¹, R², R³, or R⁴ corresponding to n1, n2, n3, or n4 are not present. In other words, regarding Formula (1), n1 being 0 means that R¹ is not present, n2 being 0 means that R² is not present, n3 being 0 means that R³ is not present, and n4 being 0 means that R⁴ is not present. Thus, in Formula (1), the ring-forming carbon atoms to which R¹, R², R³, or R⁴ may be linked are unsubstituted, and hydrogen atoms are linked to the ring-forming carbon atoms.

n1 to n4 may each independently be 0 or 1.

The halogen atom of (a) is not particularly limited, but may be, for example, a fluorine atom, a chlorine atom, a bromine atom, or an iodine atom. In an embodiment, the halogen atom of (a) may be a fluorine atom in view of device lifespan.

In (c), the alkyl group of having 1 to 20 carbon atoms is not particularly limited, and may be linear, branched, or annular. In an embodiment, the alkyl group may have a branched structure in view of device lifespan and luminescence color purity. The number of carbon atoms of the alkyl group may be 2 or more, 3 or more, or 4 or more, in view of solubility and luminescence color purity. The number of carbon atoms of the alkyl group may be 10 or less, 8 or less, or 6 or less, in view of device lifespan. The number of carbon atoms of the alkyl group may be 4 in the above point of view. Detailed examples of the alkyl group are not particularly limited, but may include a methyl group, an ethyl group, an n-propyl group, an isopropyl group, an n-butyl group, an s-butyl group (a sec-butyl group), t-butyl group (a tert-butyl group), an i-butyl group, a 2-ethyl butyl group, a 3,3-dimethyl butyl group, an n-pentyl group, an i-pentyl group, a neopentyl group, a t-pentyl group, a cyclo pentyl group, 1-methyl pentyl group, 3-methyl pentyl group, 2-ethyl pentyl group, a 4-methyl-2-pentyl group, an n-hexyl group, a 1-methyl hexyl group, a 2-ethyl hexyl group, a 2-butyl hexyl group, a cyclo hexyl group, a 4-methyl cyclo hexyl group, a 4-t-butyl cyclo hexyl group, an n-heptyl group, a 1-methylpeptyl, a 2,2-dimethyl heptyl group, a 2-ethyl heptyl group, a 2-butyl heptyl group, an n-octyl group, a t-octyl group, a 2-ethyl octyl group, a 2-butyl octyl group, a 2-hexyl octyl group, a 3,7-dimethyl octyl group, a cyclo octyl group, an n-nonyl group, an n-decyl group, an adamantyl group, a 2-ethyl decyl group, 2-butyl decyl group, a 2-hexyl decyl group, a 2-octyl decyl group, a n-undecyl group, an n-dodecyl group, a 2-ethyl dodecyl group, a 2-butyl dodecyl group, a 2-hexyl dodecyl group, a 2-octyl decyl group, an n-tridecyl group, an n-tetra decyl group, an n-pentadecyl group, an n-hexadecyl group, a 2-ethyl hexadecyl group, a 2-butyl hexadecyl group, an n-heptadecyl group, an n-octadecyl group, an n-nonadecyl group, or an n-eicosyl group. In an embodiment, the alkyl group may be a branched alkyl group, an isopropyl group or a tert-butyl group, and a tert-butyl group.

Herein, “a substituted alkyl group having 1 to 20 carbon atoms” refers to an alkyl group having 1 to 20 carbon atoms exclusive of a number of carbons in any substituents. Therefore, the number of carbon atoms of the substituted alkyl group may exceed 20.

In (d), the alkoxy group having 1 to 20 carbon atoms is not particularly limited, and the alkoxy group may be linear, branched, or annular. In an embodiment, the alkoxy group may have a linear structure in view of device lifespan. The number of carbon atoms of the alkoxy group may be 1 or more and 10 or less in view of device lifespan. In addition, the number of carbon atoms of the alkoxy group may be 1 or more and 8 or less, and 1 or more and 6 or less, in the same point of view. An example of the alkyl group constituting the alkoxy group may include, but is not limited to, the descriptions relating to the alkyl group, and the like. Detailed examples of the alkoxy group may include, but are not limited to, a methoxy group, an ethoxy group, an n-propoxy group, an isopropoxy group, a butoxy group, a pentyloxy group, a hexyloxy group, an octyloxy group, a nonyloxy group, or a decyloxy group. In an embodiment, the alkoxy group may be a methoxy group.

Herein, “a substituted alkoxy group having 1 to 20 carbon atoms” refers to an alkoxy group having 1 to 20 carbon atoms exclusive of a number of carbons in any substituents. Therefore, the number of carbon atoms of the substituted alkoxy group may be exceed 20.

A nitrogen atom of the aryl amino group having 6 to 20 carbon atoms refers to a group represented by —NA₁₀₁ (wherein A₁₀₁ is an aryl group) or —N(A₁₀₁)(A₁₀₂) (wherein A₁₀₁ and A₁₀₂ are aryl groups). An example of an aryl group included in an aryl amino group may include, but is not limited to, a group having 6 to 20 carbon atoms among monovalent aromatic hydrocarbon groups to be described later. The aryl amino group may be, but is not particularly limited to, a monoaryl amino group or diaryl amino group. Detailed examples of the aryl amino group may be, but are not limited to, an N-phenylamino group, an N-biphenylamino group, an N-terphenylamino group, an N,N-diphenylamino group, or an N-biphenyl-N-phenyl amino group.

In addition, “a substituted aryl amino group having 6 to 20 carbon atoms” refers to an aryl amino group having 6 to 20 carbon atoms wherein the number of carbons in any substituent are excluded from the 6 to 20 carbon atoms. Therefore, the number of carbon atoms of the substituted aryl amino group may exceed 20.

The monovalent aromatic hydrocarbon group of (f) refers to a monovalent group derived from at least one aromatic hydrocarbon ring. An aromatic hydrocarbon ring as used herein refers to a hydrocarbon ring that is partially aromatic or is aromatic as a whole.

When a monovalent aromatic hydrocarbon group includes two or more aromatic hydrocarbon rings, such rings may be linked by a single bond or condensed with each other. Further, when the monovalent aromatic hydrocarbon group includes two or more aromatic hydrocarbon rings, one atom may act as a ring-forming atom of any of these rings.

The number of carbon atoms of the monovalent aromatic hydrocarbon group is not particularly limited, but may be 6 or more and 30 or less in view of luminescence color purity. In addition, within that range, the number of carbon atoms of the monovalent aromatic hydrocarbon group may be 6 or more and 20 or less, 6 or more and 12 or less, or 6.

Detailed examples of the monovalent aromatic hydrocarbon group may include, but are not limited to, a phenyl group, a mesityl group, a t-butyl phenyl group, a bis (t-butyl) phenyl group, a biphenyl group, a terphenyl group, a naphthyl group, a fluorenyl group, an anthracene group, a quarterphenyl group, a quinquephenyl group, a sexyphenyl group, a triphenylenyl group, a pyrenyl group, a benzofluorenyl group, a chrysenyl group, or any combination thereof.

Herein, “a substituted monovalent aromatic hydrocarbon group” refers to a monovalent aromatic hydrocarbon group wherein the number of carbons in any substituent are excluded from the 6 to 30 carbon atoms. Therefore, when the number of carbon atoms of the monovalent aromatic hydrocarbon group is equal to or less than the upper limit value of a certain carbon atom, for example, 30 or less, the number of carbon atoms of a substituted monovalent aromatic hydrocarbon group may exceed the upper limit.

The monovalent heterocyclic group of (g) refers to a group derived from one or more hetero rings. The monovalent heterocyclic group is not particularly limited, and may be a monovalent aromatic heterocyclic group or a monovalent non-aromatic heterocyclic group. In an embodiment, the monovalent heterocyclic group may be a monovalent aromatic heterocyclic group in view of luminescence color purity.

A monovalent aromatic heterocyclic group as used herein refers to a group derived from at least one aromatic hetero ring. An aromatic hetero ring as used herein refers to a hetero ring that is partially aromatic or is aromatic as a whole. When the aromatic hetero ring is partially aromatic, aromaticity may be derived from a heterocyclic part of the ring or from the hydrocarbon ring part of the ring. An example of the aromatic hetero ring may include, but not limited to, a ring that has at least one hetero atom as a ring-forming atom (for example, a nitrogen atom (N), an oxygen atom (O), a phosphorus atom (P), a sulfur atom (S), a silicon atom (Si)), and the rest of the ring-forming atom is a carbon atom (C). In addition, an atom constituting a ring structure may be linked to an exocyclic atom via a double bond. For example, the carbon atom constituting the ring structure may be included in a ketone group (C═O group) or a thio ketone group (C═S group), or a C═NH group, or the sulfur atom forming the sulfur structure may be included in a sulfinyl group (S═O group) or a sulfonyl (S(═O)═O group). In this case, the exocyclic atom forming the double bond with an atom substituting a ring structure may be part of the aromatic hetero ring. When an exocyclic atom that forms a double bond is linked to a hydrogen atom via a single bond, the hydrogen atom may also be part of the aromatic hetero ring. Examples of the aromatic hetero ring may include, but are not limited to, a pyridine ring, a pyrazine ring, a pyridazine ring, a pyrimidine ring, a triazine ring, a quinoline ring, an isoquinoline ring, a quinoxaline ring, a quinazoline ring, a naphthyridene ring, an acridine ring, a phenazine ring, a benzoquinoline ring, a benzoisoquinoline ring, a phenanthridine ring, a phenanthroline ring, a benzoquinone ring, a coumarin ring, an anthraquinone ring, a fluorenone ring, a furan ring, a thiophene ring, a benzofuran ring, a benzothiophene ring, a dibenzofuran ring, a dibenzothiophene ring, a pyrrole ring, an indole ring, a carbazole ring, an indolecarbazole ring, an imidazole ring, benzimidazole ring, a pyrazole ring, indazole ring, an oxazole ring, isoxazole ring, a benzoxazole ring, benzoisoxazole ring, a thiazole ring, an isothiazole ring, a benzothiazole ring, benzoisothiazole ring, an imidazolinone ring, benzimidazolinone ring, an imidazolepyridine ring, an imidazolepyrimidine ring, an imidazolephenanthridine ring, a benzimidazolephenanthridine ring, an azadibenzofuran ring, an azacarbazole ring, an azadibenzothiophene ring, a diazadibenzofuran ring, a diazacarbazole ring, a diazadibenzothiophene ring, a xanthone ring, or a thioxanthone ring.

When a monovalent aromatic heterocyclic group includes two or more aromatic hetero rings, such rings may be linked by a single bond or condensed with each other. Further, when the monovalent aromatic heterocyclic group includes two or more aromatic hetero rings, one atom may act as a ring-forming atom in any of these rings.

The number of ring-forming atoms of the monovalent aromatic hetero ring (the sum of the number of ring-forming carbon atoms and the number of ring-forming hetero atoms) may be, but not limited to, 3 or more and 30 or less, in view of the peak wavelength of the emission spectrum and luminescence color purity. Further, the number of ring-forming atoms of the monovalent aromatic heterocyclic group may be 5 or more and 20 or less, or 6 or more and 14 or less, from the same point of view. The number of ring-forming hetero atoms of the monovalent aromatic heterocyclic group may be, but not limited to, 1 or more and 10 or less, in view of the peak wavelength of the emission spectrum and luminescence color purity. The number of ring-forming hetero atoms of the monovalent aromatic heterocyclic group may be 1 or more and 5 or less, and 1 or more and 3 or less, from the same point of view. In addition, ring-forming atoms represent atoms that directly form a ring structure, as described above. In this regard, when there is an exocyclic atom forming a double bond with an atom constituting a ring structure, the exocyclic atom is not included in the ring-forming atom.

Examples of the monovalent aromatic heterocyclic group may include, but are not limited to, a thienyl group, a furanyl group, a pyrrolyl group, an imidazolyl group, a thiazolyl group, an oxazolyl group, an oxadiazolyl group, a triazolyl group, a pyridyl group, a bipyridyl group, a pyrimidyl group, a triazinyl group, an acridinyl group, a pyridazinyl group, a pyrazinyl group, a quinolinyl group, a quinazolinyl group, a quinoxalinyl group, phenoxazinyl group, a phthalazinyl group, a pyridopyrimidinyl group, a pyridopyrazinyl group, a pyrazinopyrazinyl group, an isoquinolinyl group, an indolyl group, a carbazolyl group, a benzoxazolyl group, a benzimidazolyl group, a benzothiazolyl group, a benzocarbazolyl group, a benzothiophenyl group, a dibenzothiophenyl group, thienothienyl group, a benzofuranyl group, a phenanthrolinyl group, a thiazolyl group, an isoxazolyl group, an oxadiazolyl group, a thiadiazolyl group, a phenothiazinyl group, dibenzosilolyl group, a dibenzofuranyl group, or a xanthonyl group. In an embodiment, the monovalent aromatic heterocyclic group may be a triazinyl group, a carbazolyl group, a benzoxazolyl group, and a xanthonyl group.

A non-aromatic heterocyclic group as used herein refers to a group derived from at least one non-aromatic hetero ring. A non-aromatic hetero ring as used herein refers to a hetero ring that is partially non-aromatic or is non-aromatic as a whole. The non-aromatic hetero ring is not particularly limited, but may be, for example, a ring that has at least one hetero atom (for example, a nitrogen atom (N), an oxygen atom (O), a phosphorus atom (P), a sulfur atom (S), a silicon atom (Si)) as a ring-forming atom and the rest of the ring-forming atom is a carbon atom (C). The hetero atom may be a nitrogen atom (N) or an oxygen atom (O), in view of the peak wavelength of the emission spectrum and luminescence color purity. In addition, an atom constituting a ring structure may be linked to an exocyclic atom via a double bond. For example, the carbon atom constituting the ring structure may be included in a ketone group (C═O group) or a thio ketone group (C═S group), or a C═NH group, or the sulfur atom forming the sulfur structure may be included in a sulfinyl group (S═O group) or a sulfonyl (S(═O)═O group). In this case, the exocyclic atom forming the double bond with an atom constituting a ring structure may be part of the non-aromatic hetero ring. When an exocyclic atom that forms a double bond is linked to a hydrogen atom via a single bond, the hydrogen atom may also be part of the non-aromatic hetero ring. Examples of the non-aromatic hetero ring may include, but are not limited to, a pyrrolidine ring, tetrahydrofuran ring, a tetrahydrothiophene ring, a piperidine ring, tetrahydropyran ring, a tetrahydrothiopyran ring, a dioxane ring, a morpholine ring, or a dioxolane ring.

When a monovalent non-aromatic heterocyclic group includes two or more non-aromatic hetero rings, such rings may be linked by a single bond or condensed with each other. Further, when the monovalent non-aromatic heterocyclic group includes two or more non-aromatic hetero rings, one atom may act as a ring-forming atom of any of these rings.

The number of ring-forming atoms of the monovalent non-aromatic hetero ring (the sum of the number of ring-forming carbon atoms and the number of ring-forming hetero atoms) may be, but not limited to, 3 or more and 30 or less, in view of the peak wavelength of the emission spectrum and luminescence color purity. Further, the number of ring-forming atoms of the monovalent non-aromatic heterocyclic group may be 5 or more and 20 or less, or 6 or more and 14 or less, from the same point of view. The number of ring-forming hetero atoms of the monovalent non-aromatic heterocyclic group may be, but not limited to, 1 or more and 10 or less, in view of the peak wavelength of the emission spectrum and luminescence color purity. The number of ring-forming hetero atoms of the monovalent non-aromatic heterocyclic group may be 1 or more and 5 or less, and 1 or more and 3 or less, from the same point of view. In addition, ring-forming atoms represent atoms that directly form a ring structure, as described above. In this regard, when there is an exocyclic atom forming a double bond with an atom constituting a ring structure, the exocyclic atom is not included in the ring-forming atom.

Detailed examples of the monovalent non-aromatic heterocyclic group may include, but are not limited to, a pyrrolidinyl group, a tetrahydrofuranyl group, a tetrahydrothienyl group, a pyridinyl group, tetrahydropyranyl group, a tetrahydrothiopyranyl group, a dioxanyl group, a morpholinyl group, or a dioxoranyl group.

Herein, “a substituted monovalent heterocyclic group” refers to a monovalent heterocyclic group substituted with a substituent wherein the number of carbons in any substituent are excluded from the number of carbons recited for the monovalent heterocyclic group. Therefore, when the number of ring-forming atoms of the monovalent heterocyclic group is equal to or less than the upper limit value of a particular ring-forming atom, for example, 30 or less, and when a substituent constitutes a ring structure, the number of ring-forming atoms of a substituted monovalent heterocyclic group may exceed the upper limit.

The groups of (c) to (g) may be substituted. A halogen atom, a cyano group, an unsubstituted alkyl group having 1 to 20 carbon atoms, an unsubstituted alkoxy group having 1 to 20 carbon atoms, and an unsubstituted aryl amino group having 6 to 20 carbon atoms, which are substituents substituting groups of (c) to (g), are each the same as described in connection with the unsubstituted groups of (a), (c), (d), and (e). The substituents substituting groups of (c) to (g) may be the halogen atom or the unsubstituted alkyl group having 1 to 20 carbon atoms. The substituents substituting groups of (c) to (g) may be the fluorine atom or the unsubstituted linear or branched alkyl group having 1 to 20 carbon atoms. The substituents substituting groups of (c) to (g) may be the fluorine atom, the methyl group, the isopropyl group, or the tert-butyl group.

The unsubstituted alkyl group having 1 to 20 carbon atoms, which is a substituent substituting the group of (f) is the same as described in connection with the unsubstituted group of (c). The substituents substituting groups of (c) to (g) may be the unsubstituted linear or branched alkyl group having 1 to 20 carbon atoms. The substituents substituting groups of (c) to (g) may be the methyl group, the ethyl group, the isopropyl group, or the tert-butyl group.

In an unsubstituted alkyl amino group having 1 to 20 carbon atoms, which is a substituent substituting the groups of (c) to (g), refers to a group represented by —NA₁₀₃ (wherein A₁₀₃ is an alkyl group) or —N(A₁₀₃)(A₁₀₄) (wherein A₁₀₃ and A₁₀₄ are alkyl groups). The nitrogen atoms may bond with a certain number of atoms constituting the unsubstituted groups of (c) to (g) of Formula (1) via a single bond. The alkyl group constituting the alkyl amino group is not particularly limited, but may be, for example, the same as described in connection with (c). The alkyl amino group may be, but not limited to, a monoalkyl amino group or a dialkyl amino group. Detailed examples of the alkyl amino group may include, but not limited to, a N-methyl amino group, a N-ethyl amino group, a N-propyl amino group, a N-isopropyl amino group, a N-butyl amino group, a N-isobutyl amino group, a N-sec-butyl amino group, a N-tert-butyl amino group, a N-pentyl amino group, a N-hexyl amino group, a N,N-dimethyl amino group, a N-methyl-N-ethyl amino group, a N,N-diethyl amino group, a N,N-dipropyl amino group, a N,N-diisopropyl amino group, a N,N-dibutyl amino group, a N,N-diisobutyl amino group, a N,N-dipentyl amino group, or a N,N-dihexyl amino group.

An unsubstituted halo alkyl group having 1 to 20 carbon atoms, which is a substituent substituting the groups of (e) to (g) may be a group wherein at least one hydrogen atom of the alkyl group described in (c) is substituted with the halogen atom described in (a). The halogen atom may be a fluorine atom in view of device lifespan. Detailed examples of the halo alkyl group may include a trifluoromethyl group, a trichloromethyl group, a tribromomethyl group, or a triiodomethyl group. In an embodiment, the halo alkyl group may be a fluoro alkyl group, and further a trifluoromethyl group.

An unsubstituted monovalent heterocyclic group having 3 to 30 ring-forming atoms, which is a substituent substituting the group of (f) is the same as described in connection with the unsubstituted group of (g), except that the number of ring-forming atoms is limited. The unsubstituted monovalent heterocyclic group may be a monovalent heterocyclic group including oxygen or nitrogen atom as a hetero atom, particularly a dibenzofuranyl group, a carbazolyl group, or a benzoxazolyl group. The unsubstituted monovalent heterocyclic group may be, for example, a benzofuranyl group or a carbazolyl group.

The unsubstituted monovalent aromatic hydrocarbon group having 6 to 30 carbon atoms, which is a substituent substituting the groups of (c), (d), and (g), is the same as described in connection with the unsubstituted group of (f), except that the number of carbon atoms is limited. The unsubstituted monovalent aromatic hydrocarbon group may be a group derived from a benzene ring.

When the groups of (c) to (g) are substituted groups, the substituents may be groups substituted with new substituents. New substituents may include, but are not limited to, for example, substituents listed as the substituted groups of (c) to (g), and such substituents may further be substituted based on the same manner as when the groups of (c) to (g) are substituted.

Regarding the substituted monovalent aromatic hydrocarbon group having 6 to 30 carbon atoms that substitutes the groups of (c) and (d), the monovalent aromatic hydrocarbon group having 6 to 30 carbon atoms is the same as described in connection with the unsubstituted of group (g), except that the number of carbon atoms is limited. The monovalent aromatic hydrocarbon group may be a group derived from a benzene ring. Further, a substituent substituting the monovalent aromatic hydrocarbon group having 6 to 30 carbon atoms may be, but is not limited to, an unsubstituted alkyl group having 1 to 20 carbon atoms. The unsubstituted alkyl group having 1 to 20 carbon atoms is the same as described in connection with the unsubstituted group of (c). The substituent substituting the monovalent aromatic hydrocarbon group may be the unsubstituted linear or branched alkyl group having 1 to 20 carbon atoms. The unsubstituted alkyl group having 1 to 20 carbon atoms may be a methyl group, an ethyl group, an isopropyl group, or a tert-butyl group.

Regarding, the substituted alkyl group having 1 to 20 carbon atoms substituting the group of (f), the alkyl group having 1 to 20 carbon atoms is the same as described in connection with the unsubstituted group of (c). The unsubstituted alkyl group having 1 to 20 carbon atoms may be the unsubstituted linear or branched alkyl group having 1 to 20 carbon atoms. The unsubstituted alkyl group having 1 to 20 carbon atoms may be the methyl group, the ethyl group, the isopropyl group, or the tert-butyl group. A substituent substituting an alkyl group having 1 to 20 carbon atoms may be, but not limited to, a monovalent aromatic hydrocarbon group having 6 to 30 carbon atoms unsubstituted or substituted with an unsubstituted alkyl group having 1 to 20 carbon atoms. The unsubstituted alkyl group having 1 to 20 carbon atoms substituting the monovalent aromatic hydrocarbon group having 6 to 30 carbon atoms is the same as described in connection with the unsubstituted group of (c). The unsubstituted alkyl group having 1 to 20 carbon atoms may be the unsubstituted linear or branched alkyl group having 1 to 20 carbon atoms. The unsubstituted alkyl group having 1 to 20 carbon atoms may be the methyl group, the ethyl group, the isopropyl group, or the tert-butyl group.

Regarding Formula (1), at least one of the groups represented by R¹ to R⁴ may be linked to at least one ring-forming carbon atom of the core portion. The core portion of Formula (1) is represented by Formula 1-1 below. In Formula (1), R¹ to R⁴ that may be linked to such ring-forming carbon atom may be, but not limited to, for example, a terphenyl group, and a phenyl group substituted with at least two alkyl groups having 1 to 4 carbon atoms. The R¹ to R⁴ group may also be, for example, an m-terphenyl group, or a 2, 6-di-tert-butylphenyl group.

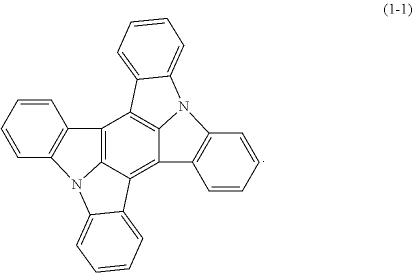 [SMILES: c1ccc2c(c1)c1c3c4ccccc4n4c5ccccc5c(c5c6ccccc6n2c15)c34]

In the nitrogen-containing condensed cyclic compound according to the present disclosure, in view of the peak wavelength of the emission spectrum and luminescence color purity, the structure represented by Formula (1) may be a structure represented by Formula (1A) or (1B). Thus, an embodiment of the present disclosure relates to a nitrogen-containing condensed cyclic compound having the structure represented by Formula (1A) or (1B). In an embodiment of a material for an organic electroluminescent device to be described later, the structure represented by Formula (1) of the nitrogen-containing condensed cyclic compound may be the structure represented by Formula (1A) or (1B). In an embodiment of a fluorescence emitter to be described later, the structure represented by Formula (1) of the nitrogen-containing condensed cyclic compound may be the structure represented by Formula (1A) or (1B).

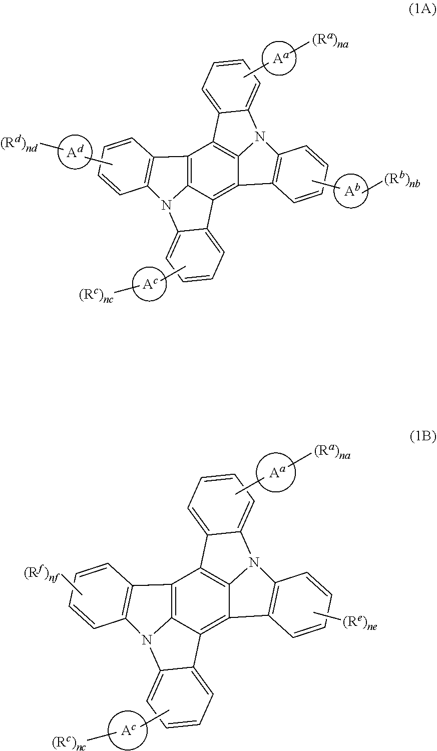

In Formulae (1A) and (1B),

A^{a} to A^{d} may each independently be a group derived from a benzene ring, a group derived from a carbazole ring, or a group represented by Formula (1C),

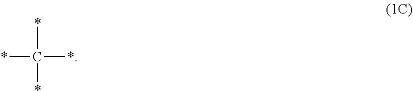

R^{a} to R^{d} may each independently be a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,

R^{e} and R^{f} may each independently be a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

when each of A^{a} to A^{d} is a group derived from a benzene ring, na to nd may each independently be 0, 1, 2, 3, 4, or 5,

when each of A^{a} to A^{d} is a group derived from a carbazole ring, na to nd may each independently be 0, 1, 2, 3, 4, 5, 6, 7, or 8,

when each of A^{a} to A^{d} is a group represented by Formula (1C), na to nd may each independently be 3,

ne and nf are each independently 0, 1, 2, 3, or 4,

wherein, when na is 2 or more, each R^{a} may be identical to or different from each other, when nb is 2 or more, each R^{b} may be identical to or different from each other, when nc is 2 or more, each R^{c} may be identical to or different from each other, when nd is 2 or more, each R^{d} may be identical to or different from each other, when ne is 2 or more, each R^{e} may be identical to or different from each other, and when nf is 2 or more, each R^{f} may be identical to or different from each other, and each of R¹ to R⁴ are optionally linked to a ring-forming carbon of the core portion,

in Formula (1C), * indicates a binding site to a neighboring atom.

In Formula (1A), when each of A^{a} to A^{d} is a group derived from a benzene ring, at least one of na to nd may be 3 or more, or at least one of R^{a} to R^{d} may be an unsubstituted branched alkyl group having 4 to 15 carbon atoms.

In Formula (1B), when each of A^{a} and A^{c} is a group derived from a benzene ring, at least one of na, nc, ne, and nf may be 3 or more, or at least one of R^{a}, R^{c}, R^{e}, and R^{f} may be an unsubstituted branched alkyl group having 4 to 15 carbon atoms.

Regarding R^{a} to R^{f}, the halogen atom, the unsubstituted alkyl group having 1 to 20 carbon atoms, the unsubstituted alkoxy group having 1 to 20 carbon atoms, or the aryl amino group having 6 to 20 carbon atoms are each the same as described in connection with the unsubstituted groups of (a), (c), (d), and (e).

Regarding R^{a} to R^{f}, the unsubstituted halo alkyl group having 1 to 20 carbon atoms may be identical to the substituent substituting the groups of (e) to (g).

Regarding R^{a} to R^{d}, the substituted or unsubstituted monovalent aromatic hydrocarbon group and the substituted or unsubstituted monovalent aromatic heterocyclic group are each the same as described in connection with the groups of (f) and (g).

Regarding R^{a} to R^{d}, the unsubstituted alkyl amino group having 1 to 20 carbon atoms is the same as described in connection with the substituent substituting the groups of (c) to (g).

Regarding R^{e} and R^{f}, the unsubstituted alkoxy group having 1 to 20 carbon atoms may be a group wherein at least one hydrogen atom of the alkoxy group described in (d) is substituted with the halogen atom described in (a). The halogen atom may be a fluorine atom in view of device lifespan. Detailed examples of the alkoxy group may include a trifluoromethoxy group, a trichloromethoxy group, a tribromomethoxy group, or a triiodomethoxy group. In an embodiment, the alkoxy group may be a fluoro alkoxy group, or a trifluoromethoxy group.

In Formulae (1A) and (1B), when na, nb, nc, nd, ne, or nf is 0, R^{a}, R^{b}, R^{c}, R^{d}, R^{e}, or R^{f} corresponding to na, nb, nc, nd, ne, or nf are not present. In other words, regarding (1A) and (1B), na being 0 means that R^{a} is not present, nb being 0 means that R^{b} is not present, nc being 0 means that R^{c} is not present, and nd being 0 means that R^{d} is not present. In addition, in Formula (1B), ne being 0 means that R^{e} is not present, and nf being 0 means that R^{f} is not present. Thus, Formulae (1A) and (1B) show that the ring-forming carbon atoms to which R^{a}, R^{b}, R^{c}, R^{d}, R^{e}, or R^{f} may be linked are unsubstituted, and hydrogen atoms are linked to the ring-forming carbon atoms.

Regarding Formulae (1A) and (1B), at least one of the groups represented by A^{a} to A^{d} may be linked by single bond or linked by a substituent of A^{a} to A^{d} to at least one ring-forming carbon atom of the core portion of Formulae (1A) and (1B). In Formulae (1A) and (1B), A^{a} to A^{d} that may be linked to such ring-forming carbon atom may each independently be, but are not limited to, for example, a terphenyl group, and a phenyl group substituted with at least two alkyl groups having 1 to 4 carbon atoms. The A^{a} to A^{d} groups may also be, for example, an m-terphenyl group, or a 2, 6-di-tert-butylphenyl group.

In the nitrogen-containing condensed cyclic compound according to the present disclosure, in view of the peak wavelength of the emission spectrum and luminescence color purity, the structure represented by Formula (1) or (1B) may be the structure represented by Formula (2) or (3). Thus, an embodiment of the present disclosure relates to a nitrogen-containing condensed cyclic compound having the structure represented by Formula (2) or (3). In an embodiment of a material for an organic electroluminescent device to be described later, the structure represented by Formula (1) of the nitrogen-containing condensed cyclic compound may be the structure represented by Formula (2) or (3). In an embodiment of a fluorescence emitter to be described later, the structure represented by Formula (1) of the nitrogen-containing condensed cyclic compound may be the structure represented by Formula (2) or (3). In the nitrogen-containing condensed cyclic compound, the material for the organic electroluminescent device, and the fluorescence emitter according to an embodiment, regarding Formulae (2) and (3), at least one of the R groups (i.e., R⁵ to R¹⁰) may be linked to at least one of a ring-forming carbon atom of the core portion of Formulae (2) and (3). In Formulae (2) and (3), at least one of the R groups (i.e., R⁵ to R¹⁰) that may be linked to such ring-forming carbon atom of the core portion may be, but not limited to, for example, a phenyl group substituted with at least two alkyl groups having 1 to 4 carbon atoms. In addition, at least one of the R groups (i.e., R⁵ to R¹⁰) linked to the core portion may be a 2, 6-di-tert-butyl phenyl group. In the nitrogen-containing condensed cyclic compound, the material for the organic electroluminescent device, and the fluorescence emitter, when the structure represented by Formula (2) or (3) is a structure represented by Formula (2′) or (3′), the photoluminescence quantum yield (PLQY) may be improved.

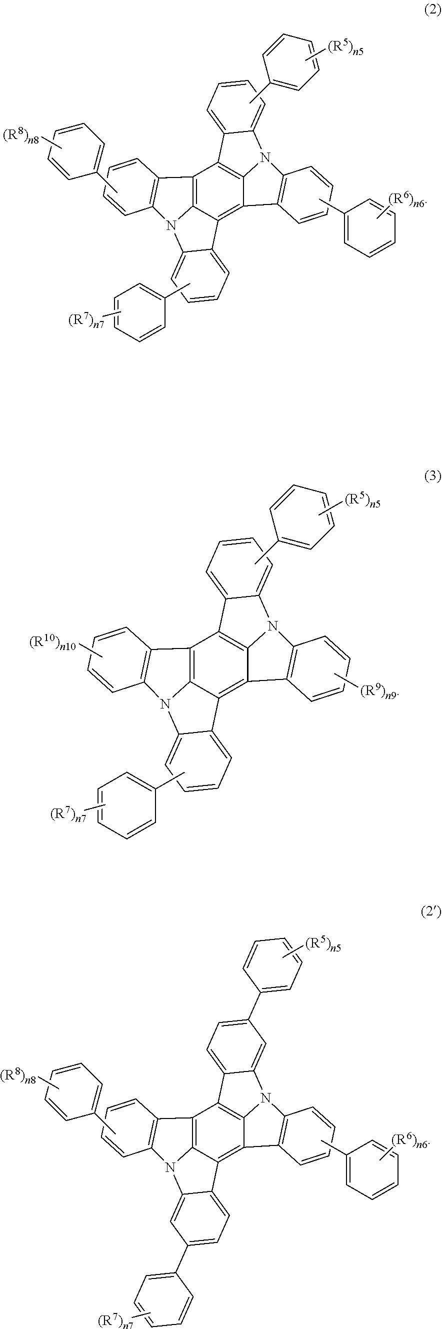

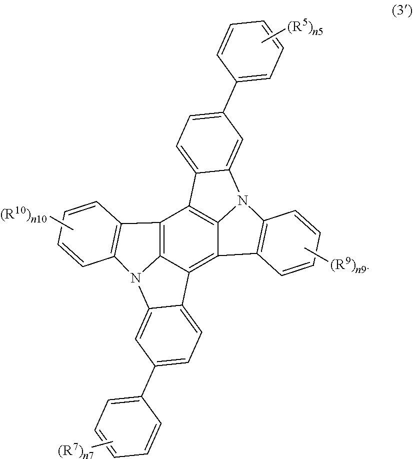

In Formulae (2) and (3), or in Formulae (2′) and (3′),

R⁵ to R⁸ are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, or an unsubstituted aryl amino group having 6 to 20 carbon atoms,

R⁹ and R¹⁰ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n5 to n8 are each independently 0, 1, 2, 3, 4, or 5,

n9 and n10 are each independently 0, 1, 2, 3, or 4, and

when any one (one, a plurality of, or all) of n5 to n10 is 2 or more, each R⁵, each R⁶, each R⁷, each R⁸, each R⁹, or each R¹⁰ may be identical to or different from each other.

In Formulae (2) and (3), or in Formula (2′) and (3′), when any one (one, a plurality of, or all) of n5 to n10 is 2 or more, each R⁵, each R⁶, each R⁷, each R⁸, each R⁹, or each R¹⁰ may be identical to or different from each other. When n5 is 2 or more, each R⁵ may be identical to or different from each other, when n6 is 2 or more, each R⁶ may be identical to or different from each other, when n7 is 2 or more, each R⁷ may be identical to or different from each other, when n8 is 2 or more, each R⁸ may be identical to or different from each other, when n9 is 2 or more, each R⁹ may be identical to or different from each other, and when n10 is 2 or more, each R¹⁰ may be identical to or different from each other.

Regarding R⁵ to R¹⁰, the halogen atom, the unsubstituted alkyl group having 1 to 20 carbon atoms, the unsubstituted alkoxy group having 1 to 20 carbon atoms, or the aryl amino group having 6 to 20 carbon atoms are each the same as described in connection with the unsubstituted groups of (a), (c), (d), and (e).

Regarding R⁵ to R¹⁰, the unsubstituted halo alkyl group having 1 to 20 carbon atoms may be identical to the substituent substituting the groups of (e) to (g).

Regarding R⁵ to R⁸, the substituted or unsubstituted monovalent aromatic hydrocarbon group and the substituted or unsubstituted monovalent aromatic heterocyclic group are each the same as described in connection with the groups of (f) and (g).

Regarding R⁵ to R⁸, the unsubstituted alkyl amino group having 1 to 20 carbon atoms is the same as described in connection with the substituent substituting the groups of (c) to (g).

Regarding R⁹ and R¹⁰, the unsubstituted alkoxy group having 1 to 20 carbon atoms may be a group wherein at least one hydrogen atom of the alkoxy group described in (d) is substituted with the halogen atom described in (a). The halogen atom may be a fluorine atom in view of device lifespan. Detailed examples of the alkoxy group may include a trifluoromethoxy group, a trichloromethoxy group, a tribromomethoxy group, or a triiodomethoxy group. In an embodiment, the alkoxy group may be a fluoro alkoxy group, or a trifluoromethoxy group.

In Formulae (2) and (3) or in Formulae (2′) and (3′), n5, n6, n7, n8, n9, or n10 being 0 means that R⁵, R⁶, R⁷, R⁸, R⁹, or R¹⁰ corresponding to n5, n6, n7, n8, n9, or n10 are not present. In other words, in Formulae (2) and (3), or Formulae (2′) and (3′), n5 being 0 means that R⁵ is not present, n6 being 0 means that R⁶ is not present, n7 being 0 means that R⁷ is not present, and n8 being 0 means that R⁸ is not present. In addition, in Formula (3) or (3′), n9 being 0 means that R⁹ is not present, and n10 being 0 means that R¹⁰ is not present. Thus, Formulae (2) and (3), or Formulae (2′) and (3′) show that ring-forming carbon atoms to which R⁵, R⁶, R⁷, R⁸, R⁹, or R¹⁰ may be linked are unsubstituted, and hydrogen atoms are linked to the ring-forming carbon atoms.

n5 to n8 may each independently be 0, 1, 2, or 3. n9 and n10 may each independently be 0 or 1.

Here, R⁵ to R⁸ may be a halogen atom or an unsubstituted alkyl group having 1 to 20 carbon atoms in view of luminescence color purity, emission efficiency, and device lifespan. In addition, R⁵ to R⁸ may be an unsubstituted alkyl group having 1 to 20 carbon atoms, and an unsubstituted linear or branched alkyl group having 1 to 20 carbon atoms. In addition, R⁵ to R⁸ may be a methyl group, an isopropyl group, or a tert-butyl group.

R⁹ and R¹⁰ may be a halogen atom, a cyano group, an unsubstituted alkyl group having 1 to 20 carbon atoms, an unsubstituted halo alkyl group having 1 to 20 carbon atoms, or an unsubstituted halo alkoxy group having 1 to 20 carbon atoms, in view of luminescence color purity, emission efficiency, and device lifespan. R⁹ and R¹⁰ may be a cyano group and an unsubstituted alkyl group having 1 to 20 carbon atoms, and an unsubstituted branched alkyl group having 1 to 20 carbon atoms. The number of carbon atoms of the branched alkyl group may be 2 or more, 3 or more, or 4 or more, in view of solubility and luminescence color purity. The number of branched carbon atoms of the alkyl group may be 10 or less, 8 or less, or 6 or less, in view of device lifespan. In addition, R⁹ and R¹⁰ may be a cyano group or a tert-butyl group.

In the nitrogen-containing condensed cyclic compound according to an embodiment, in view of luminescence color purity, emission efficiency, and device lifespan, at least one of R⁵ to R⁸ in Formula (2) (or Formula (2′)) may be an unsubstituted alkyl group having 1 to 20 carbon atoms. At least one of R⁵, R⁷, R⁹, and R¹⁰ in Formula (3) (or Formula (3′)) may be an unsubstituted alkyl group having 1 to 20 carbon atoms. In an embodiment of the material for the organic electroluminescent device to be described later, at least one of R⁵ to R⁸ in Formula (2) (or Formula (2′)) of the nitrogen-containing condensed cyclic compound may be an unsubstituted alkyl group having 1 to 20 carbon atoms. In another embodiment of the material for the organic electroluminescent device, at least one of R⁵, R⁷, R⁹, and R¹⁰ in Formula (3) (or Formula (3′)) of the nitrogen-containing condensed cyclic compound may be an unsubstituted alkyl group having 1 to 20 carbon atoms. In the nitrogen-containing condensed cyclic compound according to an embodiment, at least one of R⁵ to R⁸ in Formula (2) (or Formula (2′)) may be a methyl group, an isopropyl group, or a tert-butyl group. In addition, at least one of R⁵ to R⁸ in Formula (2) (or Formula (2′)) may be a methyl group or a tert-butyl group. In the nitrogen-containing condensed cyclic compound according to an embodiment, at least one of R⁵, R⁷, R⁹, and R¹⁰ in Formula (3) (or Formula (3′)) may be an unsubstituted linear or branched alkyl group having 1 to 20 carbon atoms. At least one of R⁵ and R⁷ in Formula (3) (or Formula (3′)) may be an unsubstituted linear or branched alkyl group having 1 to 20 carbon atoms, and at least one of R⁹ and R¹⁰ may be an unsubstituted branched alkyl group having 1 to 20 carbon atoms. At least one of R⁵ and R⁷ in Formula (3) (or Formula (3′)) may be a methyl group, an isopropyl group, or a tert-butyl group, and at least one of R⁹ and R¹⁰ may be an isopropyl group or a tert-butyl group. At least one of R⁵ and R⁷ in Formula (3) (or Formula (3′)) may be a methyl group or a tert-butyl group, and at least one of R⁹ and R¹⁰ may be a tert-butyl group.

In a nitrogen-containing condensed cyclic compound according to another embodiment, in view of the peak wavelength of the emission spectrum, luminescence color purity, emission efficiency, and device lifespan, in Formula (2) (or Formula (2′)), at least one of n5 to n8 may be 3 or more, or at least one of R⁵ to R⁸ may be an unsubstituted branched alkyl group having 4 to 15 carbon atoms. In Formula (3) (or Formula (3′)), at least one of n5, n7, n9, and n10 is 3 or more, or at least one of R5, R7, R9, and R10 is an unsubstituted branched alkyl group having 4 to 15 carbon atoms. In an embodiment of the material for the organic electroluminescent device to be described later, in Formula (2) (or Formula (2′)) of the nitrogen-containing condensed cyclic compound, at least one of n5 to n8 may be 3 or more, or at least one of R⁵ to R⁸ may be an unsubstituted branched alkyl group having 4 to 15 carbon atoms. In Formula (3) (or Formula (3′)), at least one of n5, n7, n9, and n10 may be 3 or more, or at least one of R⁵, R⁷, R⁹, and R¹⁰ may be an unsubstituted branched alkyl group having 4 to 15 carbon atoms. In the nitrogen-containing condensed cyclic compound according to such embodiment, at least one of n5 to n8 in Formula (2) (or Formula (2′)) may be 3. At least one of R⁵ to R⁸ may be an isopropyl group or a tert-butyl group. In the nitrogen-containing condensed cyclic compound according to such embodiment, at least one of n5, n7, n9, and n10 in Formula (3) (or Formula (3′)) may be 3. At least one of R⁵, R⁷, R⁹, and R¹⁰ may be an unsubstituted branched alkyl group having 4 to 15 carbon atoms. Here, at least one of R⁵ and R⁷ may be an unsubstituted linear or branched alkyl group having 4 to 15 carbon atoms, and at least one of R⁹ and R¹⁰ may be an unsubstituted branched alkyl group having 4 to 15 carbon atoms. At least one of R⁵ and R⁷ may be a methyl group, an isopropyl group, or a tert-butyl group, and at least one of R⁹ and R¹⁰ may be an isopropyl group or a tert-butyl group.

In the nitrogen-containing condensed cyclic compound according to another embodiment of the present disclosure, R⁵ to R⁸ may each independently be an unsubstituted alkyl group having 1 to 20 carbon atoms, a substituted or unsubstituted monovalent aromatic hydrocarbon group, or a substituted or unsubstituted monovalent aromatic heterocyclic group, and in Formula (2) (or Formula (2′)), not all of R⁵ to R⁸ may be an unsubstituted alkyl group having 1 to 20 carbon atoms and not all of n5 to n8 may be 0, and in Formula (3) (or Formula (3′)), not all of R⁵ and R⁷ may be an unsubstituted alkyl group having 1 to 20 carbon atoms and not all of n5 and n7 may be 0.

In the nitrogen-containing condensed cyclic compound according to the present disclosure, the structure represented by Formula (1), (1A), (1B), (2), or (3) may be a structure represented by one of Formulae (3-1) to (3-9) (a structure selected from a group represented by Formulae (3-1) to (3-9)). In this regard, in an embodiment of the material for the organic electroluminescent device, the structure represented by Formula (1), (1A), (1B), (2), or (3) may be a structure represented by one of Formulae (3-1) to (3-9) (a structure selected from a group represented by Formulae (3-1) to (3-9)). In this regard, in an embodiment of the fluorescence emitter, the structure represented by Formula (1), (1A), (1B), (2), or (3) of the nitrogen-containing condensed cyclic compound may be a structure represented by one of Formulae (3-1) to (3-9) (a structure selected from a group represented by Formulae (3-1) to (3-9)). In the nitrogen-containing condensed cyclic compound, the material for the organic electroluminescent device, and the fluorescence emitter according to an embodiment, regarding Formulae (3-1) to (3-9), at least one of the alkyl substituents are linked to the core portion.

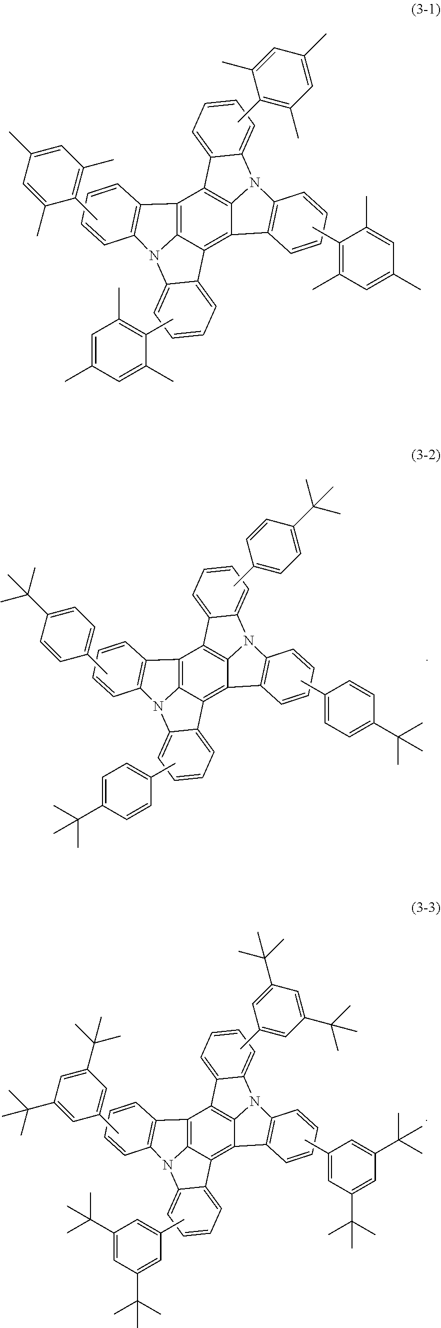

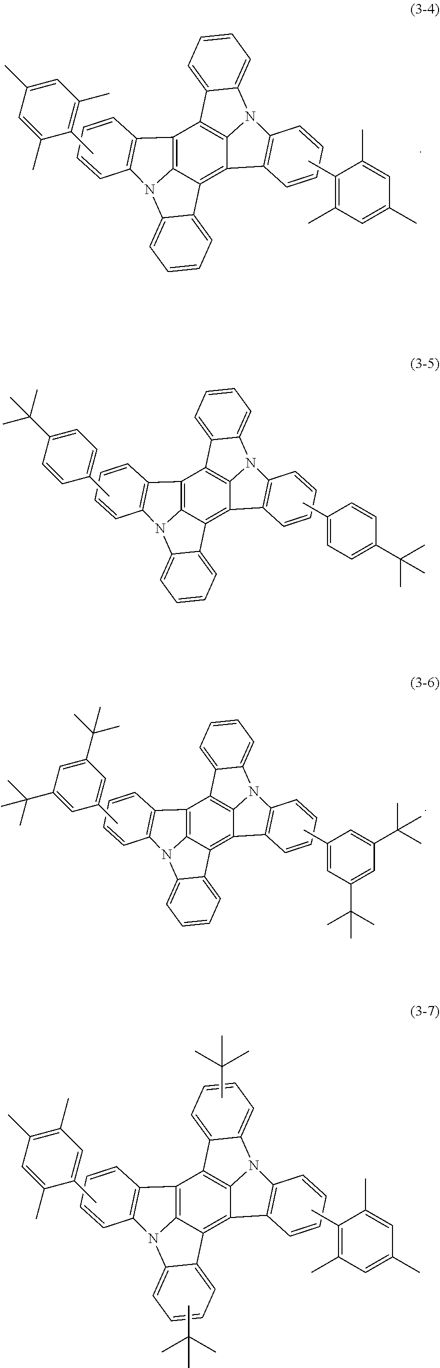

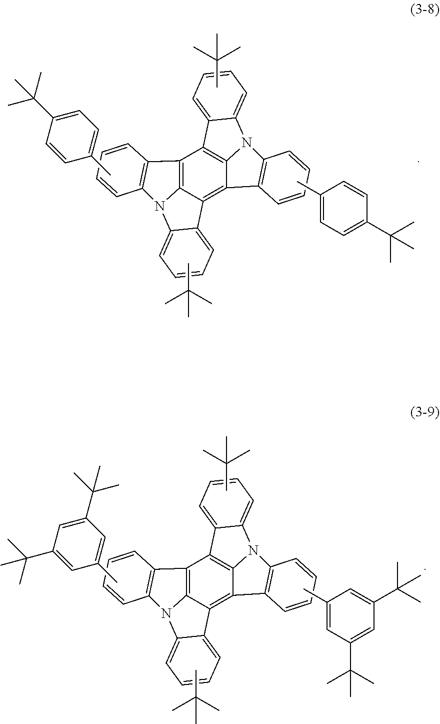

In the nitrogen-containing condensed cyclic compound according to the present disclosure, in view of the peak wavelength of the emission spectrum, luminescence color purity, emission efficiency, and device lifespan, the structure represented by Formula (1), (1A), or (1B) may be the structure represented by Formula (7) or (8). Thus, an embodiment of the present disclosure relates to a nitrogen-containing condensed cyclic compound having the structure represented by Formula (7) or (8). In an embodiment of a material for an organic electroluminescent device to be described later, the structure represented by Formula (1), (1A), or (1B) of the nitrogen-containing condensed cyclic compound may be the structure represented by Formula (7) or (8). In an embodiment of a fluorescence emitter to be described later, the structure represented by Formula (1), (1A), or (1B) of the nitrogen-containing condensed cyclic compound may be the structure represented by Formula (7) or (8).

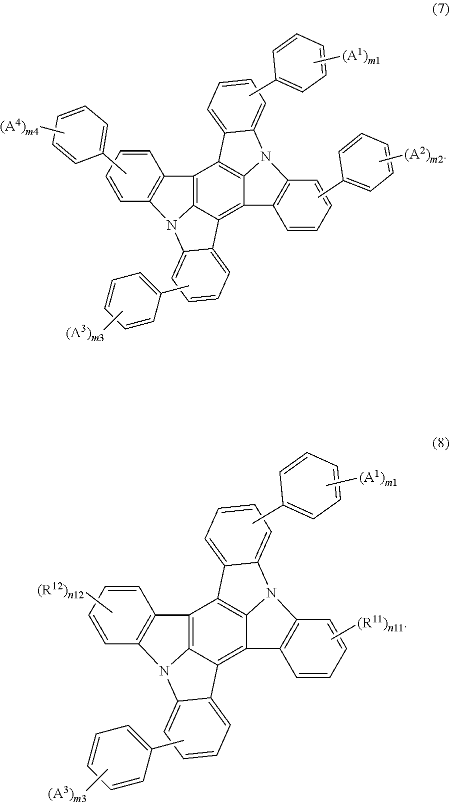

In Formulae (7) and (8),

A¹ to A⁴ may each independently be an unsubstituted alkyl group having 1 to 20 carbon atoms, a substituted or unsubstituted monovalent aromatic hydrocarbon group, or a substituted or unsubstituted monovalent aromatic hetero ring,

R¹¹ and R¹² are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n1 to n4 may each independently be 0, 1, 2, 3, 4, or 5,

n11 and n12 may each independently be 0, 1, 2, 3, or 4,

wherein, when m1 is 2 or more, each A¹ may be identical to or different from each other,

when m2 is 2 or more, each A² may be identical to or different from each other,

when m3 is 2 or more, each A³ may be identical to or different from each other,

when m4 is 2 or more, each A⁴ may be identical to or different from each other,

when n11 is 2 or more, each R¹¹ may be identical to or different from each other,

when n12 is 2 or more, each R¹² may be identical to or different from each other,

wherein, in Formula (7),

not all of A¹ to A⁴ are alkyl groups having 1 to 20 carbon atoms, and not all of m1 to m4 are 0,

wherein, in Formula (8),

not all of A¹ to A³ are alkyl groups having 1 to 20 carbon atoms, and not all of m1 and m3 are 0.

Regarding A¹ to A⁴, the substituted or unsubstituted monovalent aromatic hydrocarbon group and the substituted or unsubstituted monovalent aromatic heterocyclic group are each the same as described in connection with the groups of (f) and (g).

Regarding A¹ to A⁴, the unsubstituted alkyl group having 1 to 20 carbon atoms is the same as described in connection with the unsubstituted group of (c).

Regarding R¹¹ and R¹², the halogen atom, the unsubstituted alkyl group having 1 to 20 carbon atoms, the unsubstituted alkoxy group having 1 to 20 carbon atoms, or the aryl amino group having 6 to 20 carbon atoms are each the same as described in connection with the unsubstituted groups of (a), (c), (d), and (e).

Regarding R¹¹ and R¹², the unsubstituted halo alkyl group having 1 to 20 carbon atoms may be identical to the substituent substituting the groups of (e) to (g).

Regarding R¹¹ and R¹², the unsubstituted alkoxy group having 1 to 20 carbon atoms may be a group wherein at least one hydrogen atom of the alkoxy group described in (d) is substituted with the halogen atom described in (a). The halogen atom may be a fluorine atom in view of device lifespan. Detailed examples of the alkoxy group may include a trifluoromethoxy group, a trichloromethoxy group, a tribromomethoxy group, or a triiodomethoxy group. In an embodiment, the alkoxy group may be a fluoro alkoxy group, or a trifluoromethoxy group.

In Formulae (7) and (8), when m1 to m4, n11, and n12 are 0, A¹, A², A³, A⁴, R¹¹, and R¹² corresponding to m1 to m4, n11, and n12 are not present. In other words, regarding Formula (7) and (8), m1 being 0 means that A¹ is not present, m2 being 0 means that A² is not present, m3 being 0 means that A³ is not present, and m4 being 0 means that A⁴ is not present. In addition, in Formula (8), n11 being 0 means that R¹¹ is not present, and n12 being 0 means that R¹² is not present. Thus, Formulae (7) and (8) show that the ring-forming carbon atoms to which A¹, A², A³, A⁴, R¹¹, or R¹² may be linked are unsubstituted, and hydrogen atoms are linked to the ring-forming carbon atoms.

m1 to m4 may each independently be 2. n11 and n12 may each independently be 0 or 1.

A¹ to A⁴ may be a substituted or unsubstituted monovalent aromatic hydrocarbon group having 6 to 12 carbon atoms or a substituted or unsubstituted monovalent aromatic heterocyclic group, in view of luminescence color purity, emission efficiency, and device lifespan. In addition, A¹ to A⁴ may be a substituted or unsubstituted monovalent aromatic heterocyclic group having 6 to 12 carbon atoms or, a monovalent aromatic heterocyclic group having 6 to 12 carbon atoms unsubstituted or substituted with an unsubstituted alkyl group having 1 to 20 carbon atoms. In addition, A¹ to A⁴ may be a phenyl group substituted with an unsubstituted alkyl group having 1 to 20 carbon atoms or a phenyl group. In this regard, the number of carbon atoms of the unsubstituted alkyl group, which is a substituent, may be, but not limited to 1 or more and 10 or less, 1 or more and 8 or less, or 1 or more and 6 or less. A¹ to A⁴ may be, for example, a phenyl group substituted with a methyl group, a phenyl group substituted with a t-butyl group, or a phenyl group.

R¹¹ and R¹² may be a halogen atom, a cyano group, an unsubstituted alkyl group having 1 to 20 carbon atoms, an unsubstituted halo alkyl group having 1 to 20 carbon atoms, or an unsubstituted halo alkoxy group having 1 to 20 carbon atoms, in view of luminescence color purity, emission efficiency, and device lifespan. R⁹ and R¹⁰ may be a cyano group and an unsubstituted alkyl group having 1 to 20 carbon atoms, or an unsubstituted branched alkyl group having 1 to 20 carbon atoms. The number of carbon atoms of the branched alkyl group may be 2 or more, 3 or more, or 4 or more, in view of solubility and luminescence color purity. The number of branched carbon atoms of the alkyl group may be 10 or less, 8 or less, or 6 or less, in view of device lifespan. In addition, R¹¹ and R¹² may be, for example, a tert-butyl group.

In the nitrogen-containing condensed cyclic compound, the material for the organic electroluminescent device, and the fluorescence emitter according to an embodiment, regarding Formulae (7) and (8), at least one of A¹ to A⁴ and R¹¹ and R¹² may be linked to least one of a ring-forming carbon atom of the core portion of Formulae (7) and (8). In Formulae (7) and (8), at least one of A¹ to A⁴ and R¹¹ and R¹² that may be linked to such ring-forming carbon atom of the core portion may be, but not limited to, for example, a terphenyl group, and a phenyl group substituted with at least two alkyl groups having 1 to 4 carbon atoms. At least one of A¹ to A⁴ and R¹¹ and R¹² may also be, for example, an m-terphenyl group, or a 2, 6-di-tert-butylphenyl group.

The nitrogen-containing condensed cyclic compound may be represented by, for example, Formula (8) among Formulae (7) and (8).

In the nitrogen-containing condensed cyclic compound according to the present disclosure, the structure represented by Formula (1), (1A), (1B), (7), or (8) may be a structure selected from the groups represented by Formulae (9) to (11). In an embodiment of a material for an organic electroluminescent device to be described later, the structure represented by Formula (1), (1A), (1B), (7), or (8) of the nitrogen-containing condensed cyclic compound may be a structure selected from a group represented by Formulae (9) to (11). In this regard, in an embodiment of the fluorescence emitter, the structure represented by Formula (1), (1A), (1B), (7), or (8) of the nitrogen-containing condensed cyclic compound may be a structure selected from a group represented by Formulae (9) to (11)).

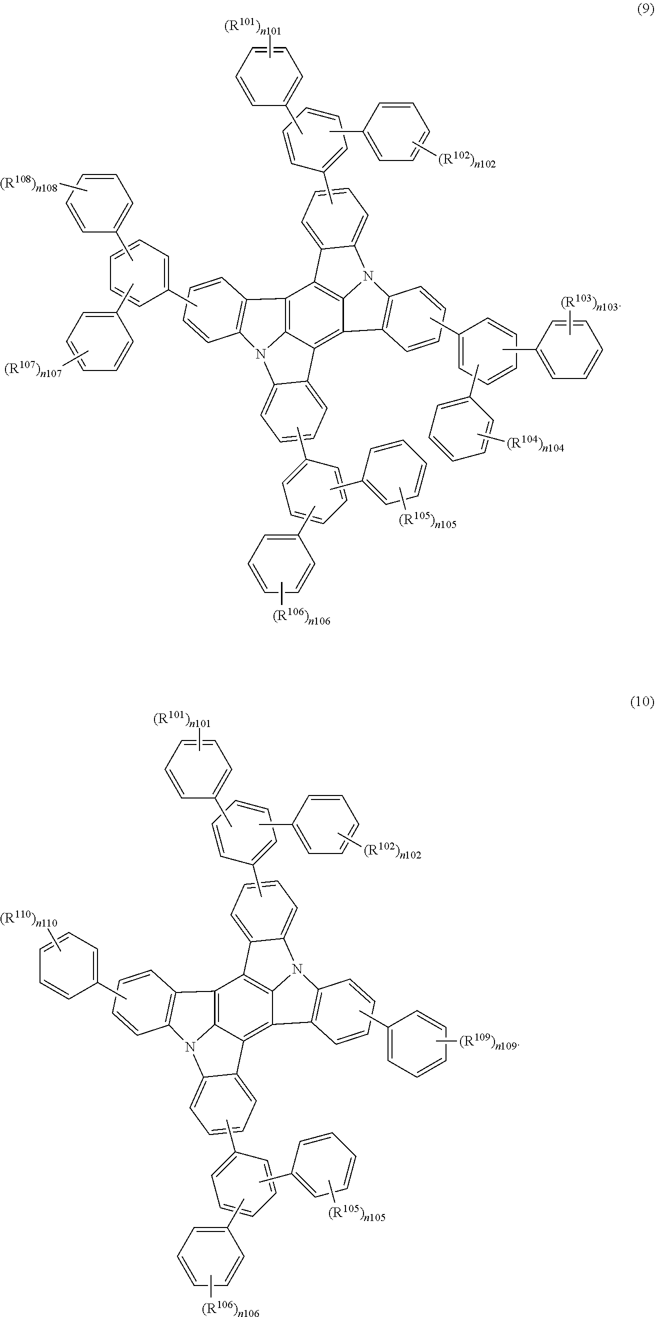

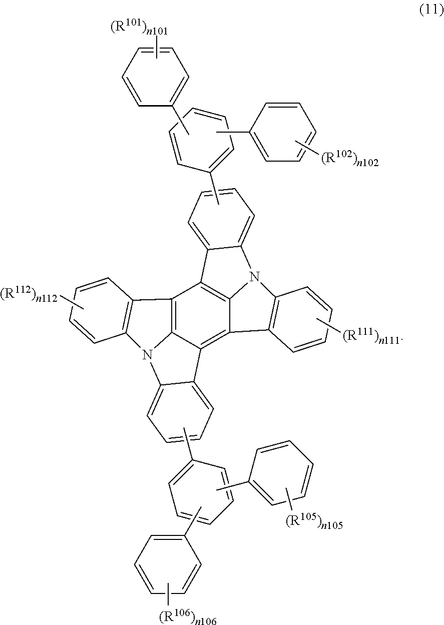

In Formulae (9) to (11),

R¹⁰¹ to R¹¹² may each independently be an unsubstituted alkyl group having 1 to 20 carbon atoms,

n101 to n112 may each independently be 0, 1, 2, or 3, and

when n101 is 2 or more, each R¹⁰¹ may be identical to or different from each other, when n102 is 2 or more, each R¹⁰² may be identical to or different from each other, when n103 is 2 or more, each R¹⁰³ may be identical to or different from each other, when n104 is 2 or more, each R¹⁰⁴ may be identical to or different from each other, when n105 is 2 or more, each R¹⁰⁵ may be identical to or different from each other, when n106 is 2 or more, each R¹⁰⁶ may be identical to or different from each other, when n107 is 2 or more, each R¹⁰⁷ may be identical to or different from each other, when n108 is 2 or more, each R¹⁰⁸ may be identical to or different from each other, when n109 is 2 or more, each R¹⁰⁹ may be identical to or different from each other, when n110 is 2 or more, each R¹¹⁰ may be identical to or different from each other, when n111 is 2 or more, each R¹¹¹ may be identical to or different from each other, and when n112 is 2 or more, each R¹¹² m may be identical to or different from each other.

When n101 is 2 or more, each R¹⁰¹ may be identical to each other, when n102 is 2 or more, each R¹⁰² may be identical to each other, when n103 is 2 or more, each R¹⁰³ may be identical to each other, when n104 is 2 or more, each R¹⁰⁴ may be identical to each other, when n105 is 2 or more, each R¹⁰⁵ may be identical to each other, when n106 is 2 or more, each R¹⁰⁶ may be identical to each other, when n107 is 2 or more, each R¹⁰⁷ may be identical to each other, when n108 is 2 or more, each R¹⁰⁸ may be identical to each other, when n109 is 2 or more, each R¹⁰⁹ may be identical to each other, when n110 is 2 or more, each R¹¹⁰ may be identical to each other, when n111 is 2 or more, each R¹¹¹ may be identical to each other, and when n112 is 2 or more, each R¹¹² m may be identical to each other.

In view of luminescence color purity, emission efficiency, and device lifespan, the nitrogen-containing condensed cyclic compound may be represented by Formula (11) among Formulae (9) to (11).

In Formulae (9) to (11), n101 to n112 being 0 means that R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, and R¹¹² corresponding to n101 to n112 do not exist. In other words, in Formulae (9) to (11), n101 being 0 means that R¹⁰¹ is not present, n102 being 0 means that R¹⁰² is not present, n103 being 0 means that R¹⁰³ is not present, n104 being 0 means that R¹⁰⁴ is not present, n105 being 0 means that R¹⁰⁵ is not present, n106 being 0 means that R¹⁰⁶ is not present, n107 being 0 means that R¹⁰⁷ is not present, and n108 being 0, means that R¹⁰⁸ is not present. In addition, in Formula (10), n109 being 0 means that R¹⁰⁹ is not present, and n110 being 0 means that R¹¹⁰ is not present. In addition, in Formula (11), n111 being 0 means that R¹¹¹ is not present, and n112 being 0 means that R¹¹² is not present. Thus, in Formulae (9) to (11), the ring-forming carbon atom to which R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, and R¹¹² may be linked are unsubstituted, and a hydrogen atom may be linked to the ring-forming carbon atom.

n101 to n108 may each independently be 0, 1, 2, or 3, and may be 0. n109 and n110 may each independently be 0, 1, or 2. n111 and n112 may each independently be 0 or 1.

Regarding R¹⁰¹ to R¹¹², the unsubstituted alkyl group having 1 to 20 carbon atoms is the same as described in connection with the unsubstituted group of (c).

Regarding R¹⁰¹ to R¹¹², the unsubstituted alkyl group having 1 to 20 carbon atoms may be an unsubstituted alkyl group having 1 to 10 carbon atoms, in view of luminescence color purity, emission efficiency, and device lifespan. R¹⁰¹ to R¹¹² may be an unsubstituted alkyl group having 1 to 8 carbon atoms, or an unsubstituted alkyl group having 1 to 6 carbon atoms. R¹⁰¹ to R¹¹² may be, for example, a t-butyl group.

In Formula (11), n101, n102, n105, n106, n1111, and n112 may each independently be 0 or 1, and the unsubstituted alkyl group having 1 to 20 carbon atoms of R¹⁰¹, R¹⁰², R¹⁰⁵, R¹⁰⁶, R¹¹¹, and R¹¹² may be an unsubstituted alkyl group having 1 to 6 carbon atoms. In addition, for example, n101, n102, n105, n106, n111, and n112 may be 0.

In the nitrogen-containing condensed cyclic compound, the material for the organic electroluminescent device, and the fluorescence emitter according to an embodiment, regarding Formulae (9) to (11), at least one of R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, and R¹¹² may be linked to at least one of a ring-forming carbon atom of a core portion of Formulae (9) to (11). In Formula (9), at least one of R¹⁰¹, R¹⁰², R¹⁰³, R¹⁰⁴, R¹⁰⁵, R¹⁰⁶, R¹⁰⁷, and R¹⁰⁸ may be linked to the ring-forming carbon atom of the core portion may be, but not limited to, for example, a terphenyl group. The at least one of R¹⁰¹, R¹⁰², R¹⁰³, R¹⁰⁴, R¹⁰⁵, R¹⁰⁶, R¹⁰⁷, and R¹⁰⁸ may also be, for example, an m-terphenyl group. In Formula (10), at least one of R¹⁰¹, R¹⁰², R¹⁰⁵, R¹⁰⁶, R¹⁰⁹, and R¹¹⁰ that may be linked to the ring-forming carbon atom may be, but not limited to, for example, a terphenyl group, and a phenyl group substituted with at least two alkyl groups having 1 to 4 carbon atoms. The at least one of R¹⁰¹, R¹⁰², R¹⁰⁵, R¹⁰⁶, R¹⁰⁹, and R¹¹⁰ may also be, for example, an m-terphenyl group, or a 2, 6-di-tert-butylphenyl group. In Formula (11), at least one of R¹⁰¹, R¹⁰², R¹⁰⁵, R¹⁰⁶, R¹⁰⁹, and R¹¹⁰ that may be linked to the ring-forming carbon atom may be, but not limited to, for example, a terphenyl group. The at least one of R¹⁰¹, R¹⁰², R¹⁰⁵, R¹⁰⁶, R¹⁰⁹, and R¹¹⁰ may also be, for example, an m-terphenyl group.

In the nitrogen-containing condensed cyclic compound according to the present disclosure, in view of the peak wavelength of the emission spectrum, luminescence color purity, emission efficiency, and device lifespan, the structure represented by Formula (1), (1A), or (1B) may be a structure selected from a group represented by Formulae (12) to (14). Thus, an embodiment of the present disclosure relates to a nitrogen-containing condensed cyclic compound including a structure selected from a group represented by Formulae (12) to (14). In an embodiment of a material for an organic electroluminescent device to be described later, the structure represented by Formula (1), (1A), or (1B) of the nitrogen-containing condensed cyclic compound may be a structure selected from a group represented by Formulae (12) to (14). In an embodiment of a fluorescence emitter to be described later, the structure represented by Formula (1), (1A), or (1B) of the nitrogen-containing condensed cyclic compound may be a structure selected from a group represented by Formulae (12) to (14).

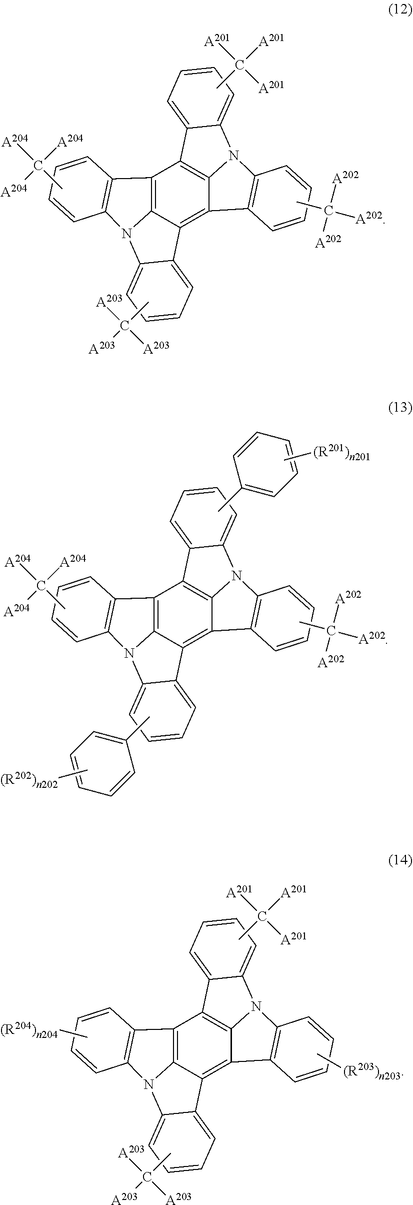

In Formulae (12) to (14),

A²⁰¹ to A²⁰⁴ may each independently be an unsubstituted alkyl group having 1 to 20 carbon atoms, a substituted or unsubstituted monovalent aromatic hydrocarbon group, or a substituted or unsubstituted monovalent aromatic heterocyclic group,

R²⁰¹ to R²⁰² are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,

R²⁰³ and R²⁰⁴ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,

n201 and n202 are each independently 0, 1, 2, 3, 4, or 5,

n203 and n204 are each independently 0, 1, 2, 3, or 4,

wherein, each A²⁰¹ may be identical to or different from each other, each A²⁰² may be identical to or different from each other, each A²⁰³ may be identical to or different from each other, and each A²⁰⁴ may be identical to or different from each other,

two or more of A²⁰¹, two or more of A²⁰², two or more of A²⁰³, and two or more of A²⁰⁴ may each form a ring,

when n201 is 2 or more, each R²⁰¹ may be identical to or different from each other, when n202 is 2 or more, each R²⁰² may be identical to or different from each other, when n203 is 2 or more, each R²⁰³ may be identical to or different from each other, and when n204 is 2 or more, each R²⁰⁴ may be identical to or different from each other,

each of A²⁰¹ to A²⁰⁴ are optionally linked to a ring-forming carbon of the core portion of Formula (12),

each of A²⁰², A²⁰⁴, R²⁰¹, and R²⁰² are optionally linked to a ring-forming carbon of the core portion of Formula (13),

each of A²⁰¹, A²⁰³, R²⁰³, and R²⁰⁴ are optionally linked to a ring-forming carbon of the core portion of Formula (14),

in Formula (12), not all of each A²⁰¹, each A²⁰², each A²⁰³, and each A²⁰⁴ are unsubstituted alkyl groups having 1 to 20 carbon atoms,

in Formula (13), not all of each A²⁰² and each A²⁰⁴ are unsubstituted alkyl groups having 1 to 20 carbon atoms, and

in Formula (14), not all of each A²⁰¹ and each A²⁰³ are unsubstituted alkyl groups having 1 to 20 carbon atoms.

In Formulae (12) to (14), C represents a carbon atom.

Regarding A²⁰¹ to A²⁰⁴, R²⁰¹, and R²⁰², the substituted or unsubstituted monovalent aromatic hydrocarbon group and the substituted or unsubstituted monovalent aromatic heterocyclic group are each the same as described in connection with the groups of (f) and (g).

Regarding A²⁰¹ to A²⁰⁴, and R²⁰¹ to R²⁰⁴, the unsubstituted alkyl group having 1 to 20 carbon atoms is the same as described in connection with the unsubstituted group of (c).

Regarding R²⁰¹ to R²⁰⁴, the halogen atom, the unsubstituted alkoxy group having 1 to 20 carbon atoms, and the unsubstituted aryl amino group having 6 to 20 carbon atoms are each the same as described in connection with the unsubstituted groups of (a), (d), and (e).

Regarding R²⁰¹ to R²⁰⁴, the unsubstituted halo alkyl group having 1 to 20 carbon atoms may be identical to the substituent substituting the groups of (e) to (g).

Regarding R²⁰¹ and R²⁰², the unsubstituted alkyl amino group having 1 to 20 carbon atoms is the same as described in connection with the substituent substituting the groups of (c) to (g).

Regarding R²⁰³ and R²⁰⁴, the unsubstituted alkoxy group having 1 to 20 carbon atoms may be a group wherein at least one hydrogen atom of the alkoxy group described in (d) is substituted with the halogen atom described in (a). The halogen atom may be a fluorine atom in view of device lifespan. Detailed examples of the alkoxy group may include a trifluoromethoxy group, a trichloromethoxy group, a tribromomethoxy group, or a triiodomethoxy group. In an embodiment, the alkoxy group may be a fluoro alkoxy group, and particularly, a trifluoromethoxy group.

In Formula (13), n201 and n202 being 0 means that R²⁰¹ and R²⁰² corresponding to n201 and n202 are not present. In other words, n201 being 0 means that R²⁰¹ is not present, and n202 being 0 means that R²⁰² is not present. Thus, in Formula (13), the ring-forming carbon atoms to which R²⁰¹ and R²⁰² may be linked are unsubstituted, and hydrogen atoms are linked to the ring-forming carbon atoms.

In Formula (14), n203 and n204 being 0 means that R²⁰³ and R²⁰⁴ corresponding to n203 and n204 are not present. In other words, n203 being 0 means that R²⁰³ is not present, and n204 being 0 means that R²⁰⁴ is not present. Thus, in Formula (14), the ring-forming carbon atoms to which R²⁰³ and R²⁰⁴ may be linked are unsubstituted, and hydrogen atoms are linked to the ring-forming carbon atoms.

n201 and n202 may each independently be 0, 1, or 2, or may be 2. n203 and n204 may each independently be 0, or 1, for example, 0.

When A²⁰¹ to A²⁰⁴ are each a substituted or unsubstituted monovalent aromatic hydrocarbon group or a substituted or unsubstituted monovalent aromatic heterocyclic group, in view of luminescence color purity and emission efficiency, A²⁰¹ to A²⁰⁴ may be a substituted or unsubstituted monovalent aromatic hydrocarbon group. In addition, A¹ to A⁴ may be a substituted or unsubstituted monovalent aromatic heterocyclic group having 6 to 12 carbon atoms or, a monovalent aromatic heterocyclic group having 6 to 12 carbon atoms unsubstituted or substituted with an unsubstituted alkyl group having 1 to 20 carbon atoms. In addition, A¹ to A⁴ may be a phenyl group substituted with an unsubstituted alkyl group having 1 to 20 carbon atoms or a phenyl group. In this regard, the number of carbon atoms of the unsubstituted alkyl group, which is a substituent, may be, but not limited to 1 or more and 10 or less, 1 or more and 8 or less, or 1 or more and 6 or less. A²⁰¹ to A²⁰⁴ may be a phenyl group substituted with a t-butyl group or a phenyl group, for example, a phenyl group.

When A²⁰¹ to A²⁰⁴ are each an alkyl group having 1 to 20 carbon atoms, in view of luminescence color purity, emission efficiency, and device lifespan, the unsubstituted alkyl group having 1 to 20 carbon atoms may be an unsubstituted alkyl group having 1 to 10 carbon atoms. A²⁰¹ to A²⁰⁴ may be an unsubstituted alkyl group having 1 to 8 carbon atoms, or an unsubstituted alkyl group having 1 to 6 carbon atoms.

In addition, A²⁰¹ to A²⁰⁴ may be, for example, a methyl group.

R²⁰¹ to R²⁰⁴ may be a halogen atom, a cyano group, an unsubstituted alkyl group having 1 to 20 carbon atoms, an unsubstituted halo alkyl group having 1 to 20 carbon atoms, or an unsubstituted halo alkoxy group having 1 to 20 carbon atoms, in view of emission wavelength and emission efficiency. R²⁰¹ to R²⁰⁴ may be an unsubstituted alkyl group having 1 to 20 carbon atoms. In this regard, the number of carbon atoms of the unsubstituted alkyl group, which is a substituent, may be, but not limited to 1 or more and 10 or less, 1 or more and 8 or less, or 1 or more and 6 or less. The unsubstituted alkyl group having 1 to 20 carbon atoms may be a branched alkyl group. R²⁰¹ to R²⁰⁴ may be, for example, a t-butyl group.

Two or more of A²⁰¹, two or more of A²⁰², two or more of A²⁰³, and two or more of A²⁰⁴ may each form a ring, and the formed ring may be, for example, a fluorene ring.

The nitrogen-containing condensed cyclic compound may be represented by, for example, Formula (13) among Formulae (12) to (14).

In the nitrogen-containing condensed cyclic compound, the material for the organic electroluminescent device, and the fluorescence emitter according to an embodiment, regarding Formulae (12) to (14), at least one of A²⁰¹ to A²⁰⁴ and R²⁰¹ to R²⁰⁴ may be linked to at least one of a ring-forming carbon atom of the core portion. In Formula (13), at least one of A²⁰², A²⁰⁴, R²⁰¹, and R²⁰² that may be linked to the ring-forming carbon atom of the core portion may be, but not limited to, for example, a terphenyl group, and a phenyl group substituted with at least two alkyl groups having 1 to 4 carbon atoms. The at least one of A²⁰², A²⁰⁴, R²⁰¹, and R²⁰² may also be, for example, an m-terphenyl group, or a 2, 6-di-tert-butylphenyl group.

In the nitrogen-containing condensed cyclic compound according to the present disclosure, the structure selected from a group represented by Formulae (1), (1A), (1B), or (12) to (13) may be a structure selected from a group represented by Formulae (15) to (17). In an embodiment of a material for an organic electroluminescent device to be described later, the structure selected from a group represented by Formula (1), (1A), (1B), or (12) to (14) of the nitrogen-containing condensed cyclic compound may be a structure selected from a group represented by Formulae (15) to (17). In this regard, in an embodiment of the fluorescence emitter, the structure selected from a group represented by Formula (1), (1A), (1B), or (12) to (14) of the nitrogen-containing condensed cyclic compound may be a structure selected from a group represented by Formulae (15) to (17).

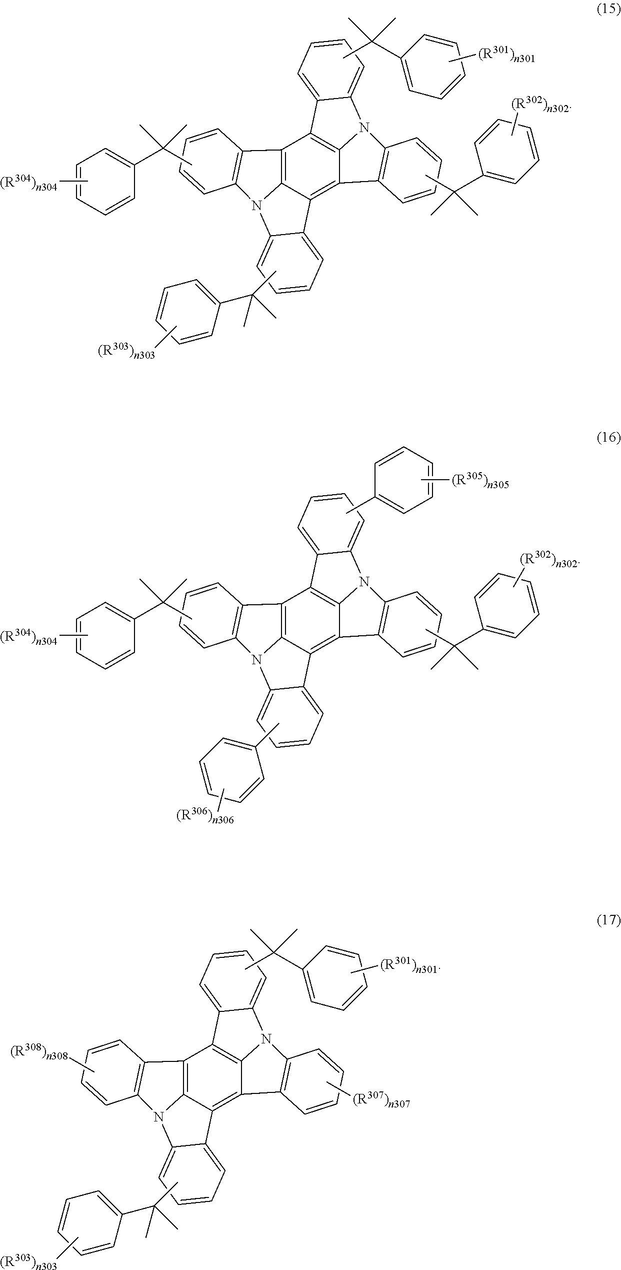

In Formulae (15) to (17),

R³⁰¹ to R³⁰⁸ may each independently be an unsubstituted alkyl group having 1 to 20 carbon atoms, and n301 to n308 may each independently be 0, 1, 2, or 3,

wherein, when n301 is 2 or more, each R³⁰¹ may be identical to or different from each other, when n302 is 2 or more, each R³⁰² may be identical to or different from each other, when n303 is 2 or more, each R³⁰³ may be identical to or different from each other, when n304 is 2 or more, each R³⁰⁴ may be identical to or different from each other, when n305 is 2 or more, each R³⁰⁵ may be identical to or different from each other, when n306 is 2 or more, each R³⁰⁶ may be identical to or different from each other, when n307 is 2 or more, each R³⁰⁷ may be identical to or different from each other, and when n308 is 2 or more, each R³⁰⁸ may be identical to or different from each other.

When n301 is 2 or more, each R³⁰¹ may be identical to each other, when n302 is 2 or more, each R³⁰² may be identical to each other, when n303 is 2 or more, each R³⁰³ may be identical to each other, when n304 is 2 or more, each R³⁰⁴ may be identical to each other, when n305 is 2 or more, each R³⁰⁵ may be identical to each other, when n306 is 2 or more, each R³⁰⁶ may be identical to each other, when n307 is 2 or more, each R³⁰⁷ may be identical to each other, and when n308 is 2 or more, each R³⁰⁸ may be identical to each other.

In view of the emission wavelength, the fluorescence emitter may be represented by Formula (16) among Formulae (15) to (17).

In Formulae (15) to (17), n301 to n308 being 0 means that R³⁰¹, R³⁰², R³⁰³, R³⁰⁴, R³⁰⁵, R³⁰⁶, R³⁰⁷, and R³⁰⁸ corresponding to n301 to n308 are not present. In other words, regarding (15) to (17), n301 being 0 means that R³⁰¹ is not present, n302 being 0 means that R³⁰² is not present, n303 being 0 means that R³⁰³ is not present, and n304 being 0 means that R³⁰⁴ is not present. In addition, in Formula (16), n305 being 0 means that R³⁰⁵ is not present, and n306 being 0 means that R³⁰⁶ is not present. In addition, in Formula (17), n307 being 0 means that R³⁰⁷ is not present, and n308 being 0 means that R³⁰⁸ is not present. Thus, in Formulae (15) to (17), the ring-forming carbon atom to which R³⁰¹, R³⁰², R³⁰³, R³⁰⁴, R³⁰⁵, R³⁰⁶, R³⁰⁷, and R³⁰⁸ may be linked are unsubstituted, and a hydrogen atom is linked to the ring-forming carbon atom.

n301 and n304 may each independently be 0, 1, or 2, or may be 0. n305 and n306 may each independently be 0, 1, or 2. n307 and n308 may each independently be 0 or 1.

Regarding R³⁰¹ to R³⁰⁸, the unsubstituted alkyl group having 1 to 20 carbon atoms is the same as described in connection with the unsubstituted group of (c).

Regarding R³⁰¹ to R³⁰⁸, the unsubstituted alkyl group having 1 to 20 carbon atoms may be an unsubstituted alkyl group having 1 to 10 carbon atoms, in view of emission wavelength, luminescence color purity, emission efficiency, and device lifespan. R³⁰¹ to R³⁰⁸ may be an unsubstituted alkyl group having 1 to 8 carbon atoms, or an unsubstituted alkyl group having 1 to 6 carbon atoms. R³⁰¹ to R³⁰⁸ may be a methyl group or a t-butyl group, for example, a t-butyl group. The unsubstituted alkyl group having 1 to 20 carbon atoms may be a branched alkyl group.

Regarding Formula (16), n302, n304, n305, and n306 may each independently be 0, 1, or 2, and the unsubstituted alkyl group having 1 to 20 carbon atoms in R³⁰², R³⁰⁴, R³⁰⁵, and R³⁰⁶ may be an unsubstituted alkyl group having 1 to 6 carbon atoms. In addition, n302 and n304 may each independently be 0 or 1, n305 and n306 may each independently be 0, 1, or 2, the unsubstituted alkyl group having 1 to 20 carbon atoms in R³⁰², R³⁰⁴, R³⁰⁵, and R³⁰⁶ may be an unsubstituted alkyl group having 1 to 6 carbon atoms. For example, n302 and n304 may be 0, n305 and n306 may each independently be 1 or 2, the unsubstituted alkyl group having 1 to 20 carbon atoms in R³⁰¹ to R³⁰⁴ may be an unsubstituted alkyl group having 1 to 6 carbon atoms. For example, n302 and n304 may be 0, n305 and n306 may each independently be 1 or 2, and the unsubstituted alkyl group having 1 to 20 carbon atoms in R³⁰¹ to R³⁰⁴ may be a t-butyl group. In addition, for example, n302 and n304 may be 0, n305 and n306 may be 2, and the unsubstituted alkyl group having 1 to 20 carbon atoms in R³⁰⁵ and R³⁰⁶ may be a t-butyl group.

In the nitrogen-containing condensed cyclic compound, the material for the organic electroluminescent device, and the fluorescence emitter according to an embodiment, regarding Formulae (15) to (17), at least one of R³⁰¹, R³⁰², R³⁰³, R³⁰⁴, R³⁰⁵, R³⁰⁶, R³⁰⁷, and R³⁰⁸ may be linked to at least one of a ring-forming carbon atom of the core portion. In Formula (16), at least one of R³⁰², R³⁰⁴, R³⁰⁵, and R³⁰⁶ that may be linked to such ring-forming carbon atom may be, but not limited to, for example, a phenyl group substituted with at least two alkyl groups having 1 to 4 carbon atoms. Particularly, at least one of R³⁰², R³⁰⁴, R³⁰⁵, and R³⁰⁶ may be a 2, 6-di-tert-butyl phenyl group.

In the nitrogen-containing condensed cyclic compound according to the present disclosure, in view of the peak wavelength of the emission spectrum, luminescence color purity, emission efficiency, and device lifespan, the structure represented by Formula (1) may be the structure represented by Formula (1A) or (1B). Thus, an embodiment of the present disclosure relates to a nitrogen-containing condensed cyclic compound having the structure represented by Formula (1A) or (1B). In an embodiment of a material for an organic electroluminescent device to be described later, the structure represented by Formula (1) of the nitrogen-containing condensed cyclic compound may be the structure represented by Formula (1A) or (1B). In an embodiment of a fluorescence emitter to be described later, the structure represented by Formula (1) of the nitrogen-containing condensed cyclic compound may be the structure represented by Formula (1A) or (1B). In this regard, the embodiments of Formulae (1A) and (1B) are each as described above. In this case, Formulae (1A) and (1B) may satisfy the conditions below.

In Formula (1A), when each of A^{a} to A^{d} is a group derived from a benzene ring, at least one of na to nd may be 3 or more, or at least one of R^{a} to R^{d} may be an unsubstituted branched alkyl group having 4 to 15 carbon atoms,

in Formula (1B), when each of A^{a} to A^{d} is a group derived from a benzene ring, at least one of na, nc, ne, and nf may be 3 or more, or at least one of R^{a}, R^{c}, R^{e}, and R^{f} may be an unsubstituted branched alkyl group having 4 to 15 carbon atoms.

The nitrogen-containing condensed cyclic compound according to an embodiment of the present disclosure may have at least one substituent (e.g., A^{a} to A^{d} of Formula (I)), wherein the at least one substituent may be an aromatic ring and is linked to a ring-forming carbon of the core portion represented by Formula (1-1). In the nitrogen-containing condensed cyclic compound, a dihedral angle between the core portion and the at least one substituent derived from an aromatic ring linked to the core portion by a single bond may be, but not limited to 50° or more. The dihedral angle may be 55° or more, and may be 60°. The dihedral angle may be 90° or less. Thus, when the nitrogen-containing condensed cyclic compound according to an embodiment of the present disclosure includes at least one of substituent wherein the at least one substituent includes an aromatic hydrocarbon group or a heterocyclic group, for example a group derived from an aromatic ring, and is linked to a ring-forming carbon of the core portion by a single bond, at least one of the dihedral angles among the dihedral angles between the core portion and for example the group derived from an aromatic ring linked to the core portion by a single bond may be within the range described above. Within the range, a difference between the FWHM of the peak of the luminescence spectrum in photoluminescence (PL) obtained in a solution state and the FWHM of the peak of the luminescence spectrum in PL obtained in a film state may decrease. As a result, in a film state, a small FWHM of a peak of a luminescence spectrum may be obtainable, and thus, a FWHM of a peak of a narrow luminescence spectrum may be obtained in an organic electroluminescent device. In addition, in the present disclosure, the dihedral angle may not have values of 0° or more and 90° or less.

In a substituent including a group derived from an aromatic ring linked to the core portion by a single bond, the group derived from an aromatic ring linked to the core portion by a single bond may be a group derived from an aromatic hydrocarbon ring or a group derived from an aromatic hetero ring. The aromatic hydrocarbon ring is the same as described in connection with the aromatic hydrocarbon ring in the description of the monovalent aromatic hydrocarbon ring of (f). Examples of the aromatic hydrocarbon ring are the same as those of the aromatic hydrocarbon ring in the description of the monovalent aromatic hydrocarbon ring of (f). The aromatic hetero ring is the same as described in connection with the aromatic hetero ring in the monovalent heterocyclic group of (g). Examples of the aromatic hetero ring are the same as those of the aromatic hetero ring in the monovalent heterocyclic group of (g).

The dihedral angle between the core portion and the group derived from an aromatic ring linked to the core portion by a single bond may be calculated by using GaussView (Gaussian Inc.) from the most stable structure calculated according to the “Calculation by DFT” section. Herein, the dihedral angle between the core portion and the group derived from an aromatic ring linked to the core portion by a single bond refers to, when α1 is a core atom linked to the substituent, α2 is an atom closest to α1 among the cores, β1 is a substituent atom linked to the core, and β2 is an atom closest to β1 among the substituents, an angle between triangle Δα2α1β1 with vertices α2, α1, and β1 and triangle Δβ2β1α1 with vertices β2, β1, and α1. Details of the calculation method are described in the embodiments.

In a substituent including a group derived from an aromatic ring linked to the core portion by a single bond, the group derived from an aromatic ring linked to the core portion by a single bond may be selected from a group derived from an aromatic hydrocarbon ring, a group derived from a carbazole ring, and a group derived from a dibenzofuran ring. Further, the group derived from an aromatic ring linked to the core portion by a single bond may be a group derived from a hydrocarbon ring that is aromatic as a whole, a group derived from a carbazole ring, and a group derived from a dibenzofuran ring. In a substituent including a group derived from an aromatic ring linked to the core portion by a single bond, the group derived from an aromatic ring linked to the core portion by a single bond may be a group derived from an aromatic ring included in the same plane. Examples of groups derived from aromatic rings within the same plane may be a group derived from a benzene ring, a group derived from a carbazole ring, a group derived from a dibenzofuran ring, and a group derived from a fluorene ring. However, examples of the groups derived from aromatic rings within the same plane are not limited to the above. In this regard, in a substituent including a group derived from an aromatic ring linked to the core portion by a single bond, the group derived from an aromatic ring linked to the core portion by a single bond may be a group derived from a benzene ring, a group derived from a carbazole ring, a group derived from a dibenzofuran ring, and a group derived from a fluorene ring. In addition, the group derived from an aromatic ring linked to the core portion by a single bond may be selected from a group derived from a benzene ring, a group derived from a carbazole ring, and a group derived from a dibenzofuran ring, for example, a group derived from a benzene ring.

The nitrogen-containing condensed cyclic compound according to an embodiment of the present disclosure may have one or two or more substituents including a group selected from a group derived from a benzene ring linked to the core portion by a single bond, a group derived from a carbazole ring linked to the core portion by a single bond, and a group derived from a dibenzofuran ring linked to the core portion by a single bond, and the dihedral angle between the core portion and at least one group selected from a group derived from a benzene ring linked to the core portion by a single bond, a group derived from a carbazole ring linked to the core portion by a single bond, and a group derived from a dibenzofuran ring linked to the core portion by a single bond may be 50° or more. The dihedral angle may be 55° or more, or may be 60°. The dihedral angle may be 90° or less. The nitrogen-containing condensed cyclic compound according to an embodiment of the present disclosure may have one or two or more substituents including a group derived from a benzene ring linked to the core portion by a single bond, and the dihedral angle between the core portion and at least one group derived from a benzene ring linked to the core portion by a single bond may be 50° or more. The dihedral angle may be 55° or more, or may be 60°. The dihedral angle may be 90° or less.

The nitrogen-containing condensed cyclic compound according to the present disclosure may have a peak wavelength of an emission spectrum in the blue wavelength region, and may realize luminescence with high color purity. The blue wavelength region as used herein refers to a wavelength region within a range of 380 nm or more and 500 nm or less. The PL peak wavelength of the nitrogen-containing condensed cyclic compound according to the present disclosure may be, but not limited to, a range of 440 nm or more and 480 nm or less. Further, the peak wavelength may emit light having a peak in a wavelength region of 445 nm or more and 470 nm or less, or 450 nm or more and 470 nm or less, for example, 450 nm or more and 465 nm or less. When the peak wavelength is within the above range, excellent luminescence, particularly, excellent blue luminescence may be obtained. The range of the FWHM of the peak of the emission spectrum in PL may be, for example, 30 nm or less, 20 nm or less, or 15 nm or less (the lower limit exceeds 0 nm). The PL peak wavelength and the FWHM of the peak of the emission spectrum of the PL may be measured using a spectro fluorescence photometer F7000 of Hitachi Hightech Inc. More specifically, 1×10⁻⁵ M(=mol/dm³ mol/L) of toluene solution of the nitrogen-containing condensed cyclic compound according to the present disclosure may be evaluated by measuring at room temperature with an excitation wavelength of 360 nm using the spectro fluorescence photometer.

Narrow FWHM, TADF characteristics, and emission wavelength that are required for molecules used as dopants may be predicted by quantum chemistry calculation.

In the condensed cyclic compound according to an embodiment of the present disclosure, the highest occupied molecular orbital (HOMO) energy may be, but not particularly limited to, −5.8 eV or more. Further, the HOMO energy may be −5.6 eV or more, or −5.4 eV or more. The HOMO energy may be −4.6 eV or less. The HOMO energy may be −4.8 eV or less, or −5.0 eV or less. In the above range, the difference between the HOMO energy of the condensed cyclic compound of the present disclosure and the HOMO energy of a general host material used for an organic electroluminescent device may decrease. As a result, the increase of a driving voltage due to formation of a hole trap may be inhibited.

In the condensed cyclic compound according to an embodiment of the present disclosure, the lowest unoccupied molecular orbital (LUMO) energy may be, but not particularly limited to, −2.4 eV or more. The LUMO energy may be −2.2 eV or more, or −2.1 eV or more. The LUMO energy may be −2.0 eV or more. The LUMO energy may be −0.8 eV or less. The LUMO energy may be −0.1 eV or less, or −1.1 eV or less. The LUMO energy may be −1.2 eV or less. In the above range, the difference between the LUMO energy of the condensed cyclic compound of the present disclosure and the LUMO energy of a general host material used as an organic electroluminescent device may decrease. As a result, the increase of a driving voltage due to formation of an electron trap may be inhibited.

In the condensed cyclic compound according to an embodiment of the present invention, an adiabatic first excitation singlet state (S1) energy (hereinafter, adiabatic S1 excitation energy) (eV) may be converted to emission wavelength (nm) and obtained. The range peak of the fluorescent wavelength may be the same as the range of the peak wavelength of the luminescence in PL.

In the condensed cyclic compound according to an embodiment of the present disclosure, the adiabatic oscillator strength f in the stable structure of the first excitation singlet state (S1) may be, but not particularly limited to, 0.22 or more. The oscillator strength f may be 0.3 or more. The oscillator strength f may be 0.4 or more, or 0.5 or more. In the above range, a greater fluorescent luminescence strength may be obtained. Further, the theoretical upper limit of the oscillator strength f is the number of electrons included in a molecule. The upper limit of the oscillator strength f may be, for example, 2 or 3, but the upper limit of the oscillator strength f is not limited thereto.

In the condensed cyclic compound according to an embodiment, the reorganization energy may be 0.12 or less. Further, the reorganization energy may be 0.1 eV or less, for example, 0.08 eV or less (lower limit: 0 eV). In the above range, the spectral width of the luminescence is narrowed, and a high luminescence color purity may be obtained.

the peak of the fluorescent wavelength, oscillator strength f, and reorganization energy, which are obtainable by converting the HOMO, LUMO, adiabatic S1 excitation energy to emission wavelength, may be calculated by DFT by using Gaussian 16 (Gaussian Inc.) as a calculation software. Details of the calculation method are described in the embodiments.

The nitrogen-containing condensed cyclic compound according to the present disclosure may satisfy Conditions (i) to (iv):

ΔE_{ST}>ΔE_{ST2}+ΔE′_{TT} Condition (i)

0 eV<ΔE_{ST2}+ΔE′_{TT}≤1.0 Condition (ii)

0 eV<ΔE′_{TT}≤0.15 eV Condition (iii)

ΔE_{ST2}>0 eV Condition (iv)

wherein, in Conditions (i) to (iv),

ΔE_{ST}(eV) indicates the difference value obtained from subtracting the lowest triplet excitation energy (eV) calculated from the T₁ equilibrium structure from the lowest singlet excitation energy (eV) calculated from the S₁ equilibrium structure,

ΔE_{ST2}(eV) indicates the difference value obtained from subtracting the second lowest triplet excitation energy (eV) calculated from the T₂ equilibrium structure from the lowest singlet excitation energy (eV) calculated from the S₁ equilibrium structure, and

ΔE′_{TT}(eV) indicates a difference value obtained by subtracting the lowest triplet excitation energy (eV) calculated from the T₂ equilibrium structure from the second lowest triplet excitation energy (eV) calculated from the T₂ equilibrium structure.

Here, each of the S₁ equilibrium structure, the T₁ equilibrium structure, and the T₂ equilibrium structure are formed when molecules are in an excited state, and these states represent the most stable structure wherein the energy for each state becomes the lowest.

Conditions (i) to (iv) may be obtained as below.

In the Tamm-Dancoff approximation, Time-Dependent Density Functional Theory (TDDFT) using PBE0 functional and def2-SVP basis set were used to optimize structure and calculate energy in the T₁, T₂, and S₁ states. By structure optimization, the equilibrium structure in the T₁, T₂, and S₁ states was obtained to thereby obtain the lowest triplet excitation energy in the T₁ equilibrium structure, the lowest triplet excitation energy and the second lowest triplet excitation energy in the T₂ equilibrium structure, and the lowest singlet excitation energy in the S₁ equilibrium structure. The Q-Chem program was used in the calculation.

Table 1 summarizes the calculation method of Conditions (i) to (iv).

TABLE 1

| Calculation | |
|---|---|
| program | Q-Chem program |
| Calculation | S₁, T₁, and T₂ structure |
| method | optimization and energy calculation through |
| | Time-Dependent Density Functional Theory by |
| | Tamm-Dancoff approximation using PBE0 functional |
| Basis Set | Def2-SVP basis set |

It has been found that a compound that satisfies Conditions (i) to (iv) may improve efficiency of an organic electroluminescent device including the compound. In the previous application, it has been shown that a compound satisfying Conditions (i) to (iv) may have TADF characteristics, and improvement of efficiency may be realized through the TADF characteristics. (KR Patent Application no. 10-2019-0107649) The nitrogen-containing condensed cyclic compound according to the present disclosure also shows that satisfying Conditions (i) to (iv) may highly improve efficiency of an organic electroluminescent device including the compound.

An embodiment of the present disclosure may be a nitrogen-containing condensed cyclic compound having the structure represented by Formula (1) and satisfying Conditions (i) to (iv). An embodiment of the present disclosure may be a nitrogen-containing condensed cyclic compound having the structure represented by Formula (2) or (3) and satisfying Conditions (i) to (iv). In Formula (2), at least one of n5 to n8 may be 3 or more or at least one of R⁵ to R⁸ may be an unsubstituted branched alkyl group having 4 to 15 carbon atoms. In Formula (3), at least one of n5, n7, n9, and n10 may be 3 or more, or at least one of R⁵, R⁷, R⁹, and R¹⁰ may be an unsubstituted branched alkyl group having 4 to 15 carbon atoms. In a nitrogen-containing condensed cyclic compound according to an embodiment of the disclosure, a structure represented by Formula (1), (2), or (3) may be represented by one of Formulae (3-1) to (3-9) (a structure selected from a group represented by Formulae (3-1) to (3-9)) and may satisfy Conditions (i) to (iv).

In an embodiment of a material for an organic electroluminescent device to be described later, a nitrogen-containing condensed cyclic compound may have the structure represented by Formula (1), and may satisfy Conditions (i) to (iv).

In an embodiment of the present disclosure, the solubility of mesitylene of the nitrogen-containing condensed cyclic compound may be, but not limited to, 0.3 g/L or more. The solubility may be 0.5 g/L or more, or 1.0 g/L or more. Luminescence color purity may be further improved within this range. The reason is assumed as below. The aggregation between the compound molecules is inhibited, and the solubility of the compound molecule itself is improved, and the degree of purification is improved, thereby improving luminescence color purity and device lifespan. Even when additives are increased due to the inhibition of aggregation between compound molecules, color purity is not easily degraded, and luminescence of high color purity may be realized. In an embodiment of the present disclosure, the solubility of mesitylene of the nitrogen-containing condensed cyclic compound may be, but not limited to, 100 g/L or more. Further, the solubility may be 50 g/L or less, for example, 30 g/L or less.

In an embodiment of the material for an organic electroluminescent device, the nitrogen-containing condensed cyclic compound may have a solubility of mesitylene of 1.0 g/L or more.

Hereinafter, the nitrogen-containing condensed cyclic compound according to an embodiment of the present disclosure will be explained in detail. The present disclosure is not limited to these examples:

[chemical structure]

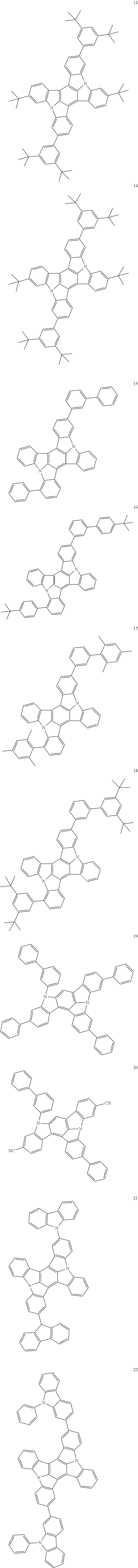

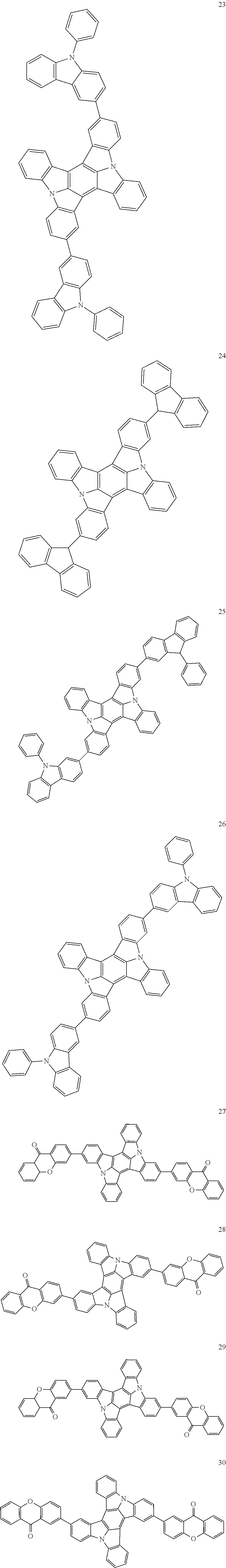

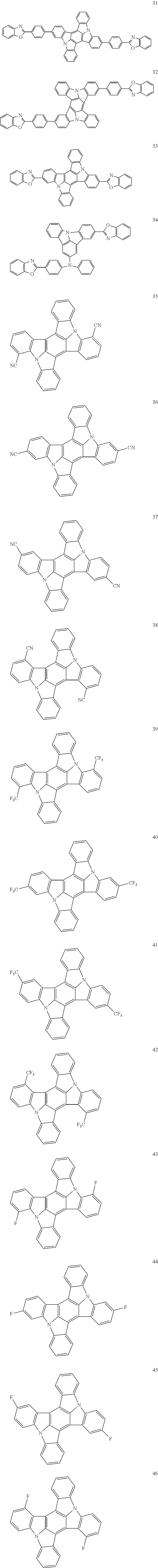

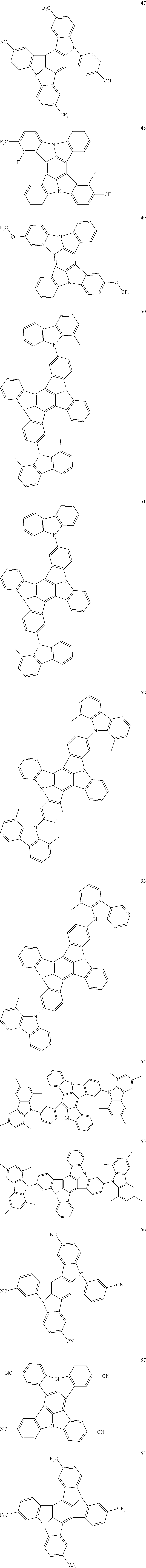

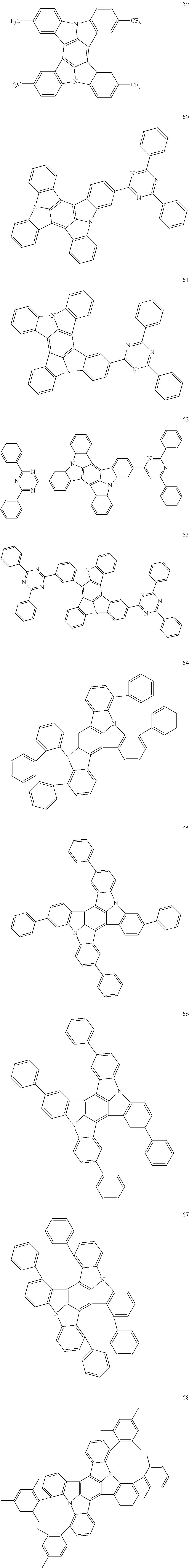

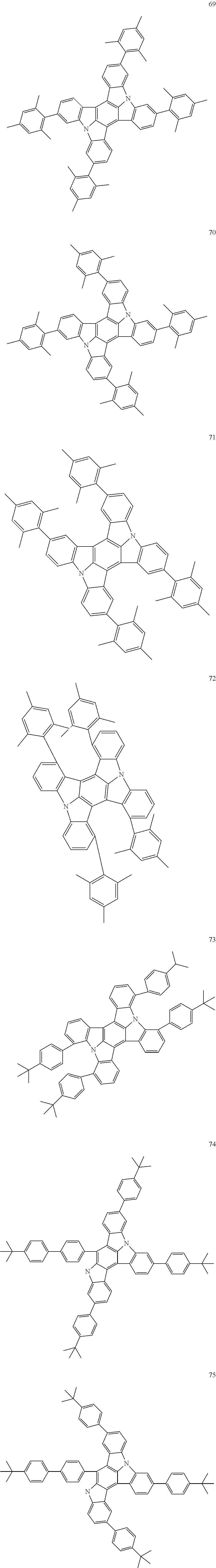

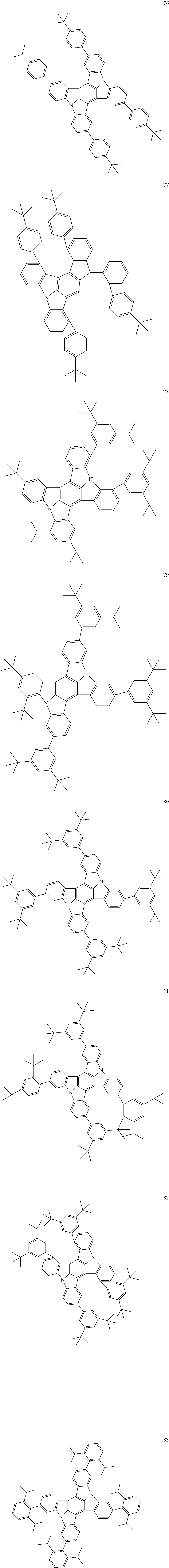

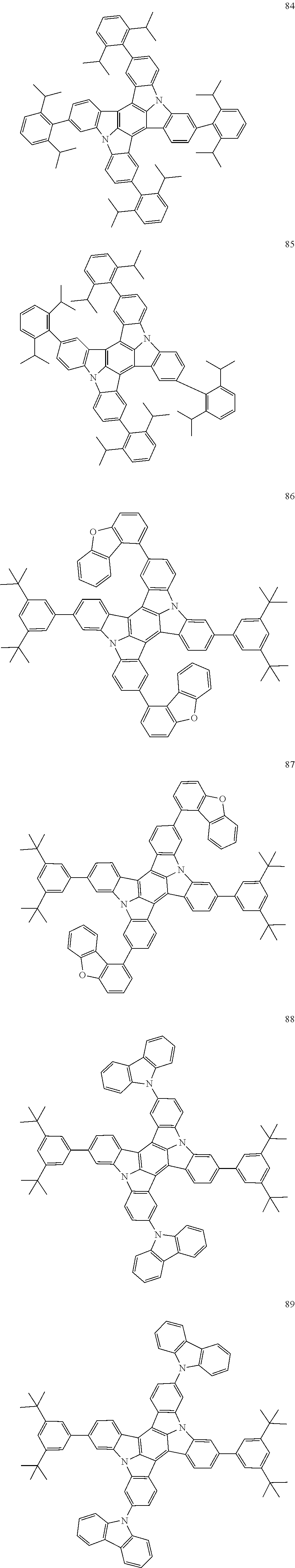

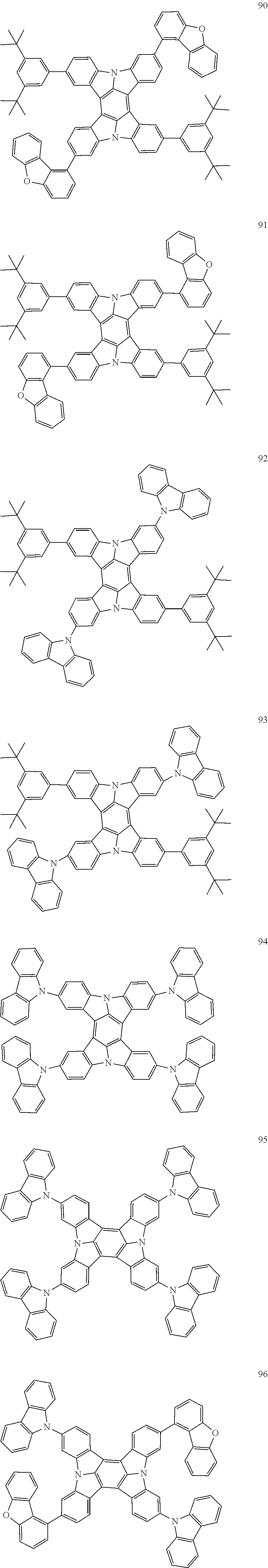

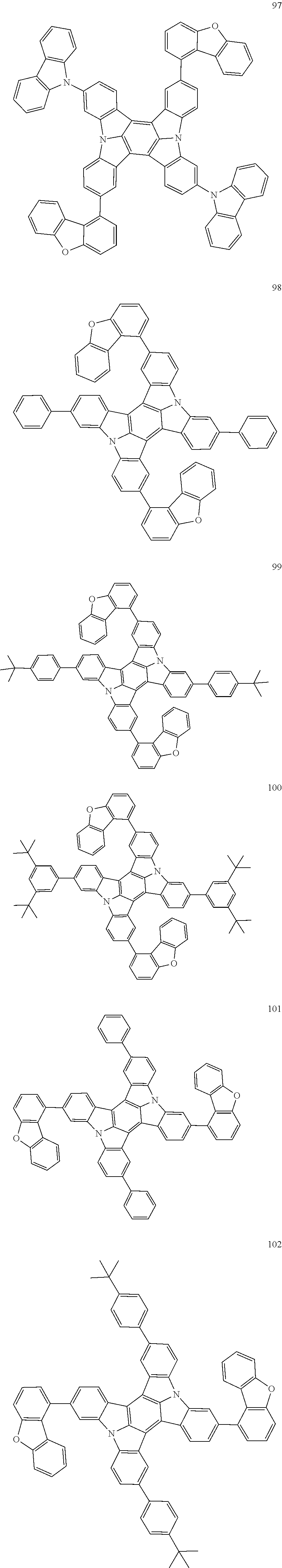

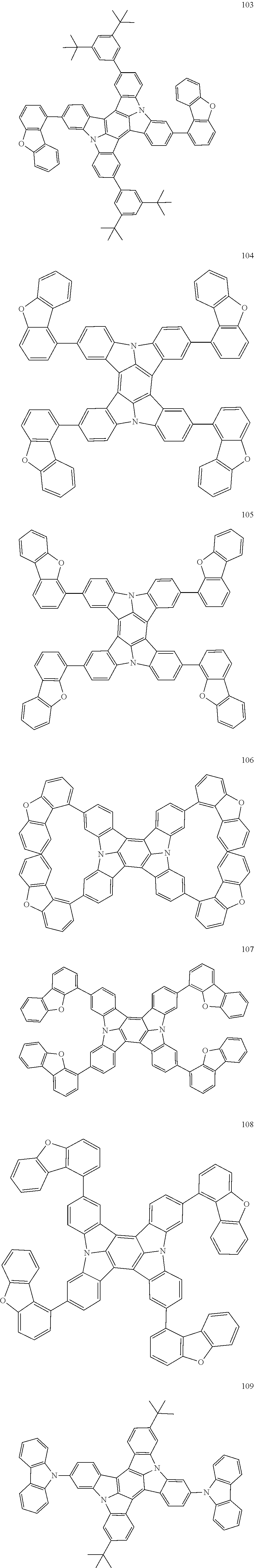

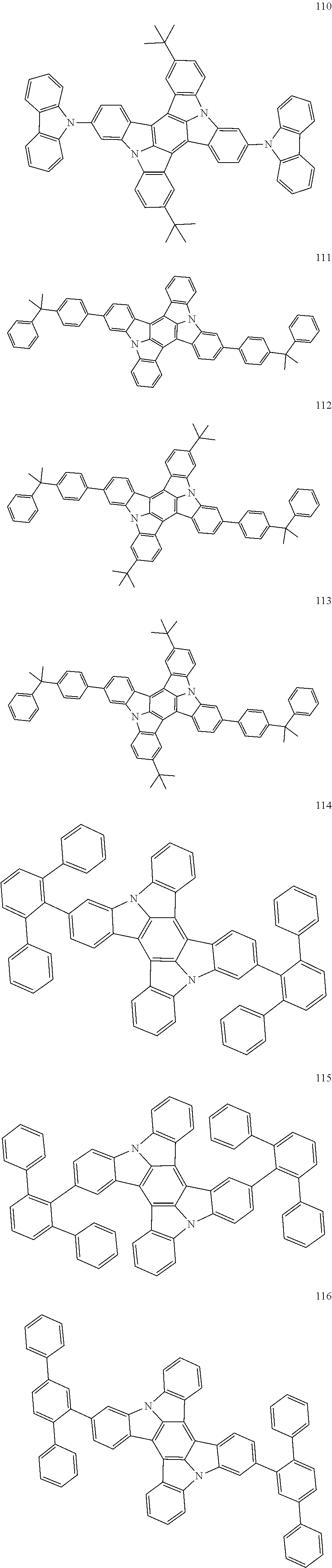

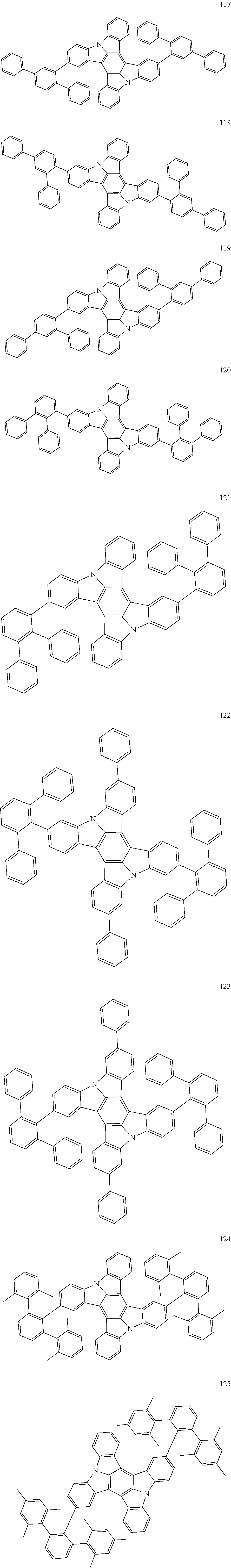

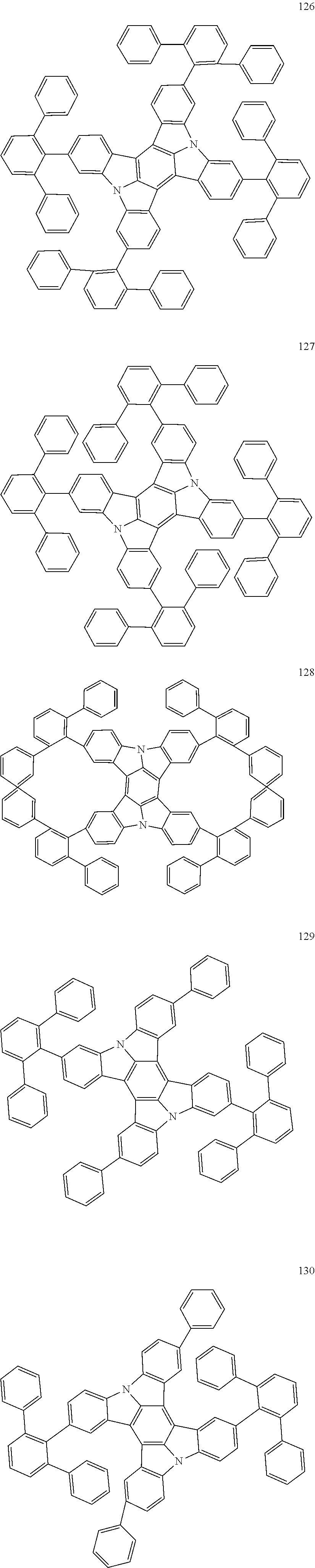

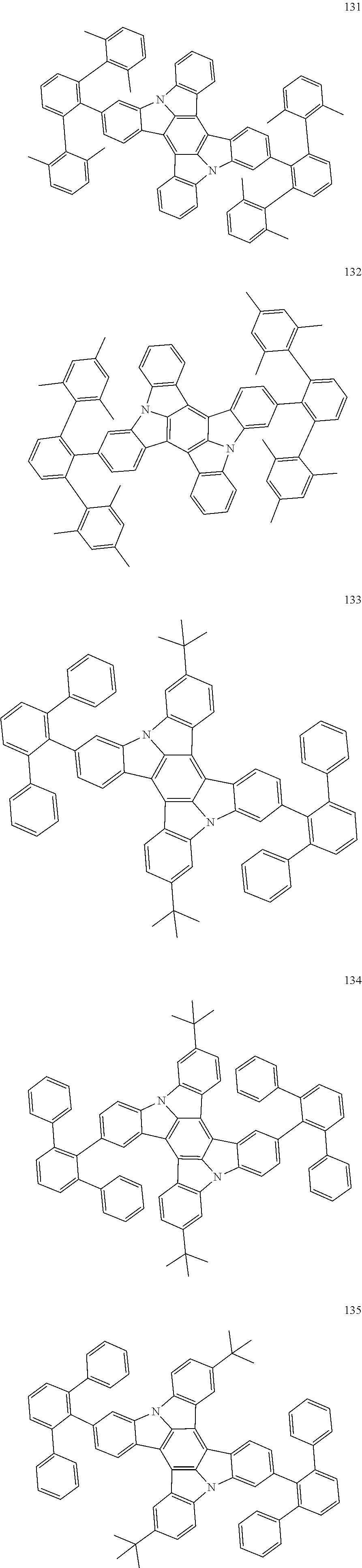

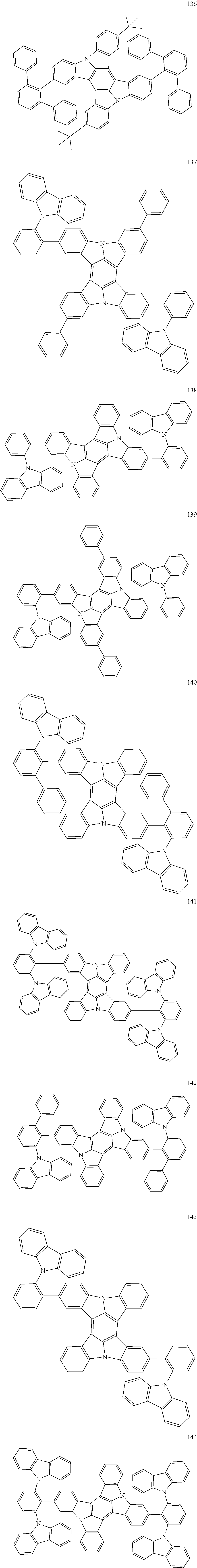

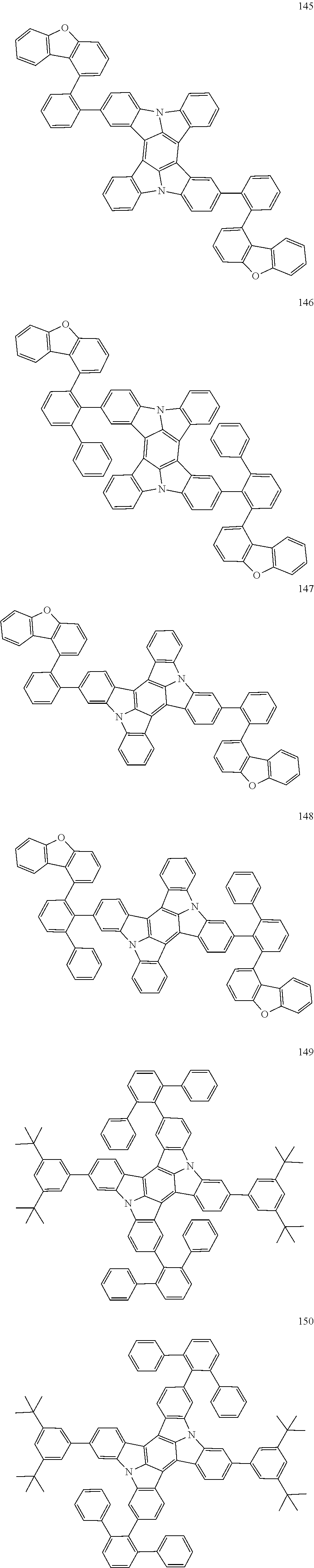

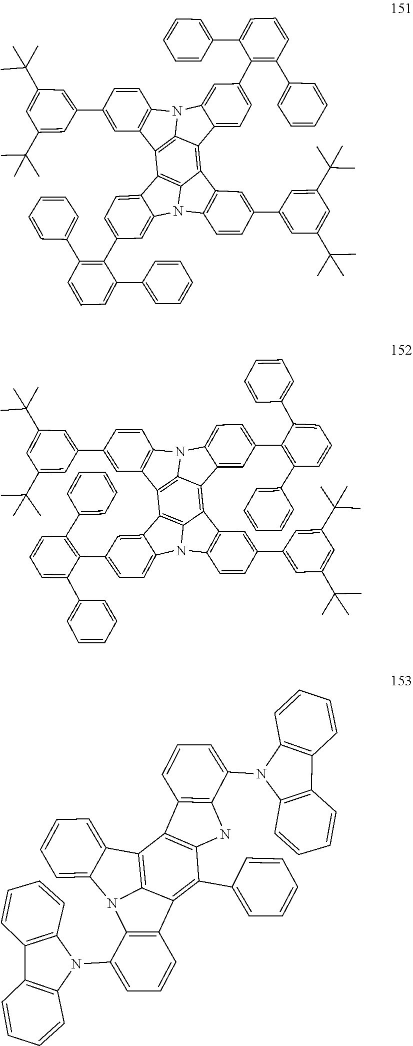

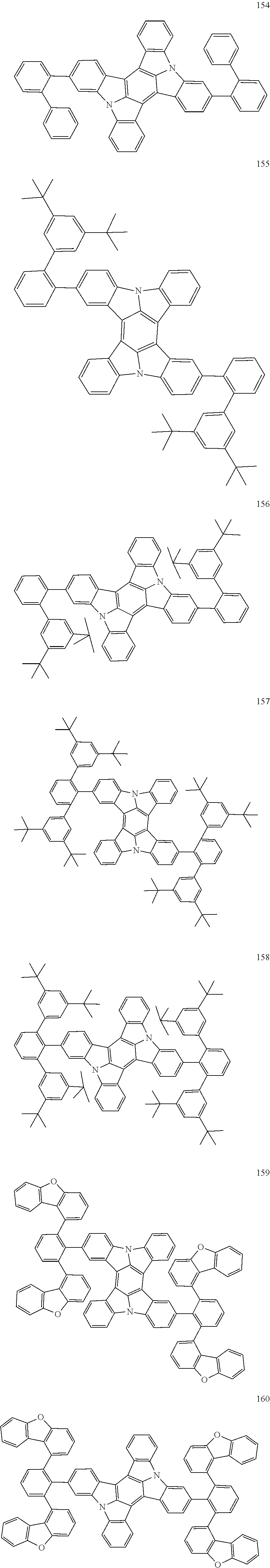

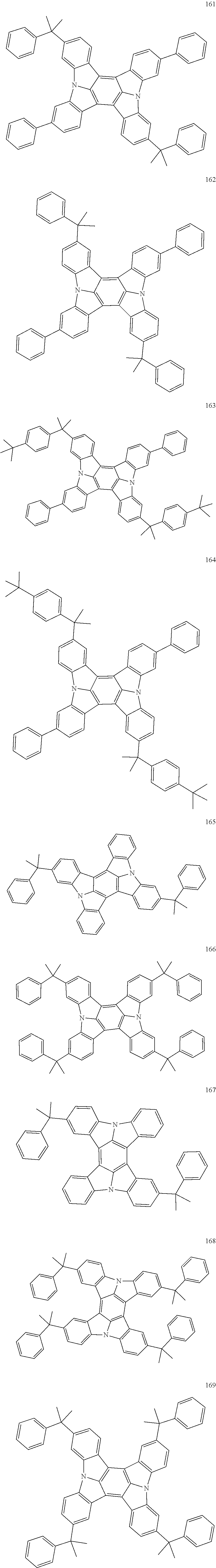

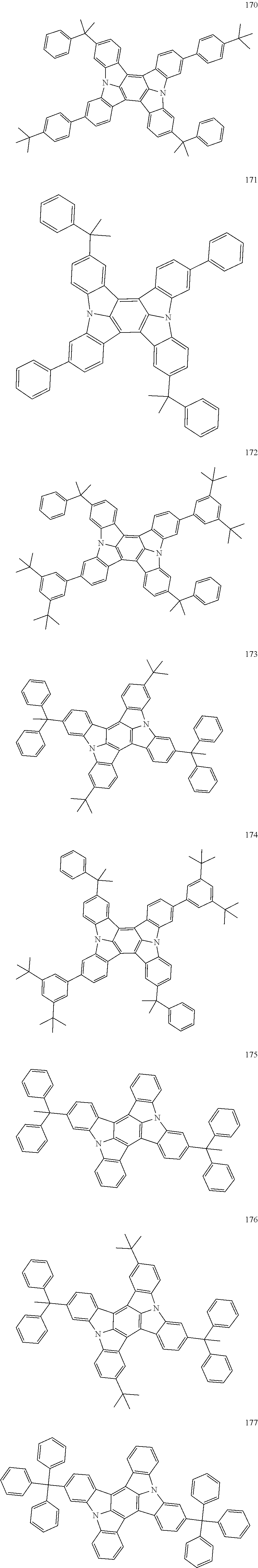

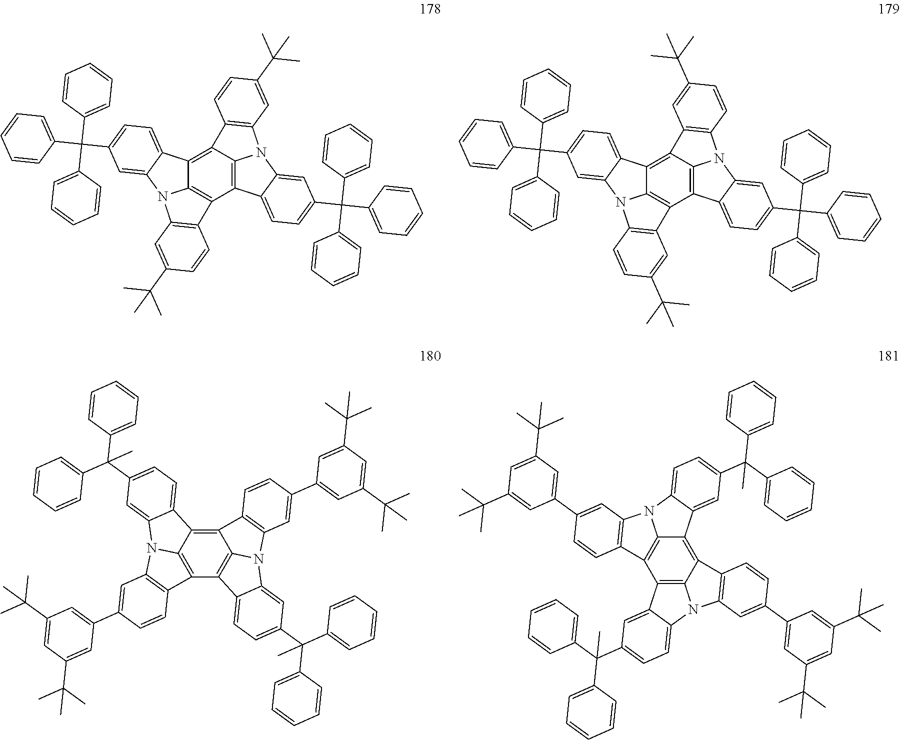

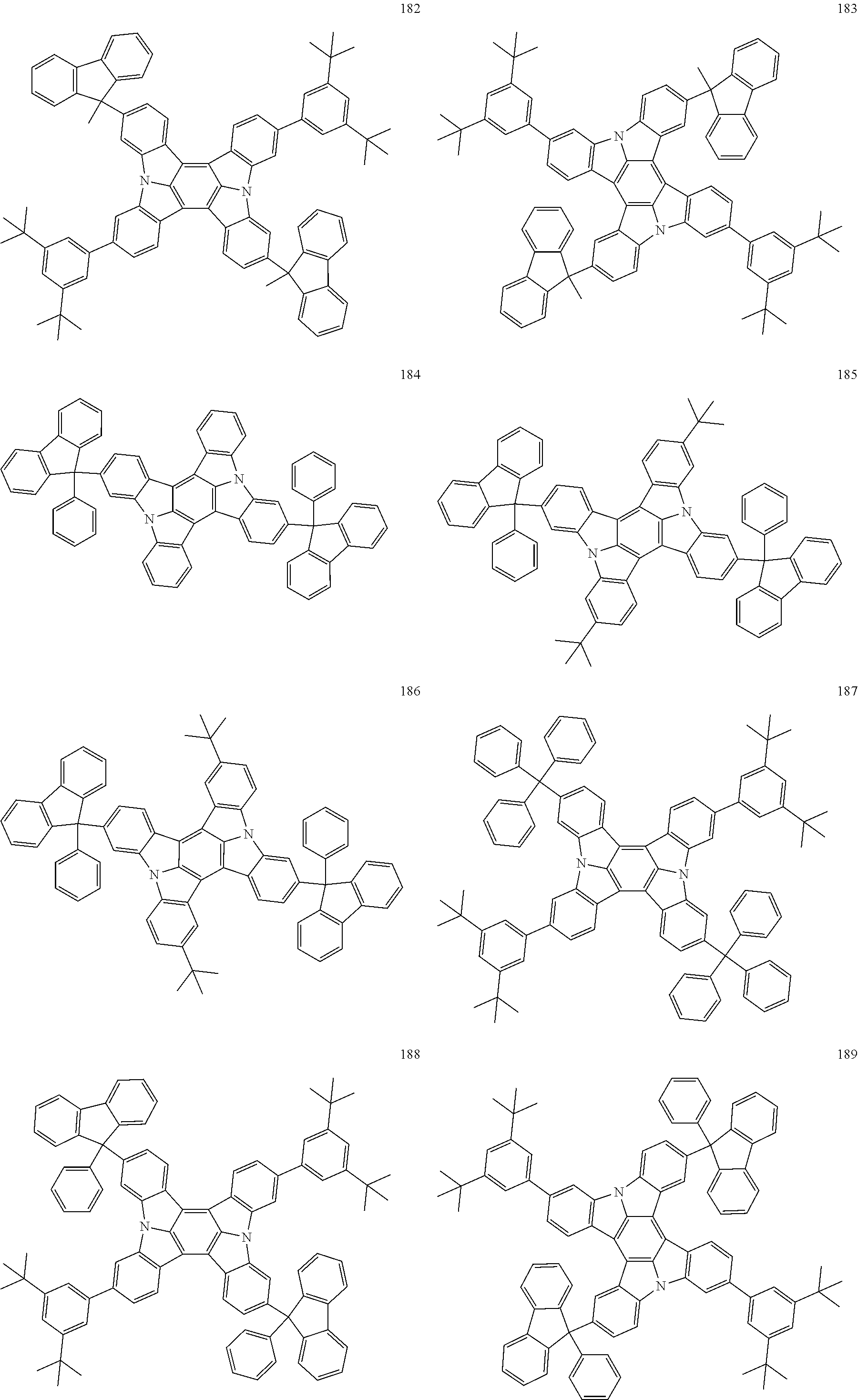

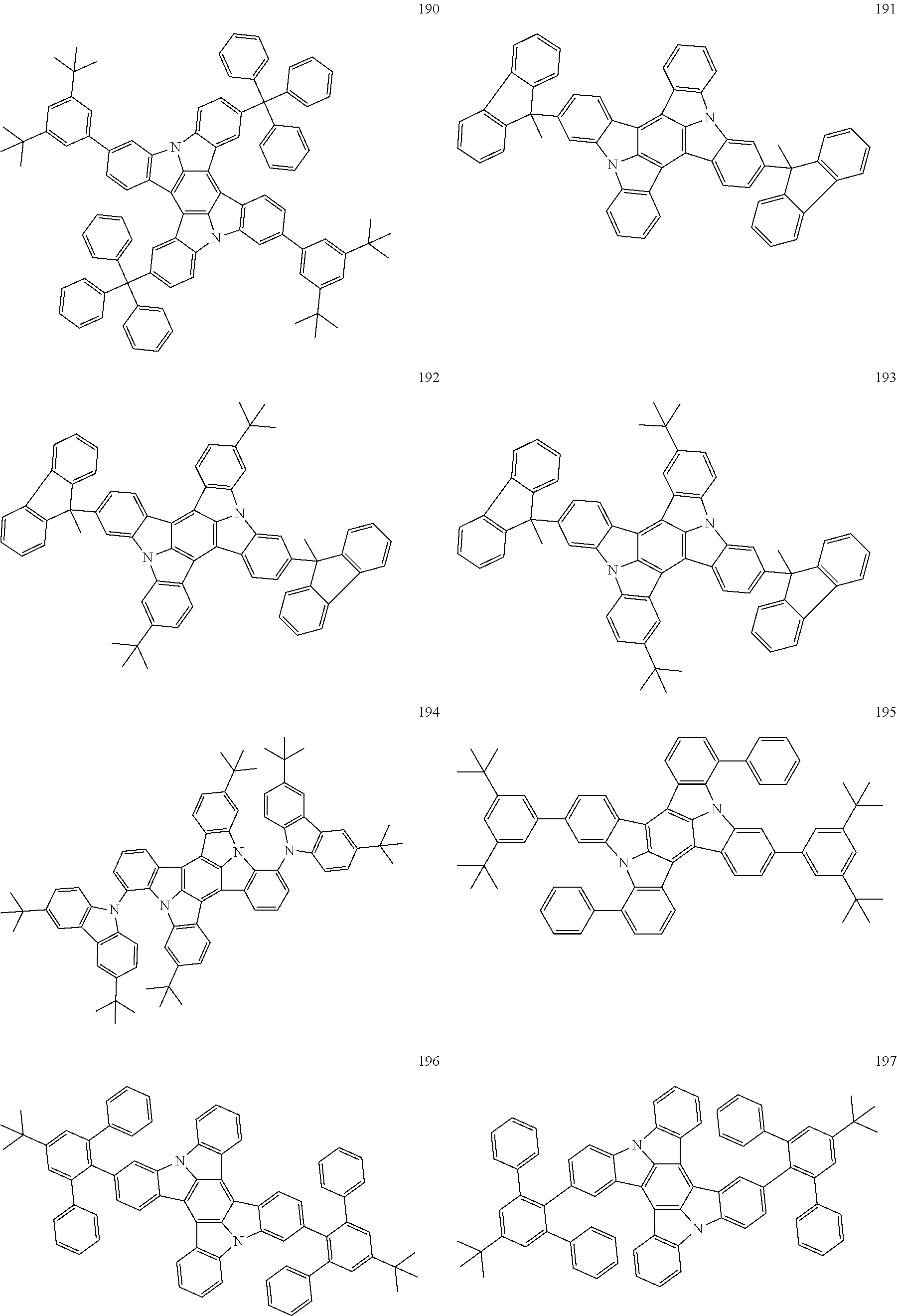

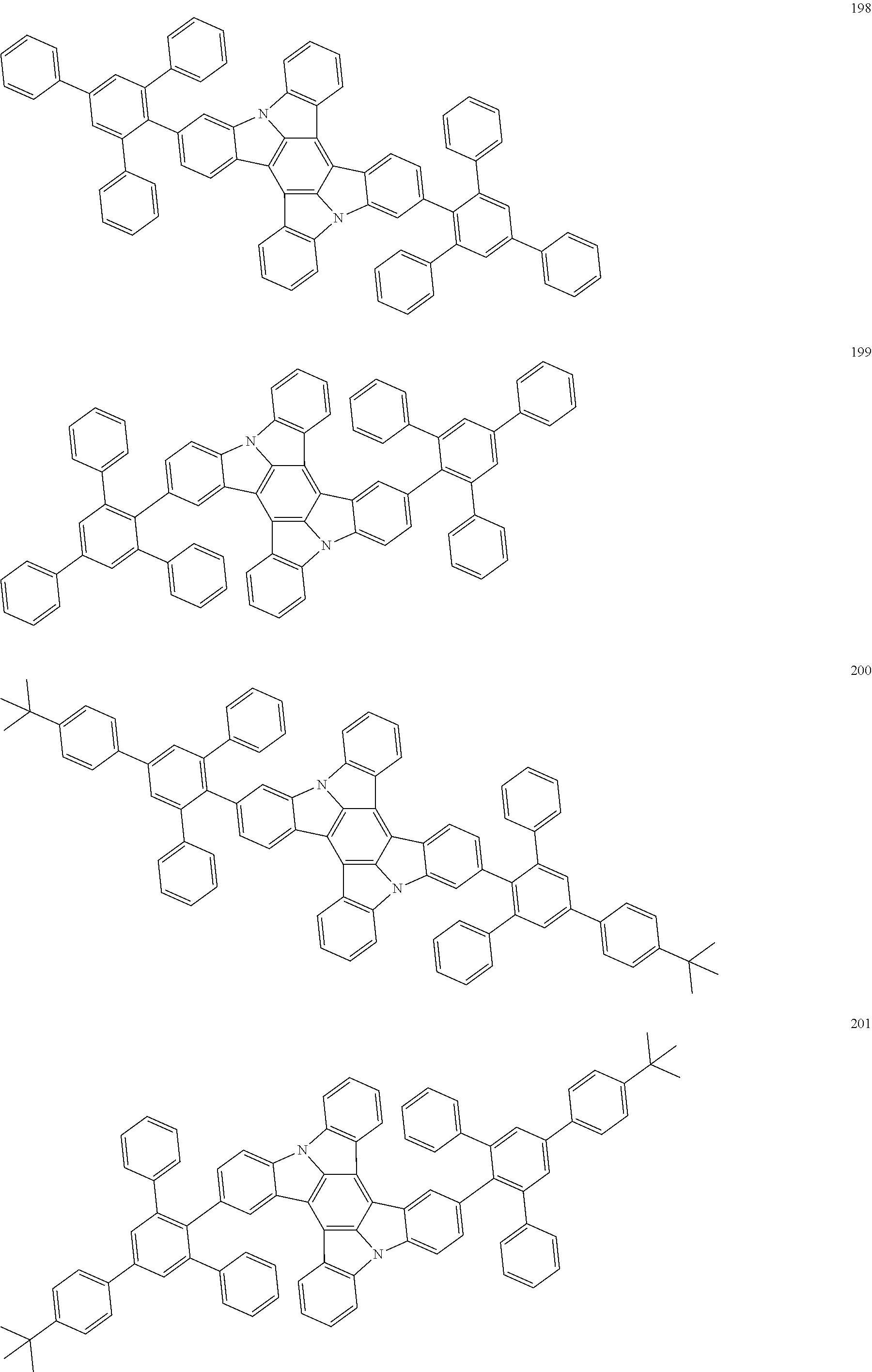

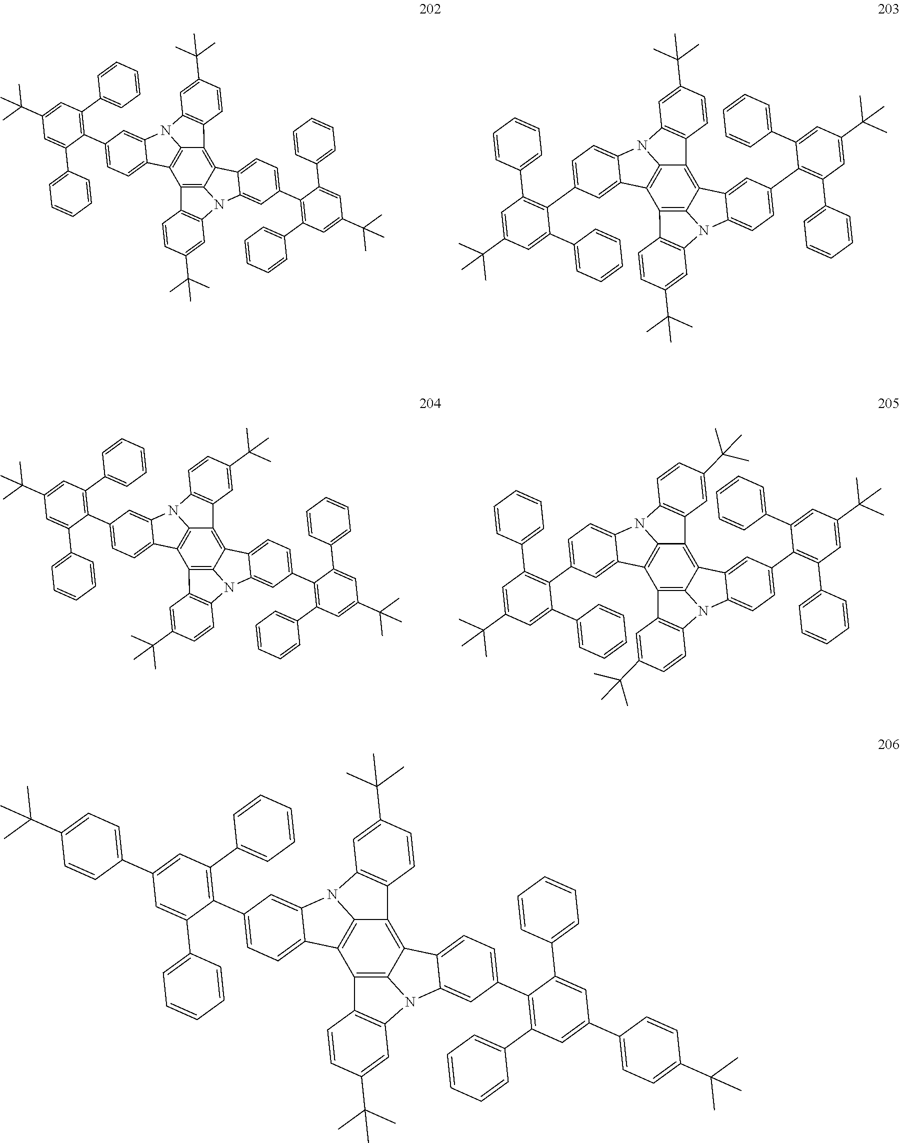

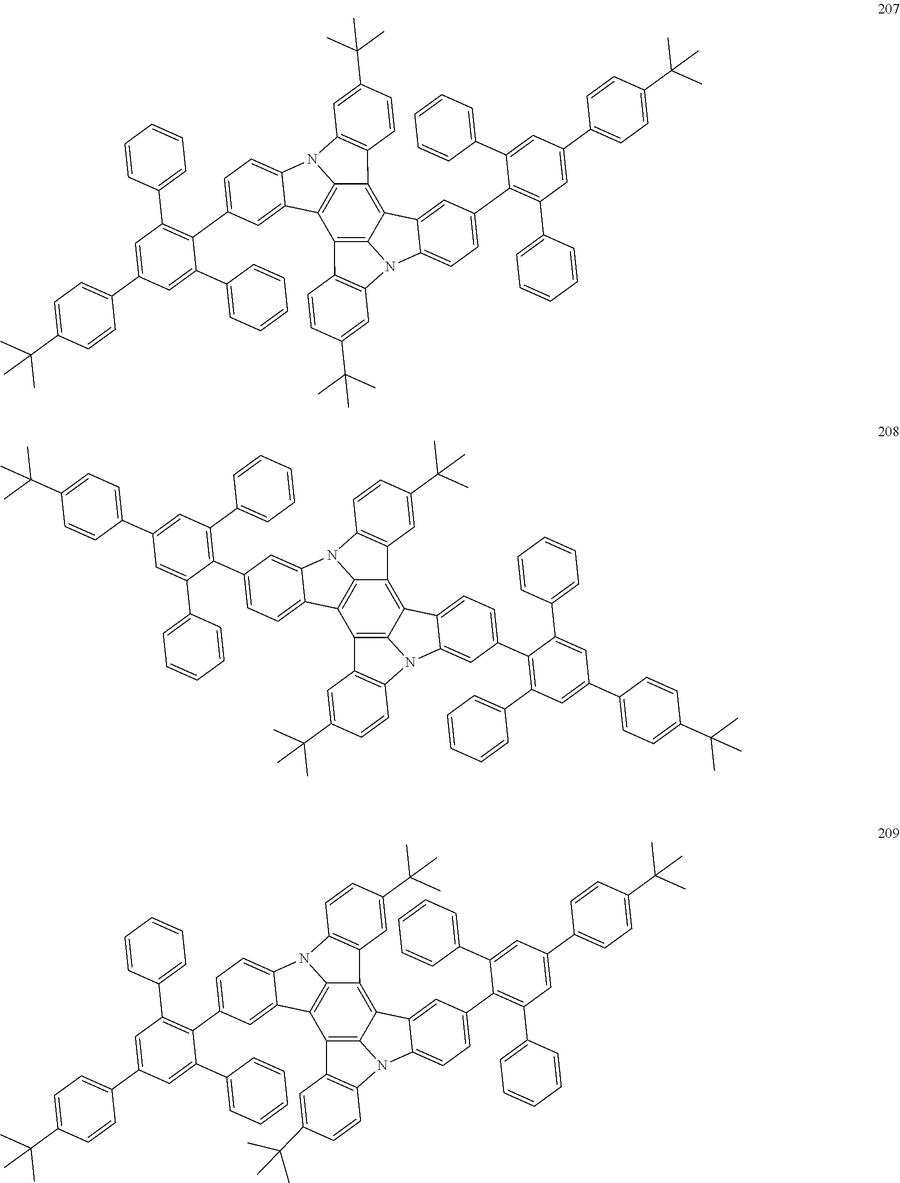

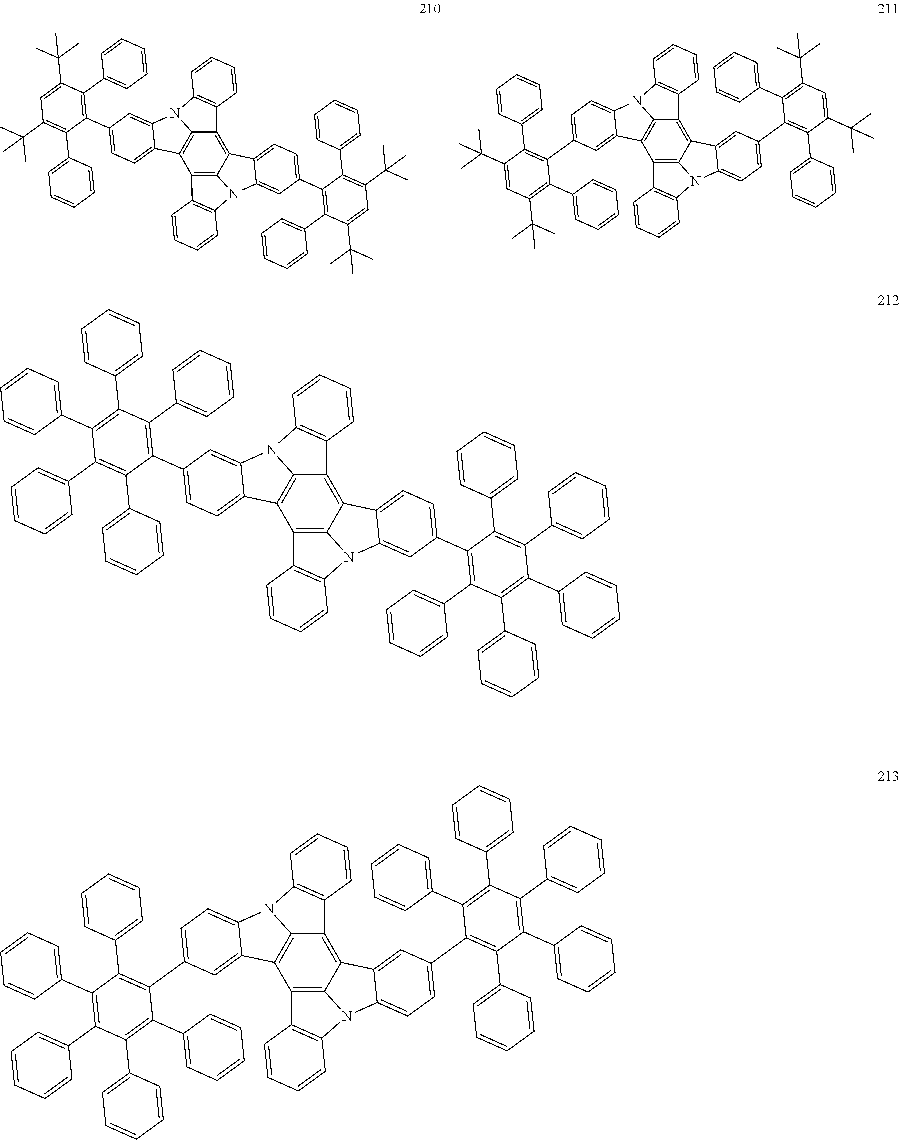

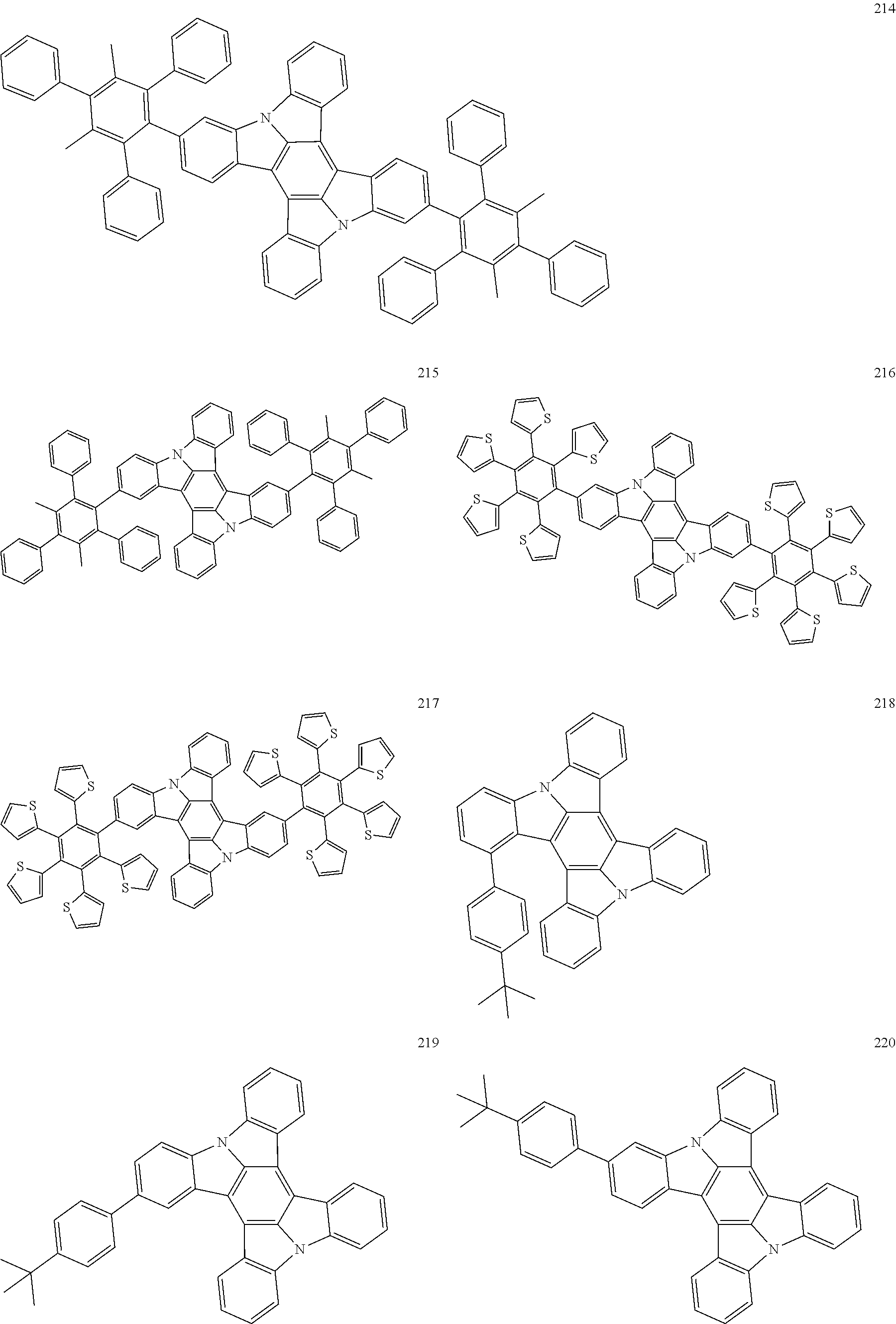

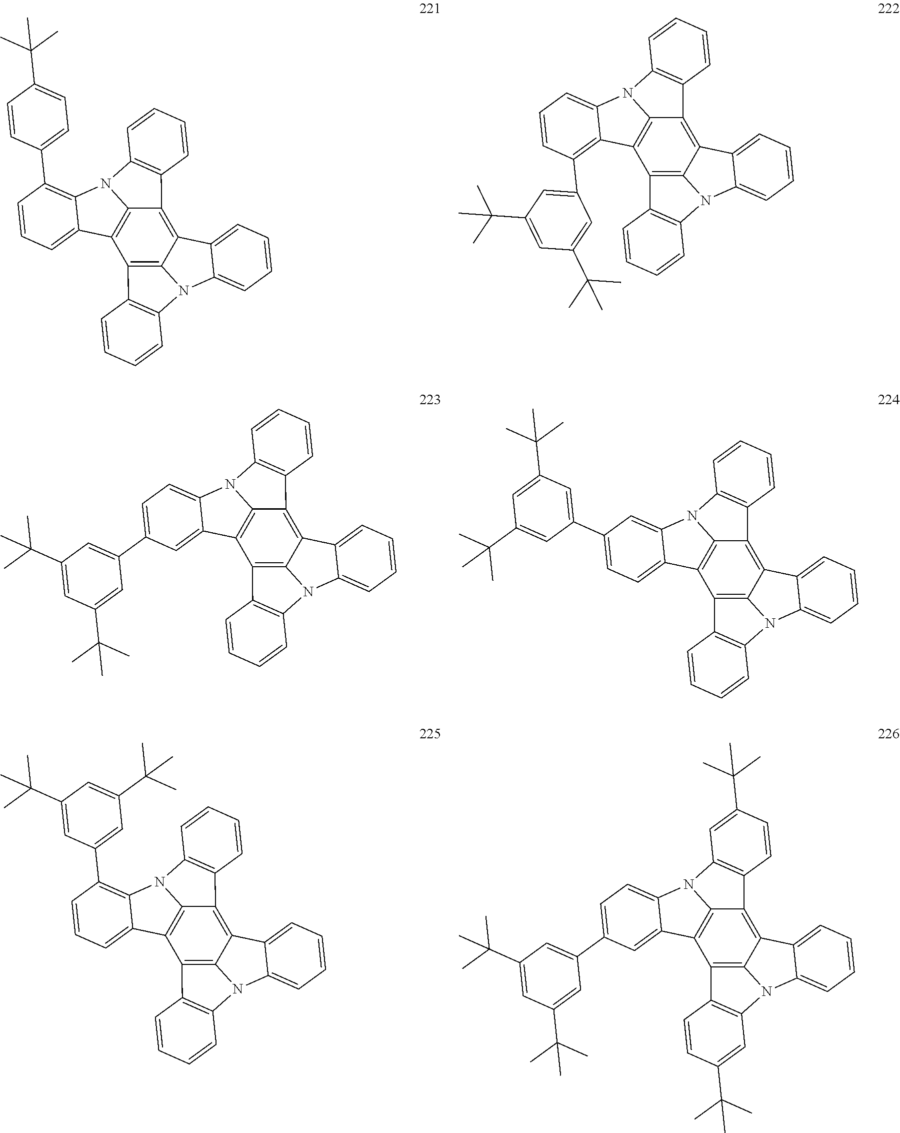

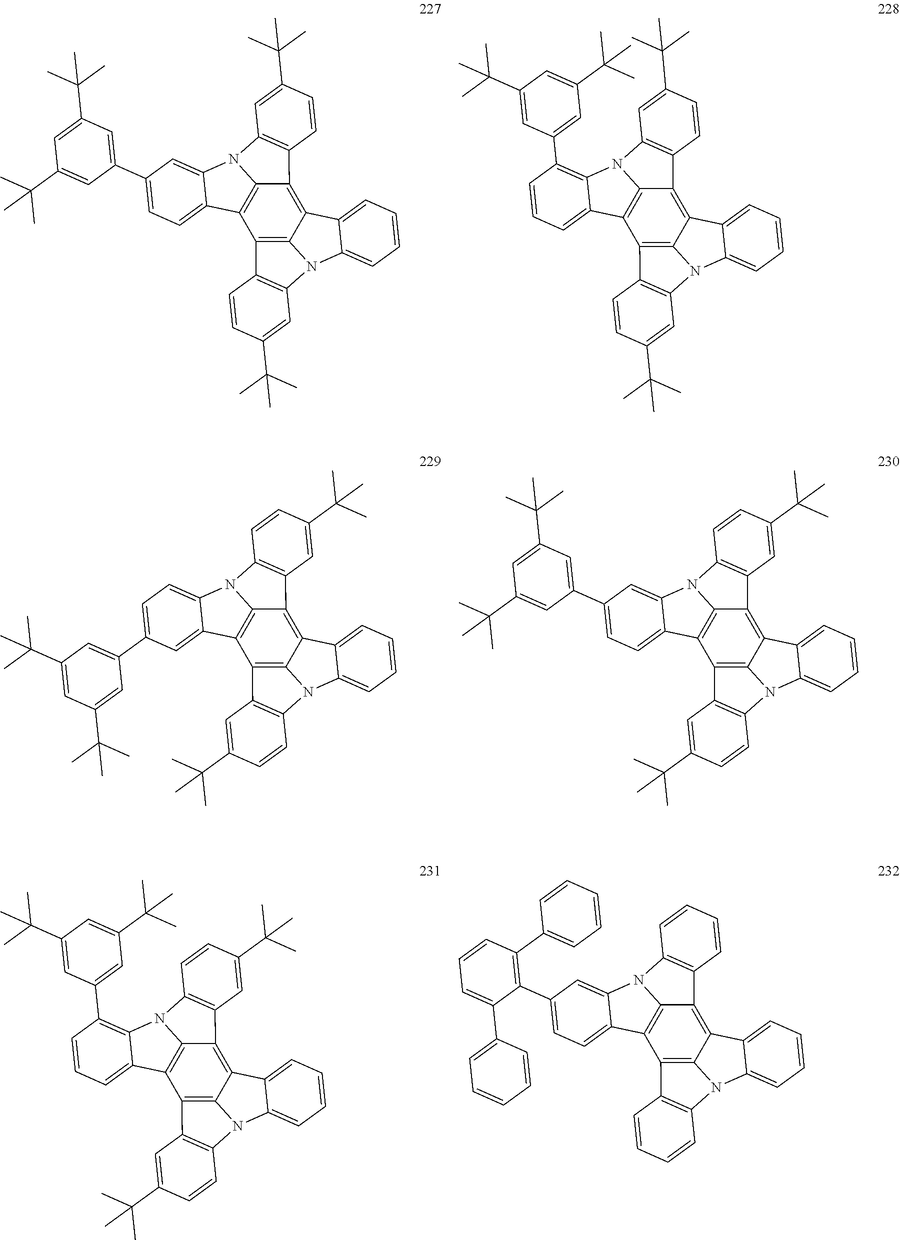

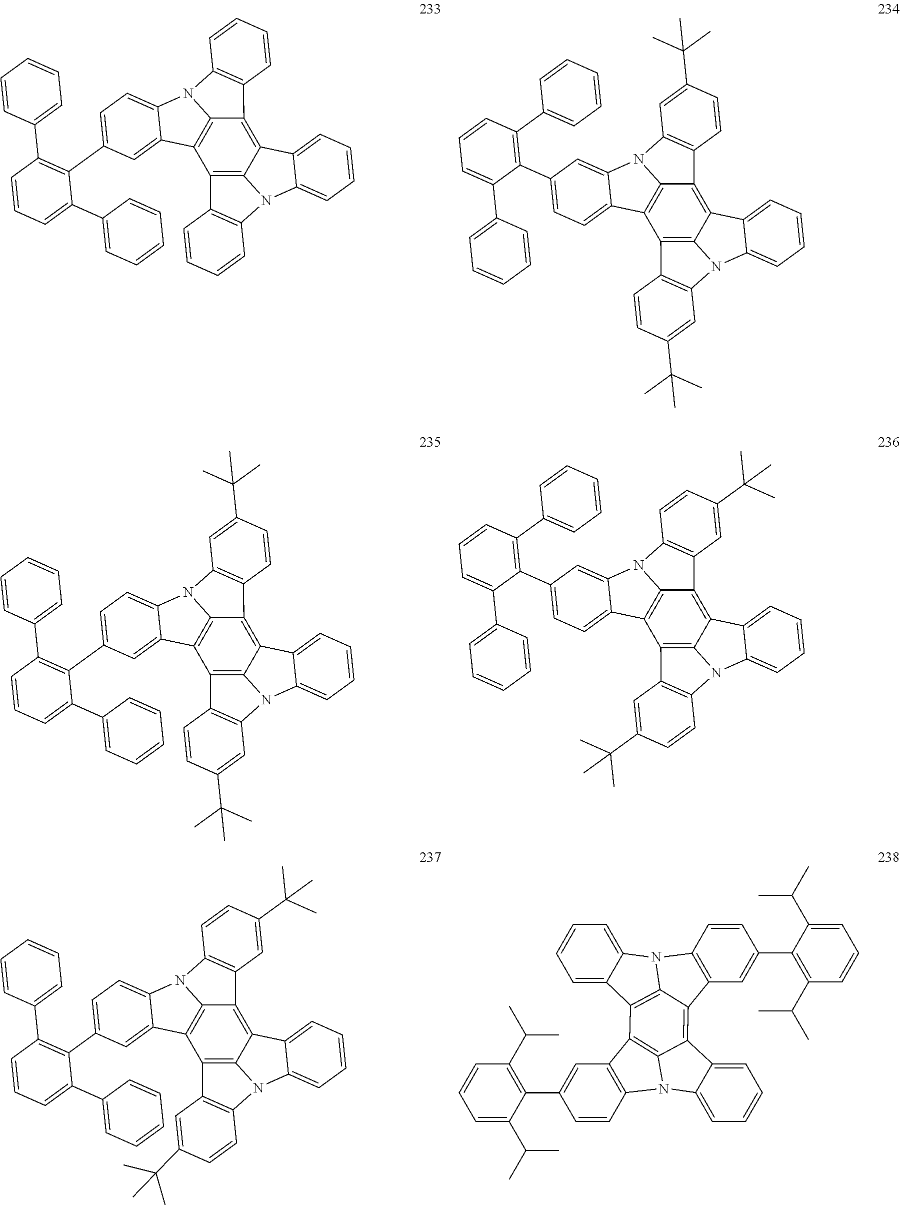

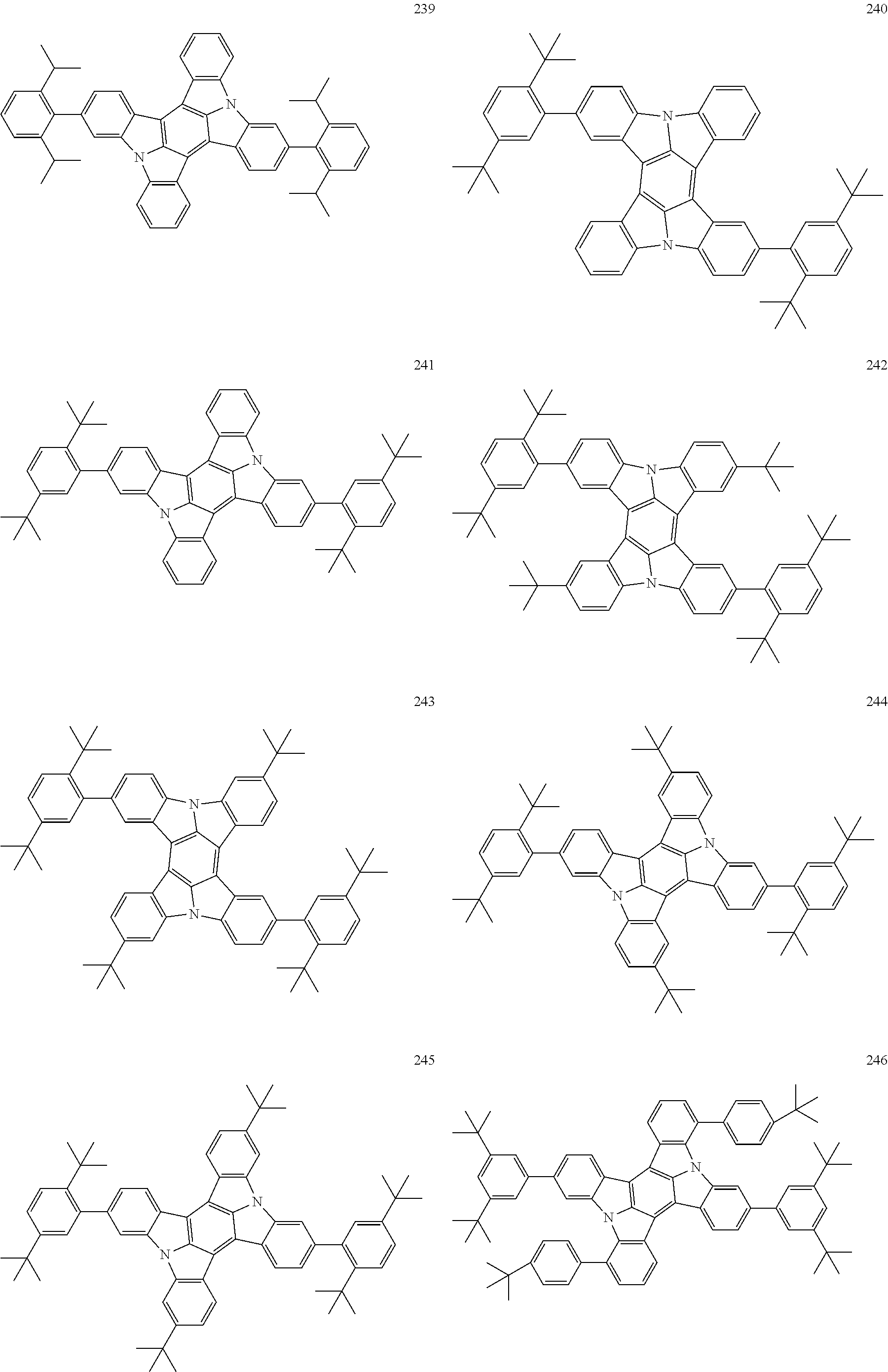

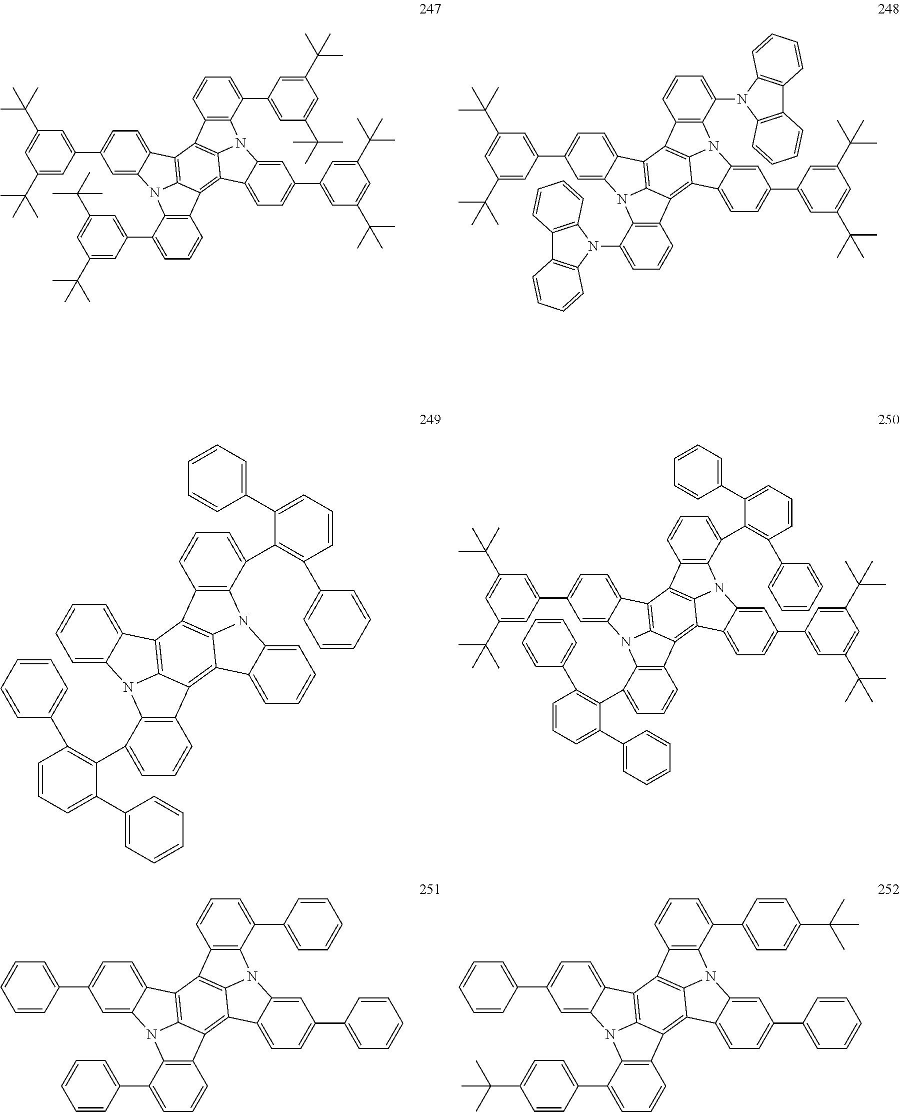

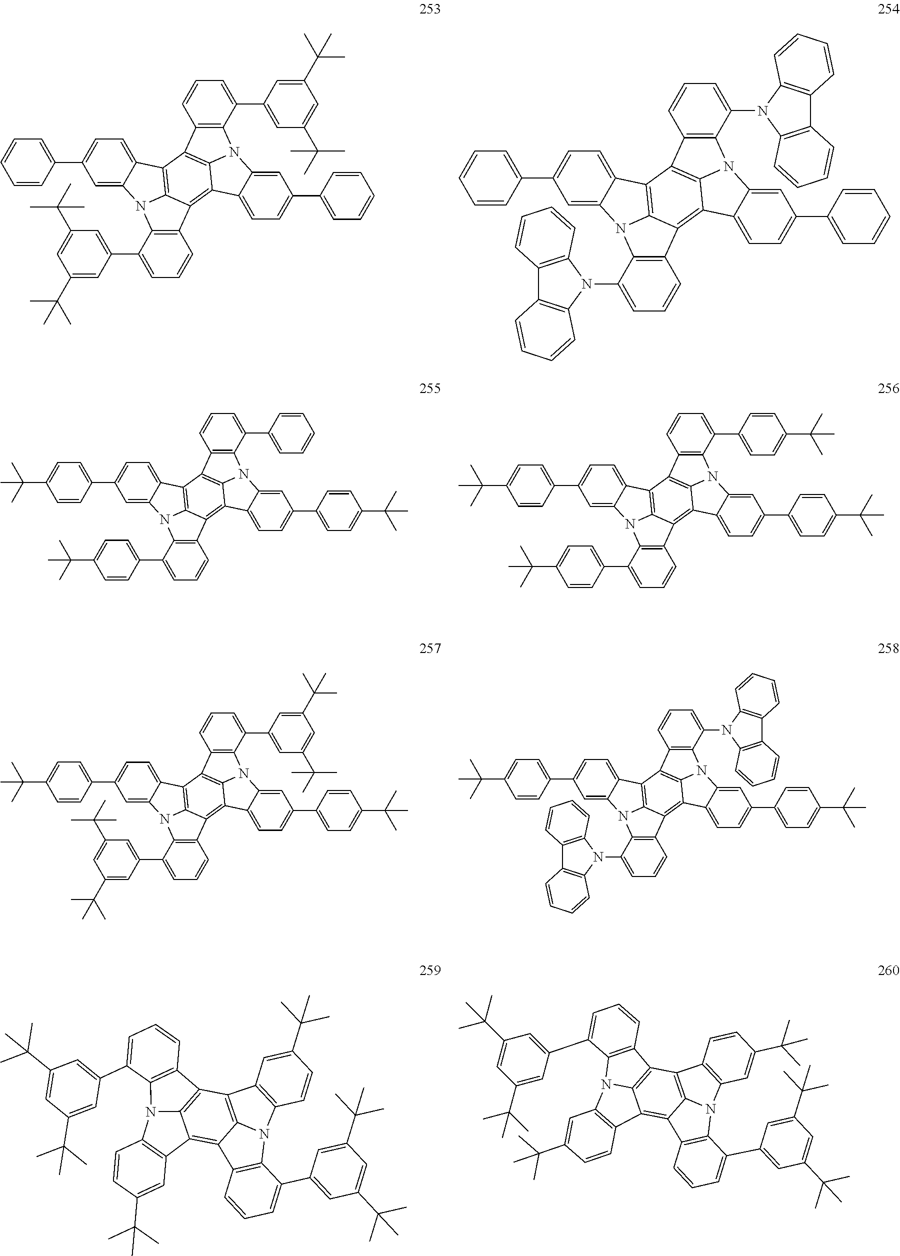

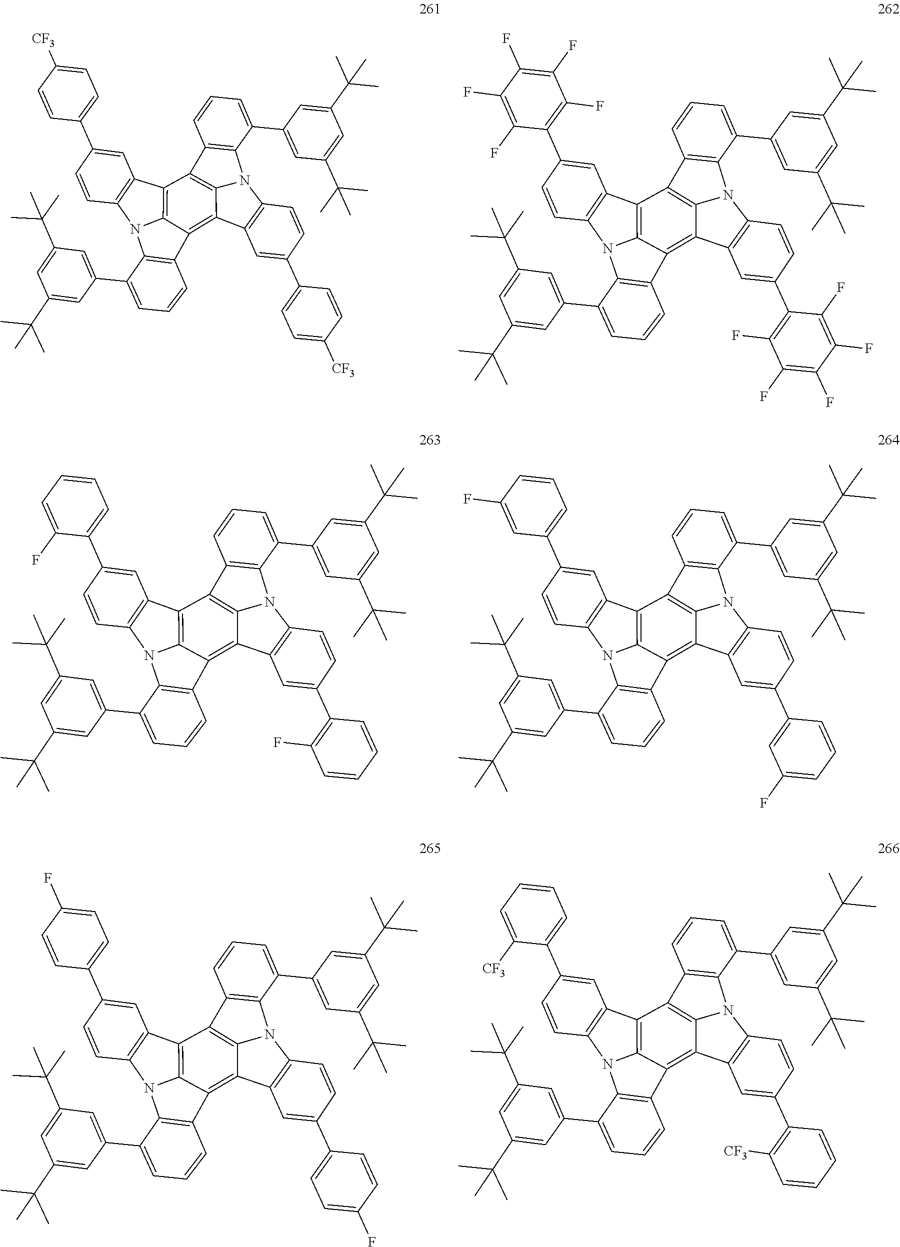

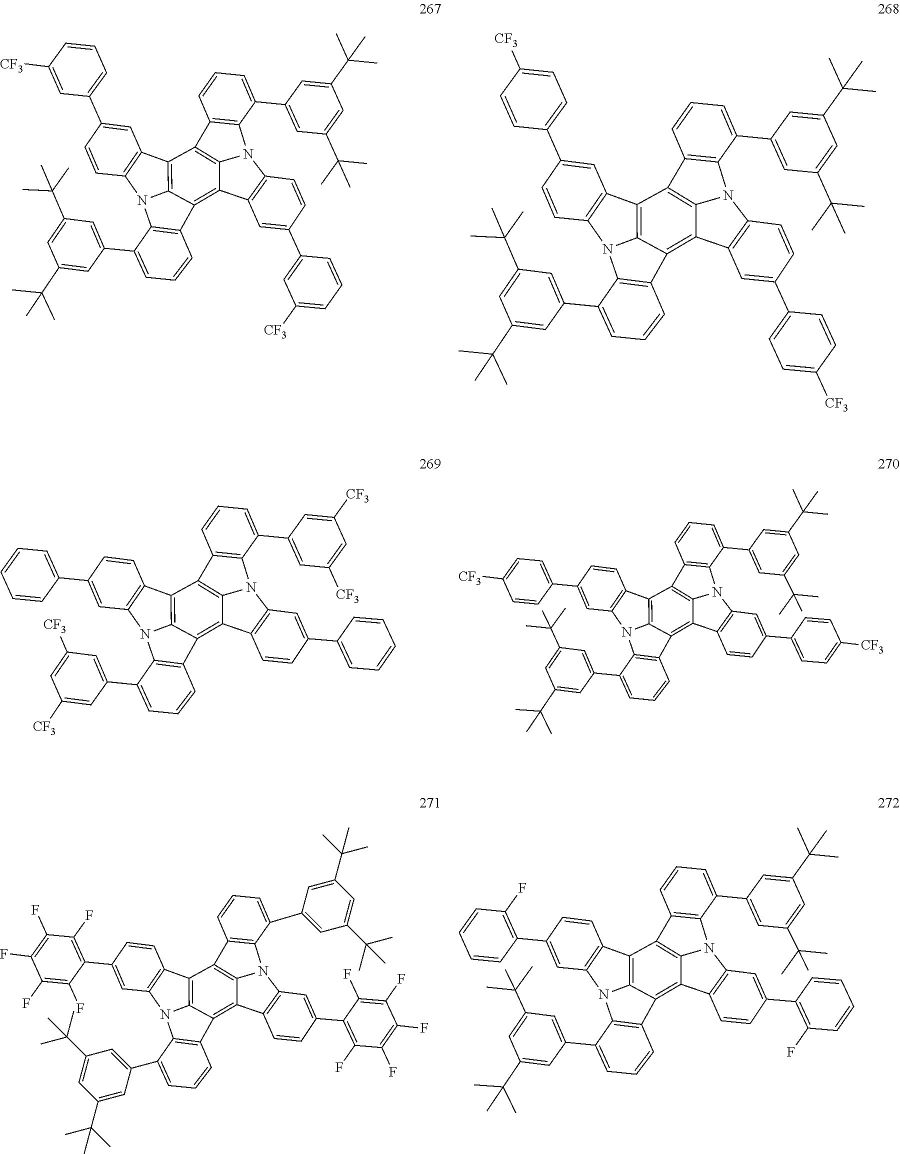

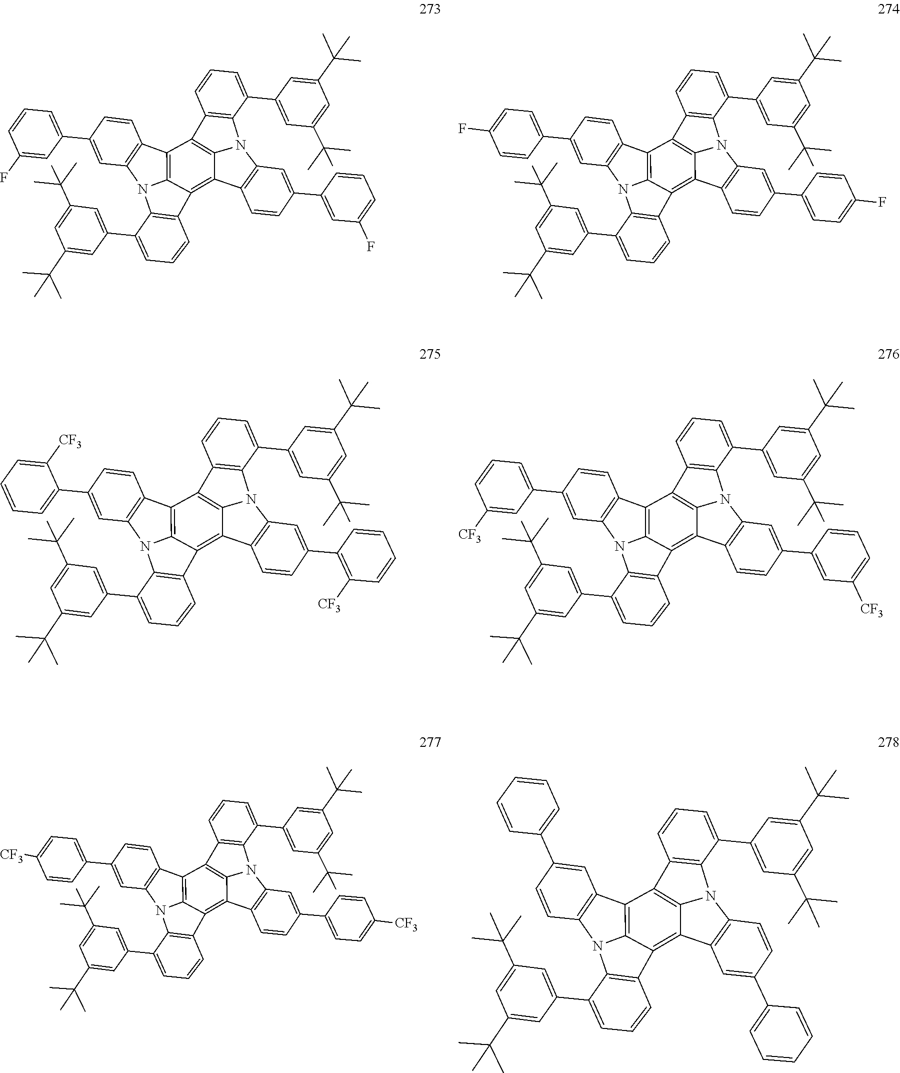

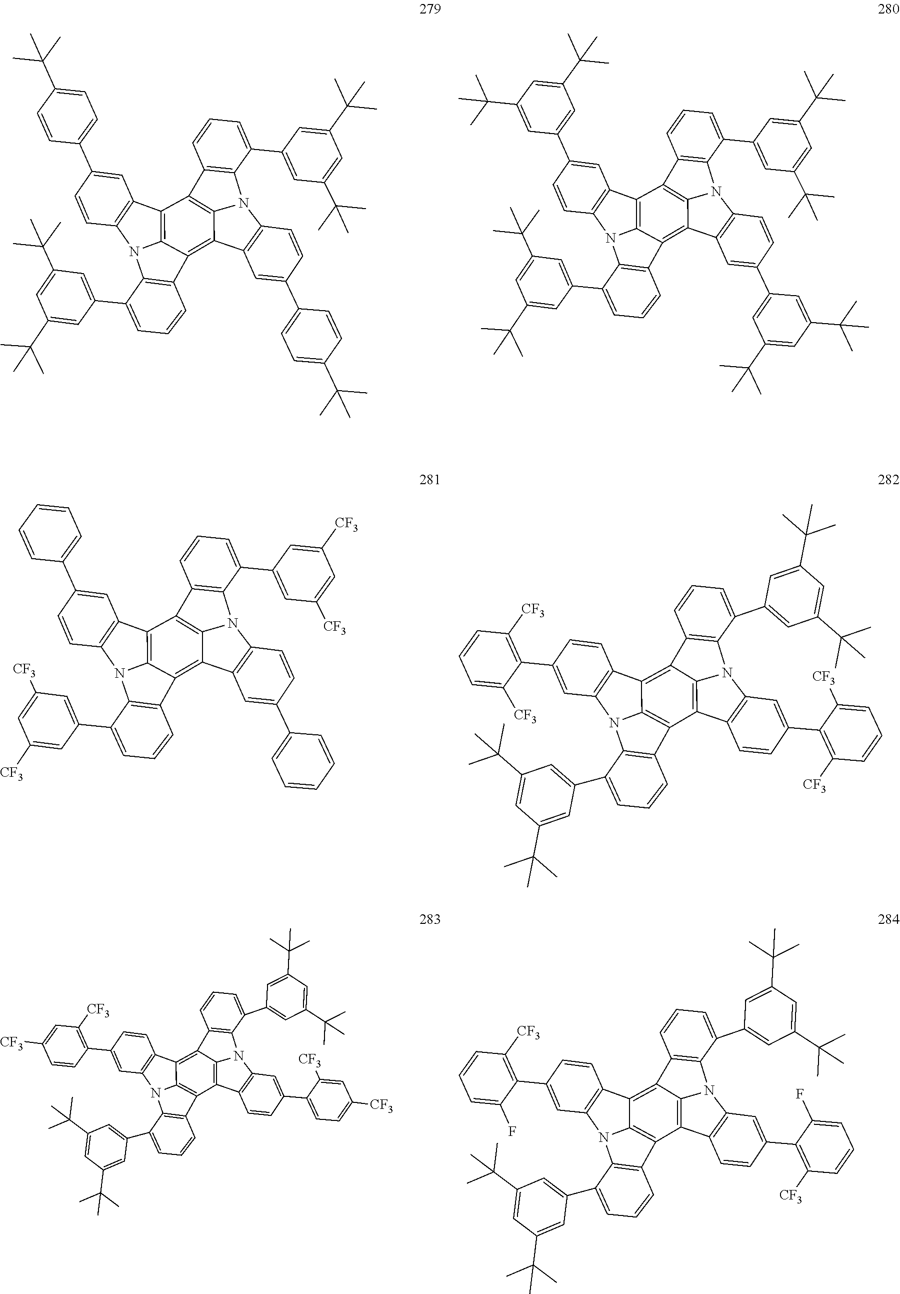

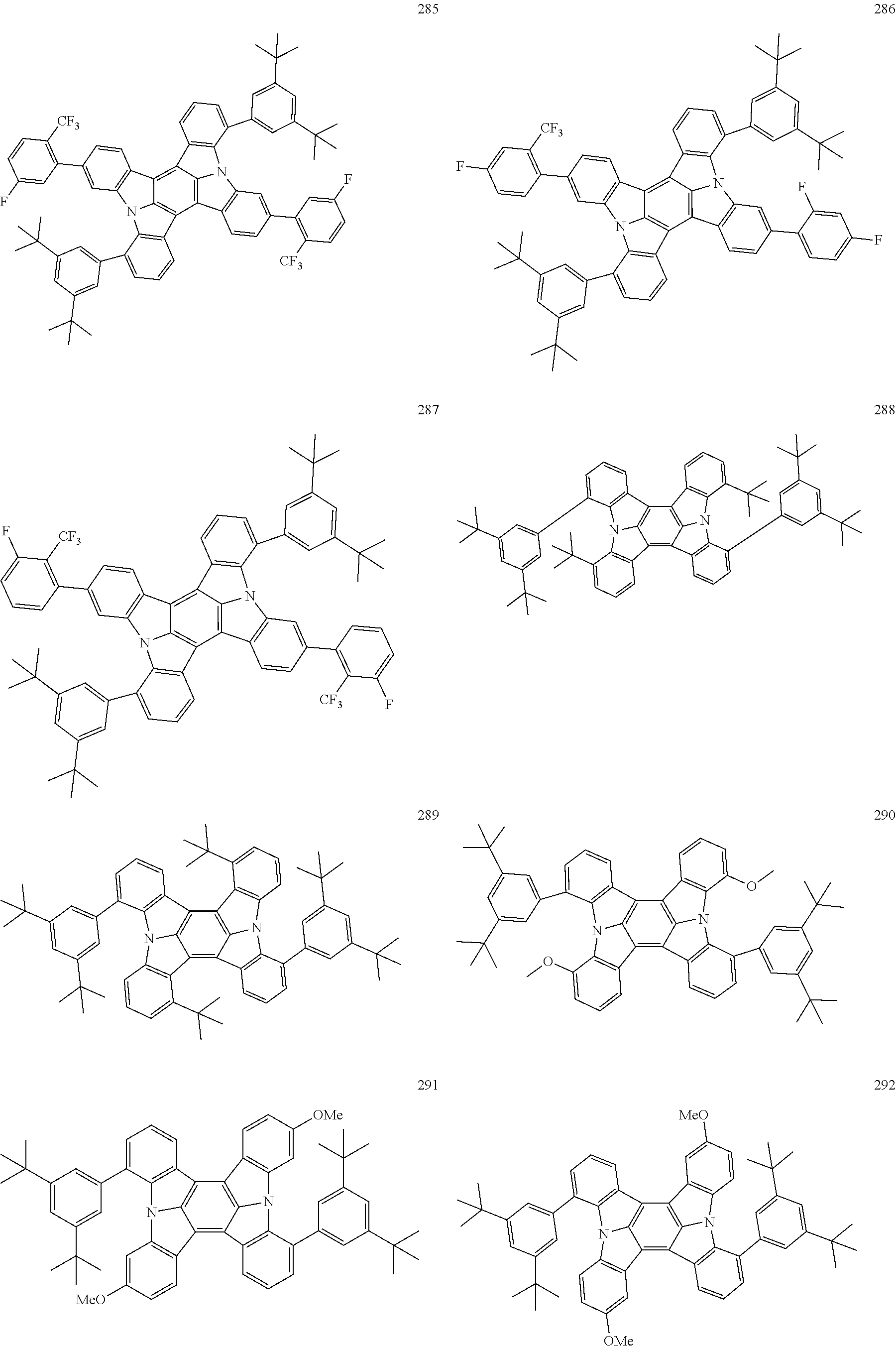

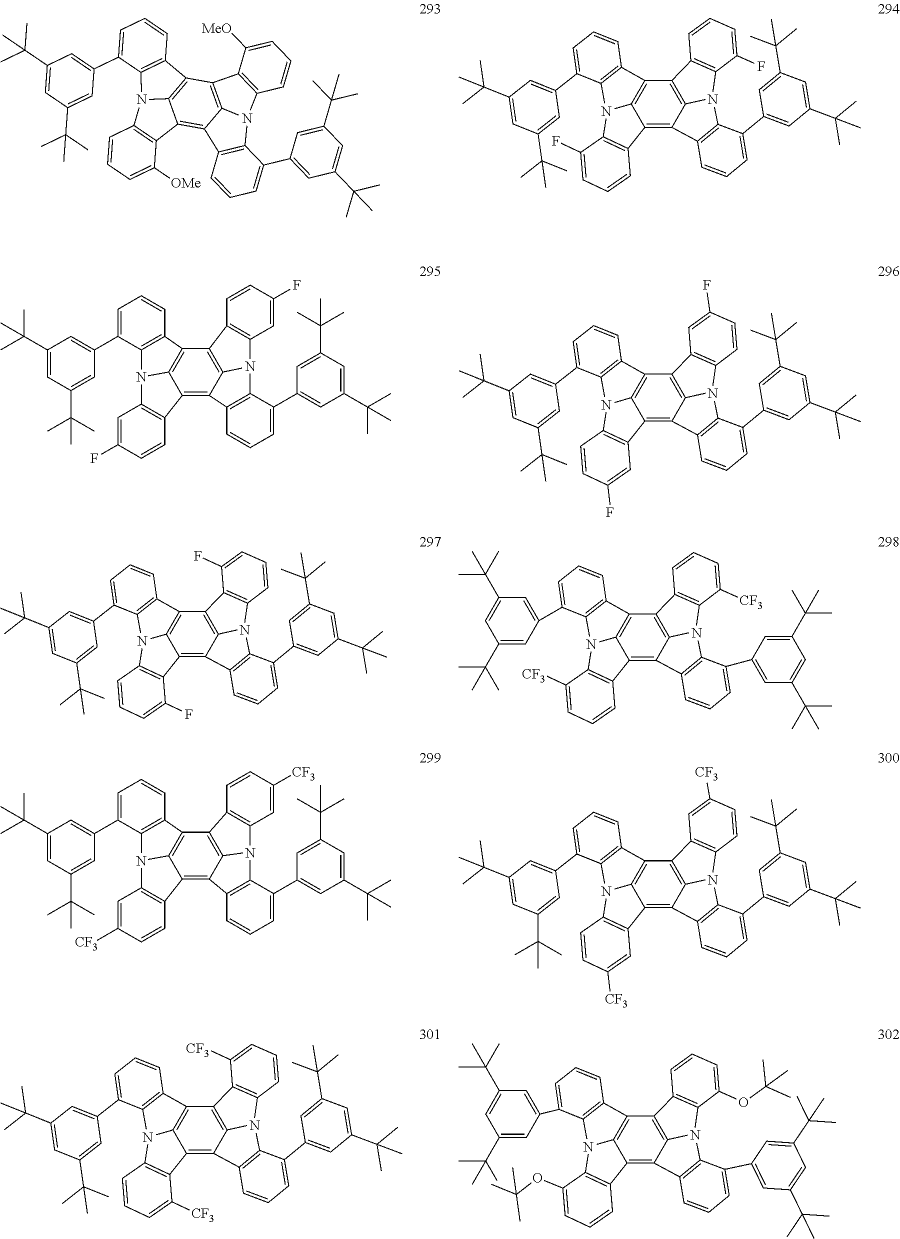

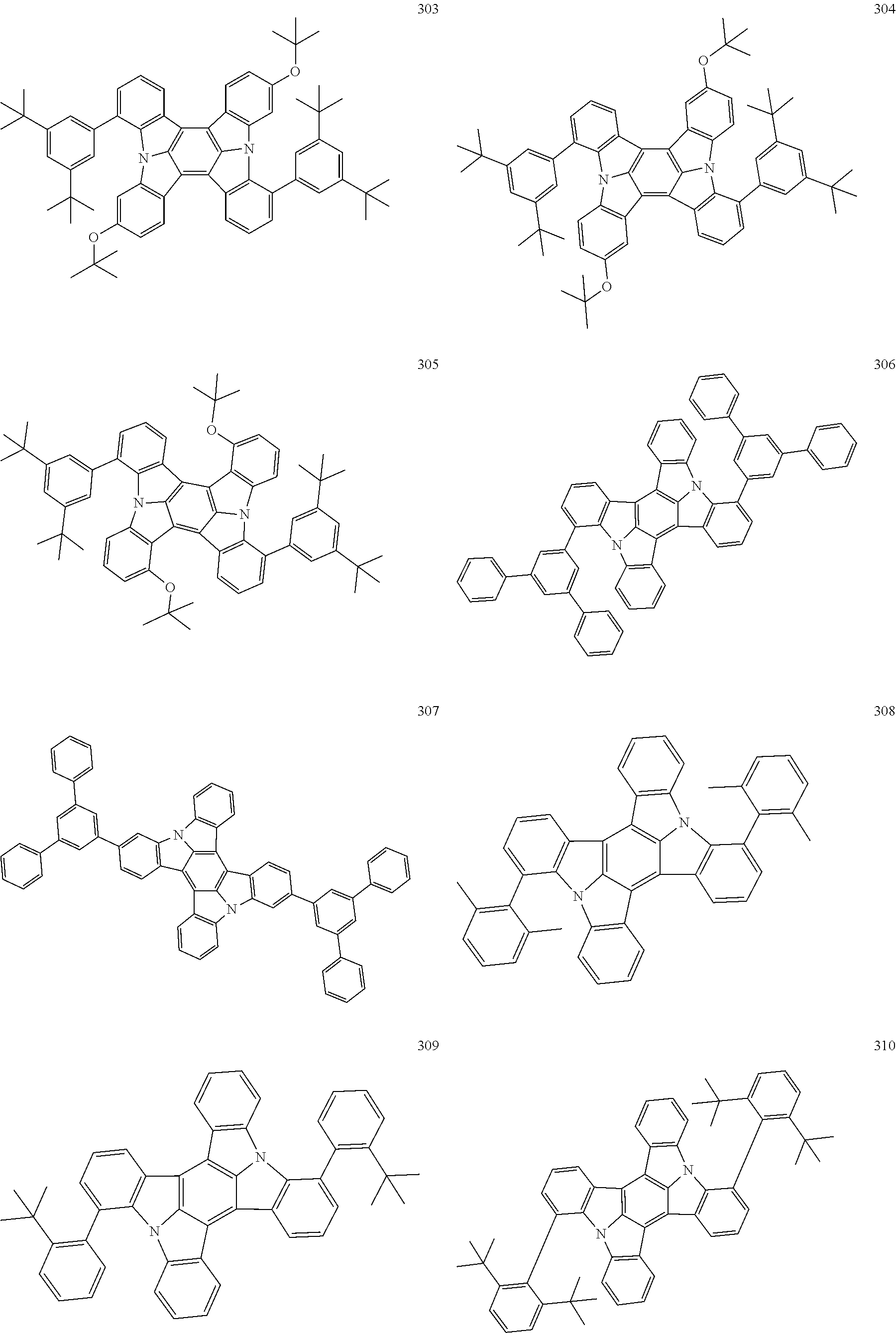

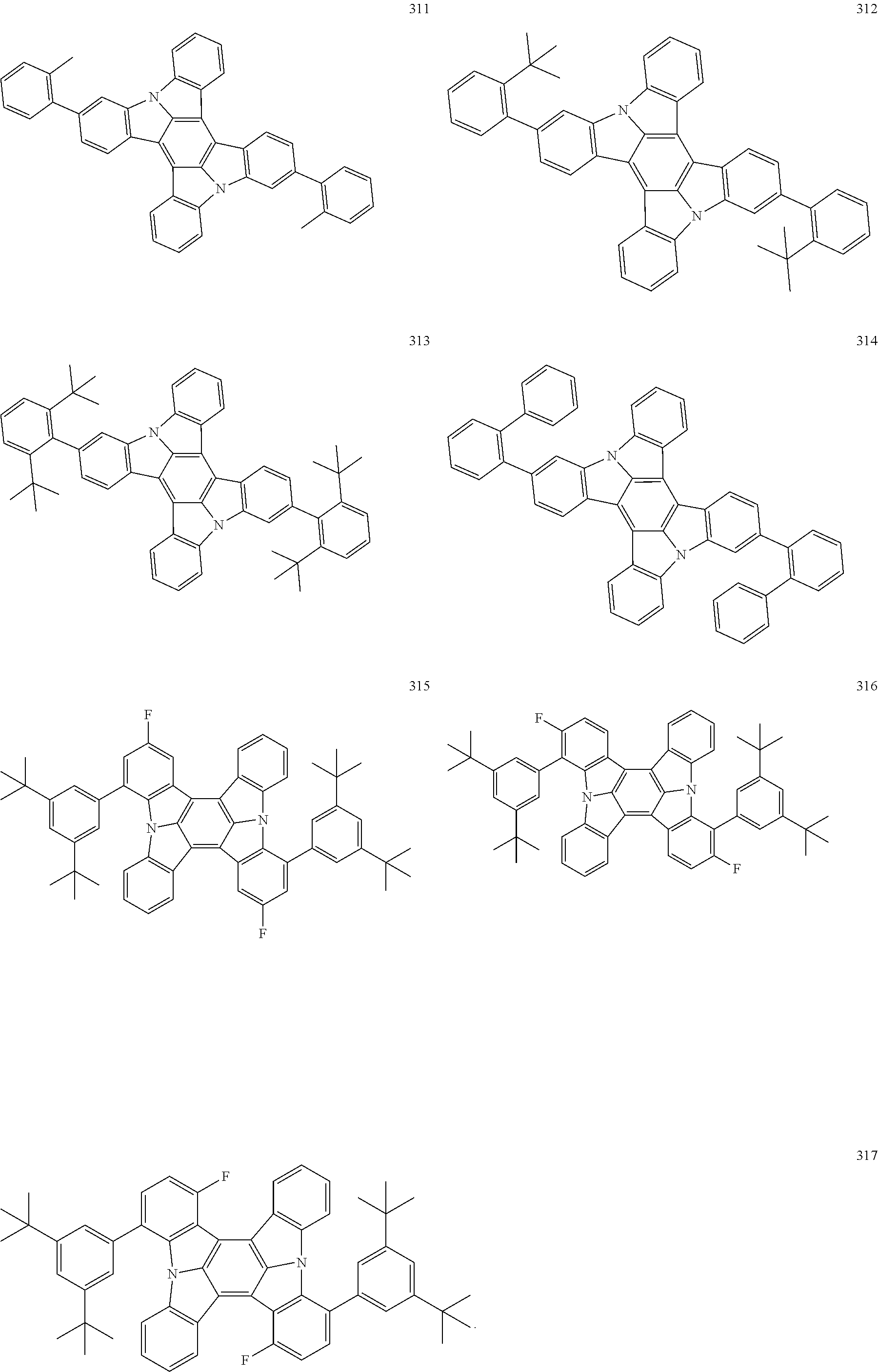

Examples of the nitrogen-containing condensed cyclic compound include Compounds 1 to 3, 5 to 10, 12 to 14, 18, 19, 65, 66, 71, 75, 76, 79 to 81, 84, 85, 89, 114, 156, 160, 172, 194, and 195. Examples of the nitrogen-containing condensed cyclic compound include Compounds 6, 7, 10, 12 to 14, 18, 89, 114, 156, 160, 172, 194, and 195. Examples of the nitrogen-containing condensed cyclic compound include Compounds 6, 7, 10, 12 to 14, 18, 114, and 195. Examples of the nitrogen-containing condensed cyclic compound include Compounds 6, 7, 10, 12 to 14, and 114. Examples of the nitrogen-containing condensed cyclic compound include Compounds 7, 10, 12 to 14, and 114.

Synthesizing a nitrogen-containing condensed cyclic compound according to the present disclosure may be performed by the known synthesis method, but is not limited thereto. More specifically, synthesizing is possible according to the methods described in the embodiments. For example, synthesizing is possible by changing the raw material, the reaction condition, and the like of, add a process to or delete a process from, or suitably combine the known synthesis method to the method described in the examples.

A method of identifying the structure of the nitrogen-containing condensed cyclic compound according to the present disclosure is not particularly limited. The structure of the nitrogen-containing condensed cyclic compound according to the present disclosure may be identified by the known method (for example, NMR, LC-MS, and the like).

### Fluorescence Emitters

An embodiment of the present disclosure relates to a fluorescence emitter which comprises the nitrogen-containing condensed cyclic compound and is used together with the phosphorescent complex to be described later. Because the fluorescence emitter is used together with the phosphorescent complex, emission efficiency and device lifespan may be significantly improved. The reason is assumed as below. The phosphorescent complex transfers energy to the nitrogen-containing condensed cyclic compound by a fluorescence resonance energy transfer (FRET) mechanism. As a result, energy may be transferred with high efficiency from the phosphorescent complex to the nitrogen-containing condensed cyclic compound.

In the fluorescence emitter according to the present disclosure, the peak wavelength of the emission spectrum may be light emitted within the blue wavelength region.

Details of the phosphorescent complex will be described later. The fluorescence emitter may be used with a host material to be described later. Details of the host material will be described later.

### Material for Organic Electroluminescent Device

Another embodiment of the present disclosure relates to a material for an organic electroluminescent device including the nitrogen-containing condensed cyclic compound. The material may be a material for an emission layer.

In an embodiment, the material for the organic electroluminescent device may include other materials used in the nitrogen-containing condensed cyclic compound and the organic electroluminescent device. Materials used in an organic electroluminescent device may be, but not limited to, a phosphorescent compound or a host material. The materials may also be a phosphorescent complex and a host material. Here, the nitrogen-containing condensed cyclic compound may be used as a dopant material, and the phosphorescent complex may be used as an auxiliary dopant. Because both the nitrogen-containing condensed cyclic compound and the phosphorescent complex or the host material (or the phosphorescent complex and the host material) are used, emission efficiency and device lifespan may be significantly improved. The reason is assumed as below. When the material for the organic electroluminescent device contains the host material, the phosphorescent complex receives energy from the host material. In addition, the phosphorescent complex transfers energy to the nitrogen-containing condensed cyclic compound by a fluorescence resonance energy transfer (FRET) mechanism. As a result, energy may be transferred with high efficiency from the phosphorescent complex to the nitrogen-containing condensed cyclic compound. Other materials known in the art may be used for the organic electroluminescent device.

An amount of the nitrogen-containing condensed cyclic compound may be, but not limited to, 0.05 wt % or more based on the total weight of the material for the organic electroluminescent device (particularly the material for the emission layer). The amount may be 0.1 wt % or more, or 0.2 wt % or more. Within this range, an organic electroluminescent device having excellent luminescence color purity and high emission efficiency may be obtained. An amount of the nitrogen-containing condensed cyclic compound relative to the total weight of the material for the organic electroluminescent device (particularly, the material for the emission layer) may be, but not limited to, 50 wt % or less. The amount may be 30 wt % or less, or 25 wt % or less. Within this range, an organic electroluminescent device having excellent luminescence color purity and high emission efficiency may be obtained. An amount of the nitrogen-containing condensed cyclic compound relative to the total weight of the emission layer of the organic electroluminescent device to be described later may be the same as above.

### Phosphorescent Complex

In an embodiment, the material for an organic electroluminescent device may include a phosphorescent complex in addition to the nitrogen-containing condensed cyclic compound. Because a phosphorescent complex is added, emission efficiency and device lifespan may significantly be improved. The reason for such improvement may be because, as described above regarding the fluorescence emitter, energy may be transferred with high efficiency from the phosphorescent complex to the nitrogen-containing condensed cyclic compound.

The phosphorescent complex may be, but not limited to, a metal complex in view of emission efficiency. The phosphorescent complex may be a platinum complex or a palladium complex, or may a platinum complex, from the same point of view. In an embodiment of the material for the organic electroluminescent device, the phosphorescent complex may be a platinum complex.

The phosphorescent complex may be, but not limited to, a compound having the structure of Formula (4), in view of luminescence color purity and emission efficiency.

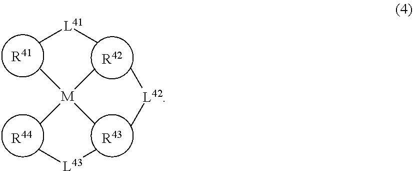

In Formula (4), M may be a metal ion having a coordination number of 4,

R⁴¹, R⁴², R⁴³, and R⁴⁴ may each independently be a substituted or unsubstituted cyclic hydrocarbon group or a substituted or unsubstituted heterocyclic group,

L⁴¹ may be a linking group linking R⁴¹ and R⁴²,

L⁴² may be a linking group linking R⁴² and R⁴³, and

L⁴³ may be a linking group linking R⁴³ and R⁴⁴.

In Formula (4), the cyclic hydrocarbon group refers to a group derived from at least one hydrocarbon ring. When the cyclic hydrocarbon group includes at least two hydrocarbon rings, parts of the rings or the rings as a whole may be linked by a single bond or condensed with each other. Further, when the cyclic hydrocarbon group includes two or more hydrocarbon rings, one atom may act as a ring-forming atom of such rings.

A heterocyclic group in Formula (4) is identical to the monovalent heterocyclic group described in (g) in Formula (1), except for having a different valence number.

In Formula (4), the substituent substituting the cyclic hydrocarbon group or the heterocyclic group may be, but not limited to, a substituent substituting the groups of (c) to (g) in Formula (1).

In Formula (4), M may be a platinum ion or a palladium ion, for example, a Pt ion.

Other known compounds may be used as the phosphorescent complex. For example, platinum complex described in Tyler Fleetham et al. Efficient “Pure” Blue OLEDs Employing Tetradentate Pt Complexes with a Narrow Spectral Bandwidth, Advanced Materials, 2014, 26, 7116-7121, the platinum complex described in European Patent Application Publication no. 3670520, the platinum complex and palladium complex described in Japanese Patent Application Publication no. 2019-029500, and the platinum complex described in US Patent Application Publication no. 2015/0162552 may be used.

Hereinafter, the phosphorescent complex according to an embodiment of the present disclosure will be explained in detail. The present disclosure is not limited to these examples:

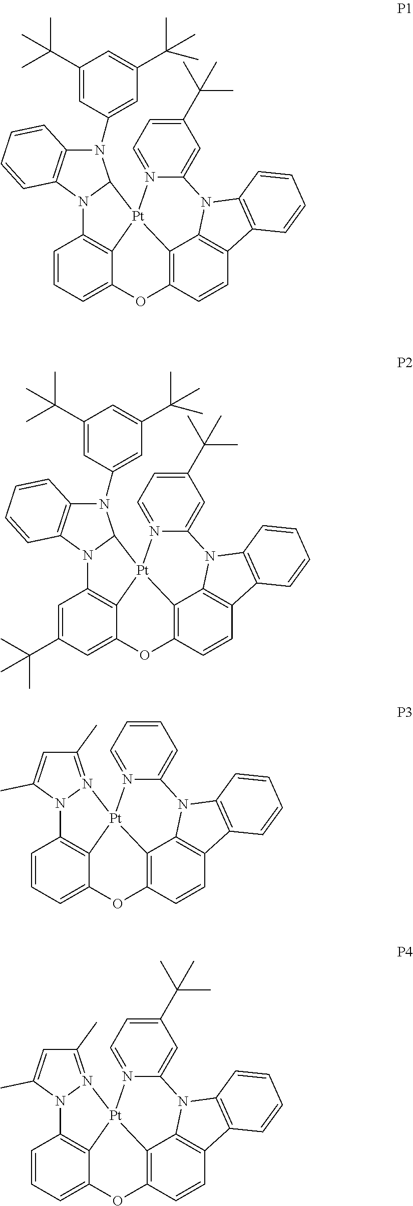

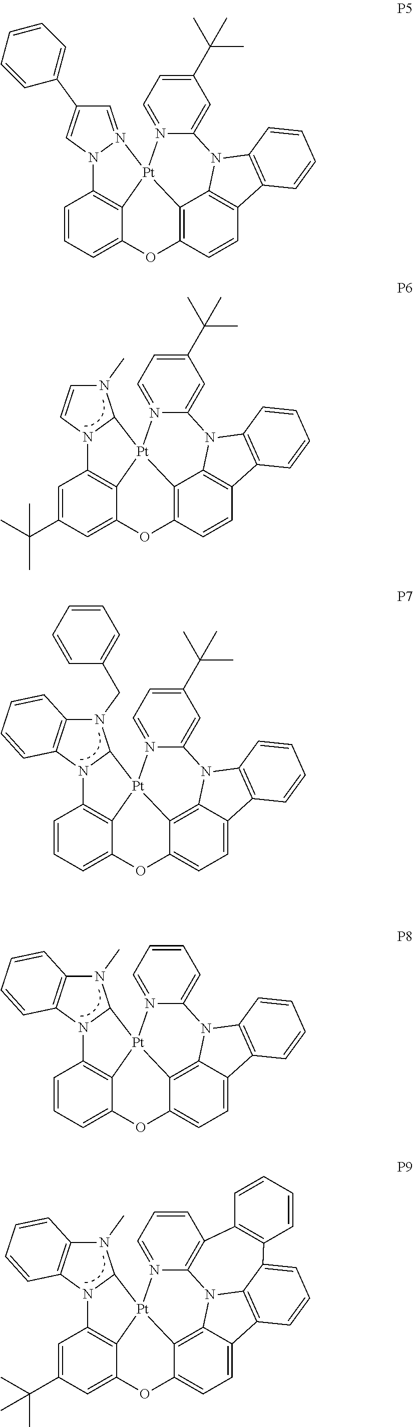

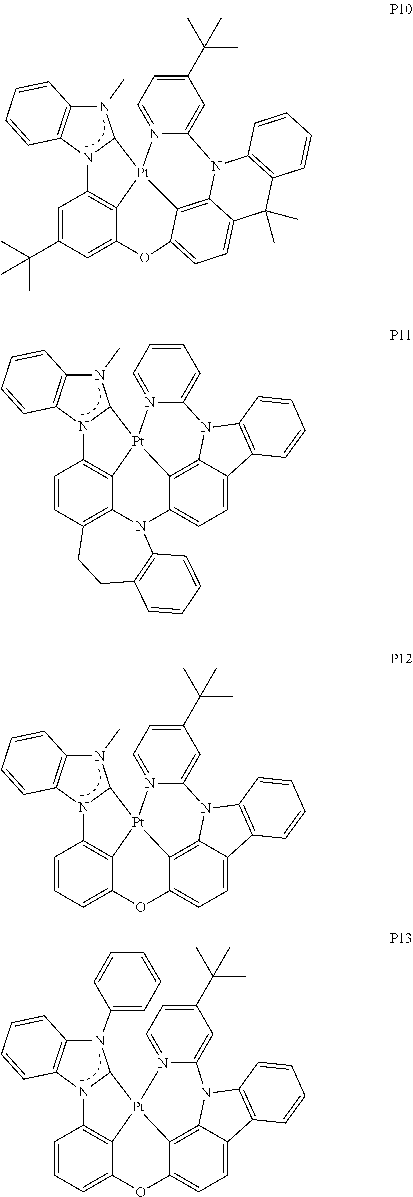

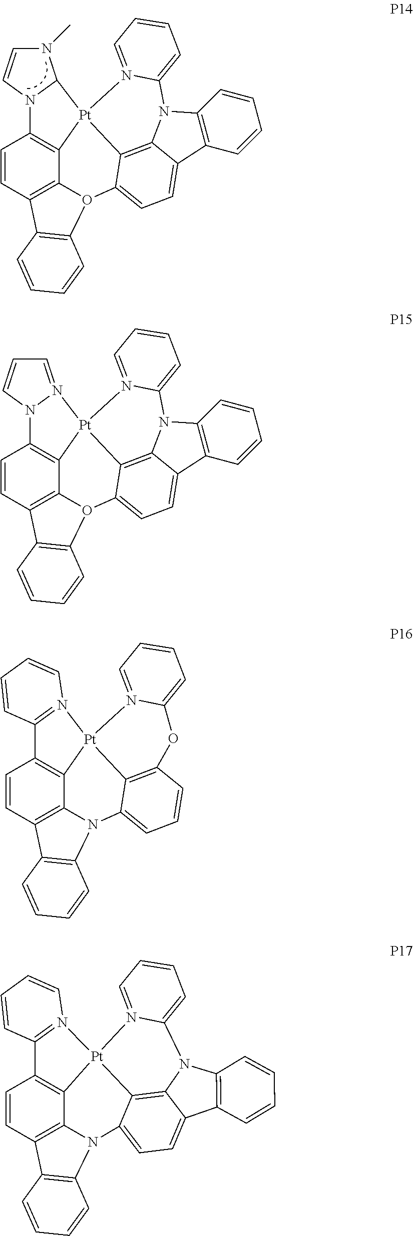

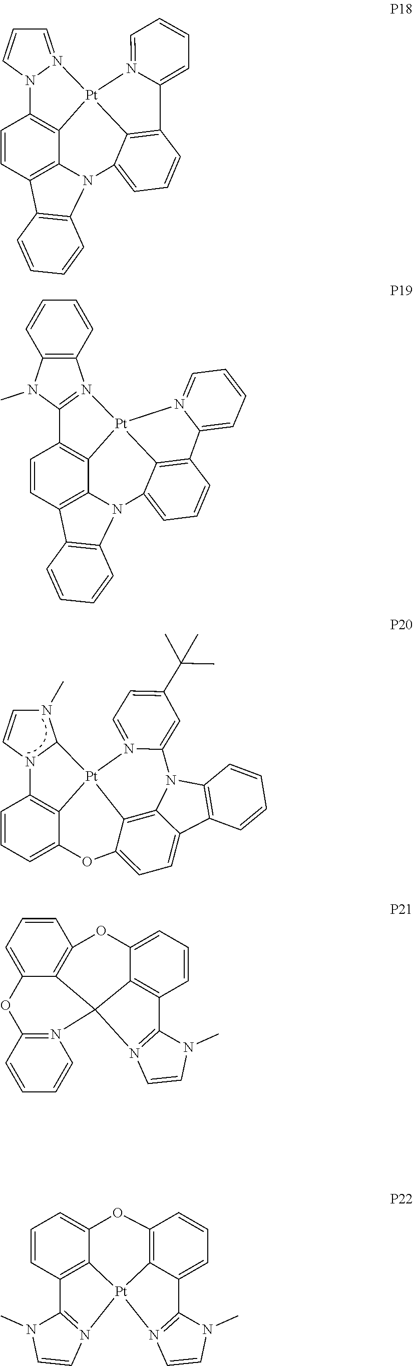

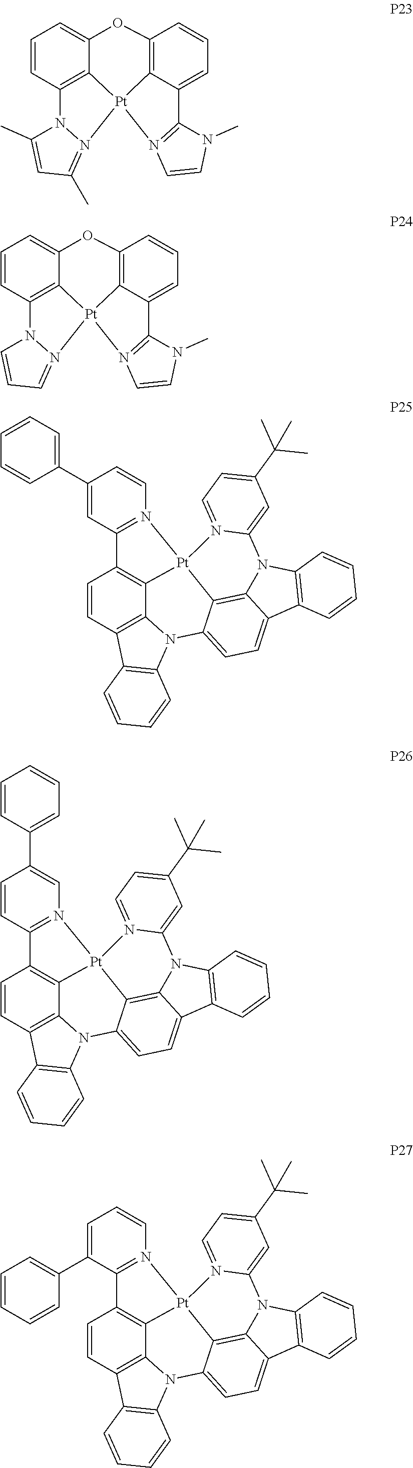

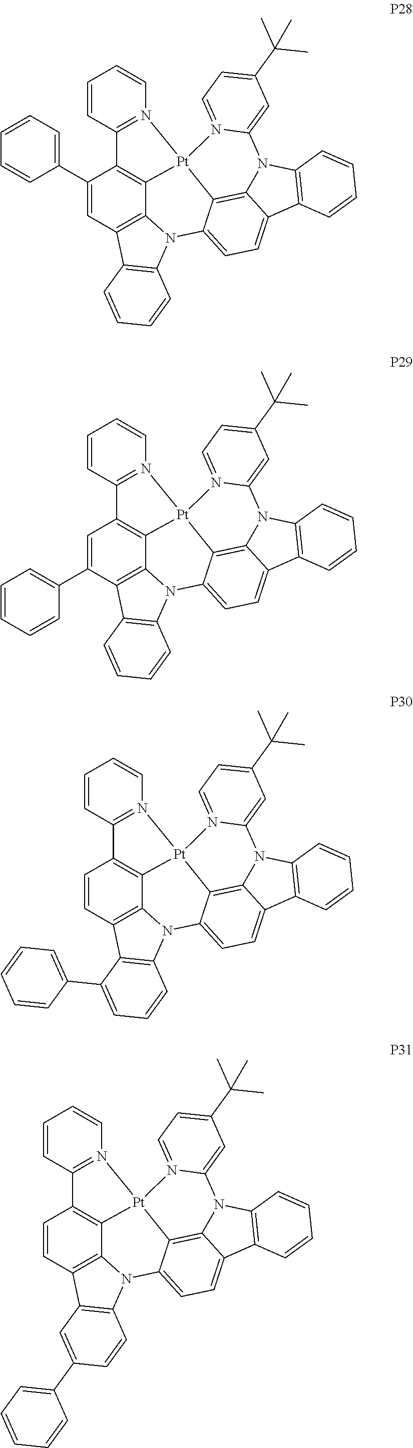

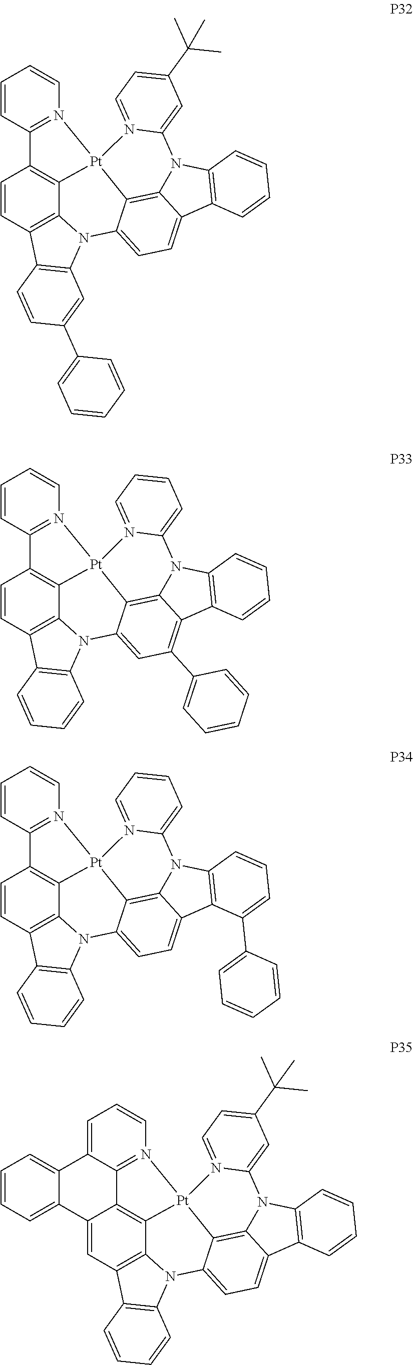

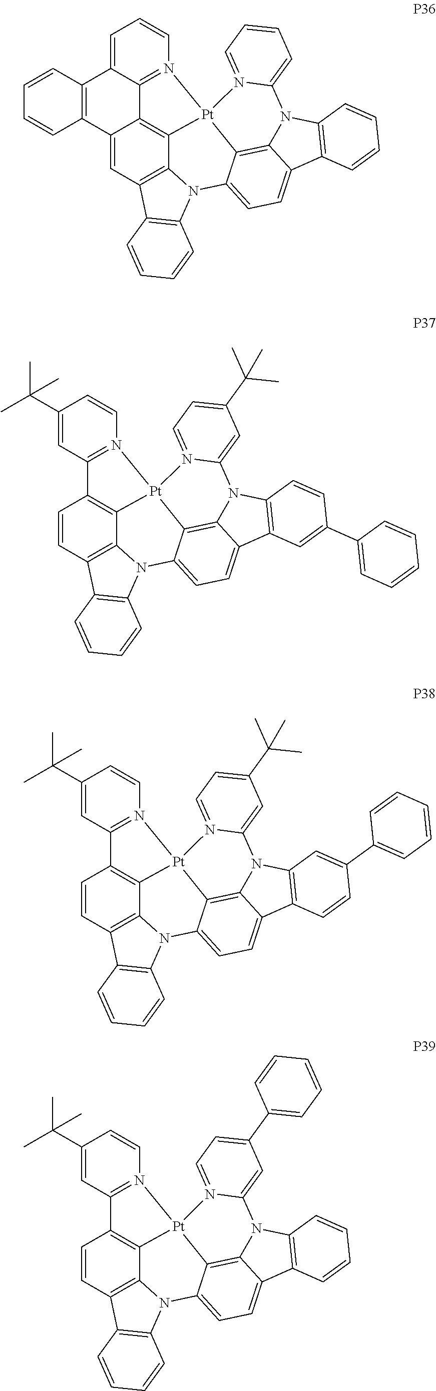

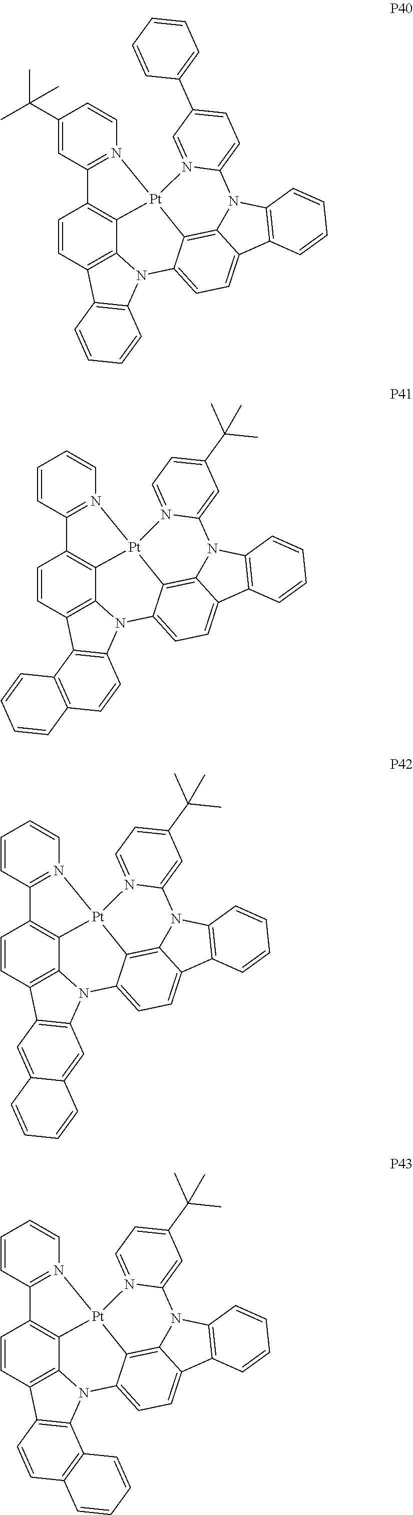

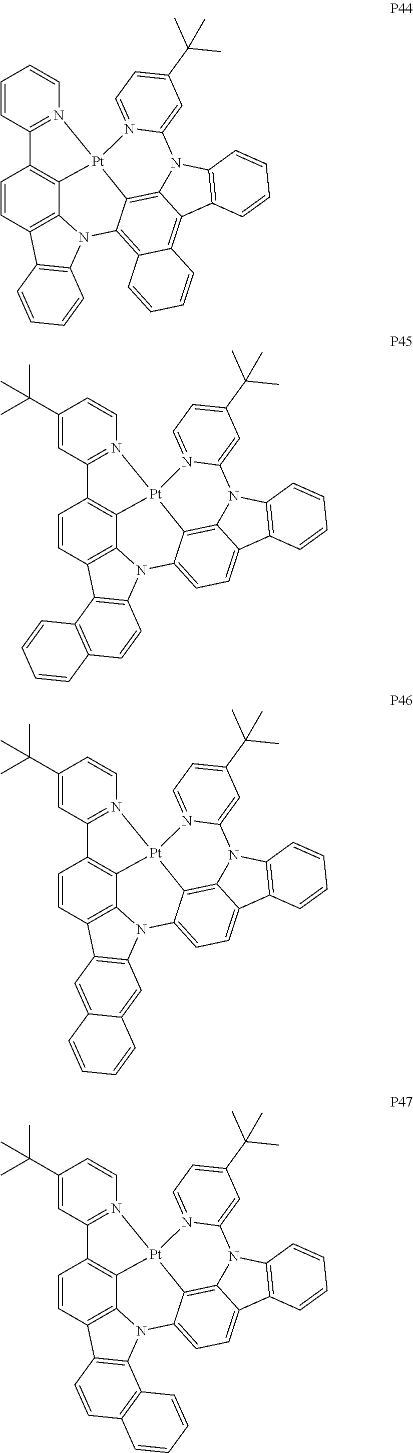

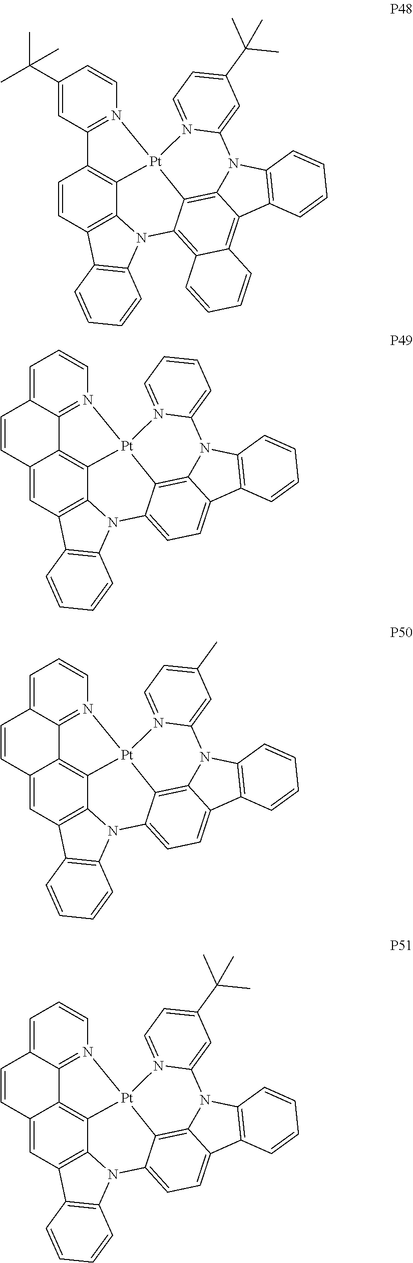

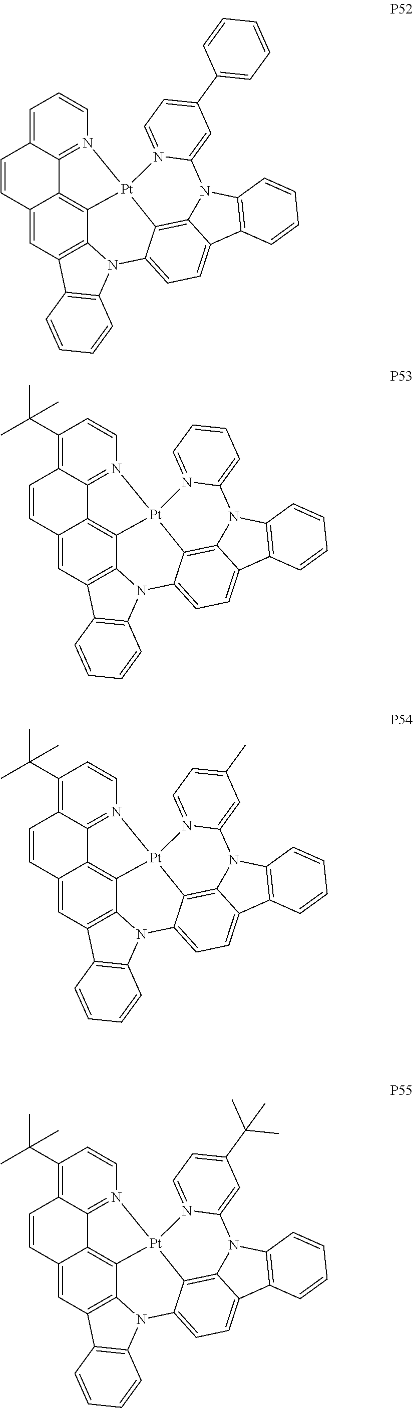

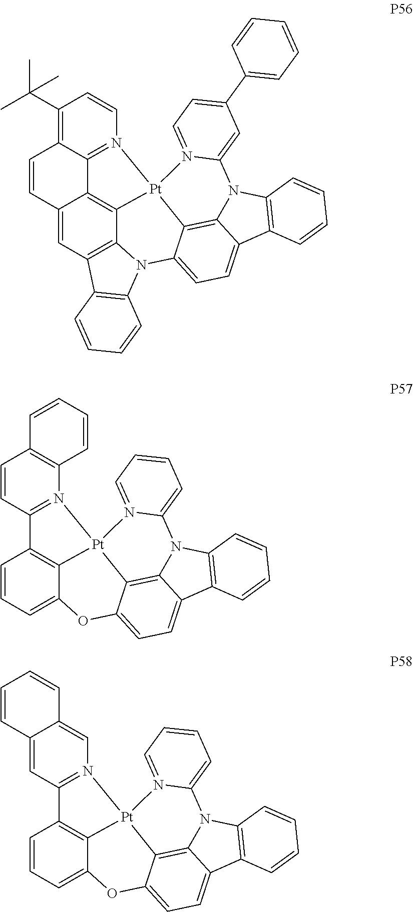

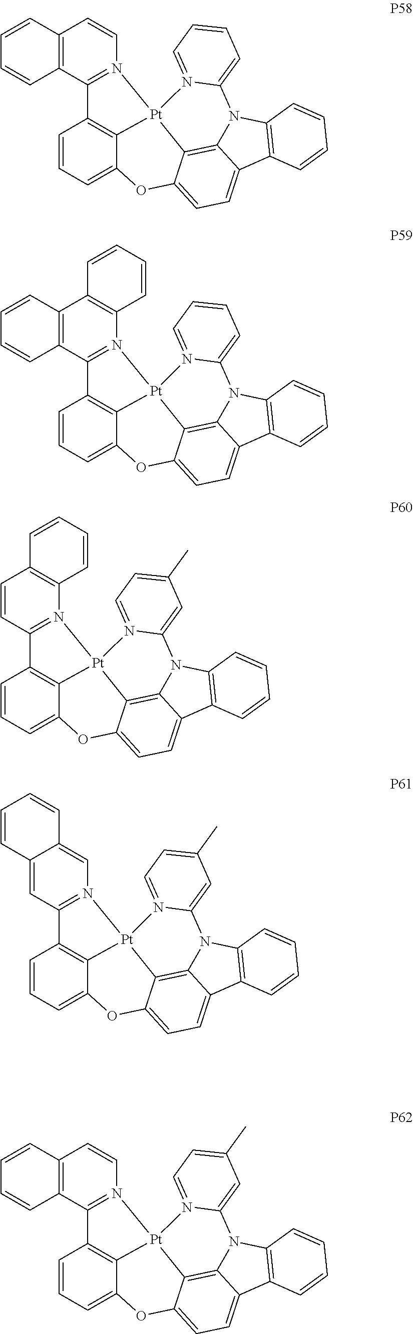

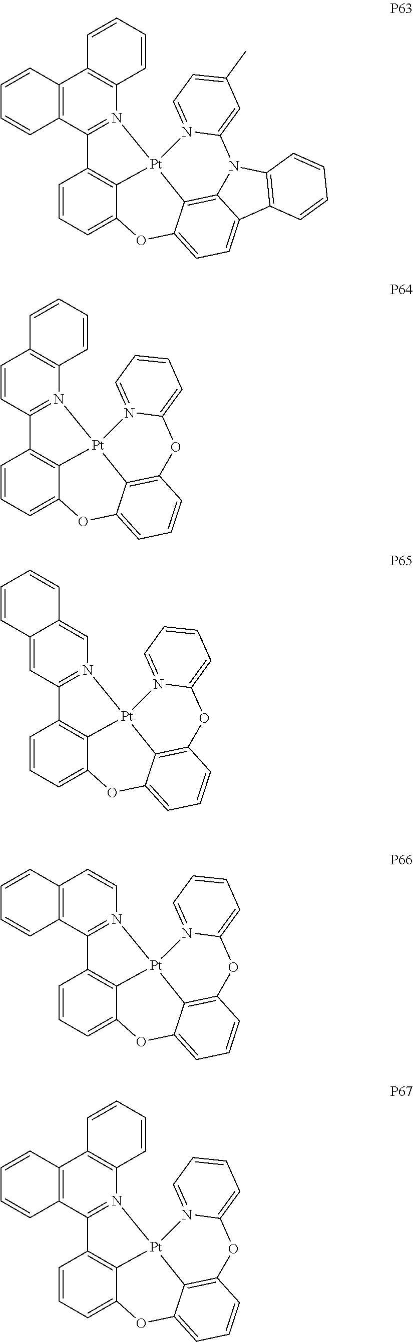

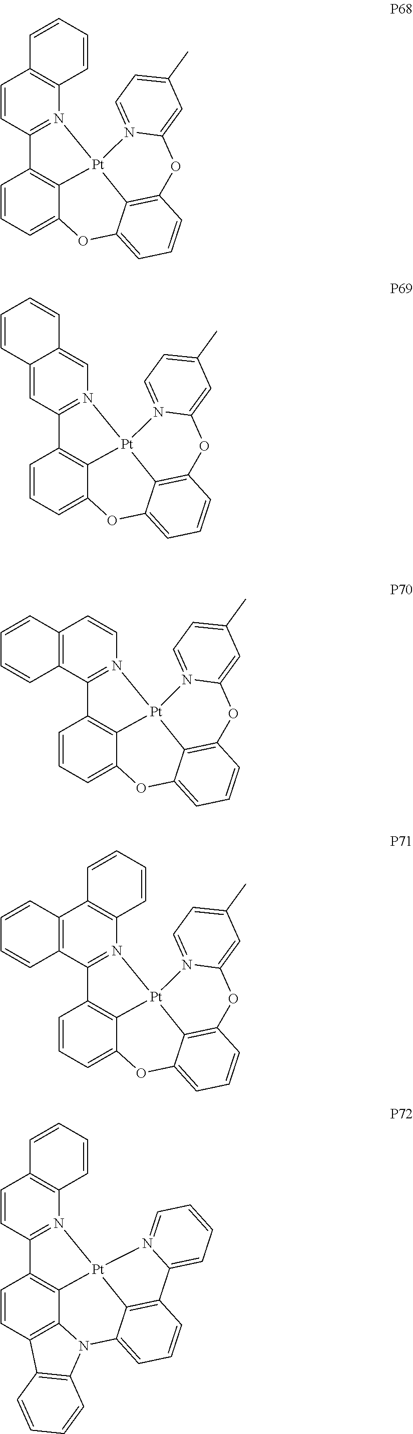

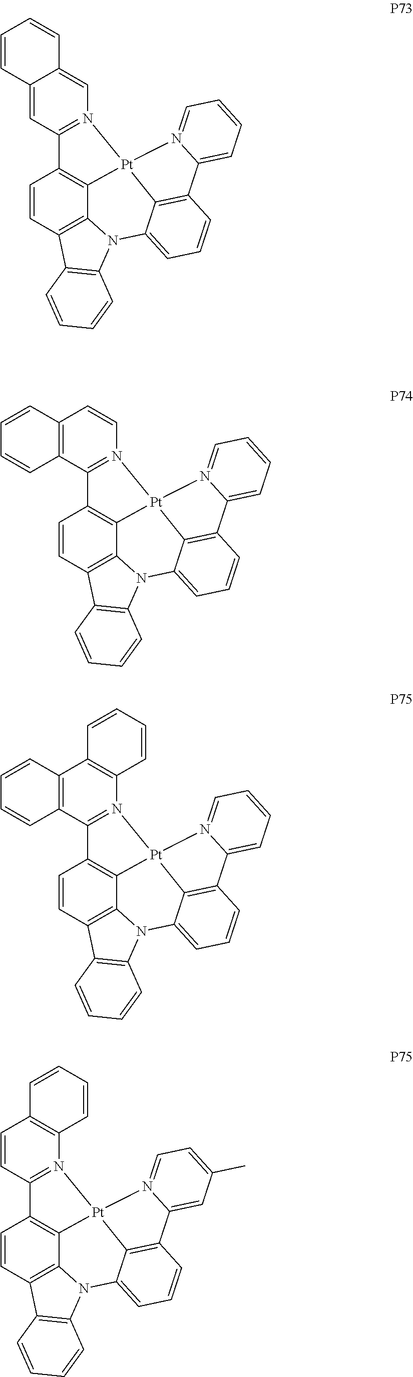

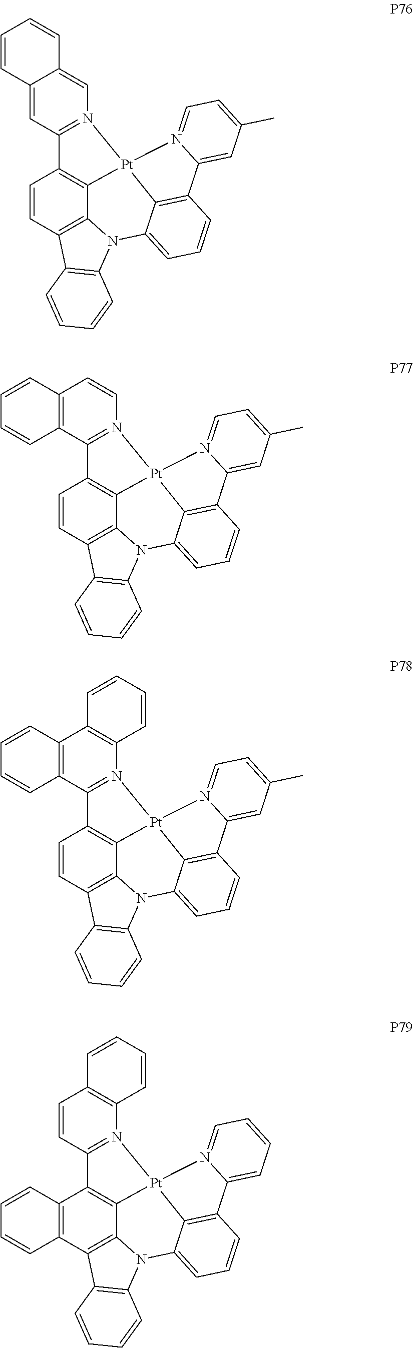

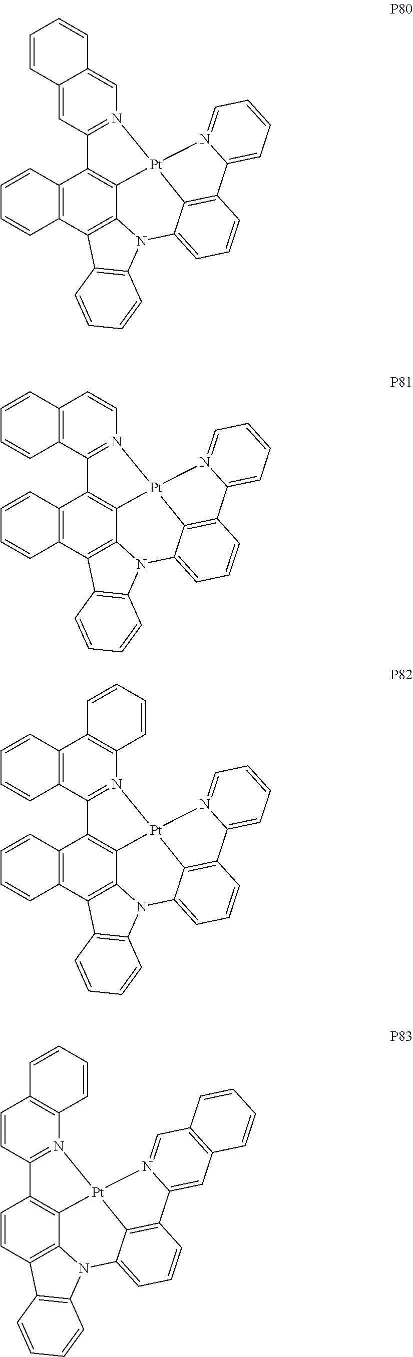

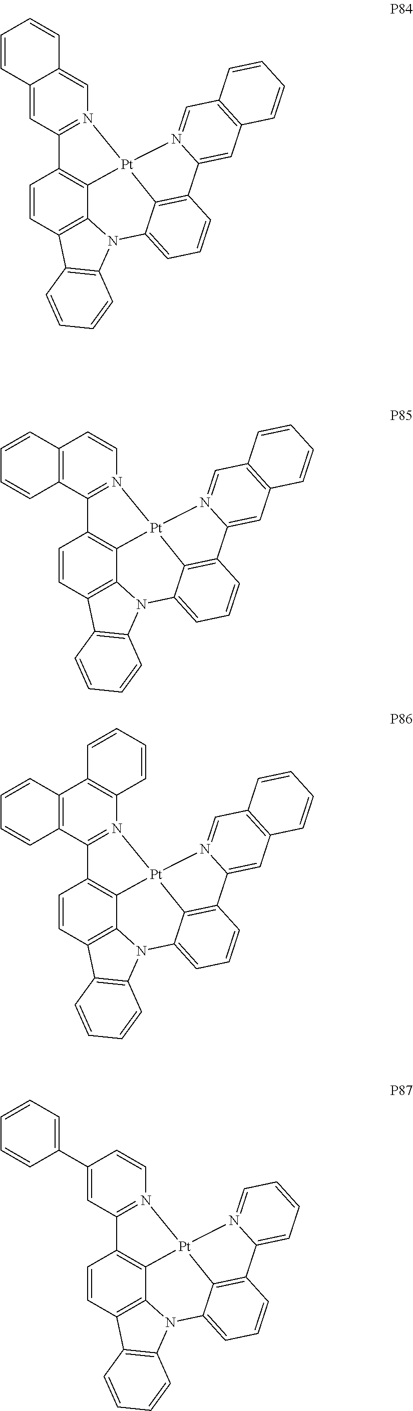

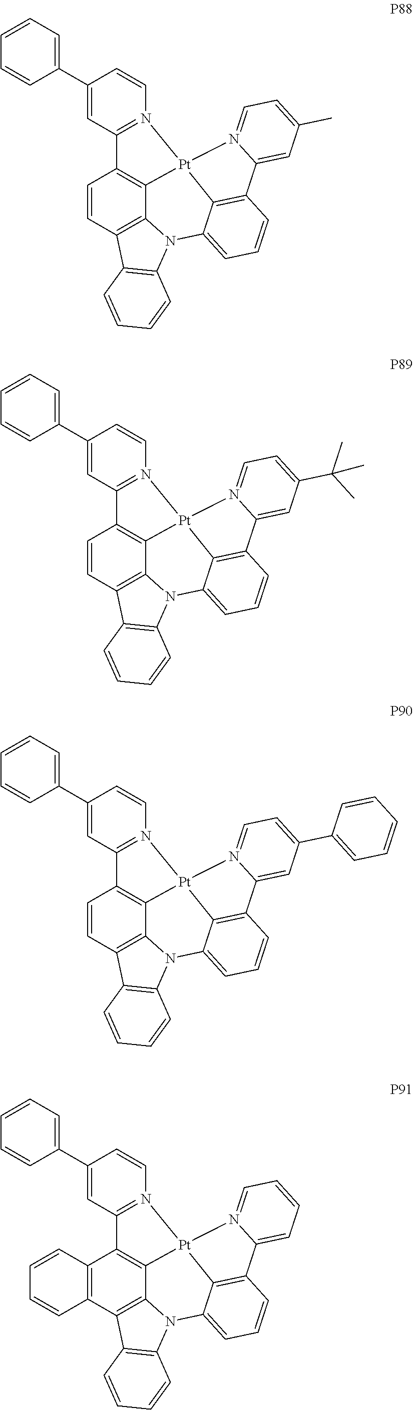

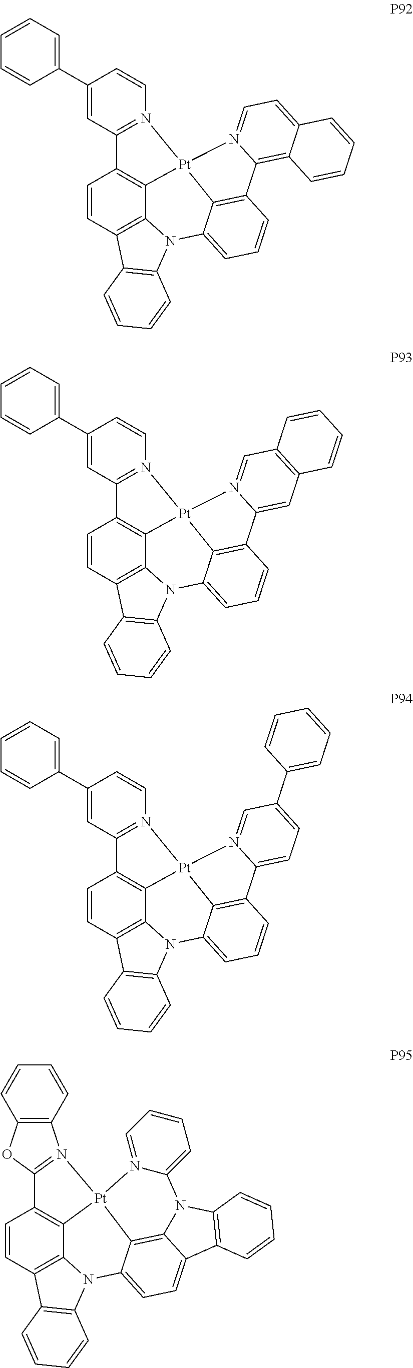

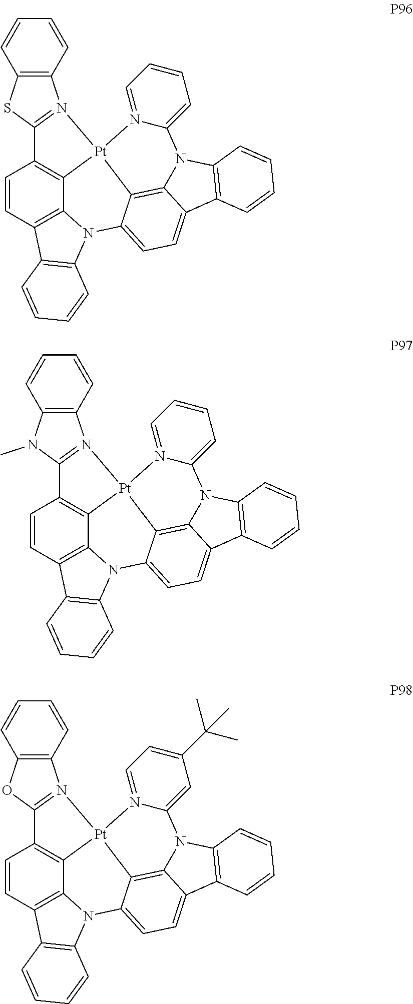

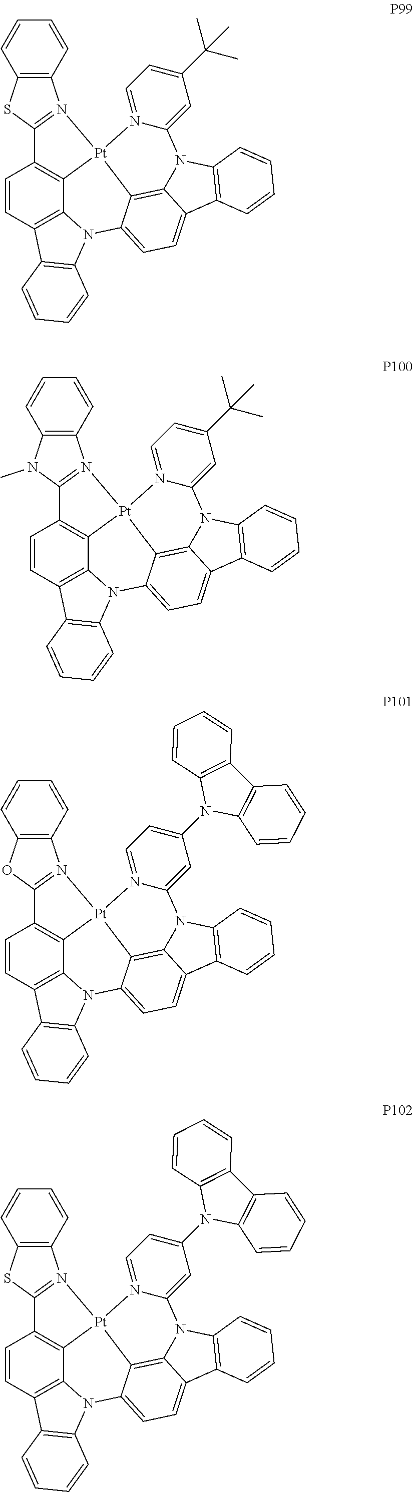

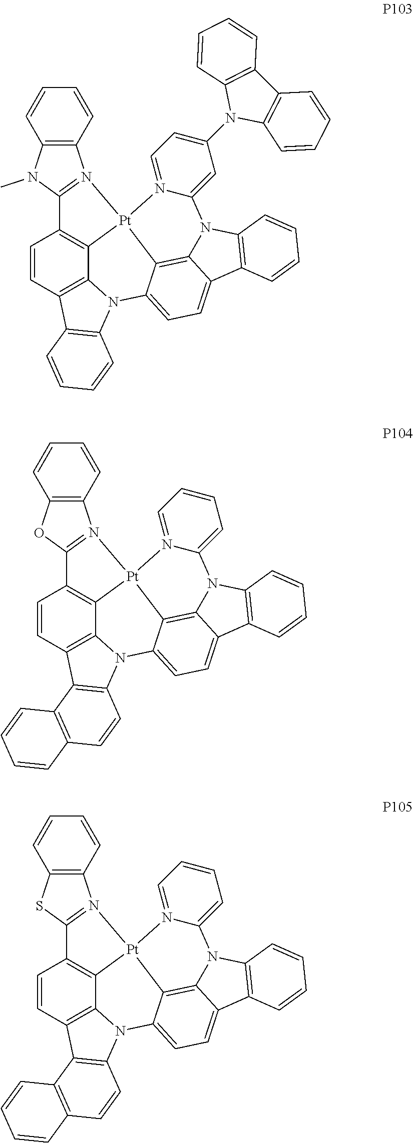

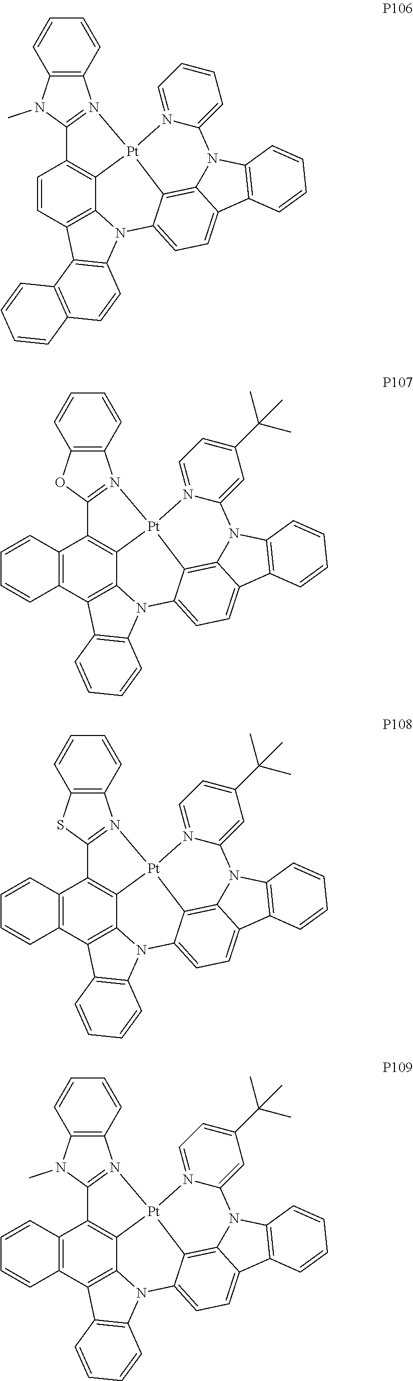

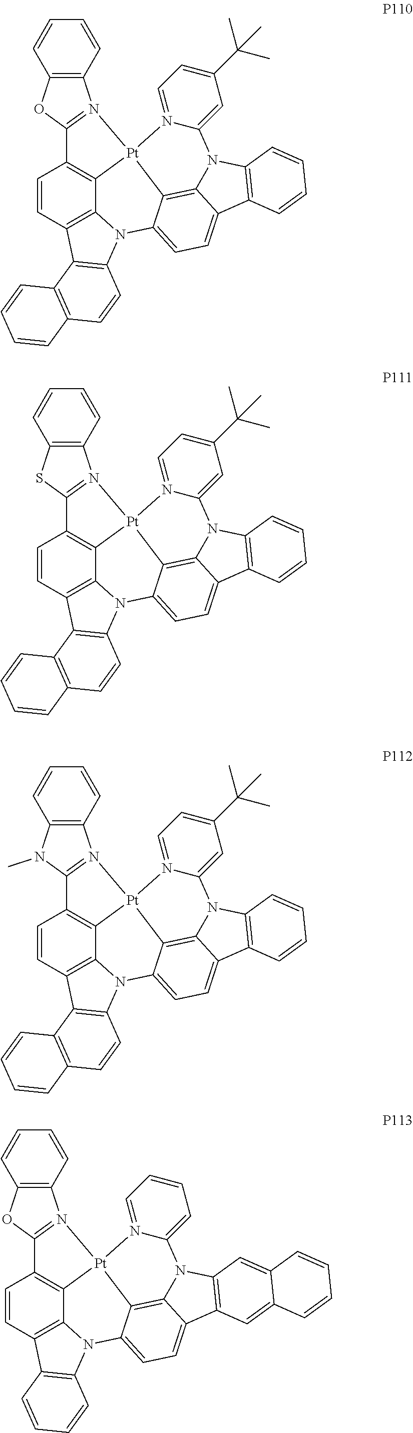

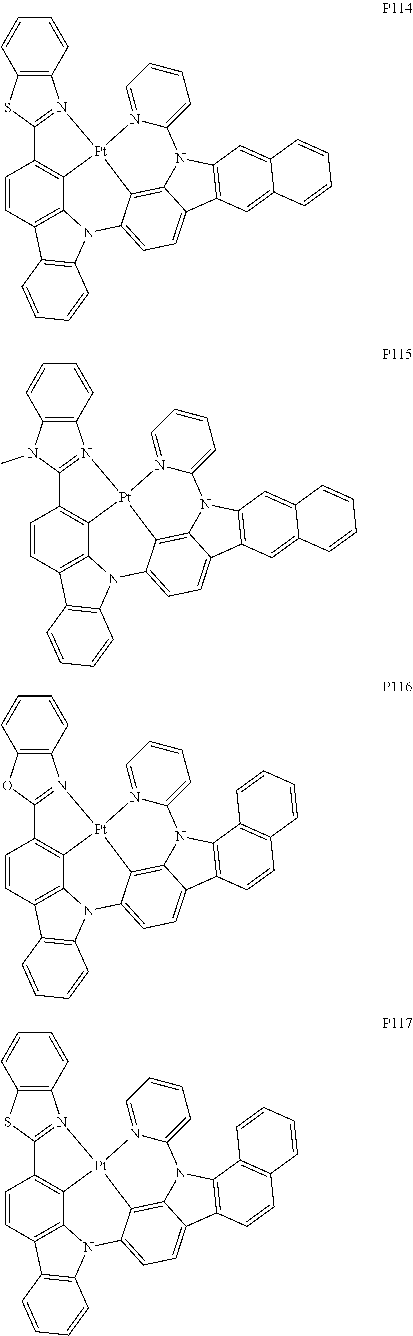

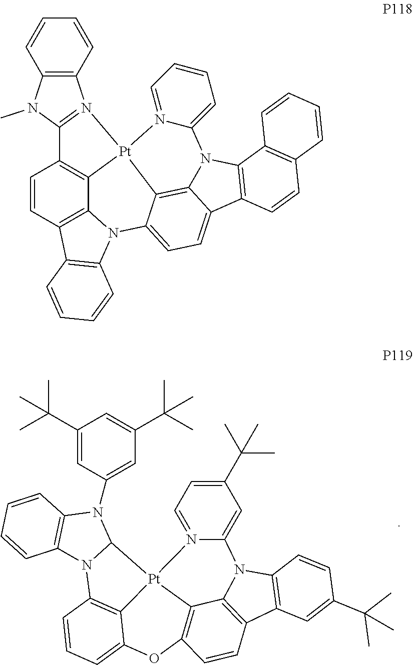

An amount of the phosphorescent complex may be, but not limited to, 0.1 wt % or more, or 0.2 wt % or more, based on the total weight of the material for the organic electroluminescent device (particularly the material for the emission layer). The amount may be 0.5 wt % or more, or 1 wt % or more. The amount may be 3 wt % or more, or 5 wt % or more. Within this range, an organic electroluminescent device having excellent luminescence color purity and high emission efficiency may be obtained. An amount of the phosphorescent complex may be, but not limited to, 50 wt % or less based on the total weight of the material for the organic electroluminescent device (particularly the material for the emission layer). The amount may be 40 wt % or less, or 30 wt % or less. Within this range, an organic electroluminescent device having excellent luminescence color purity and high emission efficiency may be obtained. An amount of the phosphorescent complex relative to the total weight of the emission layer of the organic electroluminescent device to be described later may be the same as above.

When the material for the organic electroluminescent device (particularly the material for the emission layer) includes the phosphorescent complex, the amount of the phosphorescent complex may be 100 parts by weight or more based on 100 parts by weight of the nitrogen-containing condensed cyclic compound. The amount may be, based on 100 parts by weight of the nitrogen-containing condensed cyclic compound, 150 parts by weight or more, or 200 parts by weight or more. Within this range, an organic electroluminescent device having excellent luminescence color purity and high emission efficiency may be obtained. The amount of the phosphorescent complex may be, but not limited to, 10,000 parts by weight or less based on 100 parts by weight of the nitrogen-containing condensed cyclic compound. The amount may be, based on 100 parts by weight of the nitrogen-containing condensed cyclic compound, 7,500 parts by weight or less, or 5,000 parts by weight or less. Within this range, an organic electroluminescent device having excellent luminescence color purity and high emission efficiency may be obtained. An amount (parts by weight) of the phosphorescent complex based on 100 parts by weight of the nitrogen-containing condensed cyclic compound in the emission layer of the organic electroluminescent device to be described later may be the same as above.

### Host Material

In an embodiment, the material for an organic electroluminescent device may include a host material in addition to the nitrogen-containing condensed cyclic compound. Because the nitrogen-containing condensed cyclic compound is used as a dopant material and the host material is used together in the organic electroluminescent device, excellent emission efficiency and device lifespan may be realized.

Other known host materials may be used, not being limited thereto. Preferred examples of the known host materials may include a compound having a carbazole ring structure (excluding compounds represented by Formula (1)), a compound having a ring structure wherein at least one ring-forming carbon atom of the carbazole ring is substituted into a nitrogen atom (excluding compounds represented by Formula (1) and the compound having the carbazole ring structure), or a compound having the triazine ring structure (excluding compounds represented by Formula (1), the compound having the carbazole ring structure, and the compound having a ring structure wherein at least one ring-forming carbon atom of the carbazole ring is substituted into a nitrogen atom). In an embodiment, the known host material may be a compound having a carbazole ring structure. Because such compounds are used as host materials, energy may be transferred with efficiency within the emission layer. In addition, the balance of mobility between the electrode and the hole may be improved. A hydrogen atom bonding with a ring-forming atom constituting a carbazole ring structure, a ring structure wherein at least one ring-forming carbon atom of the carbazole ring is substituted into a nitrogen atom, and a triazine ring structure among the above compound compounds may be substituted with other atoms or substituents. Two or more of such substituents may constitute a ring structure.

The compound having a carbazole ring structure or the compound having a ring structure wherein at least one ring-forming carbon atom of the carbazole ring is substituted into a nitrogen atom may have the structure of, but not limited to, Formula (5):

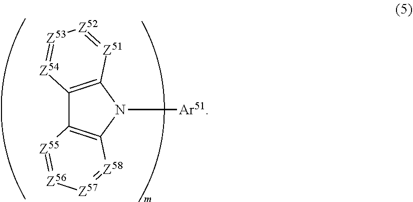

wherein, in Formula (5),

Z⁵¹ is CH, CR⁵¹, or N,

Z⁵² is CH, CR⁵², or N,

Z⁵³ is CH, CR⁵³, or N,

Z⁵⁴ is CH, CR⁵⁴, or N,

Z⁵⁵ is CH, CR⁵⁵, or N,

Z⁵⁶ is CH, CR⁵⁶, or N,

Z⁵⁷ is CH, CR⁵⁷, or N,

Z⁵⁸ is CH, CR⁵⁸, or N,

R⁵¹ to R⁵⁸ are each independently one group of (5a) to (5h),

(5a) is a cyano group,

(5b) is a substituted or unsubstituted alkyl group having 1 to 20 carbon atoms,

(5c) is a substituted or unsubstituted alkoxy group having 1 to 20 carbon atoms,

(5d) is a substituted or unsubstituted aryl amino group having 6 to 20 carbon atoms,

(5e) is a substituted or unsubstituted phosphoryl group (a —POH₂ group),

(5f) is a substituted or unsubstituted silyl group (a —SiH₃ group),

(5g) is a substituted or unsubstituted monovalent aromatic hydrocarbon group,

(5h) is a substituted or unsubstituted monovalent heterocyclic group,

Ar⁵¹ includes at least one aromatic hydrocarbon group or a heterocyclic group,

m is 1, 2, 3, 4, 5, or 6,

wherein R⁵¹ and R⁵², R⁵² and R⁵³, R⁵³ and R⁵⁴, R⁵⁵ and R⁵⁵, R⁵⁶ and R⁵⁷, or R⁵⁷ and R⁵⁸ form an aliphatic hydrocarbon ring, an aromatic hydrocarbon ring, or a hetero ring, each including bonded carbon atoms.

(5b), (5c), (5d), (5g), and (5f) in Formula (5) are respectively the same as described in connection with (c), (d), (e), (f), and (g) in Formula (1).

An aromatic hydrogen ring in Ar⁵¹ is identical to the monovalent aromatic hydrocarbon ring described in (f) in Formula (1), except for having a different valence number.

A heterocyclic group in Ar⁵¹ is identical to the monovalent heterocyclic group described in (g) in Formula (1), except for having a different valence number.

In Formula (5), Z⁵¹ to Z⁵⁸ may not be N or only one of Z⁵¹ to Z⁵⁸ may be N. Z⁵¹ to Z⁵⁸ may not be N.

When the groups of (5c) to (5g) in Formula (5) are substituents, substituents substituting these groups are not particularly limited. For example, the substituents may be the groups of (5a) to (5h). Detailed examples of the substituents substituting such group may be, but not limited to, a cyano group, an unsubstituted alkyl group having 1 to 20 carbon atoms, an alkoxy group having 1 to 20 carbon atoms substituted with a monovalent aromatic hydrocarbon group having 6 to 30 carbon atoms substituted with an unsubstituted alkenyl group having 2 to 30 carbon atoms, a substituted or unsubstituted aryl amino group having 6 to 20 carbon atoms, an unsubstituted monovalent aromatic hydrocarbon group having 6 to 30 carbon atoms, a monovalent aromatic hydrocarbon group having 6 to 30 carbon atoms substituted with a cyano group, a monovalent aromatic hydrocarbon group having 6 to 30 carbon atoms substituted with an unsubstituted alkenyl group having 2 to 30 carbon atoms, a monovalent aromatic hydrocarbon group having 6 to 30 carbon atoms substituted with an unsubstituted amino group having 6 to 20 carbon atoms, an unsubstituted monovalent heterocyclic group having 3 to 30 ring-forming atoms, a monovalent heterocyclic group having 3 to 30 ring-forming atoms substituted with an unsubstituted monovalent aromatic hydrocarbon group having 6 to 30 carbon atoms, and the like.

In Formula (5), Ar⁵¹ may be, but not limited to, an aromatic hydrocarbon group or a heterocyclic group. Examples of Ar⁵¹ may be a substituted or unsubstituted aromatic hydrocarbon group, a substituted or unsubstituted heterocyclic group, a group wherein at least one substituted or unsubstituted aromatic hydrocarbon group is linked to at least one substituted or unsubstituted heterocyclic group via a single bond, or at least two substituted or unsubstituted aromatic hydrocarbon groups or substituted or unsubstituted heterocyclic groups linked via a linking group other than the two groups.

Here, in a group wherein at least two substituted or unsubstituted aromatic hydrocarbon groups or substituted or unsubstituted heterocyclic groups are linked via a linking group other than the two groups, the linking group is not particularly limited. Detailed examples of the linking group may include a Si group, an N group, a P═O group, an S(═O)═O group, a C═O group, and the like.

When the groups constituting Ar⁵¹ in Formula (5) are substituents, substituents substituting such groups are not particularly limited. For example, the substituents may be the groups of (5a) to (5h). Detailed examples of the substituents substituting such groups may include, but are not limited to, a cyano group, an unsubstituted alkoxy group having 1 to 20 carbon atoms, a monovalent heterocyclic group having 3 to 30 ring-forming atoms substituted with an unsubstituted alkoxy group having 1 to 20 carbon atoms, and the like.

In the substituents of the groups of (5c) to (5h) or the substituent of the groups constituting Ar⁵¹, an alkyl group having 1 to 20 carbon atoms, an alkoxy group having 1 to 20 carbon atoms, an aryl amino group having 6 to 20 carbon atoms, a monovalent aromatic hydrocarbon group having 6 to 30 carbon atoms, a monovalent heterocyclic group having 3 to 30 ring-forming atoms are respectively the same as described in connection with the groups of (c), (d), (e), (f), and (g) in Formula (1).

The substituents of the groups of (5c) to (5h), the alkenyl group having 2 to 30 carbon atoms of the substituent of the group constituting Ar⁵¹ may be linear, branched, or annular, but forms of the substituents are not limited thereto. Detailed examples of the alkenyl group may include, but are not limited to, a vinyl group, a 2-prophenyl group, a 2-butenyl group, a 3-butenyl group, a 1-methyl-2-prophenyl group, a 2-methyl-2-prophenyl group, a 2-pentenyl group, a 3-pentenyl group, a 4-pentenyl group, a 1-methyl-2-butenyl group, a 2-methyl-2-butenyl group, a 3-methyl-2-butenyl group, a 1-methyl-3-butenyl group, a 2-methyl-3-butenyl group, a 3-methyl-3-butenyl group, a 1,1-dimethyl-2-prophenyl group, a 1,2-dimethyl-2-prophenyl group, a 1-ethyl-2-prophenyl group, and the like.

m may be 1, 2, 3, or 4, or m may be 2.

Hereinafter, as an embodiment of the host material, a compound having a carbazole ring structure and a compound having a ring structure wherein at least one ring-forming carbon atom of the carbazole ring is substituted into a nitrogen atom will be explained in detail. The present disclosure is not limited to these examples:

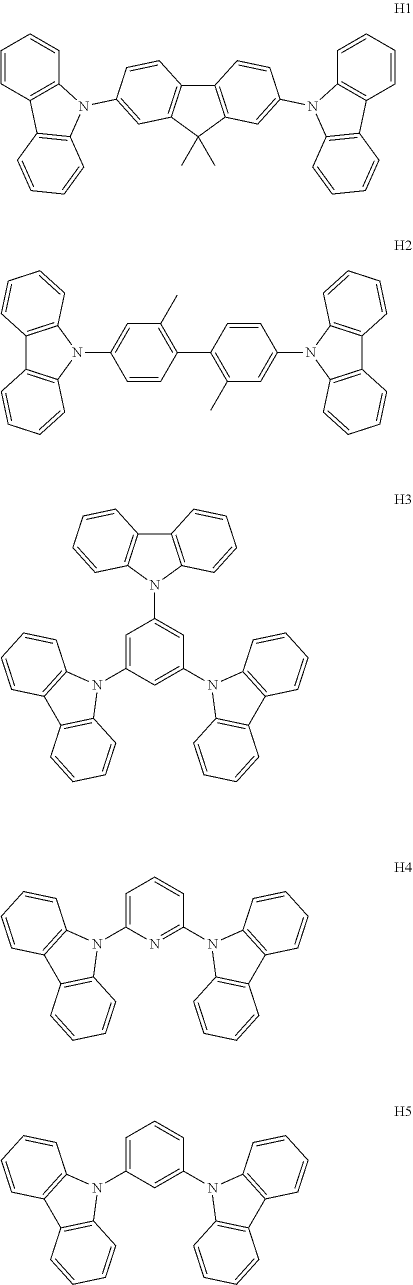 [SMILES: CC1(C)c2cc(-n3c4ccccc4c4ccccc43)ccc2-c2ccc(-n3c4ccccc4c4ccccc43)cc21 ; Cc1cc(-n2c3ccccc3c3ccccc32)ccc1-c1ccc(-n2c3ccccc3c3ccccc32)cc1C ; c1ccc2c(c1)c1ccccc1n2-c1cc(-n2c3ccccc3c3ccccc32)cc(-n2c3ccccc3c3ccccc32)c1 ; c1cc(-n2c3ccccc3c3ccccc32)nc(-n2c3ccccc3c3ccccc32)c1 ; c1cc(-n2c3ccccc3c3ccccc32)cc(-n2c3ccccc3c3ccccc32)c1]

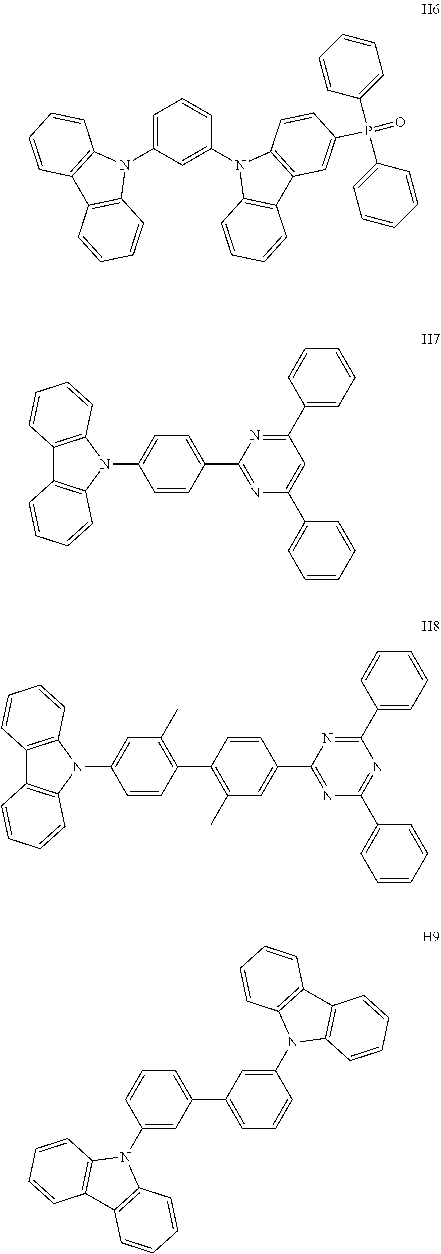 [SMILES: O=P(c1ccccc1)(c1ccccc1)c1ccc2c(c1)c1ccccc1n2-c1cccc(-n2c3ccccc3c3ccccc32)c1 ; c1ccc(-c2cc(-c3ccccc3)nc(-c3ccc(-n4c5ccccc5c5ccccc54)cc3)n2)cc1 ; Cc1cc(-c2nc(-c3ccccc3)nc(-c3ccccc3)n2)ccc1-c1ccc(-n2c3ccccc3c3ccccc32)cc1C ; c1cc(-c2cccc(-n3c4ccccc4c4ccccc43)c2)cc(-n2c3ccccc3c3ccccc32)c1]

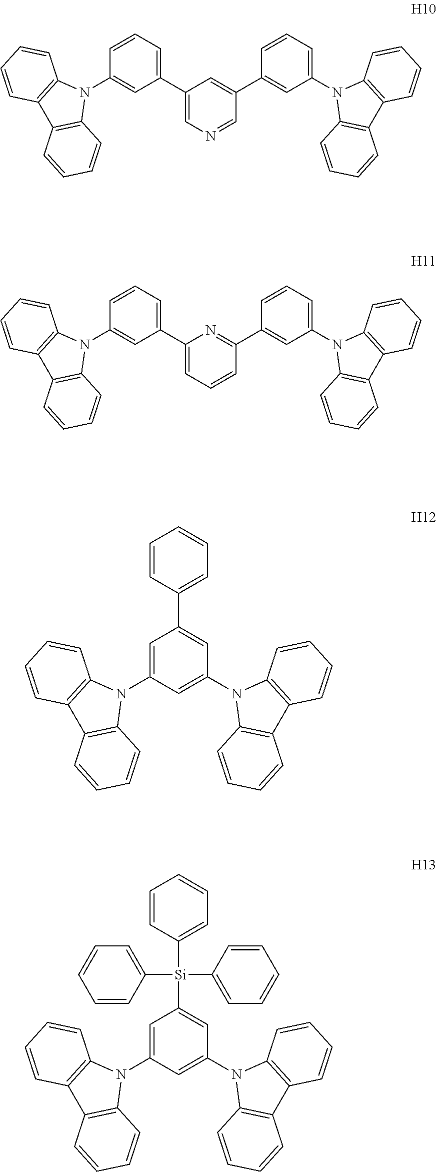 [SMILES: c1cc(-c2cncc(-c3cccc(-n4c5ccccc5c5ccccc54)c3)c2)cc(-n2c3ccccc3c3ccccc32)c1 ; c1cc(-c2cccc(-c3cccc(-n4c5ccccc5c5ccccc54)c3)n2)cc(-n2c3ccccc3c3ccccc32)c1 ; c1ccc(-c2cc(-n3c4ccccc4c4ccccc43)cc(-n3c4ccccc4c4ccccc43)c2)cc1 ; c1ccc([Si](c2ccccc2)(c2ccccc2)c2cc(-n3c4ccccc4c4ccccc43)cc(-n3c4ccccc4c4ccccc43)c2)cc1]

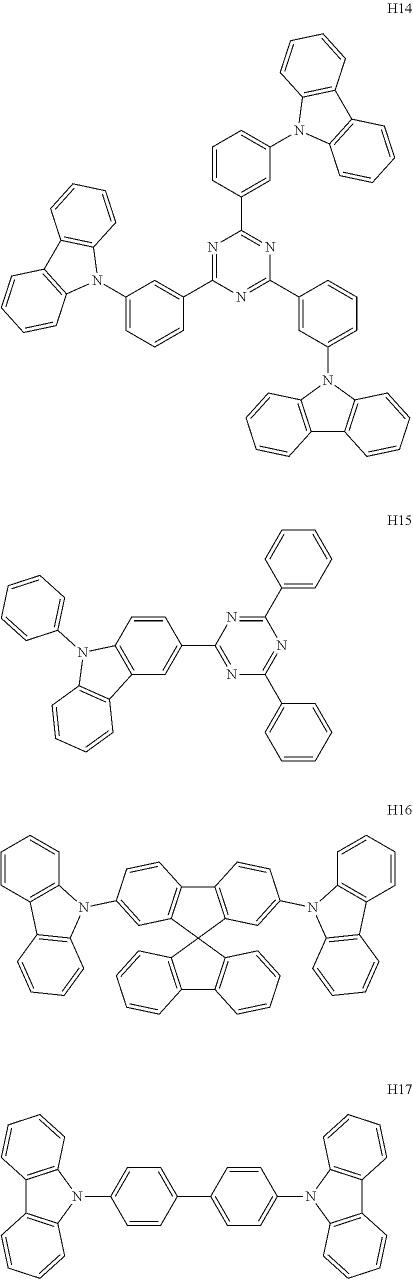 [SMILES: c1cc(-c2nc(-c3cccc(-n4c5ccccc5c5ccccc54)c3)nc(-c3cccc(-n4c5ccccc5c5ccccc54)c3)n2)cc(-n2c3ccccc3c3ccccc32)c1 ; c1ccc(-c2nc(-c3ccccc3)nc(-c3ccc4c(c3)c3ccccc3n4-c3ccccc3)n2)cc1 ; c1ccc2c(c1)-c1ccccc1C21c2cc(-n3c4ccccc4c4ccccc43)ccc2-c2ccc(-n3c4ccccc4c4ccccc43)cc21 ; c1ccc2c(c1)c1ccccc1n2-c1ccc(-c2ccc(-n3c4ccccc4c4ccccc43)cc2)cc1]

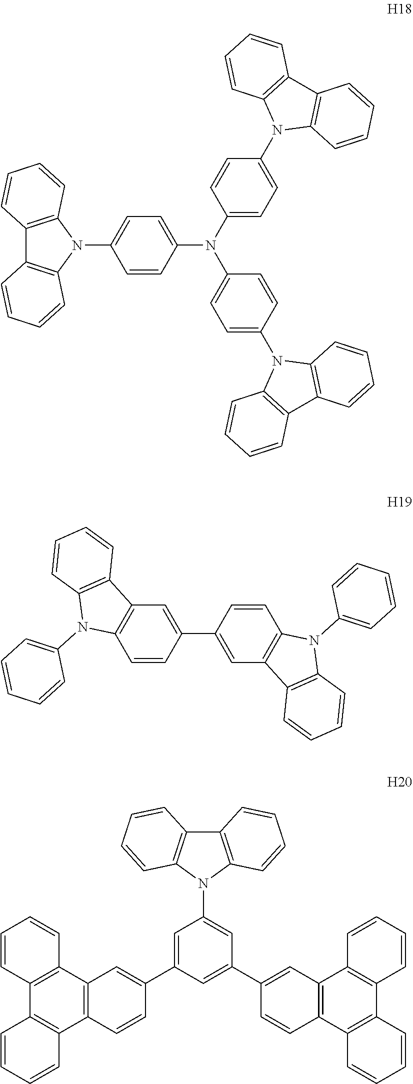 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(-n2c3ccccc3c3cc(-c4ccc5c(c4)c4ccccc4n5-c4ccccc4)ccc32)cc1 ; c1ccc2c(c1)c1ccccc1c1cc(-c3cc(-c4ccc5c6ccccc6c6ccccc6c5c4)cc(-n4c5ccccc5c5ccccc54)c3)ccc21]

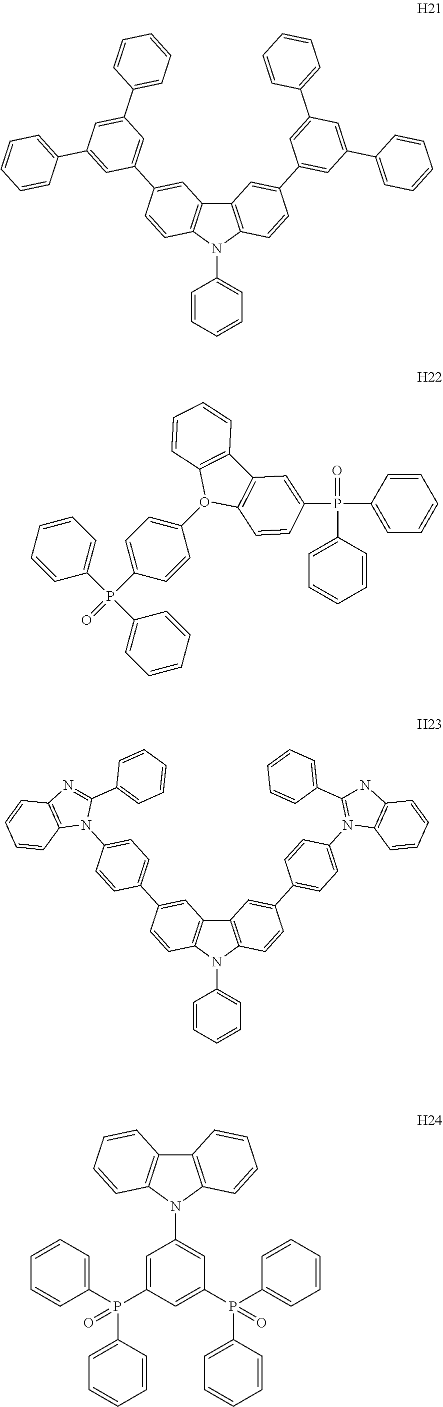

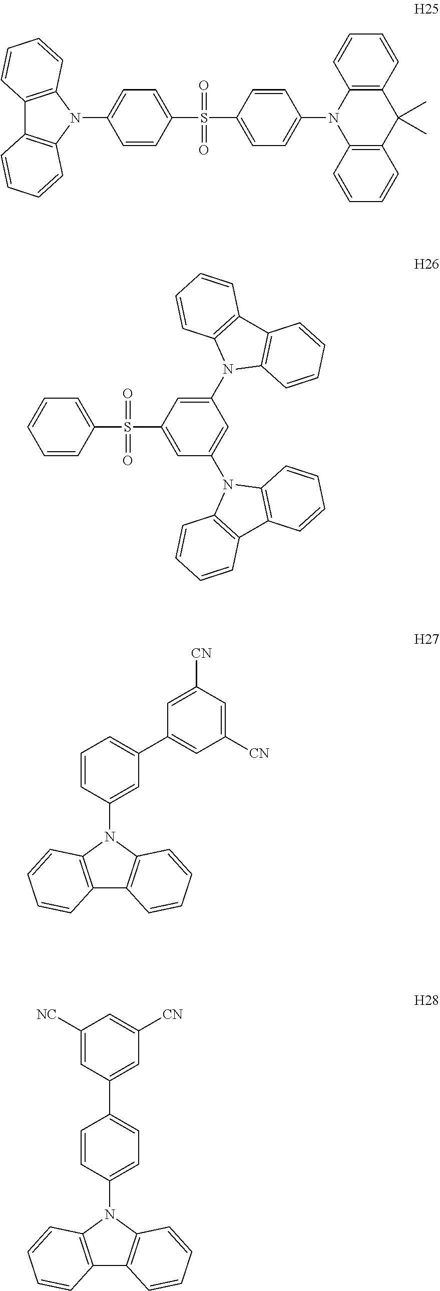 [SMILES: CC1(C)c2ccccc2N(c2ccc(S(=O)(=O)c3ccc(-n4c5ccccc5c5ccccc54)cc3)cc2)c2ccccc21 ; O=S(=O)(c1ccccc1)c1cc(-n2c3ccccc3c3ccccc32)cc(-n2c3ccccc3c3ccccc32)c1 ; N#Cc1cc(C#N)cc(-c2cccc(-n3c4ccccc4c4ccccc43)c2)c1 ; N#Cc1cc(C#N)cc(-c2ccc(-n3c4ccccc4c4ccccc43)cc2)c1]

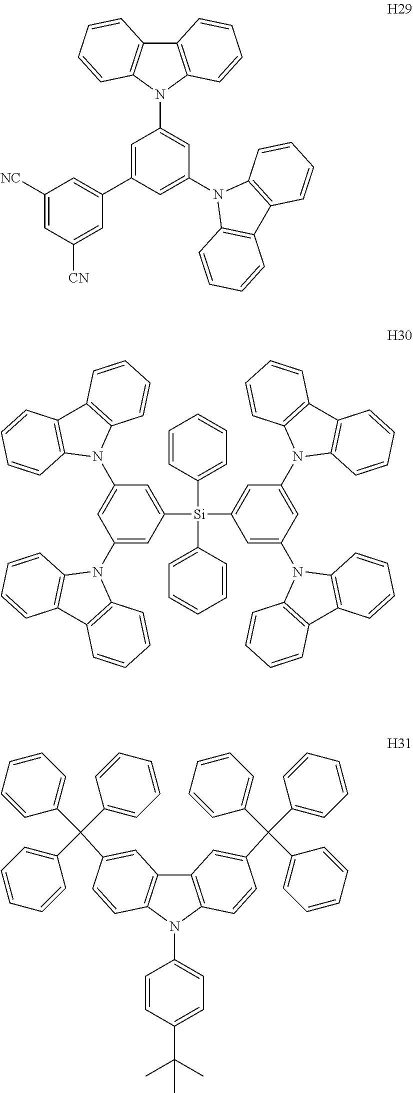 [SMILES: N#Cc1cc(C#N)cc(-c2cc(-n3c4ccccc4c4ccccc43)cc(-n3c4ccccc4c4ccccc43)c2)c1 ; c1ccc([Si](c2ccccc2)(c2cc(-n3c4ccccc4c4ccccc43)cc(-n3c4ccccc4c4ccccc43)c2)c2cc(-n3c4ccccc4c4ccccc43)cc(-n3c4ccccc4c4ccccc43)c2)cc1 ; CC(C)(C)c1ccc(-n2c3ccc(C(c4ccccc4)(c4ccccc4)c4ccccc4)cc3c3cc(C(c4ccccc4)(c4ccccc4)c4ccccc4)ccc32)cc1]

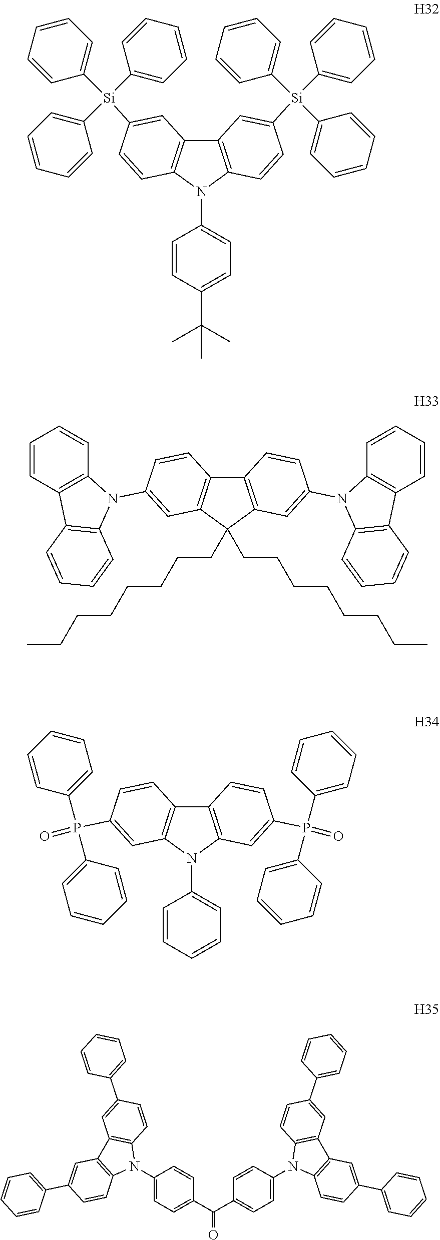

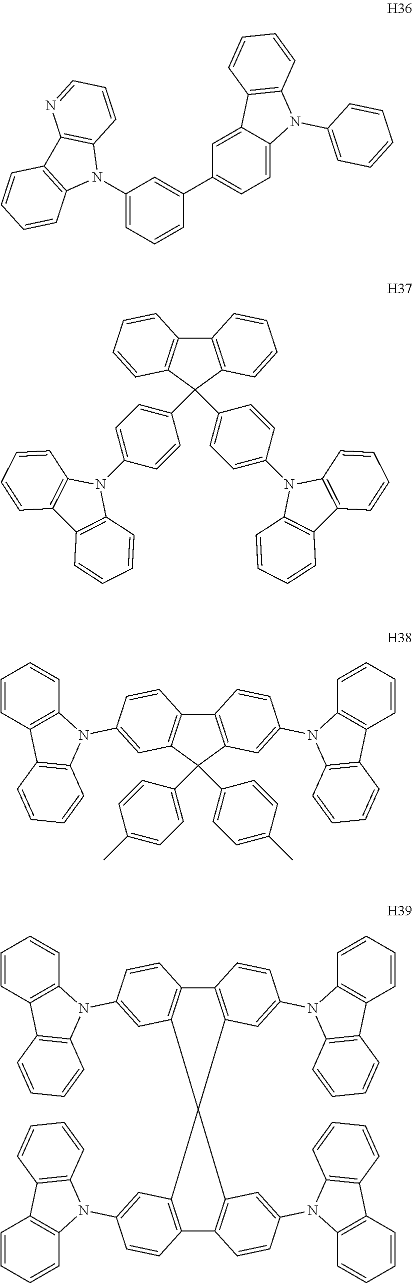 [SMILES: c1ccc(-n2c3ccccc3c3cc(-c4cccc(-n5c6ccccc6c6ncccc65)c4)ccc32)cc1 ; c1ccc2c(c1)-c1ccccc1C2(c1ccc(-n2c3ccccc3c3ccccc32)cc1)c1ccc(-n2c3ccccc3c3ccccc32)cc1 ; Cc1ccc(C2(c3ccc(C)cc3)c3cc(-n4c5ccccc5c5ccccc54)ccc3-c3ccc(-n4c5ccccc5c5ccccc54)cc32)cc1 ; c1ccc2c(c1)c1ccccc1n2-c1ccc2c(c1)C1(c3cc(-n4c5ccccc5c5ccccc54)ccc3-2)c2cc(-n3c4ccccc4c4ccccc43)ccc2-c2ccc(-n3c4ccccc4c4ccccc43)cc21]

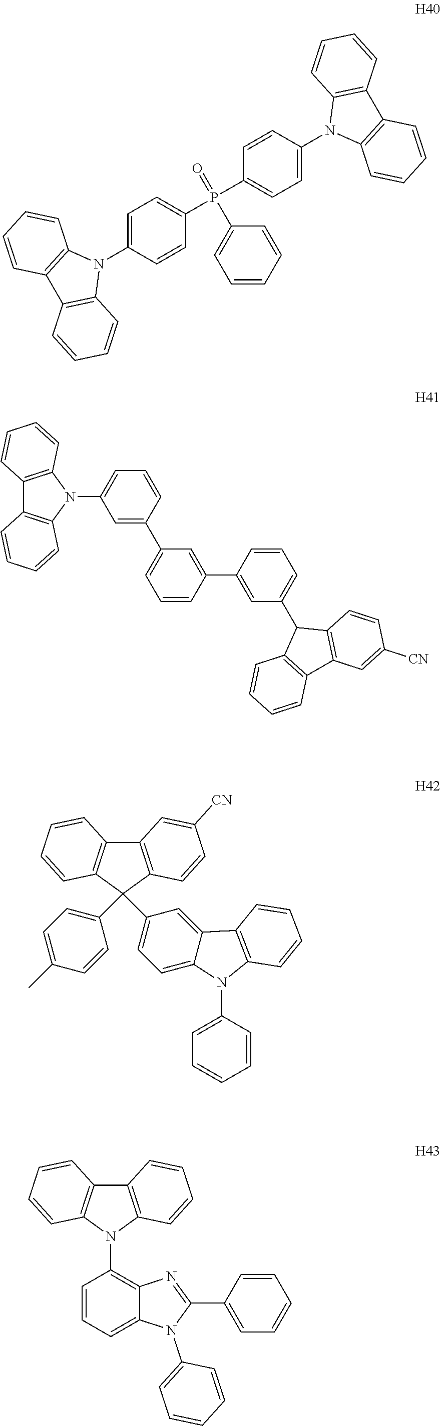 [SMILES: O=P(c1ccccc1)(c1ccc(-n2c3ccccc3c3ccccc32)cc1)c1ccc(-n2c3ccccc3c3ccccc32)cc1 ; N#Cc1ccc2c(c1)-c1ccccc1C2c1cccc(-c2cccc(-c3cccc(-n4c5ccccc5c5ccccc54)c3)c2)c1 ; Cc1ccc(C2(c3ccc4c(c3)c3ccccc3n4-c3ccccc3)c3ccccc3-c3cc(C#N)ccc32)cc1 ; c1ccc(-c2nc3c(-n4c5ccccc5c5ccccc54)cccc3n2-c2ccccc2)cc1]

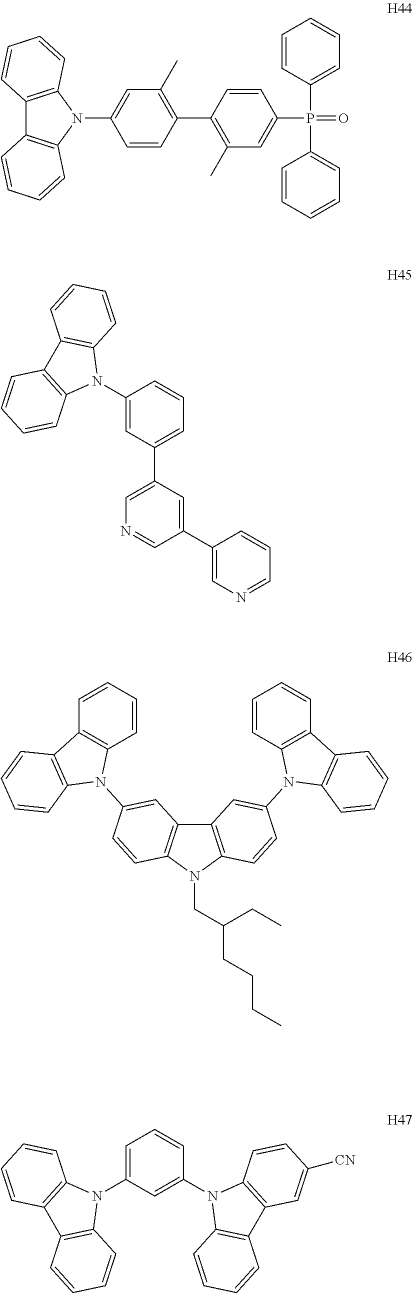 [SMILES: Cc1cc(-n2c3ccccc3c3ccccc32)ccc1-c1ccc(P(=O)(c2ccccc2)c2ccccc2)cc1C ; c1cncc(-c2cncc(-c3cccc(-n4c5ccccc5c5ccccc54)c3)c2)c1 ; CCCCC(CC)Cn1c2ccc(-n3c4ccccc4c4ccccc43)cc2c2cc(-n3c4ccccc4c4ccccc43)ccc21 ; N#Cc1ccc2c(c1)c1ccccc1n2-c1cccc(-n2c3ccccc3c3ccccc32)c1]

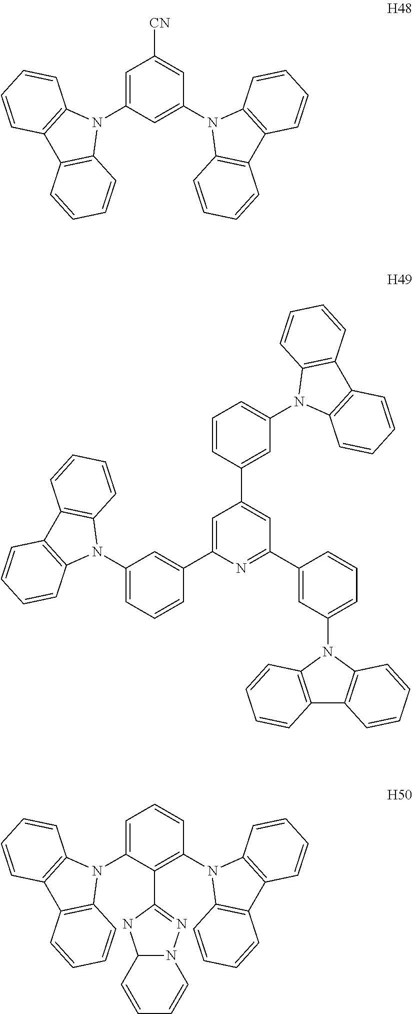 [SMILES: N#Cc1cc(-n2c3ccccc3c3ccccc32)cc(-n2c3ccccc3c3ccccc32)c1 ; c1cc(-c2cc(-c3cccc(-n4c5ccccc5c5ccccc54)c3)nc(-c3cccc(-n4c5ccccc5c5ccccc54)c3)c2)cc(-n2c3ccccc3c3ccccc32)c1 ; C1=CC2NC(c3c(-n4c5ccccc5c5ccccc54)cccc3-n3c4ccccc4c4ccccc43)=NN2C=C1]

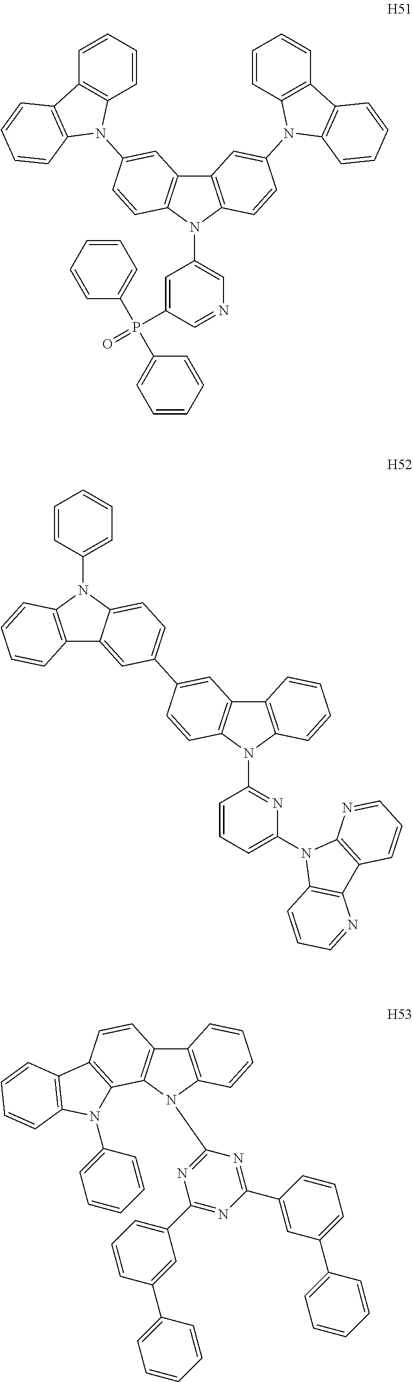 [SMILES: O=P(c1ccccc1)(c1ccccc1)c1cncc(-n2c3ccc(-n4c5ccccc5c5ccccc54)cc3c3cc(-n4c5ccccc5c5ccccc54)ccc32)c1 ; c1ccc(-n2c3ccccc3c3cc(-c4ccc5c(c4)c4ccccc4n5-c4cccc(-n5c6cccnc6c6cccnc65)n4)ccc32)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1]

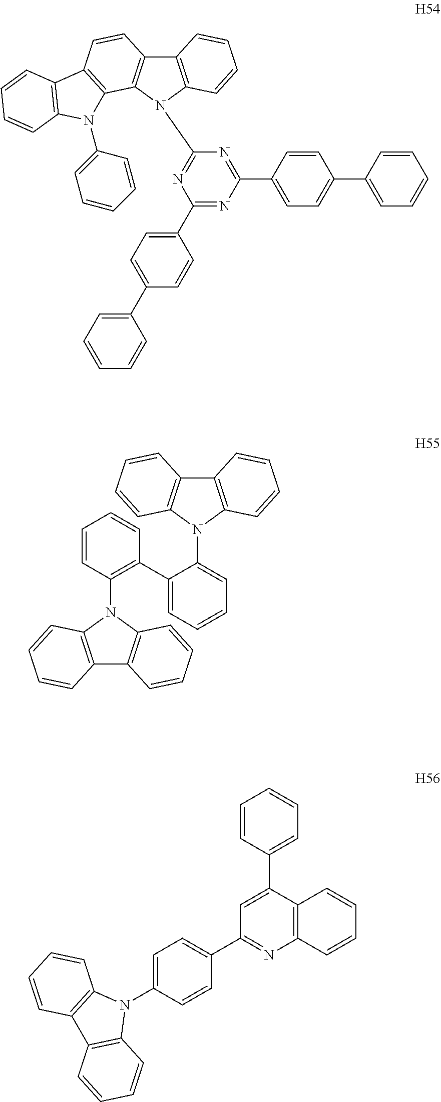 [SMILES: c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-n2c3ccccc3c3ccccc32)c(-c2ccccc2-n2c3ccccc3c3ccccc32)c1 ; c1ccc(-c2cc(-c3ccc(-n4c5ccccc5c5ccccc54)cc3)nc3ccccc23)cc1]

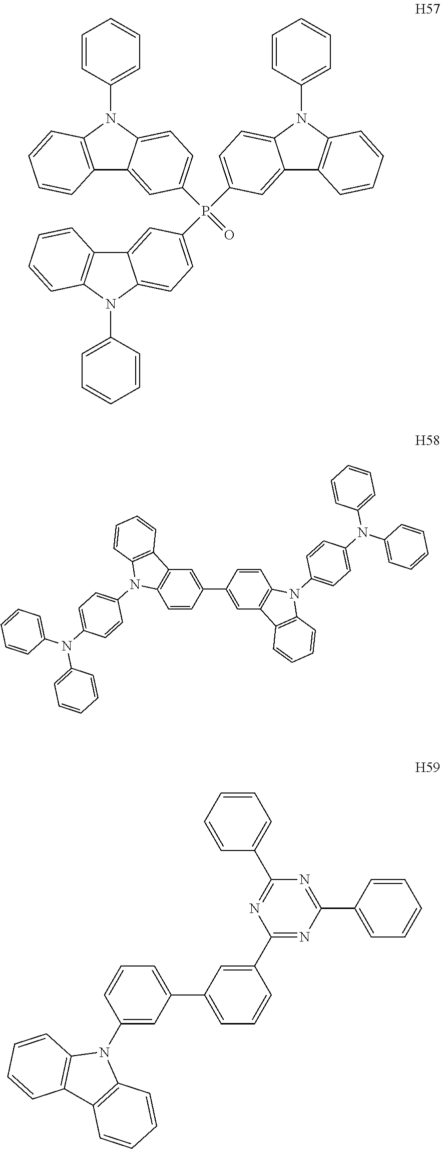 [SMILES: O=P(c1ccc2c(c1)c1ccccc1n2-c1ccccc1)(c1ccc2c(c1)c1ccccc1n2-c1ccccc1)c1ccc2c(c1)c1ccccc1n2-c1ccccc1 ; c1ccc(N(c2ccccc2)c2ccc(-n3c4ccccc4c4cc(-c5ccc6c(c5)c5ccccc5n6-c5ccc(N(c6ccccc6)c6ccccc6)cc5)ccc43)cc2)cc1 ; c1ccc(-c2nc(-c3ccccc3)nc(-c3cccc(-c4cccc(-n5c6ccccc6c6ccccc65)c4)c3)n2)cc1]

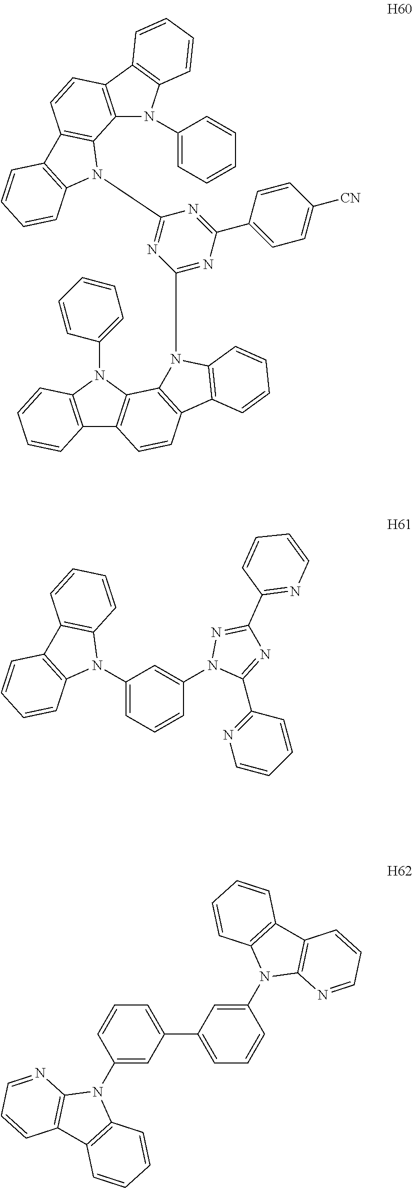 [SMILES: N#Cc1ccc(-c2nc(-n3c4ccccc4c4ccc5c6ccccc6n(-c6ccccc6)c5c43)nc(-n3c4ccccc4c4ccc5c6ccccc6n(-c6ccccc6)c5c43)n2)cc1 ; c1ccc(-c2nc(-c3ccccn3)n(-c3cccc(-n4c5ccccc5c5ccccc54)c3)n2)nc1 ; c1cc(-c2cccc(-n3c4ccccc4c4cccnc43)c2)cc(-n2c3ccccc3c3cccnc32)c1]

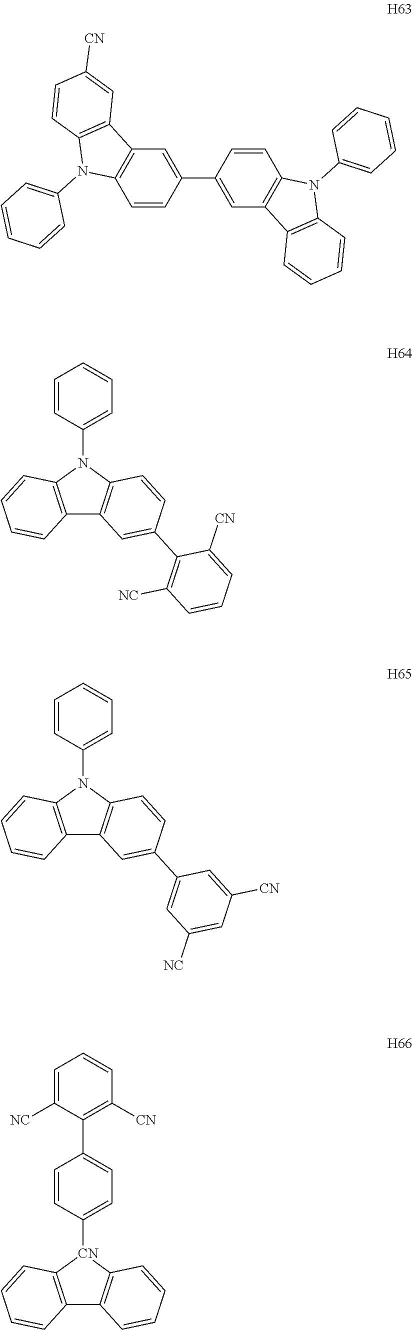

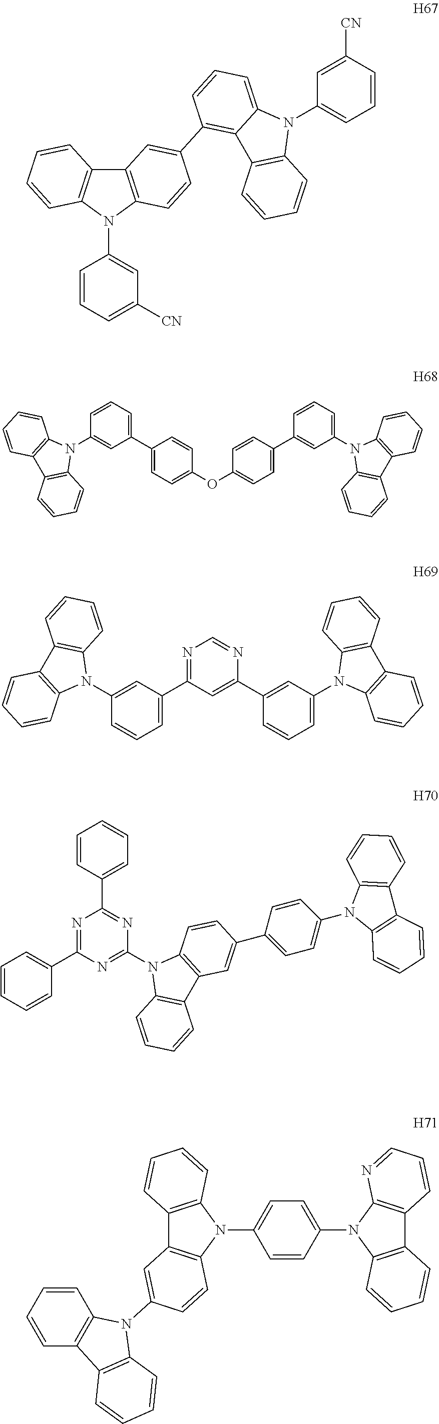 [SMILES: N#Cc1cccc(-n2c3ccccc3c3cc(-c4cccc5c4c4ccccc4n5-c4cccc(C#N)c4)ccc32)c1 ; c1cc(-c2ccc(Oc3ccc(-c4cccc(-n5c6ccccc6c6ccccc65)c4)cc3)cc2)cc(-n2c3ccccc3c3ccccc32)c1 ; c1cc(-c2cc(-c3cccc(-n4c5ccccc5c5ccccc54)c3)ncn2)cc(-n2c3ccccc3c3ccccc32)c1 ; c1ccc(-c2nc(-c3ccccc3)nc(-n3c4ccccc4c4cc(-c5ccc(-n6c7ccccc7c7ccccc76)cc5)ccc43)n2)cc1 ; c1ccc2c(c1)c1ccccc1n2-c1ccc2c(c1)c1ccccc1n2-c1ccc(-n2c3ccccc3c3cccnc32)cc1]

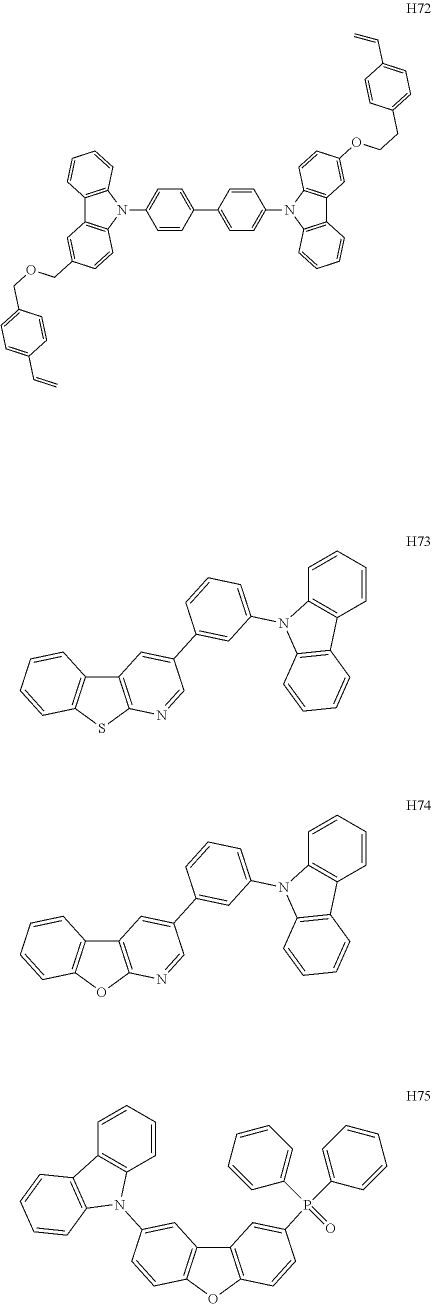 [SMILES: C=Cc1ccc(CCOc2ccc3c(c2)c2ccccc2n3-c2ccc(-c3ccc(-n4c5ccccc5c5cc(COCc6ccc(C=C)cc6)ccc54)cc3)cc2)cc1 ; c1cc(-c2cnc3sc4ccccc4c3c2)cc(-n2c3ccccc3c3ccccc32)c1 ; c1cc(-c2cnc3oc4ccccc4c3c2)cc(-n2c3ccccc3c3ccccc32)c1 ; O=P(c1ccccc1)(c1ccccc1)c1ccc2oc3ccc(-n4c5ccccc5c5ccccc54)cc3c2c1]

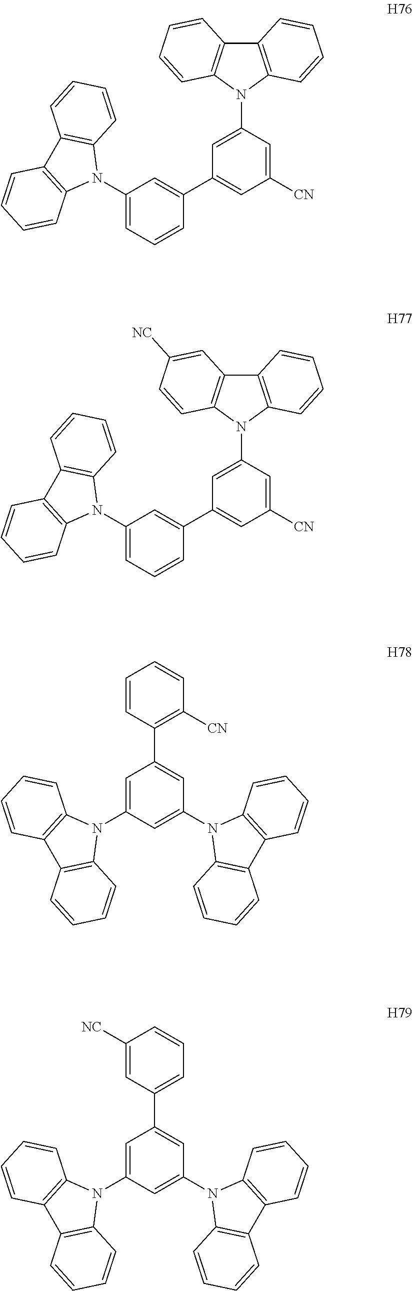 [SMILES: N#Cc1cc(-c2cccc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1ccccc1-c1cc(-n2c3ccccc3c3ccccc32)cc(-n2c3ccccc3c3ccccc32)c1 ; N#Cc1cccc(-c2cc(-n3c4ccccc4c4ccccc43)cc(-n3c4ccccc4c4ccccc43)c2)c1]

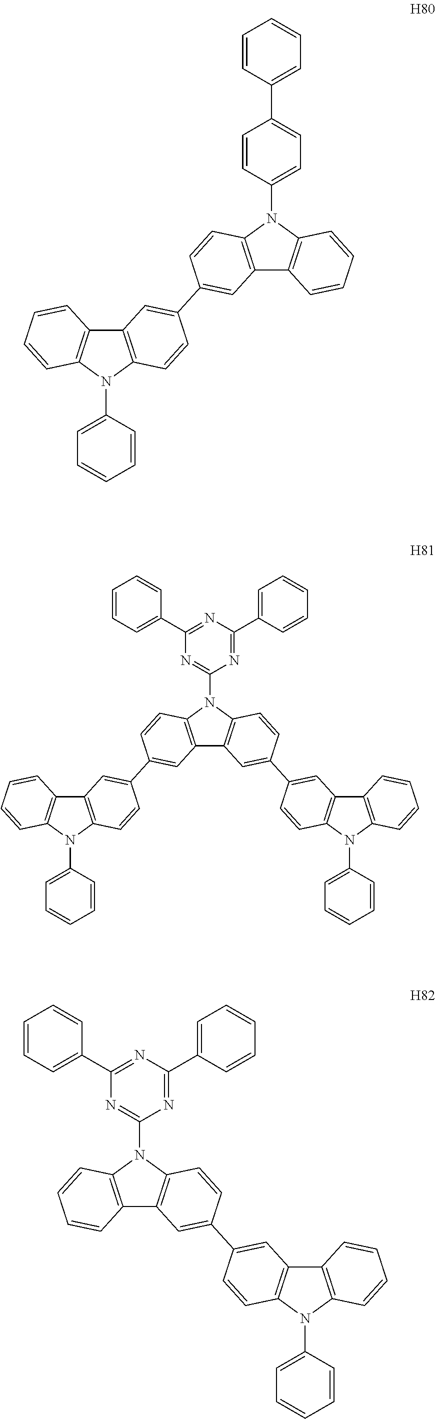 [SMILES: c1ccc(-c2ccc(-n3c4ccccc4c4cc(-c5ccc6c(c5)c5ccccc5n6-c5ccccc5)ccc43)cc2)cc1 ; c1ccc(-c2nc(-c3ccccc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

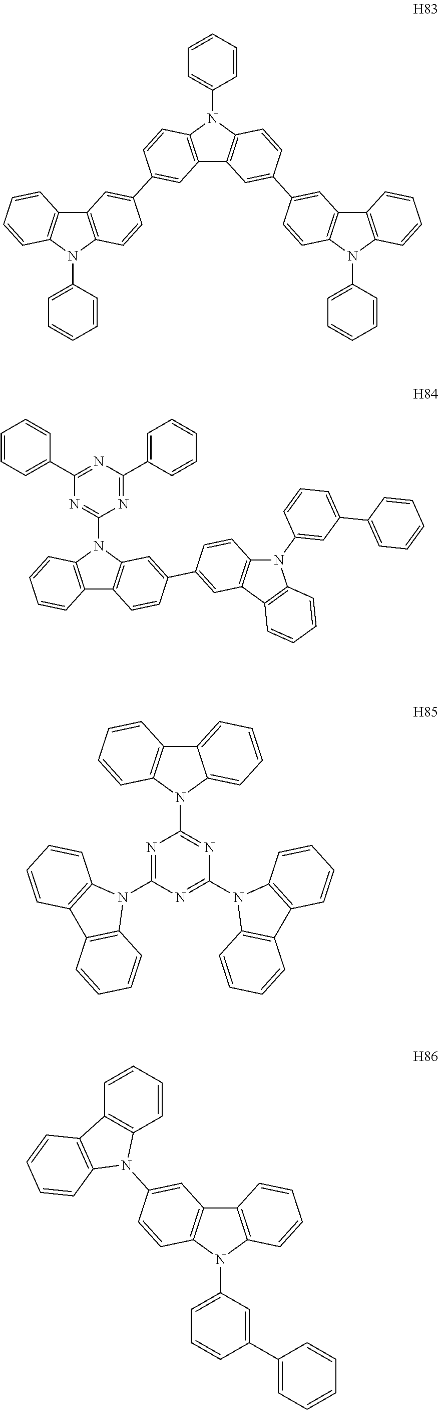 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2cccc(-n3c4ccccc4c4cc(-c5ccc6c7ccccc7n(-c7nc(-c8ccccc8)nc(-c8ccccc8)n7)c6c5)ccc43)c2)cc1 ; c1ccc2c(c1)c1ccccc1n2-c1nc(-n2c3ccccc3c3ccccc32)nc(-n2c3ccccc3c3ccccc32)n1 ; c1ccc(-c2cccc(-n3c4ccccc4c4cc(-n5c6ccccc6c6ccccc65)ccc43)c2)cc1]

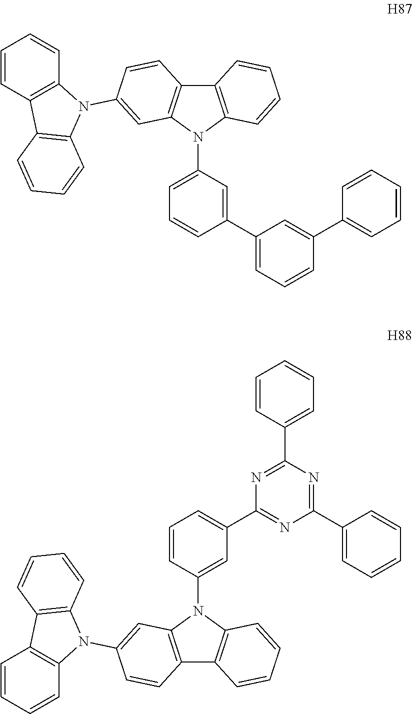 [SMILES: c1ccc(-c2cccc(-c3cccc(-n4c5ccccc5c5ccc(-n6c7ccccc7c7ccccc76)cc54)c3)c2)cc1 ; c1ccc(-c2nc(-c3ccccc3)nc(-c3cccc(-n4c5ccccc5c5ccc(-n6c7ccccc7c7ccccc76)cc54)c3)n2)cc1]

The material for the organic electroluminescent device according to an embodiment may include the nitrogen-containing condensed cyclic compound, the phosphorescent complex, and the host material, wherein the host material may include the compound having the structure represented by Formula (5). In an embodiment of the organic electroluminescent device, the device may include the fluorescence emitter or the nitrogen-containing condensed cyclic compound and the host material, wherein the host material may include the compound having the structure represented by Formula (5).

The compound having the triazine ring structure may be, but not limited to, the compound having the structure represented by Formula (6):

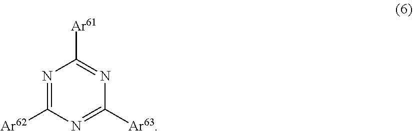

wherein, in Formula (6),

Ar⁶¹ to Ar⁶³ are each independently a substituted or unsubstituted monovalent aromatic hydrocarbon group or a substituted or unsubstituted monovalent heterocyclic group.

The substituted or unsubstituted monovalent aromatic hydrocarbon group in Formula (6) is the same as described in connection with the group of (f) of Formula (1). The substituted or unsubstituted monovalent heterocyclic group is the same as described in connection with the group of (g) of Formula (1).

In Formula (6), the substituent substituting the monovalent aromatic hydrocarbon group or the monovalent heterocyclic group may be, but not limited to, substituents listed as substituting the groups of (c) to (g) in Formula (1). The substituent may also be a silyl group substituted with an unsubstituted monovalent aromatic hydrocarbon group. In addition, the unsubstituted monovalent aromatic hydrocarbon group is the same as described in connection with the unsubstituted group that may be listed as group (f).

The compounds having a triazine ring structure may be a compound containing a silyl group (compound having a triazine ring structure having a silyl group).

In addition, the compound having a triazine ring structure may be used in combination with the compound having the carbazole ring structure or the compound having a ring structure wherein at least one of the ring-forming carbon atom of the carbazole ring is substituted into a nitrogen atom.

Hereinafter, the compound having a triazine structure, which is the host material according to an embodiment, will be explained in detail. The present disclosure is not limited to these examples:

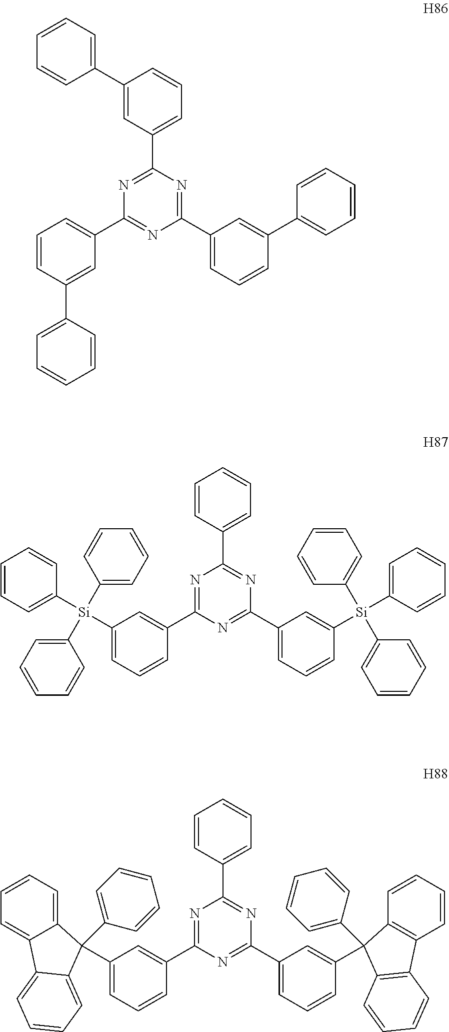 [SMILES: c1ccc(-c2cccc(-c3nc(-c4cccc(-c5ccccc5)c4)nc(-c4cccc(-c5ccccc5)c4)n3)c2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2nc(-c3cccc(C4(c5ccccc5)c5ccccc5-c5ccccc54)c3)nc(-c3cccc(C4(c5ccccc5)c5ccccc5-c5ccccc54)c3)n2)cc1]

In an embodiment, the material for the organic electroluminescent device may include the nitrogen-containing condensed cyclic compound, the phosphorescent complex, and the host material, wherein the host material may include the compound having the structure represented by Formula (6). In an embodiment, the material for the organic electroluminescent device may include the nitrogen-containing condensed cyclic compound, the phosphorescent complex, and the host material, wherein the host material may include the compound having the structure represented by Formula (5) and the compound having the structure represented by Formula (6). In an embodiment of the organic electroluminescent device, the device may include the fluorescence emitter or the nitrogen-containing condensed cyclic compound and the host material, wherein the host material may include the compound having the structure represented by Formula (6). In a more preferred embodiment, the organic electroluminescent device may include the fluorescence emitter or the nitrogen-containing condensed cyclic compound and the host material, wherein the host material may include the compound having the structure represented by Formula (5) and the compound having the structure represented by Formula (6).

An amount of the host material may be, but not limited to, 5 wt % or more based on the total weight of the material for the organic electroluminescent device (particularly the material for the emission layer). The amount may be 10 wt % or more, or 20 wt % or more. Within this range, an organic electroluminescent device having excellent luminescence color purity and high emission efficiency may be obtained. An amount of the host material may be, but not limited to, 99 wt % or less based on the total weight of the material for the organic electroluminescent device (particularly the material for the emission layer). The amount may be 98 wt % or less, or 95 wt % or less. Within this range, an organic electroluminescent device having excellent luminescence color purity and high emission efficiency may be obtained. An amount of the host material relative to the total weight of the emission layer of the organic electroluminescent device to be described later may be the same as above.

When the material for the organic electroluminescent device includes the host material, the amount thereof may be 100 parts by weight or more based on 100 parts by weight of the nitrogen-containing condensed cyclic compound. The amount may be, based on 100 parts by weight of the nitrogen-containing condensed cyclic compound, 2,000 parts by weight or more, or 3,000 parts by weight or more. Within this range, an organic electroluminescent device having excellent luminescence color purity and high emission efficiency may be obtained. The amount of the host material may be, but not limited to, 200,000 parts by weight or less based on 100 parts by weight of the nitrogen-containing condensed cyclic compound. The amount may be, based on 100 parts by weight of the nitrogen-containing condensed cyclic compound, 150,000 parts by weight or less, or 100,000 parts by weight or less. Within this range, an organic electroluminescent device having excellent luminescence color purity and high emission efficiency may be obtained. An amount (parts by weight) of the host material relative to 100 parts by mass of the nitrogen-containing condensed cyclic compound in the emission layer of the organic electroluminescent device to be described later may be the same as above.

### Liquid Composition

Another embodiment of the present disclosure relates to a liquid composition including the nitrogen-containing heterocyclic compound, the fluorescence emitter, or the material for the organic electroluminescent device, and a solvent.

The solvent is not particularly limited, but the solvent may have the boiling point of 100° C. or more and 350° C. or less in atmospheric pressure (101.3 kPa, 1 atm). The boiling point of the solvent in atmospheric pressure may be 150° C. or more and 320° C. or less, or 180° C. or more and 300° C. or less. When the boiling point of the solvent in an atmospheric pressure is within the above range, the processability or film-forming capability of a wet film forming method may be improved, especially in an inkjet method. Not being limited to the solvent of which the boiling point in an atmospheric pressure is 100° C. or more and 350° C. or less, a known solvent may be used accordingly. The solvent of which the boiling point in an atmospheric pressure is 100° C. or more and 350° C. or less is explained in detail below, but the present disclosure is not limited to these examples. Examples of a hydrocarbon-based solvent may include octane, nonane, decane, undecane, dodecane, and the like. Examples of an aromatic hydrocarbon-based solvent may include toluene, xylene, ethylbenzene, n-propylbenzene, isopropylbenzene, n-propylbenzene, mesitylene, n-butylbenzene, sec-butylbenzene, 1-phenylpentane, 2-phenylpentane, 3-phenylpentane, phenylcyclopentane, phenylcyclohexane, 2-ethylbiphenyl, 3-ethylbiphenyl, and the like. Examples of an ether-based solvent may include 1,4-dioxane, 1,2-diethoxyethane, diethyleneglycoldimethyl ether, diethyleneglycoldiethyl ether, anisole, ethoxybenzene, 3-methylanisole, m-dimethoxybenzene, and the like. Examples of a ketone-based solvent may include 2-hexanone, 3-hexanone, cyclohexanone, 2-heptanone, 3-heptanone, 4-heptanone, cycloheptanone, and the like. Examples of an ester-based solvent may include butyl acetate, butyl propionate, butyl butyrate, propylenecarbonate, methylbenzoate, ethylbenzoate, 1-propylbenzoate, 1-butylbenzoate, and the like. Examples of a nitrile-based solvent may include benzonitrile, 3-methylbenzonitrile, and the like. Examples of an amide-based solvent may be dimethylformamide, dimethylacetamide, N-methylpyrrolidone, and the like. Such solvent may be used alone or in combination of two or more.

In an embodiment of the present disclosure, the amount of the nitrogen-containing heterocyclic compound, the fluorescence emitter, or the material for the organic electroluminescent device in the liquid composition are not particularly limited.

In an embodiment of the present disclosure, the liquid composition may be used as a coating liquid for forming an organic layer of the organic electroluminescent device. The liquid composition may be used as a coating liquid for forming an emission layer among the coating liquid for forming an organic layer.

### Organic Electroluminescent Device

Another embodiment of the present disclosure relates to an organic electroluminescent device including the nitrogen-containing condensed cyclic compound or the fluorescence emitter. The organic electroluminescent device may include the nitrogen-containing condensed cyclic compound or the fluorescence emitter and the host material. The organic electroluminescent device may include the nitrogen-containing condensed cyclic compound or the fluorescence emitter and the phosphorescent complex. The phosphorescent complex may be a platinum complex.

Another aspect of the present disclosure relates to an organic electroluminescent device including the material for the organic electroluminescent device. The material for the organic electroluminescent device may include the host material. The phosphorescent complex included in the organic electroluminescent device may be a platinum complex.

The host material in the organic electroluminescent device may be the compound having the carbazole ring structure, the compound having a ring structure wherein at least one ring-forming carbon atom of the carbazole ring is substituted into a nitrogen atom, or the compound having the triazine ring structure. The host material included in the organic electroluminescent device may include the compound having the structure represented by Formula (5). The host material included in the organic electroluminescent device may include the compound having the structure represented by Formula (6). The host material included in the organic electroluminescent device may include the compound having the structure represented by Formula (5) and the compound having the structure represented by Formula (6).

The phosphorescent complex included in the organic electroluminescent device may include the compound having the structure represented by Formula (4).

The organic electroluminescent device according to an embodiment may include, but is not limited to, a first electrode, a second electrode, and a single-layered or multi-layered organic layer. The second electrode may be located on the first electrode.

When a portion of a layer, film, region, plate, etc. is said to be “on” or “above” another portion in the present specification, this includes not only the case in which the portion is “directly on” another portion, but also the case in which an intervening layer is placed therebetween. When a portion of a layer, film, region, plate, etc. is said to be “under” or “below” another portion in the present specification, this includes not only the case in which the portion is “directly under” another portion, but also the case in which an intervening layer is placed therebetween. In the present disclosure, being located “on” includes not only being located on the top surface but also on the lower or bottom surface.

The organic electroluminescent device according to an embodiment may include a first electrode, a second electrode, and a single or plurality of layers between the first electrode and the second electrode. Here, the layer may include at least one organic layer, and the at least one organic layer may include the nitrogen-containing condensed cyclic compound and the fluorescence emitter or the material for the organic electroluminescent device. The organic layer including the nitrogen-containing condensed cyclic compound and the fluorescence emitter or the material for the organic electroluminescent device may include an emission layer. Such organic electroluminescent device may realize luminescence with high color purity.

Such emission layer may include at least one of the nitrogen-containing condensed cyclic compound.

The emission layer may be single-layered including a single material, or single-layered including a plurality of different materials. In addition, the emission layer may be multi-layered including a plurality of different materials.

The emission layer is not particularly limited, and may include a host material and a dopant material, for example. The nitrogen-containing condensed cyclic compound and the fluorescence emitter may be used as a host material or a dopant material, but may probably be used as a dopant material.

In an embodiment of the present disclosure, the organic electroluminescent device may include the emission layer, wherein the emission layer may include the nitrogen-containing condensed cyclic compound, the fluorescence emitter, or the material for the organic electroluminescent device. The emission layer may be formed from the material for the organic electroluminescent device. In view of the peak wavelength, luminescence color, emission efficiency, and device lifespan of the emission spectrum, the material for the organic electroluminescent device may include the host material in addition to the nitrogen-containing condensed cyclic compound. In addition, the material for the organic electroluminescent device may include the phosphorescent compound and the host material in addition to the nitrogen-containing condensed cyclic compound. A preferred range of the amount or amount ratio of the nitrogen-containing condensed cyclic compound, phosphorescent complex, and the host material of the emission layer may each be the same as the preferred amount or amount ratio of the material for the organic electroluminescent device.

A thickness of the emission layer is not particularly limited, and may be 1 nm or more and 100 nm or less, and 10 nm or more and 50 nm or less.

Examples of the film forming method of the emission layer may include, but not limited to, vacuum deposition, spin coating, Langmuir-Blodgett (LB) deposition, ink-jet printing, laser-printing, and laser-induced thermal imaging (LITI).

The emission wavelength of the organic electroluminescent device is not particularly limited. A preferred range of the emission wavelength of the organic electroluminescent device may be, for example, the PL peak wavelength of the nitrogen-containing condensed cyclic compound according to the present disclosure. In specifications of products currently being commercialized, the blue luminescence may emit light that has a peak in the wavelength region of 445 nm or more and 470 or less, or may emit light that has a peak in the wavelength region of 450 nm or more and 470 or less, or may emit light that has a peak in the wavelength region of 450 nm or more and 465 or less.

In addition, the FWHM of the peak of the emission spectrum of the organic electroluminescent device may be smaller. Further, the FWHM of the peak of the luminescence spectrum may be 30 nm or less, or 25 nm or less. The FWHM of the peak of the emission spectrum may be 20 nm or less (the lower limit exceeds 0 nm).

Hereinafter, the case where the organic electroluminescent device has another organic layer in addition to the emission layer according to an embodiment of the present disclosure will be described in detail with reference to the accompanying drawings. The same components in the description of drawings will be denoted by the same reference numerals, and thus redundant description thereof will be omitted. The dimension ratio of the drawings may be exaggerated for convenience of explanation, and thus may differ from the actual ratio.

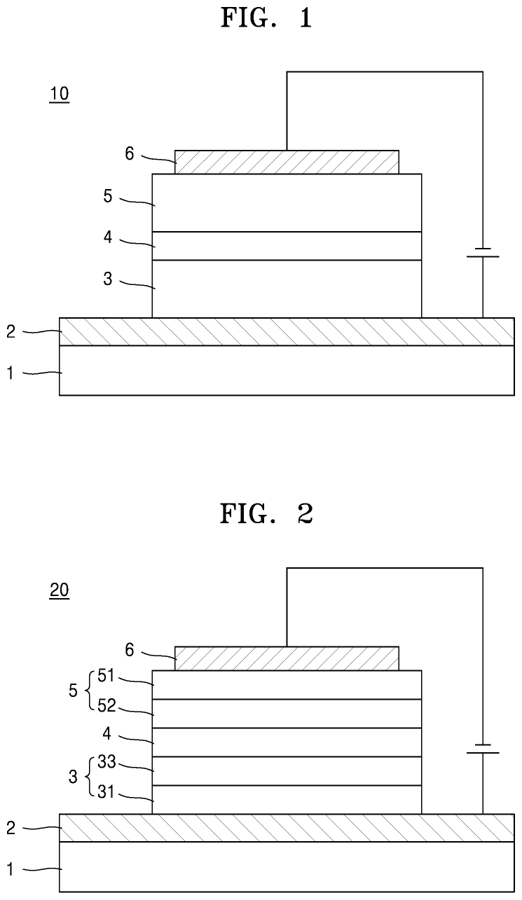

Each of FIGS. 1 to 3 are schematic cross-sectional views of an organic electroluminescent device according to an embodiment. However, the structure of the organic electroluminescent device according to the present disclosure may not be limited to the embodiments shown in FIGS. 1 to 3.

FIG. 1 is a schematic cross-sectional view of an organic electroluminescent device according to an exemplary embodiment. The organic electroluminescent device 10 according to an embodiment may include a substrate 1, a first electrode 2, a hole transport region 3, an emission layer 4, an electron transport region 5, and a second electrode 6, which are sequentially stacked in this stated order.

FIG. 2 is a schematic cross-sectional view of an organic electroluminescent device according to another exemplary embodiment. The organic electroluminescent device 20 according to an embodiment may include a substrate 1, a first electrode 2, a hole transport region 3, an emission layer 4, an electron transport region 5, and a second electrode 6, which are sequentially stacked in this stated order. In FIG. 2, the hole transport region 3 includes a hole injection layer 31 and a hole transport layer 32, which are sequentially stacked in this stated order. In FIG. 2, the electron transport region 5 includes an electron transport layer 52 and an electron injection layer 51, which are sequentially stacked in this stated order.

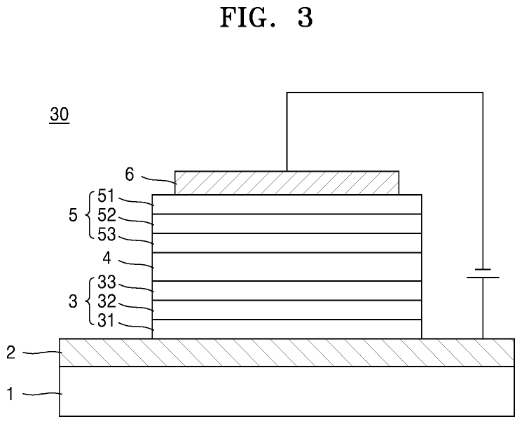

FIG. 3 is a schematic cross-sectional view of an organic electroluminescent device according to another embodiment. The organic electroluminescent device 30 according to an embodiment may include a substrate 1, a first electrode 2, a hole transport region 3, an emission layer 4, an electron transport region 5, and a second electrode 6, which are sequentially stacked in this stated order. In FIG. 3, the hole transport region 3 includes a hole injection layer 31, a hole transport layer 32, and an electron blocking layer 33, which are sequentially stacked in this stated order. In FIG. 3, the electron transport region 5 includes an electron blocking layer 53, an electron transport layer 52, and an electron injection layer 51, which are sequentially stacked in this stated order.

Hereinafter, the substrate, each region, and each layer will be explained in detail.

### Substrate 1

The organic electroluminescent device 10 may have a substrate 1. A substrate that is used as a general organic electroluminescent device may be used as the substrate 1. For example, the substrate 1 may be a glass substrate, a semiconductor substrate such as a silicon substrate, or a transparent plastic substrate.

### First Electrode 2

The first electrode 2 has conductivity. In the organic electroluminescent device according to an embodiment, the first electrode 2 may be an anode. The first electrode 2 may be a pixel electrode. The first electrode 2 may be a transmissive electrode, a semi-transmissive electrode, or a reflective electrode.

Examples of the material forming the first electrode 2 may include, but is not limited to, a metal alloy or a conductive compound. When the first electrode 2 is a transmissive electrode, the first electrode 2 may include a transparent metal oxide, for example, indium tin oxide (ITO), indium zinc oxide (IZO), zinc oxide (ZnO), indium tin zinc oxide (ITZO), and the like. When the first electrode 2 is a semi-transmissive electrode or a reflective electrode, the first electrode 2 may include Ag, Mg, Cu, Al, Pt, Pd, Au, Ni, Nd, Ir, Cr, Li, Ca, LiF/Ca, LiF/Al, Mo, Ti, compounds or mixtures thereof (for example, a mixture of Ag and Mg), and the like.

The first electrode 2 may be single-layered including a single material, or single-layered including a plurality of different materials. In addition, the first electrode 2 may be multi-layered including a plurality of different materials.

The thickness of the first electrode 2 is not particularly limited, but may be 10 nm or more and 1,000 nm or less, or may be 50 nm or more and 300 nm or less.

### Hole Transport Region 3

The hole transport region 3 may be provided on the first electrode 2. The hole transport region 3 may include at least one of the hole injection layer 31, the hole transport layer 32, the buffer layer (not shown), and the electron blocking layer 33.

The hole transport region 3 may be single-layered including a single material, or single-layered including a plurality of different materials. In addition, the hole transport region 3 may be multi-layered including a plurality of different materials.

For example, the hole transport region 3 may have a single-layered structure consisting of the hole injection layer 31 or the hole transport layer 32. For example, the hole transport region 3 may have a single-layered structure consisting of the hole injection material and the hole transport material. For example, the hole transport region 3 may have a hole injection layer 31/hole transport layer 32 structure, wherein constituting layers are sequentially stacked from the first electrode 2. For example, the hole transport region 3 may have a hole injection layer 31/hole transport layer 32/hole buffer layer (not shown) structure. For example, the hole transport region 3 may have a hole injection layer 31/hole buffer layer (not shown) structure, wherein constituting layers are sequentially stacked from the first electrode 2. For example, the hole transport region 3 may have a hole transport layer 32/hole buffer layer (not shown) structure, wherein constituting layers are sequentially stacked from the first electrode 2. For example, the hole transport region 3 may have a hole injection layer 31/hole transport layer 32/electron blocking layer 33 structure, wherein constituting layers are sequentially stacked from the first electrode 2. The structure of the hole transport region is not limited to the above embodiments.

The hole injection layer 31 or other layers constituting the hole transport region 3 are not particularly limited, and, for example, a known hole injection material may be used. Examples of the hole injection layer may include a phthalocyanin compound such as copper phthalocyanin, N,N′-diphenyl-N,N′-bis-4-(phenyl-m-tolyl-amino)-phenyl-biphenyl-4,4′-diamine) (DNTPD), (4,4′,4″-tris(3-methylphenylphenylamino)triphenylamine) (m-MTDATA), 4,4′,4″-tris(N,N-diphenylamino)triphenylamine) (TDATA), (4,4′,4″-tris{N-(2-naphthyl)-N-phenyl amino}-triphenylamine) (2-TNATA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), N,N′-di(naphthalene-1-yl)-N,N′-diphenylbenzidin (NPB), polyetherketone including triphenylamine (TPAPEK), 4-isopropyl-4′-methyldiphenyliodoniumtetrakis(pentafluorophenyl)borate, dipyrazino[2,3-f:2′,3′-h]quinoxaline-2,3,6,7,10,11-hexacarbonitrile (HAT-CN), 1,3,4,5,7,8-hexafluorotetracyano-2,6-naphthoquinodimethane (F6-TCNNQ), and the like.

The hole transport layer 32 or other layers constituting the hole transport region 3 are not particularly limited, and, for example, a known hole transport material may be used. Examples of the hole transport material may include N-phenylcarbazole, carbazole-based derivatives such as polyvinyl carbazole, fluorene-based derivatives, triphenylamine-based derivatives such as N,N′-bis(3-methyl phenyl)-N,N′-diphenyl-1,1-biphenyl]-4,4′-diamine) (TPD), 4,4′,4″-tris(N-carbazolyl)triphenylamine (TCTA), N,N′-di(a naphthalene-1-yl)-N,N′-diphenylbenzidine (NPB), 4,4′-cyclohexylidenbis[N,N-bis(4-methylphenyl)benzenamine (TAPC), 4,4′-bisN,N′-(3-tolyl)amino]-3,3′-dimethylbiphenyl (HMTPD), 1,3-bis(N-carbazolyl)benzene (mCP), Compound HTM1, Compound HTM2, Compound HT1, and the like:

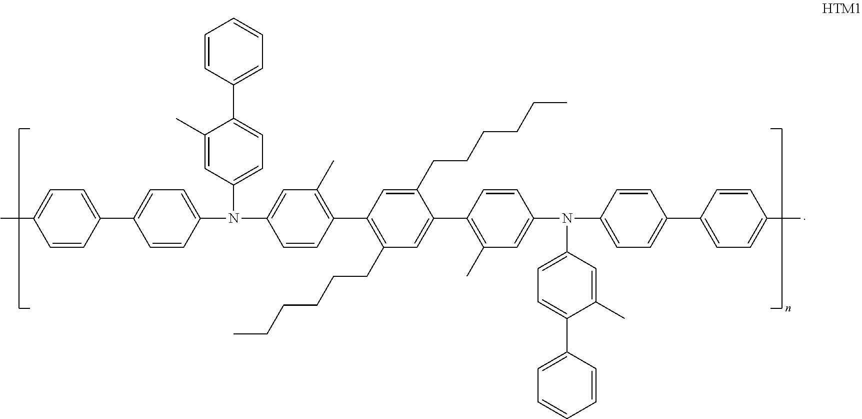

In HTM1, n may be an integer of 1 or more.

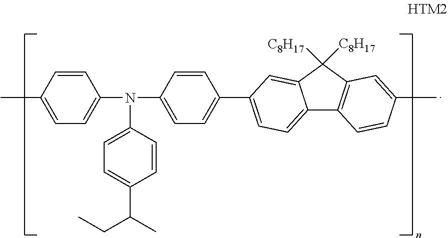

In HTM2, n may be an integer of 1 or more.

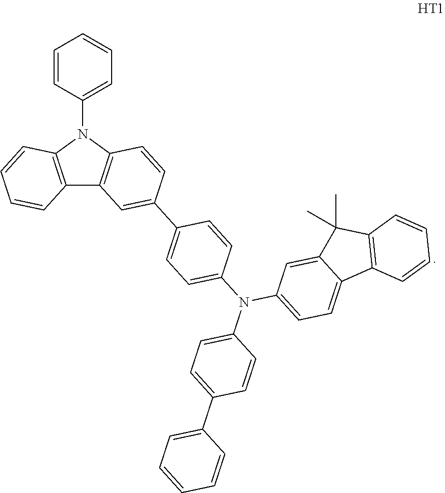 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

The hole transport region 3 may further include a charge generation material, in addition to the hole injection material or the hole transport material to improve conductivity. The charge generation material may be homogeneously or non-homogeneously dispersed in the hole transport region 3 or each layer thereof. An example of the charge generation material may include, but is not particularly limited to, a known charge generation material. An example of the charge generation material may include a p-dopant. Examples of the p-dopant may include a quinone derivative such as tetracyanoquinonedimethane (TCNQ), 2,3,5,6-tetrafluorotetracyanoquinodimethane) (F4-TCNQ), and the like, metal oxide such as tungsten oxide, molybdenum oxide, and the like, and a cyano group-containing compound, etc.

The buffer layer (not shown) may compensate for an optical resonance distance according to a wavelength of light emitted from the emission layer 4 to improve photoluminescence emission efficiency. Materials included in the hole buffer layer (not shown) are not particularly limited, and materials used in the hole transport layer may be used. For example, the compounds that may be included in the hole transport region 3 may be used.

The electron blocking layer 33 may block the flow of electrons from the electron transport region 5 to the hole transport region 3. Materials included in the electron blocking layer 33 are not particularly limited, and materials used in the known electron blocking layer may be used. For example, the host material etc. included in the emission layer (the material for the organic electroluminescent device) may be used, and host materials such as Compounds H55, H86, and H87 may be examples thereof.

A thickness of the hole transport region 3 is not particularly limited, and may be 1 nm or more and 1,000 nm or less, and 10 nm or more and 500 nm or less. Among each layer constituting the hole transport region 3, a thickness of the hole injection layer 31 may be, but not particularly limited to, 3 nm or more and 200 nm or less. A thickness of the hole transport layer 32 may be, but not limited to, 3 nm or more and 200 nm or less. A thickness of the electron blocking layer 33 may be, but not limited to, 1 nm or more and 100 nm or less. In addition, the thickness of the hole buffer layer (not shown) is not particularly limited as long as the hole buffer layer functions as a hole buffer layer and does not interfere with the function as an organic electroluminescent device. When the thicknesses of the hole transport region 3, the hole injection layer 31, the hole transport layer 32, or the electron blocking layer 33 are within these ranges, excellent hole transporting characteristics may be obtained while substantially suppressing the increase in driving voltage.

Examples of the film forming method of the hole transport region 3 or each layer thereof may include, but not limited to, vacuum deposition, spin coating, LB deposition, ink-jet printing, laser-printing, and LITI.

### Emission Layer 4

The emission layer 4 may be located on the hole transport region 3. Details of the emission layer 4 are the same as described above.

### Electron Transport Region 5

The electron transport region 5 may be located on the emission layer 4. The electron transport region 5 may include at least one of the electron injection layer 51, the election transport layer 52, and the hole blocking layer 53, but embodiments of the present disclosure are not limited thereto.

The electron transport region 5 may be single-layered including a single material, or single-layered including a plurality of different materials. In addition, the electron transport region 5 may be multi-layered including a plurality of different materials. For example, the electron transport region 5 may have a single-layered structure consisting of the electron injection layer 51 or the electron transport layer 52. For example, the electron transport region 5 may have a single-layered structure consisting of the electron injection material and the electron transport material. For example, the electron transport region 5 may have an electron transport layer 52/electron injection layer 51 structure, wherein constituting layers are sequentially stacked from the emission layer 4. For example, the electron transport region 5 may have a hole blocking layer 53/electron transport layer 52/electron injection layer 51 structure, wherein constituting layers are sequentially stacked from the emission layer 4. The structure of the electron transport region 5 is not limited to the above embodiments.

The electron injection layer 51 or other layers constituting the electron transport region 5 are not particularly limited, and, for example, a known electron injection material may be used. Examples of the electron injection material may include a lanthanide metal such as LiF, LiQ (Lithum quinolate), Li₂O, BaO, NaCl, CsF, and Yb, or a metal halide such as RbCl. Examples of the electron injection layer 51 may include, but is not limited to, the electron transport material and an insulative organometallic salt. The organometallic salt is not particularly limited, and may be a material having an energy band gap of 4 eV or more. Examples of the organometallic salt may include an acetate metallic salt, a benzoate metallic salt, an acetoacetic metallic salt, an acetylacetonate metallic salt, or a stearate metallic salt.

The electron transport layer 52 or other layers constituting the electron transport region 5 are not particularly limited, and, for example, a known electron transport material may be used. Examples of the electron transport material may include an anthracene-based compound, tris(8-hydroxyquinolinate)aluminum) (Alq₃), 1,3,5-tri[(3-pyridyl)-pen-3-yl]benzene, 2,4,6-tris(3′-pyridine-3-yl)biphenyl-3-yl)-1,3,5-triazine, 2-(4-(N-phenylbenzimidazole-1-ylphenyl)-9,10-dinaphthylan anthracene, 1,3,5-tri(1-phenyl-1H-benzo [d] imidazole-2-yl)phenyl) (TPBi), 2,9-dimethyl-4,7-diphenyl-1,10-phenanthroline (BCP), 4,7-diphenyl-1,10-phenanthroline (Bphen), 3-(4-biphenyl)-4-phenyl-5-tert-butylphenyl-1,2,4-triazole (TAZ), 4-(naphthalene-1-yl)-3,5-diphenyl-4H-1,2,4-triazole (NTAZ), 2-(4-biphenyl)-5-(4-tert-butylphenyl)-1,3,4-oxadiazole (tBu-PBD), bis(2-methyl-8-quinolinorato-N1,O8)-(1,1′-biphenyl-4-orato)aluminum (BAlq), beryllium bis(benzoquinoline-10-orate) (Bebq2), 9,10-di(naphthalene-2-yl)anthracene (ADN), Lithum quinolate (LiQ), Compound ET1, and the like: In addition, TRE314 (manufactured by Toray Industries, Inc., electron transport material) may be an example of the electron transport material.

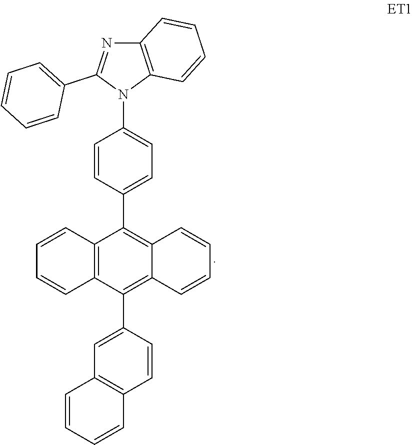 [SMILES: c1ccc(-c2nc3ccccc3n2-c2ccc(-c3c4ccccc4c(-c4ccc5ccccc5c4)c4ccccc34)cc2)cc1]

The hole blocking layer 53 may block the flow of holes from the hole transport region 3 to the electron transport region 5. Materials included in the hole blocking layer 53 are not particularly limited, and materials used in the known hole blocking layer 53 may be used. The hole blocking layer 53 may include a known hole blocking material, for example. Examples of the hole blocking material may include 2,9-dimethyl-4,7-diphenyl-1,10-phenanthroline (BCP), 4,7-diphenyl-1,10-phenanthroline (BPhen), and the like. For example, the host material such as Compound H77 included in the emission layer (the material for the organic electroluminescent device) may be used.

A thickness of the electron transport region 5 is not particularly limited, and may be 0.1 nm or more and 200 nm or less, and 30 nm or more and 150 nm or less. Among each layer constituting the electron transport region 5, a thickness of the electron transport layer 52 may be, but not particularly limited to, 10 nm or more and 100 nm or less. A thickness of the hole blocking layer 53 is not particularly limited, and may be 1 nm or more and 100 nm or less, and 5 nm or more and 30 nm or less. A thickness of the electron injection layer 51 is not particularly limited, and may be 0.1 nm or more and 10 nm or less, and 0.3 nm or more and 9 nm or less. When the thickness of the electron injection layer 51 is within the above range, excellent hole transporting characteristics may be obtained while substantially suppressing the increase in driving voltage. When the thicknesses of the electron transport region 5, the electron injection layer 51, the electron transport layer 52, or the hole blocking layer 53 are within these ranges, excellent hole transporting characteristics may be obtained while substantially suppressing the increase in driving voltage.

Examples of the film forming method of the electron transport region 5 or each layer thereof may include, but not limited to, vacuum deposition, spin coating, LB deposition, ink-jet printing, laser-printing, and LITI.

The second electrode 6 may be located on the electron transport region 5. The second electrode 6 may have conductivity. In the organic electroluminescent device according to an embodiment, the second electrode 6 may be a common electrode or a cathode. The second electrode 6 may be a transmissive electrode, a semi-transmissive electrode, or a reflective electrode.

Examples of the material forming the second electrode 6 may include, but is not limited to, a metal alloy or a conductive compound. When the second electrode 6 is a reflective electrode, the second electrode 6 may include a transparent metal oxide, for example, ITO, IZO, ZnO, ITZO, and the like. When the second electrode 6 is a semi-transmissive electrode or a reflective electrode, the second electrode 6 may include Ag, Mg, Cu, Al, Pt, Pd, Au, Ni, Nd, Ir, Cr, Li, Ca, LiF/Ca, LiF/Al, Mo, Ti, compounds or mixtures thereof (for example, a mixture of Ag and Mg), and the like.

The second electrode 6 may be single-layered including a single material, or single-layered including a plurality of different materials. In addition, the second electrode 6 may be multi-layered including a plurality of different materials.

A thickness of the second electrode 6 may be, but not limited to, 10 nm or more and 1,000 nm or less.

The second electrode 6 may be connected to an auxiliary electrode (not shown). By connecting the second electrode 6 to the auxiliary electrode, the resistance of the second electrode 6 may be reduced.

A capping layer (not shown) may be additionally located on the second electrode 6. Examples of the capping layer (not shown) may include, but are not limited to, α-NPD, NPB, TPD, m-MTDATA, Alq3, CuPc, N4,N4,N4′,N4′-tetra(biphenyl-4-yl), biphenyl 4,4′-diamine (TPD15), 4,4′,4″-tri-9-carbazolyltriphenylamine (TCTA), N,N′-bis(naphthalene-1-yl), and the like.

The material constituting each layer and each electrode may be used alone or in combination of two or more.

In the organic electroluminescent device 10 of FIGS. 1 to 3, the nitrogen-containing condensed cyclic compound, the fluorescence emitter, or the material for the organic electroluminescent device may be included in the emission layer 4, but may also be included in organic layers other than the emission layer 4. The nitrogen-containing condensed cyclic compound, the fluorescence emitter, or the material for the organic electroluminescent device may be included in the emission layer 4 and the organic layers other than the emission layer 4.

In the organic electroluminescent device 10 of FIGS. 1 to 3, because voltage is applied to each of the first electrode 2 and the second electrode 6, the hole provided from the first electrode 2 may move toward the emission layer 4 through the hole transport region 3, and the electron provided from the second electrode 6 may move toward the emission layer 4 through the electron transport region 5. The hole and the electron may recombine in the emission layer 4 to produce excitons, and these excitons may transition from an excited state to a ground state to thereby generate light.

### EXAMPLES

Hereinafter, the present disclosure will be described in more detail with reference to the examples and comparative examples, but the technical scope of the present disclosure is not limited to the following examples.

### Simulation Evaluation of Condensed Cyclic Compound

In “High-Performance Dibenzoheteraborin-Based Thermally Activated Delayed Fluorescence Emitters: Molecular Architectonics for Concurrently Achieving Narrowband Emission and Efficient Triplet-Singlet Spin Conversion” In Seob Park, Kyohei Matsuo, Naoya Aizawa, and Takuma Yasuda, Advanced Functional Materials 2018, 28, 1802031, a spectral width of a fluorescent luminescence (Full Width at Half Maximum (FWHM)) is described to be closely related to a reorganization energy [E(S₀@S₁)−E(S₀@S₀)], which is represented by a difference between an energy of the ground state (S₀) in a stable structure of the first excitation singlet state (S₁) [E(S₀@S₁)] and an energy of the ground state (S₀) in a stable structure of the ground state (S₀) [E(S₀@S₀)]. Verification of relation of reorganization energy and spectral width of fluorescent luminescence

First, a relation of the reorganization energy [E(S₀@S₁)−E(S₀@S₀)] and the spectral width (FWHM) of the fluorescent luminescence is explained as below.

### Calculation by DFT

The calculation below was conducted regarding known condensed cyclic compounds R1 to R3 by DFT.

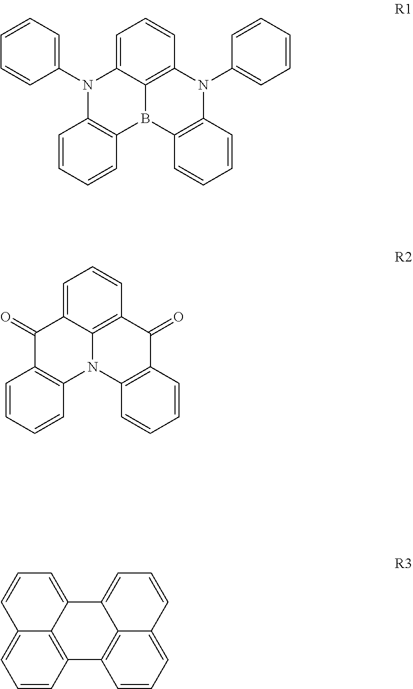

An energy of the ground state (S₀) in a stable structure of the first excitation singlet state (S₁) [E(S₀@S₁)] and an energy of the ground state (S₀) in a stable structure of the ground state (S₀) [E(S₀@S₀)] were calculated, and from a difference between the two values, the reorganization energy [E(S₀@S₁)]−[E(S₀@S₀)] (eV) was calculated.

In addition, an energy of the first excitation singlet state (S₁) in a stable structure of the first excitation singlet state (S₁) [E(S₁@S₁)] was calculated, and from a difference between the calculated value and an energy of the ground state (S₀) in a stable structure of the ground state (S₀) [E(S₀@S₀)], an adiabatic first excitation singlet state (S1) energy [E(S₁@S₁)]−[E(S₀@S₀)] (eV) was calculated.

Further, the adiabatic first excitation singlet state (S₁) energy (eV) converted to light wavelength (nm), that is, a fluorescent wavelength (nm) was calculated.

Further, an oscillator strength f in the stable structure of the first excitation singlet state (S₁) was calculated.

Further, a highest occupied molecular orbital (HOMO) energy and a lowest unoccupied molecular orbital (LUMO) energy were calculated.

The calculation through the DFT was performed using Gaussian 16 (Gaussian Inc.) as a calculation software and using calculation methods (I), (II), and (III):

(I) S₀ calculation method: calculation of structure optimization through DFT including functional B3LYP, basis function 6-31 G(d, p), and toluene solvent effect (PCM);

(II) S₁ calculation method: calculation of structure optimization through time-dependent DFT (TDDFT) including functional B3LYP, basis function 6-31 G(d, p), and toluene solvent effect (PCM);

(III) S₀ calculation method: calculation of input structure through DFT including functional B3LYP, basis function 6-31 G(d, p), and toluene solvent effect (PCM).

In particular, calculation of each item was performed by using the calculation methods below:
energy of the ground state (S₀) in a stable structure of the ground state (S₀) [E(S₀@S₀)]: calculation method of (I);
energy of the first excitation singlet state (S₁) in a stable structure of the first excitation singlet state (S₁) [E(S₁@S₁)]: calculation method of (II);
energy of the ground state (S₀) in a stable structure of the first excitation singlet state (S₁) [E(S₀@S₁)]: calculation method of (II) and (III);
reorganization energy [E(S₀@S₁)]−[E(S₀@S₀)]: calculation method of (I), (II), and (III);
adiabatic first excitation singlet state (S₁) energy [E(S₁@S₁)]−[E(S₀@S₀)]: calculation method of (I) and (II);
fluorescent wavelength (nm): calculation method of (I) and (II);
oscillator strength f in the stable structure of the first excitation singlet state (S₁): calculation method of (II);
HOMO and LUMO: calculation method of (I).

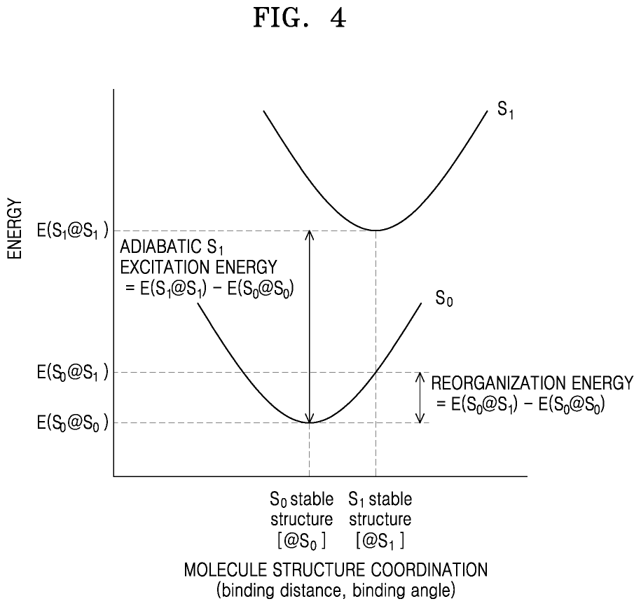

In addition, FIG. 4 is a diagram illustrating a qualitative relation of each energy.

### Measurement of Spectral Width (FWHM) of Fluorescent Luminescence

Each toluene solution of 1×10⁻⁵ M(=mol/dm³, mol/L) of condensed cyclic compounds R1 to R3 were measured using a spectro fluorescence photometer F7000 of Hitachi Hightech Science Corporation at room temperature with an excitation wavelength of 320 nm, and the fluorescent luminescence peak wavelength (nm) in PL and the spectral width (FWHM) of the fluorescent luminescence were evaluated. Results of the evaluation is shown in Table 2.

TABLE 2
Calculation Result according to DFT and Measurement Result of FWHM of fluorescent luminescence

| | Calculation by DFT | | | | | | Measurement | |
|---|---|---|---|---|---|---|---|---|
| | | | Adiabatic first | | | | PL | |
| | | | excitation singlet | Oscillator | Reorganization | Fluorescent | Peak | PL |
| | HOMO | LUMO | state (S₁) | strength | energy | wavelength | wavelength | FWHM |
| Cmpd. | (eV) | (eV) | energy (eV) | f | (eV) | (nm) | (nm) | (nm) |
| R1 | −4.88 | −1.23 | 2.99 | 0.214 | 0.109 | 415 | 453 | 22 |
| R2 | −5.94 | −2.36 | 2.96 | 0.161 | 0.132 | 419 | 451 | 26 |
| R3 | −5.02 | −1.96 | 2.62 | 0.491 | 0.164 | 474 | 445 | 42 |

From the results of Table 2, it can be seen that the values of the color of the fluorescent wavelength (nm) calculated through the DFT and the color of the measured peak wavelength are somewhat close. In this regard, it was confirmed that the color predicted by the calculation according to the DFT and the measured color have the same tone.

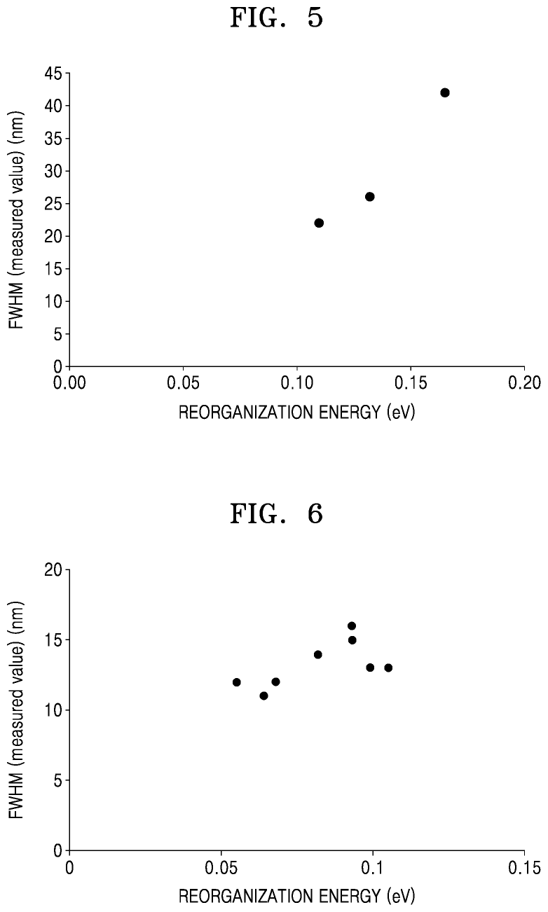

A graph regarding known condensed cyclic compounds R1 to R3 showing the FWHM of a fluorescent luminescence in a measured PL relative to the reorganization energy (eV) calculated according to the DFT is shown in FIG. 5. From the results of FIG. 5, it can be seen that the relocation energy (eV) calculated according to the DFT is related to the FWHM of the fluorescent luminescence, and the smaller the reorganization energy (eV), the smaller the FWHM of the fluorescent luminescence, that is, the spectral width of the fluorescent luminescence is narrowed.

Evaluation of Compounds by Calculation 1: Calculation of E(S₁) in Ground State of Compound of Present Disclosure

The singlet energy E(S₁) (adiabatic first excitation singlet state (S₁) energy) of the nitrogen-containing condensed cyclic compound of the present disclosure was calculated. In an embodiment, for some of the nitrogen-containing condensed cyclic compounds 1 to 317 of Tables 3 to 9 exemplified as nitrogen-containing condensed cyclic compounds of the present disclosure, the singlet energy E(S₁) was calculated through DFT. In addition, the number of the compounds shown in Tables 3 to 9 indicate the number of the compounds specifically exemplified above.

### Calculation Method

E(S₁): calculate through TDDFT through functional B3LYP and basis function 6-31 G(d, p) in ground state optimization structure calculated by DFT using functional B3LYP and basis function 6-31 G(d, p) (Calculation software used: Gaussian 16 (Gaussian Inc.)).

### Evaluation of Compounds by Calculation 2: Calculation of Oscillator Strength f, Reorganization Energy, and Fluorescent Wavelength of Compound of Present Disclosure

Through the method described in the [Verification of relation of reorganization energy and spectral width of fluorescent luminescence] section, the oscillator strength f, reorganization energy, and fluorescent luminescence of the nitrogen-containing condensed cyclic compounds 1 to 317, which were described as examples of nitrogen-containing condensed cyclic compounds of the present disclosure, were calculated.

The results of calculation are shown in Tables 3 to 9 below.

TABLE 3
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 1 | −5.19 | −1.90 | 2.76 | 450 | 0.403 | 0.076 |
| 2 | −5.16 | −1.88 | 2.75 | 451 | 0.460 | 0.080 |
| 3 | −5.18 | −1.85 | 2.80 | 443 | 0.347 | 0.062 |
| 5 | −5.28 | −1.94 | 2.80 | 442 | 0.200 | 0.058 |
| 6 | −5.24 | −1.91 | 2.78 | 446 | 0.179 | 0.068 |
| 7 | −5.30 | −1.92 | 2.86 | 434 | 0.303 | 0.055 |
| 8 | −5.23 | −1.90 | 2.79 | 445 | 0.189 | 0.065 |
| 9 | −5.31 | −2.00 | 2.77 | 447 | 0.692 | 0.096 |
| 10 | −5.26 | −1.97 | 2.76 | 449 | 0.982 | 0.105 |
| 11 | −5.31 | −1.90 | 2.88 | 430 | 0.318 | 0.060 |
| 12 | −5.26 | −1.95 | 2.78 | 446 | 0.823 | 0.099 |
| 13 | −5.19 | −1.88 | 2.78 | 447 | 0.578 | 0.093 |
| 14 | −5.18 | −1.90 | 2.76 | 450 | 0.608 | 0.082 |
| 15 | −5.26 | −1.87 | 2.87 | 432 | 0.254 | 0.062 |
| 16 | −5.23 | −1.84 | 2.86 | 433 | 0.242 | 0.063 |
| 17 | −5.23 | −1.84 | 2.87 | 432 | 0.262 | 0.060 |
| 18 | −5.22 | −1.83 | 2.86 | 433 | 0.239 | 0.064 |
| 19 | −5.32 | −2.09 | 2.70 | 459 | 0.556 | 0.101 |
| 24 | −5.30 | −2.10 | 2.68 | 463 | 1.053 | 0.105 |
| 28 | −5.45 | −2.10 | 2.83 | 439 | 0.389 | 0.048 |
| 29 | −5.33 | −2.06 | 2.73 | 454 | 0.866 | 0.107 |
| 30 | −5.32 | −1.99 | 2.80 | 443 | 0.186 | 0.079 |
| 32 | −5.36 | −2.04 | 2.79 | 444 | 0.339 | 0.058 |
| 34 | −5.42 | −2.09 | 2.81 | 442 | 0.310 | 0.051 |
| 35 | −5.73 | −2.31 | 2.88 | 431 | 0.249 | 0.081 |
| 37 | −5.78 | −2.37 | 2.89 | 429 | 0.301 | 0.064 |
| 39 | −5.61 | −2.13 | 2.95 | 420 | 0.317 | 0.075 |
| 40 | −5.60 | −2.28 | 2.80 | 444 | 0.282 | 0.069 |
| 41 | −5.61 | −2.19 | 2.90 | 428 | 0.306 | 0.068 |
| 42 | −5.48 | −2.22 | 2.73 | 455 | 0.246 | 0.072 |
| 43 | −5.39 | −2.05 | 2.83 | 438 | 0.262 | 0.051 |
| 44 | −5.44 | −1.95 | 2.96 | 418 | 0.306 | 0.071 |
| 45 | −5.39 | −2.07 | 2.79 | 444 | 0.189 | 0.049 |
| 46 | −5.38 | −1.99 | 2.88 | 430 | 0.306 | 0.057 |
| 47 | −6.07 | −2.63 | 2.91 | 425 | 0.343 | 0.067 |
| 48 | −5.68 | −2.27 | 2.89 | 428 | 0.326 | 0.065 |
| 49 | −5.55 | −2.18 | 2.85 | 434 | 0.260 | 0.057 |
| 53 | −5.37 | −2.16 | 2.69 | 461 | 0.535 | 0.108 |

TABLE 4
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 56 | −6.22 | −2.98 | 2.73 | 454 | 0.358 | 0.068 |
| 57 | −6.23 | −2.81 | 2.90 | 427 | 0.335 | 0.056 |
| 58 | −5.91 | −2.55 | 2.84 | 437 | 0.318 | 0.069 |
| 59 | −5.94 | −2.49 | 2.92 | 424 | 0.338 | 0.075 |
| 60 | −5.38 | −2.26 | 2.60 | 477 | 0.302 | 0.101 |
| 61 | −5.40 | −2.01 | 2.87 | 432 | 0.394 | 0.058 |
| 63 | −5.48 | −2.09 | 2.87 | 432 | 0.548 | 0.057 |
| 64 | −5.18 | −1.92 | 2.73 | 455 | 0.184 | 0.058 |
| 65 | −5.28 | −2.04 | 2.72 | 455 | 0.499 | 0.069 |
| 66 | −5.26 | −1.99 | 2.72 | 456 | 0.190 | 0.067 |
| 69 | −5.32 | −1.91 | 2.88 | 430 | 0.347 | 0.065 |
| 70 | −5.31 | −1.92 | 2.86 | 434 | 0.363 | 0.060 |
| 71 | −5.30 | −1.94 | 2.83 | 438 | 0.337 | 0.050 |
| 73 | −5.09 | −1.87 | 2.69 | 460 | 0.162 | 0.060 |
| 74 | −5.25 | −2.03 | 2.69 | 461 | 0.620 | 0.106 |
| 75 | −5.20 | −1.98 | 2.70 | 458 | 0.581 | 0.069 |
| 76 | −5.18 | −1.94 | 2.68 | 463 | 0.144 | 0.111 |
| 79 | −5.25 | −1.99 | 2.72 | 456 | 0.574 | 0.098 |
| 80 | −5.19 | −1.95 | 2.72 | 456 | 0.526 | 0.066 |
| 81 | −5.17 | −1.91 | 2.71 | 458 | 0.137 | 0.114 |
| 83 | −5.37 | −1.97 | 2.87 | 432 | 0.362 | 0.072 |
| 84 | −5.35 | −1.98 | 2.84 | 436 | 0.355 | 0.059 |
| 85 | −5.34 | −2.00 | 2.81 | 442 | 0.319 | 0.050 |
| 86 | −5.30 | −2.04 | 2.74 | 452 | 0.711 | 0.079 |
| 87 | −5.33 | −2.09 | 2.70 | 459 | 0.573 | 0.109 |
| 89 | −5.28 | −2.13 | 2.65 | 469 | 0.984 | 0.103 |
| 90 | −5.30 | −2.07 | 2.70 | 460 | 0.412 | 0.074 |
| 91 | −5.27 | −2.00 | 2.71 | 458 | 0.154 | 0.109 |
| 92 | −5.28 | −2.10 | 2.67 | 464 | 0.992 | 0.097 |
| 98 | −5.34 | −2.09 | 2.73 | 453 | 0.593 | 0.075 |
| 99 | −5.30 | −2.06 | 2.73 | 455 | 0.868 | 0.088 |
| 100 | −5.30 | −2.04 | 2.74 | 452 | 0.738 | 0.081 |
| 101 | −5.34 | −2.10 | 2.71 | 457 | 0.502 | 0.075 |
| 102 | −5.30 | −2.07 | 2.69 | 461 | 0.387 | 0.075 |
| 103 | −5.30 | −2.07 | 2.70 | 459 | 0.432 | 0.073 |
| 104 | −5.39 | −2.10 | 2.74 | 453 | 0.138 | 0.113 |
| 105 | −5.25 | −1.97 | 2.73 | 455 | 0.157 | 0.100 |
| 106 | −5.45 | −2.20 | 2.71 | 458 | 0.674 | 0.108 |

TABLE 5
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 107 | −5.30 | −2.12 | 2.65 | 468 | 0.677 | 0.106 |
| 108 | −5.40 | −2.15 | 2.72 | 456 | 0.563 | 0.084 |
| 109 | −5.27 | −2.03 | 2.72 | 455 | 1.067 | 0.103 |
| 110 | −5.27 | −2.05 | 2.71 | 458 | 1.046 | 0.101 |
| 111 | −5.27 | −1.98 | 2.76 | 450 | 1.038 | 0.107 |
| 112 | −5.21 | −1.91 | 2.76 | 449 | 0.781 | 0.101 |
| 113 | −5.21 | −1.93 | 2.75 | 451 | 0.885 | 0.095 |
| 114 | −5.25 | −1.92 | 2.80 | 443 | 0.530 | 0.093 |
| 115 | −5.23 | −1.88 | 2.82 | 440 | 0.235 | 0.052 |
| 122 | −5.26 | −2.01 | 2.71 | 457 | 0.486 | 0.099 |
| 123 | −5.23 | −1.98 | 2.73 | 454 | 0.509 | 0.071 |
| 124 | −5.23 | −1.86 | 2.87 | 431 | 0.308 | 0.086 |
| 125 | −5.20 | −1.84 | 2.83 | 437 | 0.302 | 0.052 |
| 126 | −5.21 | −1.94 | 2.73 | 454 | 0.410 | 0.095 |
| 127 | −5.18 | −1.90 | 2.75 | 451 | 0.405 | 0.070 |
| 128 | −5.15 | −1.85 | 2.76 | 450 | 0.239 | 0.053 |
| 129 | −5.23 | −1.96 | 2.74 | 452 | 0.393 | 0.067 |
| 130 | −5.20 | −1.92 | 2.73 | 453 | 0.209 | 0.061 |
| 133 | −5.17 | −1.85 | 2.78 | 446 | 0.416 | 0.089 |
| 134 | −5.14 | −1.81 | 2.81 | 441 | 0.298 | 0.054 |
| 135 | −5.17 | −1.87 | 2.77 | 448 | 0.420 | 0.079 |
| 136 | −5.14 | −1.83 | 2.78 | 446 | 0.237 | 0.051 |
| 137 | −5.27 | −2.01 | 2.75 | 452 | 0.613 | 0.080 |
| 138 | −5.28 | −1.99 | 2.76 | 450 | 0.466 | 0.087 |
| 139 | −5.25 | −2.03 | 2.69 | 460 | 0.285 | 0.080 |
| 140 | −5.23 | −1.86 | 2.84 | 436 | 0.286 | 0.057 |
| 141 | −5.23 | −1.85 | 2.86 | 434 | 0.330 | 0.056 |
| 142 | −5.23 | −1.93 | 2.77 | 448 | 0.405 | 0.085 |
| 143 | −5.28 | −1.91 | 2.84 | 437 | 0.265 | 0.052 |
| 144 | −5.22 | −1.95 | 2.73 | 454 | 0.360 | 0.088 |
| 145 | −5.26 | −1.90 | 2.83 | 439 | 0.250 | 0.052 |
| 146 | −5.23 | −1.87 | 2.83 | 439 | 0.261 | 0.051 |
| 147 | −5.31 | −1.98 | 2.80 | 443 | 0.639 | 0.092 |
| 148 | −5.24 | −1.91 | 2.79 | 444 | 0.505 | 0.089 |
| 149 | −5.19 | −1.93 | 2.74 | 453 | 0.603 | 0.074 |
| 150 | −5.21 | −1.96 | 2.72 | 456 | 0.522 | 0.100 |
| 151 | −5.18 | −1.92 | 2.73 | 454 | 0.346 | 0.067 |
| 152 | −5.16 | −1.88 | 2.73 | 455 | 0.172 | 0.085 |

TABLE 6
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 153 | −5.47 | −2.05 | 2.82 | 440 | 0.238 | 0.089 |
| 154 | −5.27 | −1.95 | 2.78 | 446 | 0.621 | 0.094 |
| 155 | −5.24 | −1.90 | 2.80 | 443 | 0.192 | 0.072 |
| 156 | −5.25 | −1.94 | 2.78 | 446 | 0.655 | 0.096 |
| 157 | −5.21 | −1.88 | 2.80 | 443 | 0.198 | 0.057 |
| 159 | −5.21 | −1.84 | 2.83 | 438 | 0.296 | 0.053 |
| 160 | −5.24 | −1.91 | 2.79 | 444 | 0.493 | 0.092 |
| 161 | −5.21 | −1.90 | 2.77 | 447 | 0.656 | 0.098 |
| 162 | −5.21 | −1.92 | 2.76 | 449 | 0.794 | 0.089 |
| 165 | −5.21 | −1.84 | 2.85 | 435 | 0.345 | 0.069 |
| 166 | −5.21 | −1.80 | 2.88 | 430 | 0.370 | 0.069 |
| 167 | −5.20 | −1.84 | 2.83 | 438 | 0.284 | 0.053 |
| 168 | −5.19 | −1.83 | 2.82 | 439 | 0.245 | 0.048 |
| 169 | −5.17 | −1.81 | 2.84 | 437 | 0.328 | 0.057 |
| 172 | −5.21 | −1.90 | 2.77 | 447 | 0.656 | 0.098 |
| 173 | −5.21 | −1.92 | 2.76 | 449 | 0.794 | 0.089 |
| 174 | −5.28 | −1.88 | 2.87 | 432 | 0.344 | 0.069 |
| 175 | −5.19 | −1.81 | 2.86 | 434 | 0.339 | 0.071 |
| 176 | −5.19 | −1.81 | 2.86 | 434 | 0.293 | 0.052 |
| 177 | −5.29 | −1.90 | 2.87 | 433 | 0.319 | 0.060 |
| 178 | −5.17 | −1.84 | 2.81 | 442 | 0.336 | 0.073 |
| 179 | −5.22 | −1.84 | 2.84 | 436 | 0.289 | 0.050 |
| 180 | −5.24 | −1.94 | 2.77 | 448 | 0.745 | 0.101 |
| 181 | −5.23 | −1.94 | 2.76 | 449 | 0.717 | 0.088 |
| 182 | −5.22 | −1.92 | 2.76 | 449 | 0.632 | 0.096 |
| 183 | −5.20 | −1.92 | 2.75 | 450 | 0.680 | 0.082 |
| 184 | −5.27 | −1.89 | 2.85 | 435 | 0.385 | 0.070 |
| 185 | −5.21 | −1.81 | 2.86 | 433 | 0.371 | 0.071 |
| 186 | −5.20 | −1.84 | 2.84 | 437 | 0.361 | 0.059 |
| 187 | −5.22 | −1.95 | 2.74 | 453 | 0.539 | 0.092 |
| 188 | −5.24 | −1.93 | 2.78 | 446 | 0.617 | 0.093 |
| 189 | −5.23 | −1.94 | 2.77 | 448 | 0.886 | 0.097 |
| 190 | −5.23 | −1.94 | 2.77 | 448 | 0.886 | 0.097 |
| 191 | −5.25 | −1.87 | 2.85 | 434 | 0.371 | 0.068 |
| 192 | −5.20 | −1.80 | 2.87 | 431 | 0.362 | 0.069 |
| 193 | −5.18 | −1.82 | 2.84 | 437 | 0.351 | 0.059 |
| 194 | −5.33 | −1.95 | 2.77 | 447 | 0.063 | 0.171 |
| 195 | −5.23 | −1.92 | 2.78 | 446 | 0.673 | 0.093 |

TABLE 7
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 223 | −5.27 | −1.90 | 2.80 | 443 | 0.085 | 0.145 |
| 224 | −5.29 | −1.93 | 2.84 | 437 | 0.422 | 0.079 |
| 226 | −5.18 | −1.83 | 2.82 | 440 | 0.155 | 0.100 |
| 227 | −5.23 | −1.85 | 2.84 | 436 | 0.418 | 0.081 |
| 229 | −5.20 | −1.85 | 2.79 | 444 | 0.100 | 0.121 |
| 230 | −5.21 | −1.87 | 2.81 | 441 | 0.374 | 0.066 |
| 232 | −5.28 | −1.91 | 2.84 | 436 | 0.351 | 0.074 |
| 233 | −5.27 | −1.89 | 2.84 | 437 | 0.157 | 0.080 |
| 234 | −5.20 | −1.83 | 2.84 | 437 | 0.345 | 0.077 |
| 235 | −5.18 | −1.82 | 2.84 | 437 | 0.289 | 0.058 |
| 236 | −5.20 | −1.86 | 2.81 | 441 | 0.321 | 0.064 |
| 237 | −5.19 | −1.84 | 2.82 | 440 | 0.134 | 0.089 |
| 238 | −5.32 | −1.95 | 2.85 | 435 | 0.301 | 0.054 |
| 239 | −5.33 | −1.93 | 2.88 | 430 | 0.337 | 0.066 |
| 240 | −5.28 | −1.91 | 2.84 | 436 | 0.308 | 0.055 |
| 241 | −5.31 | −1.91 | 2.87 | 432 | 0.333 | 0.064 |
| 242 | −5.18 | −1.85 | 2.80 | 443 | 0.286 | 0.054 |
| 243 | −5.19 | −1.84 | 2.83 | 438 | 0.354 | 0.062 |
| 244 | −5.20 | −1.83 | 2.84 | 436 | 0.305 | 0.058 |
| 245 | −5.21 | −1.82 | 2.87 | 433 | 0.342 | 0.072 |
| 246 | −5.19 | −1.88 | 2.77 | 447 | 0.481 | 0.088 |
| 247 | −5.19 | −1.88 | 2.78 | 446 | 0.482 | 0.088 |
| 249 | −5.22 | −1.84 | 2.85 | 435 | 0.235 | 0.060 |
| 250 | −5.17 | −1.88 | 2.77 | 448 | 0.615 | 0.081 |
| 251 | −5.26 | −1.97 | 2.76 | 450 | 0.711 | 0.096 |
| 252 | −5.24 | −1.96 | 2.75 | 451 | 0.530 | 0.088 |
| 253 | −5.23 | −1.94 | 2.76 | 450 | 0.578 | 0.091 |
| 255 | −5.21 | −1.94 | 2.74 | 453 | 1.155 | 0.116 |
| 256 | −5.20 | −1.93 | 2.74 | 453 | 0.823 | 0.101 |
| 257 | −5.19 | −1.91 | 2.75 | 452 | 0.955 | 0.108 |
| 258 | −5.34 | −2.09 | 2.70 | 459 | 1.468 | 0.146 |
| 259 | −5.15 | −1.78 | 2.84 | 437 | 0.229 | 0.056 |
| 260 | −5.16 | −1.76 | 2.87 | 432 | 0.256 | 0.066 |
| 261 | −5.35 | −2.01 | 2.80 | 442 | 0.248 | 0.058 |
| 262 | −5.39 | −2.02 | 2.83 | 437 | 0.272 | 0.061 |
| 263 | −5.21 | −1.87 | 2.81 | 442 | 0.223 | 0.058 |
| 264 | −5.27 | −1.94 | 2.79 | 444 | 0.223 | 0.058 |
| 266 | −5.25 | −1.89 | 2.83 | 438 | 0.267 | 0.059 |

TABLE 8
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 267 | −5.32 | −1.99 | 2.80 | 443 | 0.235 | 0.058 |
| 268 | −5.35 | −2.01 | 2.80 | 442 | 0.248 | 0.058 |
| 269 | −5.43 | −2.14 | 2.76 | 450 | 1.044 | 0.118 |
| 270 | −5.36 | −2.12 | 2.71 | 458 | 0.426 | 0.092 |
| 271 | −5.40 | −2.17 | 2.70 | 459 | 0.346 | 0.089 |
| 272 | −5.25 | −1.96 | 2.75 | 451 | 0.433 | 0.088 |
| 273 | −5.29 | −2.02 | 2.74 | 452 | 0.456 | 0.089 |
| 274 | −5.27 | −1.98 | 2.76 | 450 | 0.565 | 0.091 |
| 275 | −5.27 | −1.96 | 2.76 | 450 | 0.343 | 0.094 |
| 277 | −5.36 | −2.12 | 2.71 | 458 | 0.425 | 0.092 |
| 282 | −5.27 | −1.94 | 2.80 | 444 | 0.266 | 0.082 |
| 283 | −5.38 | −2.11 | 2.68 | 462 | 0.282 | 0.094 |
| 284 | −5.27 | −1.95 | 2.77 | 448 | 0.292 | 0.094 |
| 285 | −5.33 | −2.06 | 2.73 | 454 | 0.316 | 0.090 |
| 286 | −5.30 | −2.00 | 2.76 | 450 | 0.376 | 0.090 |
| 287 | −5.32 | −2.00 | 2.76 | 448 | 0.300 | 0.106 |
| 288 | −5.13 | −1.82 | 2.76 | 449 | 0.211 | 0.070 |
| 289 | −5.09 | −1.83 | 2.70 | 459 | 0.243 | 0.070 |
| 293 | −5.00 | −1.69 | 2.80 | 443 | 0.381 | 0.071 |
| 295 | −5.35 | −1.89 | 2.93 | 423 | 0.257 | 0.072 |
| 297 | −5.28 | −1.92 | 2.84 | 436 | 0.258 | 0.062 |
| 298 | −5.43 | −2.12 | 2.76 | 449 | 0.211 | 0.083 |
| 299 | −5.50 | −2.21 | 2.75 | 450 | 0.238 | 0.082 |
| 300 | −5.51 | −2.12 | 2.86 | 434 | 0.258 | 0.076 |
| 301 | −5.36 | −2.15 | 2.66 | 466 | 0.219 | 0.088 |
| 303 | −5.20 | −1.79 | 2.88 | 430 | 0.302 | 0.073 |
| 305 | −5.09 | −1.77 | 2.79 | 445 | 0.290 | 0.065 |
| 306 | −5.28 | −1.89 | 2.87 | 432 | 0.237 | 0.061 |
| 307 | −5.33 | −2.03 | 2.77 | 448 | 0.796 | 0.099 |
| 308 | −5.26 | −1.87 | 2.87 | 432 | 0.265 | 0.059 |
| 309 | −5.25 | −1.86 | 2.80 | 443 | 0.261 | 0.015 |
| 310 | −5.26 | −1.88 | 2.86 | 434 | 0.242 | 0.059 |
| 311 | −5.31 | −1.96 | 2.82 | 440 | 0.525 | 0.086 |
| 312 | −5.33 | −1.93 | 2.87 | 432 | 0.328 | 0.063 |
| 313 | −5.33 | −1.95 | 2.85 | 435 | 0.345 | 0.073 |
| 314 | −5.27 | −1.95 | 2.78 | 446 | 0.624 | 0.095 |

TABLE 9
Results of calculation by DFT

| | Calculation by DFT | | | | | |
|---|---|---|---|---|---|---|
| | | | Adiabatic first | | | |
| | | | excitation singlet | Fluorescent | Oscillator | Reorganization |
| | HOMO | LUMO | state (S₁) | wavelength | strength | energy |
| Compound | (eV) | (eV) | energy (eV) | (nm) | f | (eV) |
| 315 | −5.30 | −1.99 | 2.78 | 446 | 0.173 | 0.056 |
| 316 | −5.33 | −1.86 | 2.94 | 422 | 0.278 | 0.077 |
| 317 | −5.27 | −1.92 | 2.83 | 437 | 0.264 | 0.063 |

As shown in Tables 3 to 9, the reorganization energy of the nitrogen-containing condensed cyclic compound of the present disclosure is low. In this regard, referring to FIG. 5, the FWHM of the nitrogen-containing condensed cyclic compound of the present disclosure may be predicted to become lower, and the color purity may be predicted to become higher.

Moreover, the nitrogen-containing condensed cyclic compound of the present disclosure has been confirmed to have a sufficient oscillator strength f and an excellent fluorescent luminescence efficiency.

From the above results, it can be seen that the nitrogen-containing condensed cyclic compound of the present disclosure has low reorganization energy, great oscillator strength f, and an appropriate blue fluorescent wavelength. In this regard, because the nitrogen-containing condensed cyclic compound of the present disclosure has narrow luminescence spectrum and high color purity, the nitrogen-containing condensed cyclic compound may be used as a blue luminescence material, which may improve the luminescence efficiency of an organic (electroluminescent) EL device.

### Evaluation of Compounds by Calculation 3

Whether some of nitrogen-containing condensed cyclic compounds 1 to 317 described as examples of the nitrogen-containing condensed cyclic compounds according to the present disclosure and Comparative Compound C1 satisfy Conditions (i) to (iv) was confirmed. The Q-Chem program was used in the calculation. Details of the calculation method are the same as described in connection with the nitrogen-containing condensed cyclic compound according to the present disclosure.

ΔE_{ST}>ΔE_{ST2}+ΔE′_{TT} Condition (i)

0 eV<ΔE_{ST2}+ΔE′_{TT}≤1.0 eV Condition (ii)

0 eV<ΔE′_{TT}≤0.15 eV Condition (iii)

ΔE_{ST2}>0 eV Condition (iv)

wherein, in Conditions (i) to (iv),

ΔE_{ST}(eV) indicates a difference value obtained by subtracting the lowest triplet excitation energy (eV) calculated from a T₁ equilibrium structure from the lowest singlet excitation energy (eV) calculated from an S₁ equilibrium structure,

ΔE_{ST2}(eV) indicates a difference value obtained by subtracting the second lowest triplet excitation energy (eV) calculated from a T₂ equilibrium structure from the lowest singlet excitation energy (eV) calculated from the S₁ equilibrium structure, and

ΔE′_{TT}(eV) indicates the difference value obtained from subtracting the lowest triplet excitation energy (eV) calculated from the T₂ equilibrium structure from the second lowest triplet excitation energy (eV) calculated from the T₂ equilibrium structure.

Here, each of the S₁ equilibrium structure, the T₁ equilibrium structure, and the T₂ equilibrium structure are formed when molecules are in an excited state, and these states represent the most stable structure wherein the energy for each state becomes the lowest.

The results of evaluation are shown in Table 10.

TABLE 10
Results of Evaluation of Conditions (i) to (iv) regarding
Nitrogen-containing Condensed Cyclic Compound

| Compound | ΔE_{ST} | ΔE_{ST2} | ΔE′_{TT} | Condition | Condition | Condition | Condition |
|---|---|---|---|---|---|---|---|
| No. | [eV] | [eV] | [eV] | (i) | (ii) | (iii) | (iv) |
| 1 | 0.738 | 0.553 | 0.110 | Satisfied | Satisfied | Satisfied | Satisfied |
| 2 | 0.739 | 0.542 | 0.111 | Satisfied | Satisfied | Satisfied | Satisfied |
| 3 | 0.747 | 0.559 | 0.086 | Satisfied | Satisfied | Satisfied | Satisfied |
| 5 | 0.647 | 0.463 | 0.133 | Satisfied | Satisfied | Satisfied | Satisfied |
| 6 | 0.641 | 0.432 | 0.130 | Satisfied | Satisfied | Satisfied | Satisfied |
| 7 | 0.717 | 0.528 | 0.146 | Satisfied | Satisfied | Satisfied | Satisfied |
| 8 | 0.643 | 0.461 | 0.133 | Satisfied | Satisfied | Satisfied | Satisfied |
| 9 | 0.821 | 0.591 | 0.118 | Satisfied | Satisfied | Satisfied | Satisfied |
| 10 | 0.829 | 0.595 | 0.096 | Satisfied | Satisfied | Satisfied | Satisfied |
| 12 | 0.827 | 0.593 | 0.107 | Satisfied | Satisfied | Satisfied | Satisfied |
| 14 | 0.772 | 0.547 | 0.133 | Satisfied | Satisfied | Satisfied | Satisfied |
| 19 | 0.762 | 0.579 | 0.133 | Satisfied | Satisfied | Satisfied | Satisfied |
| 65 | 0.714 | 0.500 | 0.100 | Satisfied | Satisfied | Satisfied | Satisfied |
| 66 | 0.571 | 0.402 | 0.109 | Satisfied | Satisfied | Satisfied | Satisfied |
| 71 | 0.716 | 0.529 | 0.134 | Satisfied | Satisfied | Satisfied | Satisfied |
| 75 | 0.702 | 0.484 | 0.091 | Satisfied | Satisfied | Satisfied | Satisfied |
| 76 | 0.521 | 0.357 | 0.098 | Satisfied | Satisfied | Satisfied | Satisfied |
| 79 | 0.772 | 0.547 | 0.199 | Satisfied | Satisfied | Satisfied | Satisfied |
| 80 | 0.706 | 0.491 | 0.096 | Satisfied | Satisfied | Satisfied | Satisfied |
| 81 | 0.548 | 0.377 | 0.109 | Satisfied | Satisfied | Satisfied | Satisfied |
| 84 | 0.757 | 0.556 | 0.149 | Satisfied | Satisfied | Satisfied | Satisfied |
| 85 | 0.697 | 0.510 | 0.129 | Satisfied | Satisfied | Satisfied | Satisfied |
| 89 | 0.674 | 0.454 | 0.131 | Satisfied | Satisfied | Satisfied | Satisfied |
| 114 | 0.794 | 0.576 | 0.113 | Satisfied | Satisfied | Satisfied | Satisfied |
| 156 | 0.754 | 0.544 | 0.148 | Satisfied | Satisfied | Satisfied | Satisfied |
| 160 | 0.625 | 0.436 | 0.141 | Satisfied | Satisfied | Satisfied | Satisfied |
| 172 | 0.816 | 0.657 | 0.046 | Satisfied | Satisfied | Satisfied | Satisfied |
| 194 | 0.708 | 0.471 | 0.069 | Satisfied | Satisfied | Satisfied | Satisfied |
| 195 | 0.823 | 0.595 | 0.089 | Satisfied | Satisfied | Satisfied | Satisfied |
| C1 | 0.775 | 0.582 | 0.157 | Satisfied | Satisfied | Satisfied | Satisfied |

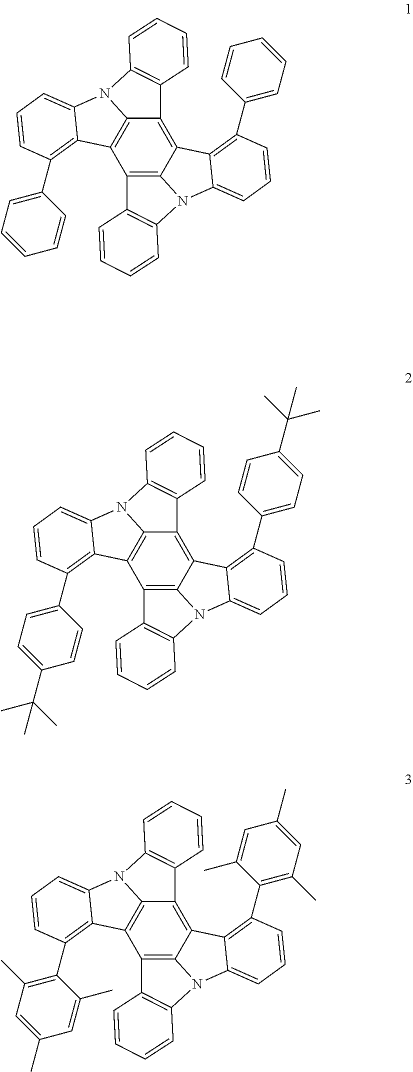

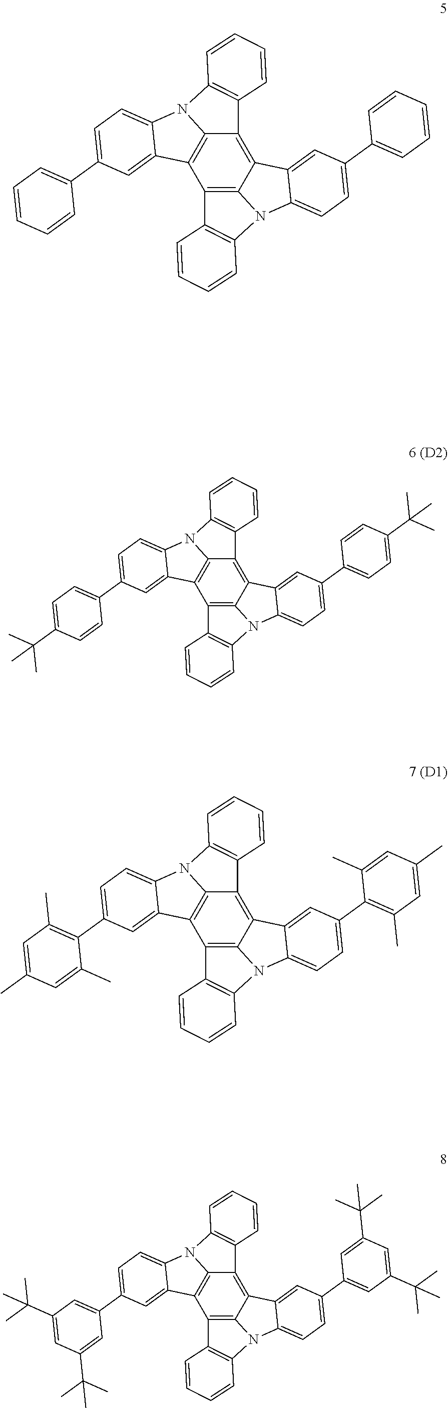

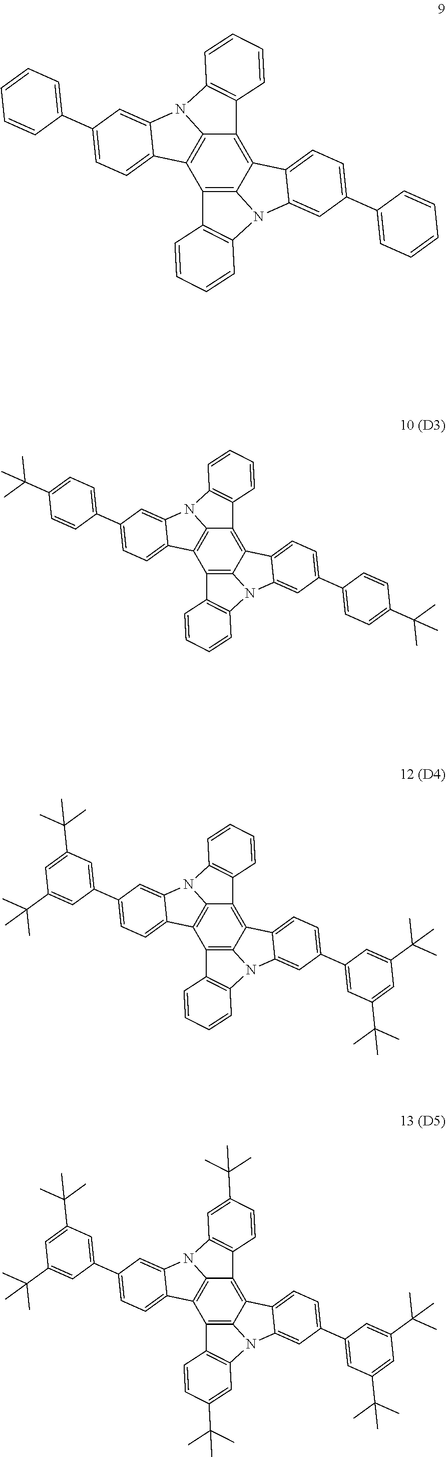

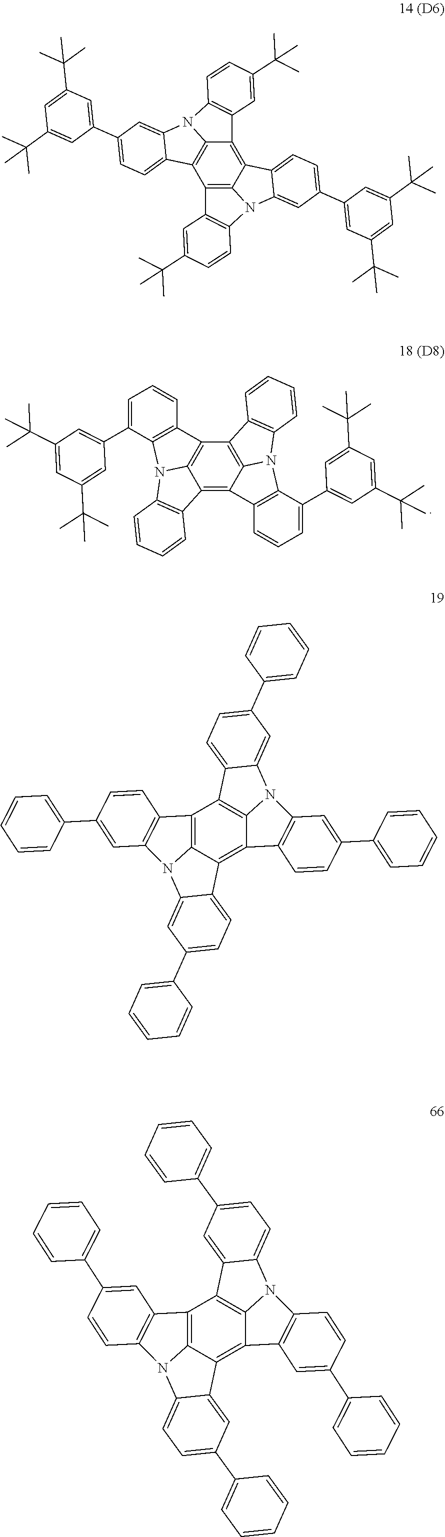

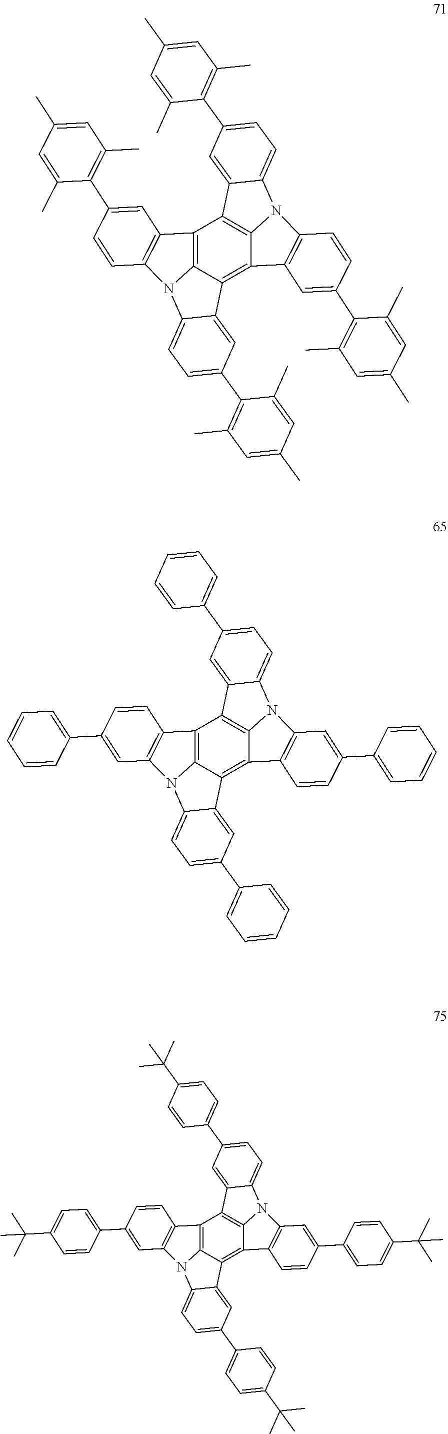

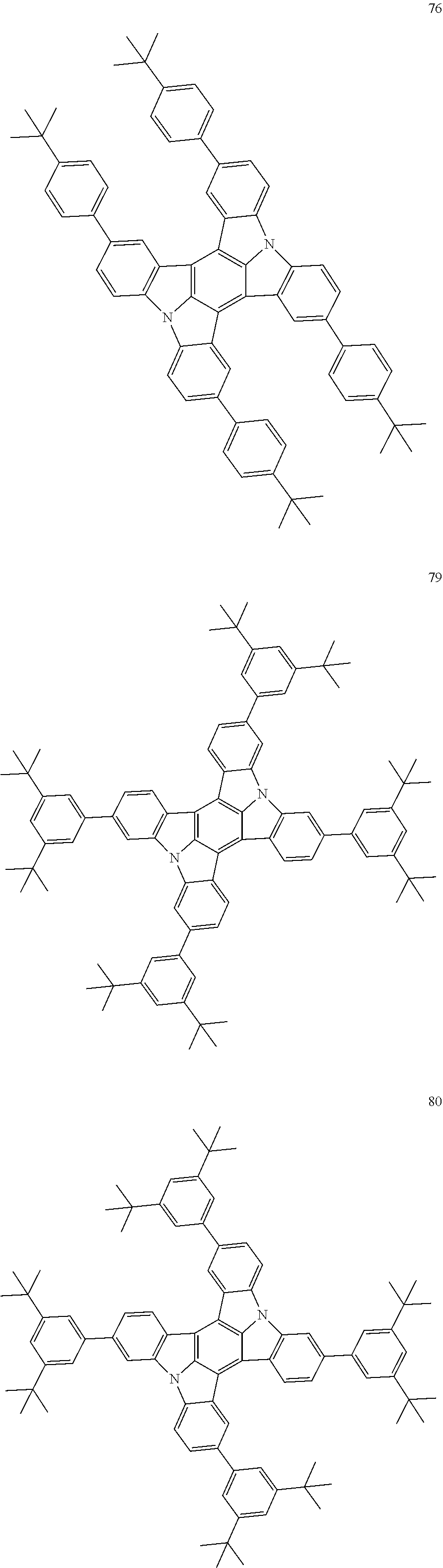

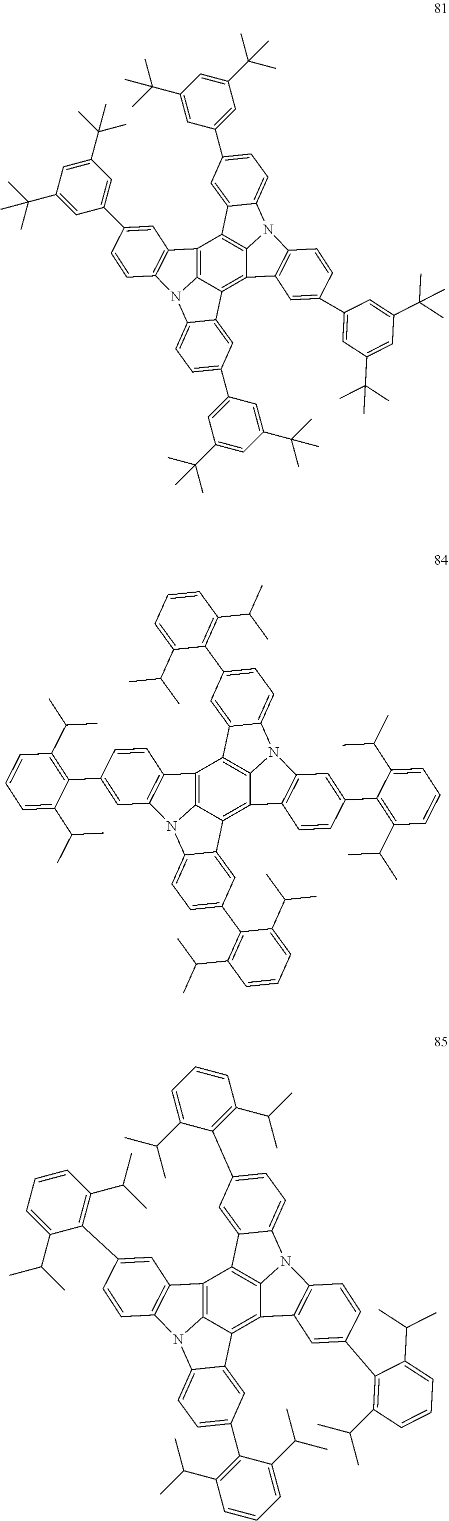

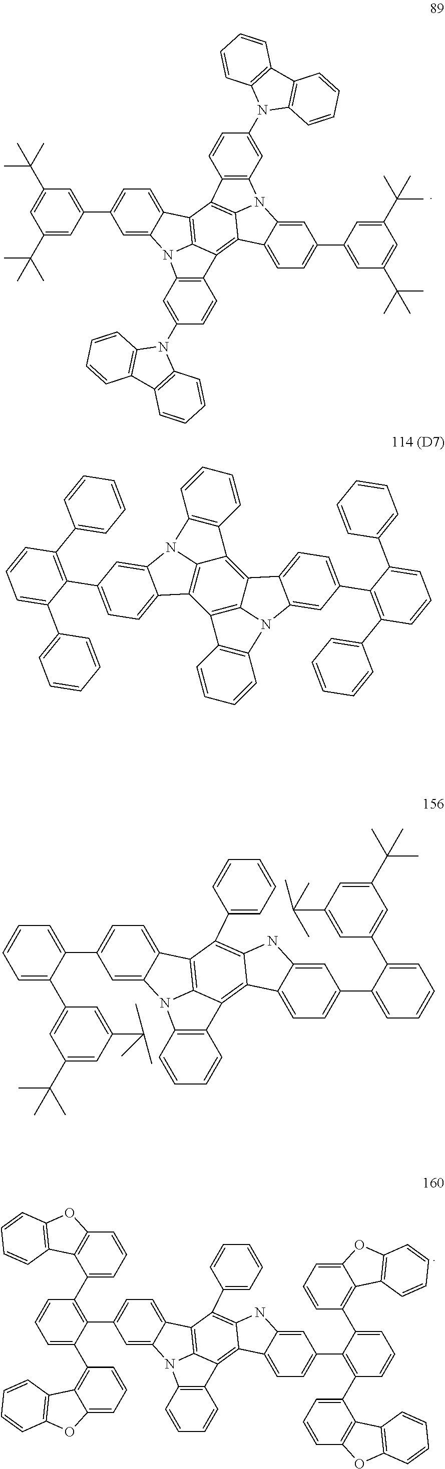

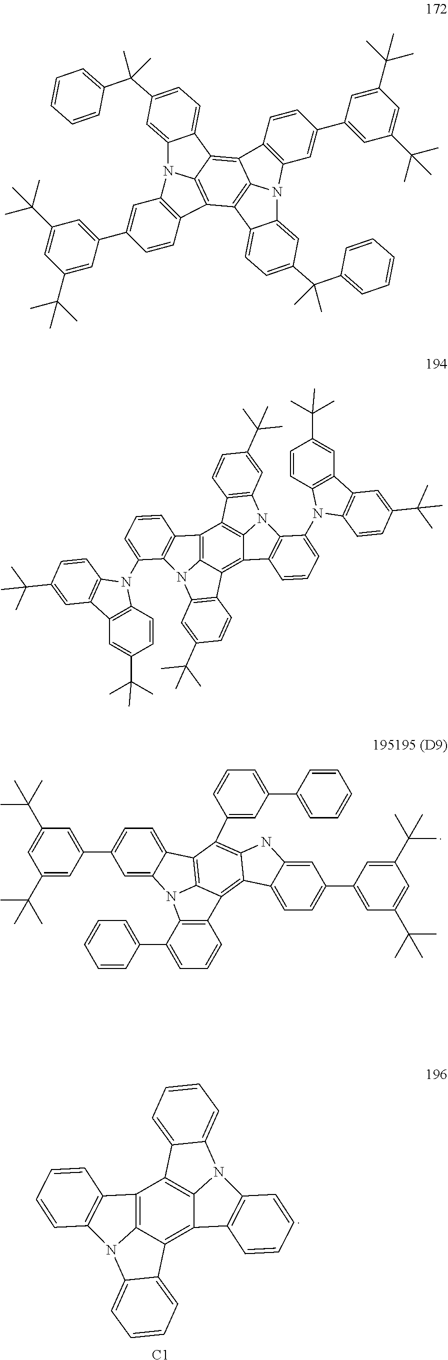

### Synthesis of Nitrogen-Containing Condensed Cyclic Compound

Compounds D1, D2, D3, D4, D5, D6, D7, D8, and D9 were synthesized to be used in manufacturing an organic EL device. Compounds D1, D2, D3, D4, D5, D6, D7, D8, and D9 are each the same compounds as the nitrogen-containing condensed cyclic compounds 7, 6, 10, 12, 13, 14, 114, 18, and 195. In addition, Comparative Compound C1 was prepared for use in manufacturing the organic EL device of the comparative example.

### Synthesis of Compound D1 (Compound 7)

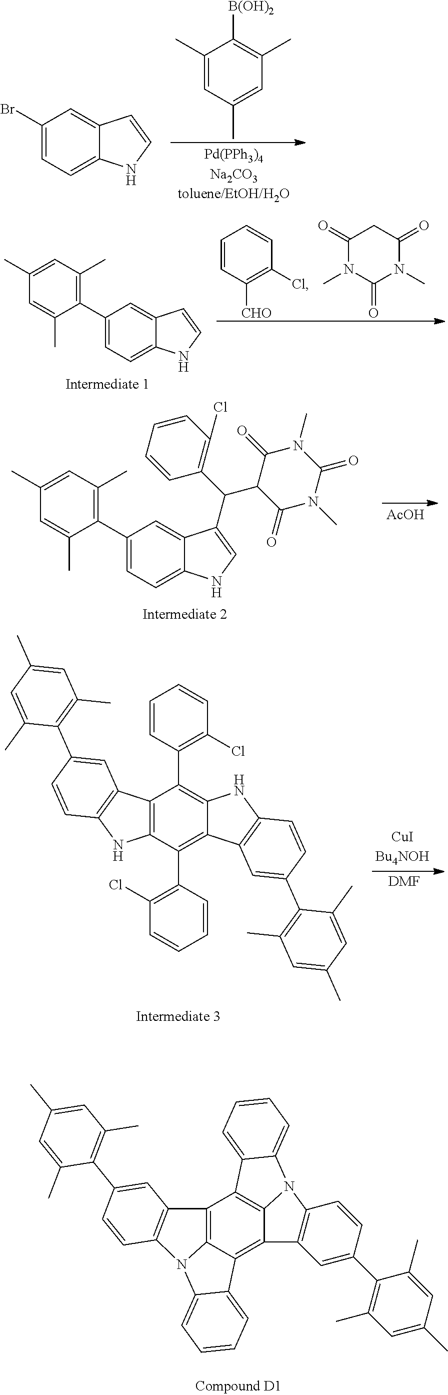

### Synthesis of Intermediate 1

In an inert gas atmosphere, tetrakis triphenyl phosphine palladium (5.80 g, 5.0 mmol) was added to a mixture of 5-bromoindole (19.61 g, 100 mmol), 2,4,6-trimethylboron acid (21.40 g, 130.4 mmol), toluene (400 ml), ethanol (100 ml), and 1 M of sodium carbonate aqueous solution (200 ml) and refluxed while heating for 5 hours. The mixture was cooled in room temperature and an organic layer was extracted therefrom with toluene. The organic layer was dried with anhydrous magnesium sulfate, filtered, and concentrated. The obtained product was filtered by silica gel column chromatography to thereby obtain Intermediate 1 as a colorless oil (16.54 g, 70.34 mmol, yield of 70%).

### Synthesis of Intermediate 2

A mixture of Intermediate 1 (16.54 g, 70.34 mmol), 2-chlorobenzaldehyde (7.91 ml, 70.34 mmol), and 1,3-dimethylbarbital acid (10.98 g, 70.32 mmol) was heated while stirring at a temperature of 100° C. for 30 minutes. The reaction was washed with hexane and ethanol to thereby obtain Intermediate 2 as a white solid (32.62 g, 63.45 mmol, yield of 90%).

### Synthesis of Intermediate 3

Acetic acid (100 ml) was added to Intermediate 2 (32.54 g, 63.31 mmol) and refluxed while heating for 24 hours. The precipitated solid was washed with ethanol to thereby obtain Intermediate 3 as a colorless solid (11.81 g, 15.92 mmol, yield of 50%).

### Synthesis of Compound D1

In an inert gas atmosphere, tetrabutylammonium hydroxide (37% of methanol solution) (100 ml, 118 mmol) was added to a mixture of Intermediate 3 (11.80 g, 15.92 mmol), copper iodide (I) (23.65 g, 124.2 mmol), and N,N-dimethylformamide (160 ml) and heated while stirring at a temperature of 120° C. for 33 hours. The precipitated solid was washed with acetonitrile and ethylenediamine aqueous solution. The product was recrystallized with benzonitrile to thereby obtain Compound D1 as a yellow solid (8.99 g, 14.0 mmol, yield of 88%).

The structure of the obtained Compound D1 was determined by nuclear magnetic resonance (¹H-NMR): ¹H NMR (300 MHz, THF-d8), Δ2.09 (s, 12H), 2.32 (s, 6H), 6.91 (s, 4H), 7.31-7.43 (m, 4H), 7.56 (dd, J=8.1 Hz, 2H), 8.11 (d, J=8.1 Hz, 2H), 8.16 (d, J=8.1 Hz, 2H), 8.25 (s, 2H), 8.42 (d, J=7.5 Hz, 2H).

### Synthesis of Compound D2 (Compound 6)

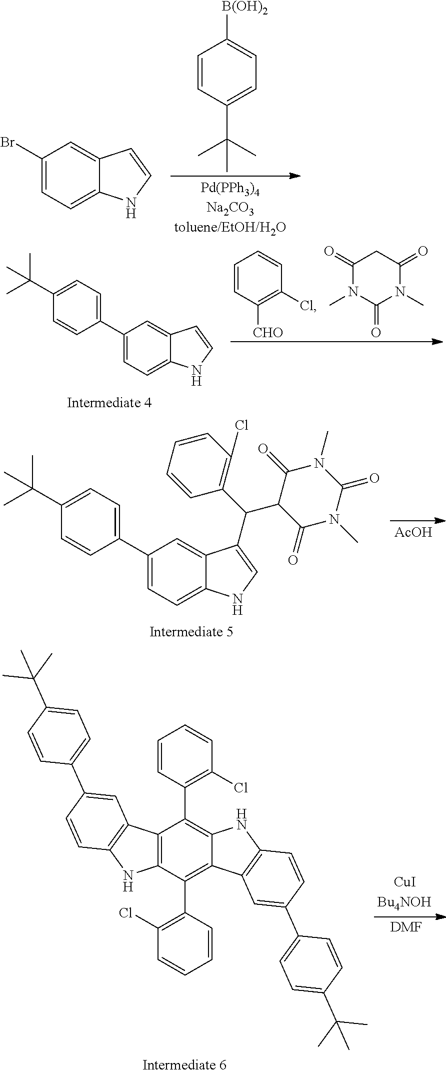

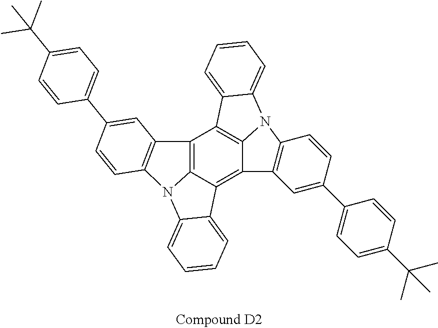 [SMILES: CC(C)(C)c1ccc(-c2ccc3c(c2)c2c4c5ccccc5n5c6ccc(-c7ccc(C(C)(C)C)cc7)cc6c(c6c7ccccc7n3c62)c45)cc1]

### Synthesis of Intermediate 4

Intermediate 4 (yield of 73%) was obtained in the same manner as in synthesizing Intermediate 1, except for the change in the corresponding reagent.

### Synthesis of Intermediate 5

Intermediate 5 (yield of 33%) was obtained in the same manner as in synthesizing Intermediate 2, except for the change in the corresponding reagent.

### Synthesis of Intermediate 6

Intermediate 6 (yield of 44%) was obtained in the same manner as in synthesizing Intermediate 3, except for the change in the corresponding reagent.

### Synthesis of Compound D2

Compound D2 (yield of 67%) was obtained in the same manner as in synthesizing Compound D1, except for the change in the corresponding reagent.

The structure of the obtained Compound D2 was determined by nuclear magnetic resonance (¹H-NMR): ¹H NMR (300 MHz, THF-d₈), Δ1.39 (s, 18H), 7.42-7.53 (m, 6H), 7.61 (br, 2H), 7.70 (br, 4H), 7.81 (br, 2H), 8.08-8.16 (m, 4H), 8.55 (br, 2H), 8.65 (br, 2H).

### Synthesis of Compound D3 (Compound 10)

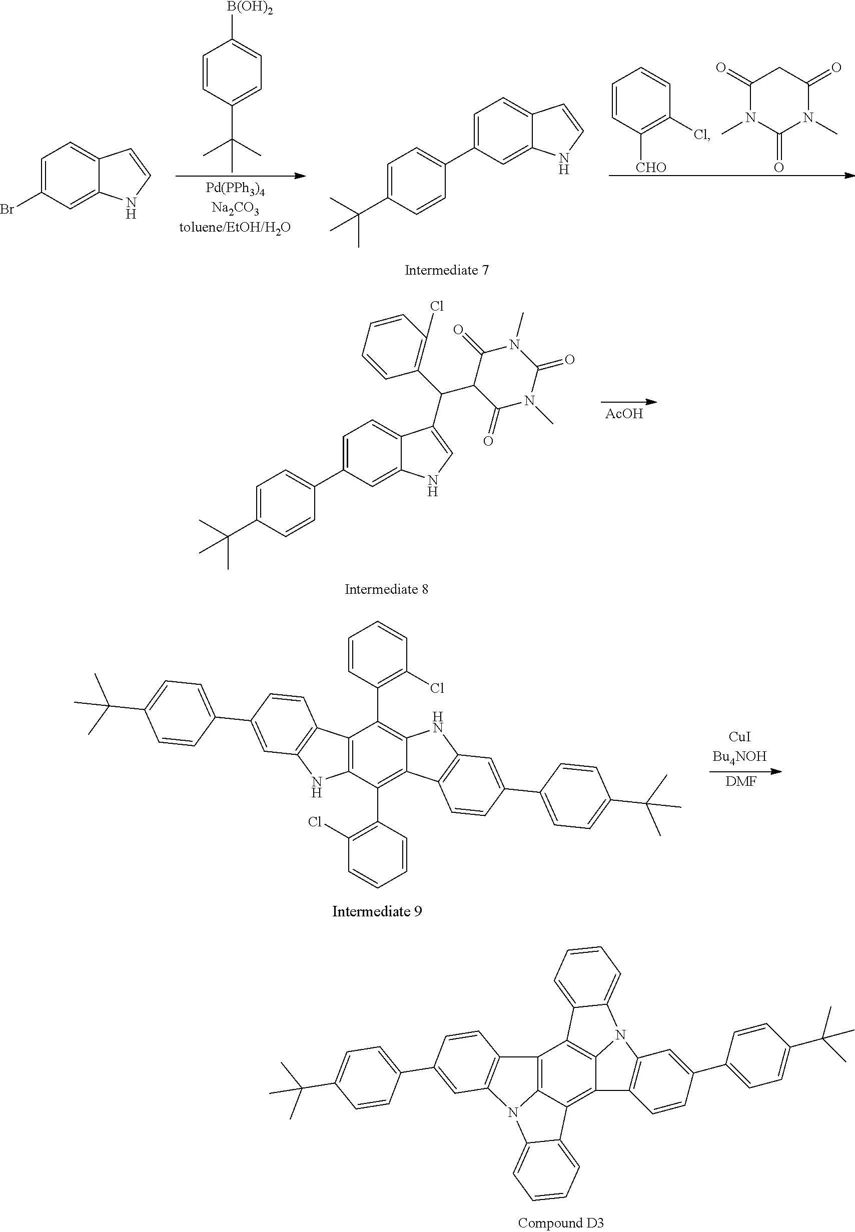

### Synthesis of Intermediate 7

Intermediate 7 (yield of 35%) was obtained in the same manner as in synthesizing Intermediate 1, except for the change in the corresponding reagent.

### Synthesis of Intermediate 8

Intermediate 8 (yield of 89%) was obtained in the same manner as in the synthesis Intermediate 2, except for the change in the corresponding reagent.

### Synthesis of Intermediate 9

Intermediate 9 (yield of 62%) was obtained in the same manner as in synthesizing Intermediate 3, except for the change in the corresponding reagent.

### Synthesis of Compound D3

Compound D3 (yield of 64%) was obtained in the same manner as in synthesizing Compound D1, except for the change in the corresponding reagent.

The structure of the obtained Compound D3 was determined by nuclear magnetic resonance (¹H-NMR): ¹H NMR (300 MHz, THF-d₈), Δ1.39 (s, 18H), 7.42-7.52 (m, 6H), 7.60 (br, 2H), 7.65-7.75 (m, 6H), 8.16 (br, 2H), 8.25 (s, 2H), 8.54 (br, 4H).

### Synthesis of Compound D4 (Compound 12)

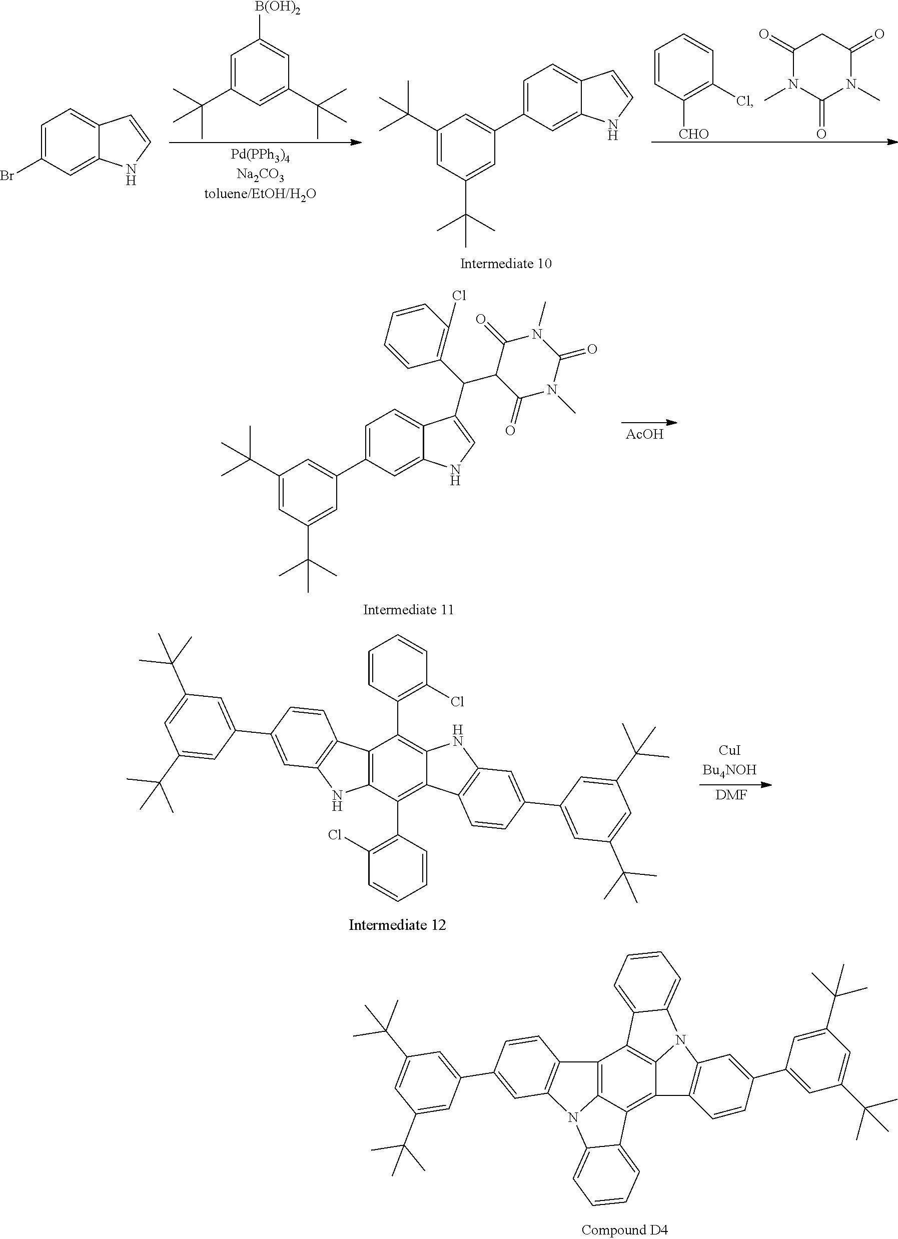

### Synthesis of Intermediate 10

Intermediate 10 (yield of 79%) was obtained in the same manner as in synthesizing Intermediate 1, except for the change in the corresponding reagent.

### Synthesis of Intermediate 11

A mixture of Intermediate 10 (18.01 g, 58.93 mmol), 2-chlorobenzaldehyde (6.62 mL, 58.93 mmol), and 1,3-dimethylbarbituric acid (9.20 g, 58.92 mmol) was heated while stirring at a temperature of 100° C. for 30 minutes. The obtained product was filtered by silica gel column chromatography to thereby obtain Intermediate 11 as a white solid (29.59 g, 50.65 mmol, yield of 86%).

### Synthesis of Intermediate 12

Intermediate 12 (yield of 40%) was obtained in the same manner as in synthesizing Intermediate 3, except for the change in the corresponding reagent.

### Synthesis of Compound D4

Compound D4 (yield of 84%) was obtained in the same manner as in synthesizing Compound D1, except for the change in the corresponding reagent.

The structure of the obtained Compound D4 was determined by nuclear magnetic resonance (¹H-NMR): ¹H NMR (300 MHz, THF-d₈), Δ1.52 (s, 36H), 7.51-7.61 (m, 4H), 7.70 (br, 2H), 7.75 (br, 4H), 7.79 (br, 2H), 8.25 (br, 2H), 8.35 (s, 2H), 8.60 (br, 2H), 8.64 (br, 2H).

### Synthesis of Compound D5 (Compound 13)

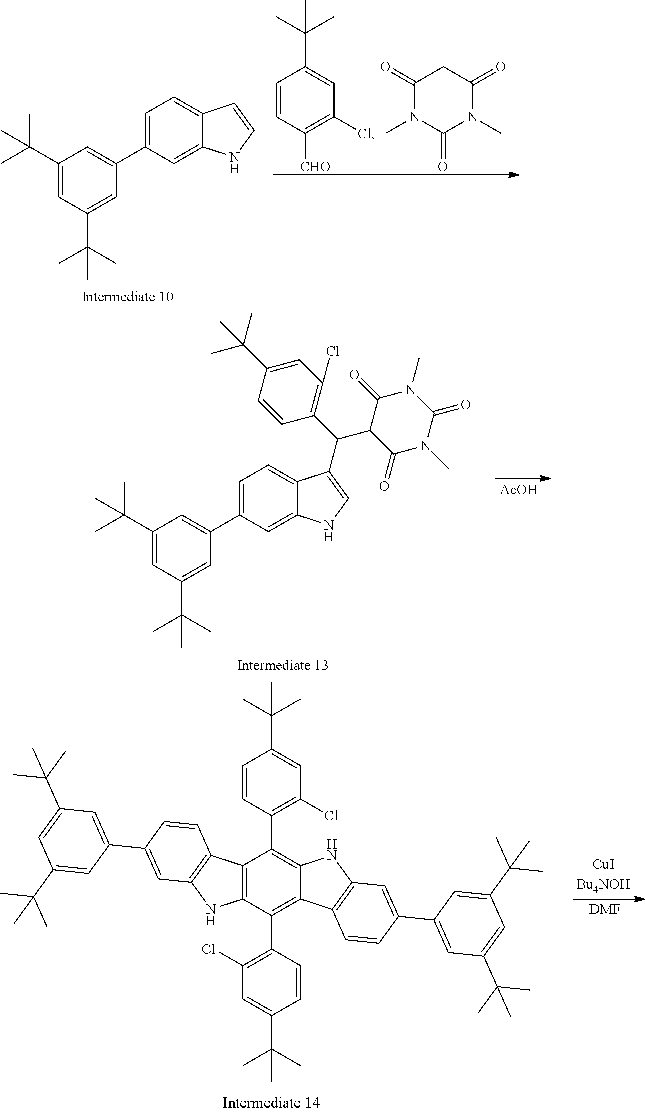

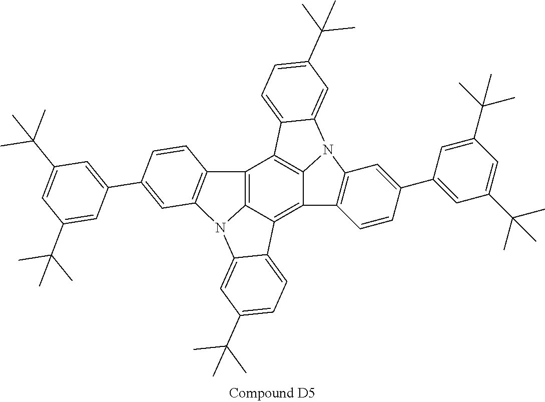 [SMILES: CC(C)(C)c1cc(-c2ccc3c4c5c6ccc(C(C)(C)C)cc6n6c7cc(-c8cc(C(C)(C)C)cc(C(C)(C)C)c8)ccc7c(c7c8ccc(C(C)(C)C)cc8n(c3c2)c47)c56)cc(C(C)(C)C)c1]

### Synthesis of Intermediate 13

Intermediate 13 (yield of 67%) was obtained in the same manner as in synthesizing Intermediate 11, except for the change in the corresponding reagent.

### Synthesis of Intermediate 14

Intermediate 14 (yield of 67%) was obtained in the same manner as in synthesizing Intermediate 3, except for the change in the corresponding reagent.

### Synthesis of Compound D5

Compound D5 (yield of 72%) was obtained in the same manner as in synthesizing Compound D1, except for the change in the corresponding reagent.

The structure of the obtained Compound D5 was determined by nuclear magnetic resonance (¹H-NMR): ¹H NMR (300 MHz, THF-d₈), Δ1.53 (s, 36H), 1.57 (s, 18H), 7.58 (d, J=8.1 Hz, 2H), 7.61 (br, 2H), 7.78 (s, 4H), 7.82 (d, J=8.1 Hz, 2H), 8.18 (s, 2H), 8.25 (s, 2H), 8.44 (br, 2H), 8.65 (d, J=8.1 Hz, 2H).

### Synthesis of Compound D6 (Compound 14)

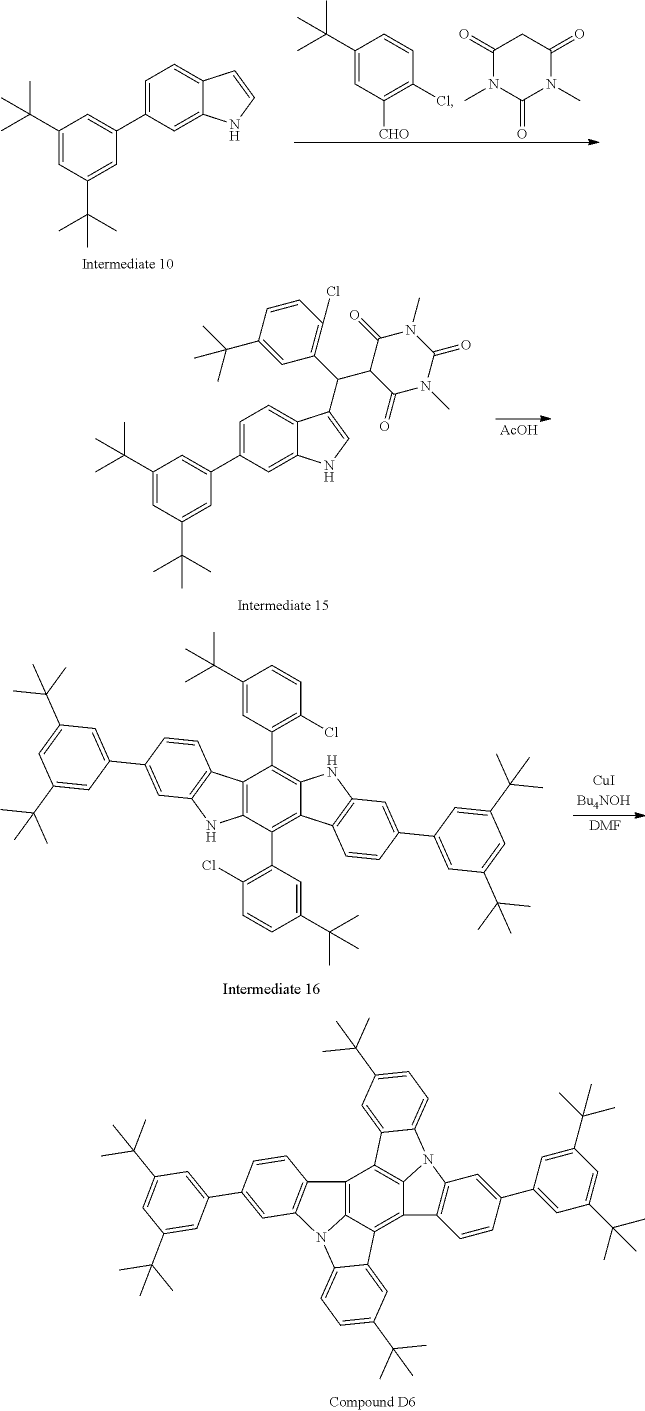

### Synthesis of Intermediate 15

Intermediate 15 (yield of 98%) was obtained in the same manner as in the synthesis of Intermediate 11, except for the change in the corresponding reagent.

### Synthesis of Intermediate 16

Intermediate 16 (yield of 51%) was obtained in the same manner as in synthesizing Intermediate 3, except for the change in the corresponding reagent.

### Synthesis of Compound D6

Compound D6 (yield of 61%) was obtained in the same manner as in synthesizing Compound D1, except for the change in the corresponding reagent.

The structure of the obtained Compound D6 was confirmed by nuclear magnetic resonance (¹H-NMR): ¹H NNMR (00 MHz, THF-d8) δ1.51 (s, 36H), 1.67 (s, 18H), 7.57 (br, 2H), 7.74-7.78 (m, 4H), 7.80-7.88 (m, 4H), 8.21-8.28 (m, 2H), 8.41 (br, 2H), 8.63-8.71 (m, 4H).

### Synthesis of Compound D7 (Compound 114)

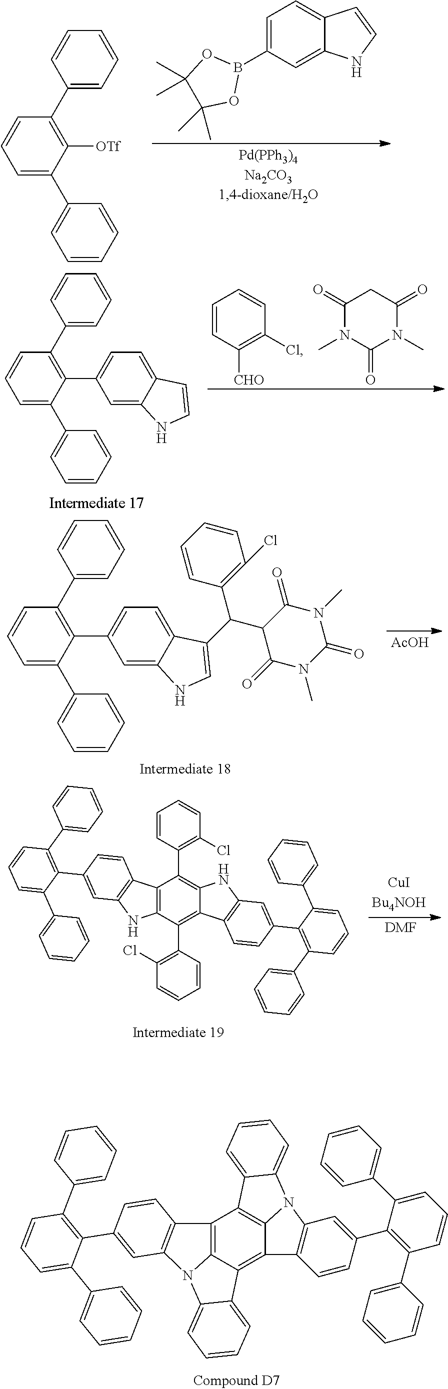

### Synthesis of Intermediate 17

In an inert gas atmosphere, tetrakis triphenyl phosphine palladium (2.32 g, 2.0 mmol) was added to a mixture of (m-terphenyl 2′-yl)triflate (15.13 g, 40.00 mmol), 6-(4, 4, 5, 5-tetramethyl-1, 3, 2-dioxaborolane-2-yl)-1H-indole (11.65 g, 47.93 mmol), 1, 4-dioxane (200 ml), and 1M of sodium carbonate aqueous solution (80 ml), and refluxed while heating for 8 hours. After cooling the temperature to room temperature, an organic layer was extracted by using chloroform. The organic layer was dried with anhydrous magnesium sulfate, filtered, and concentrated. The obtained product was purified by silica gel column chromatography to thereby obtain Intermediate 17 as a white solid (9.46 g, 37.4 mmol, yield of 69%).

### Synthesis of Intermediate 18

Intermediate 18 (yield of 72%) was obtained in the same manner as in synthesizing Intermediate 11, except for the change in the corresponding reagent.

### Synthesis of Intermediate 19

Intermediate 19 (yield of 57%) was obtained in the same manner as in synthesizing Intermediate 3, except for the change in the corresponding reagent.

### Synthesis of Compound D7

Compound D7 (yield of 72%) was obtained in the same manner as in synthesizing Compound D1, except for the change in the corresponding reagent.

The structure of the obtained Compound D7 was confirmed by nuclear magnetic resonance (¹H-NMR): ¹H NMR (300 MHz, THF-d8) δ6.95-7.22 (m, 18H), 7.26 (d, J=7.5 Hz, 4H), 7.43-7.63 (m, 10H), 7.76 (br, 2H), 7.82-7.89 (m, 2H), 8.32 (d, J=8.1 Hz, 2H), 8.52-8.59 (m, 2H).

### Synthesis of Compound D8 (Compound 18)

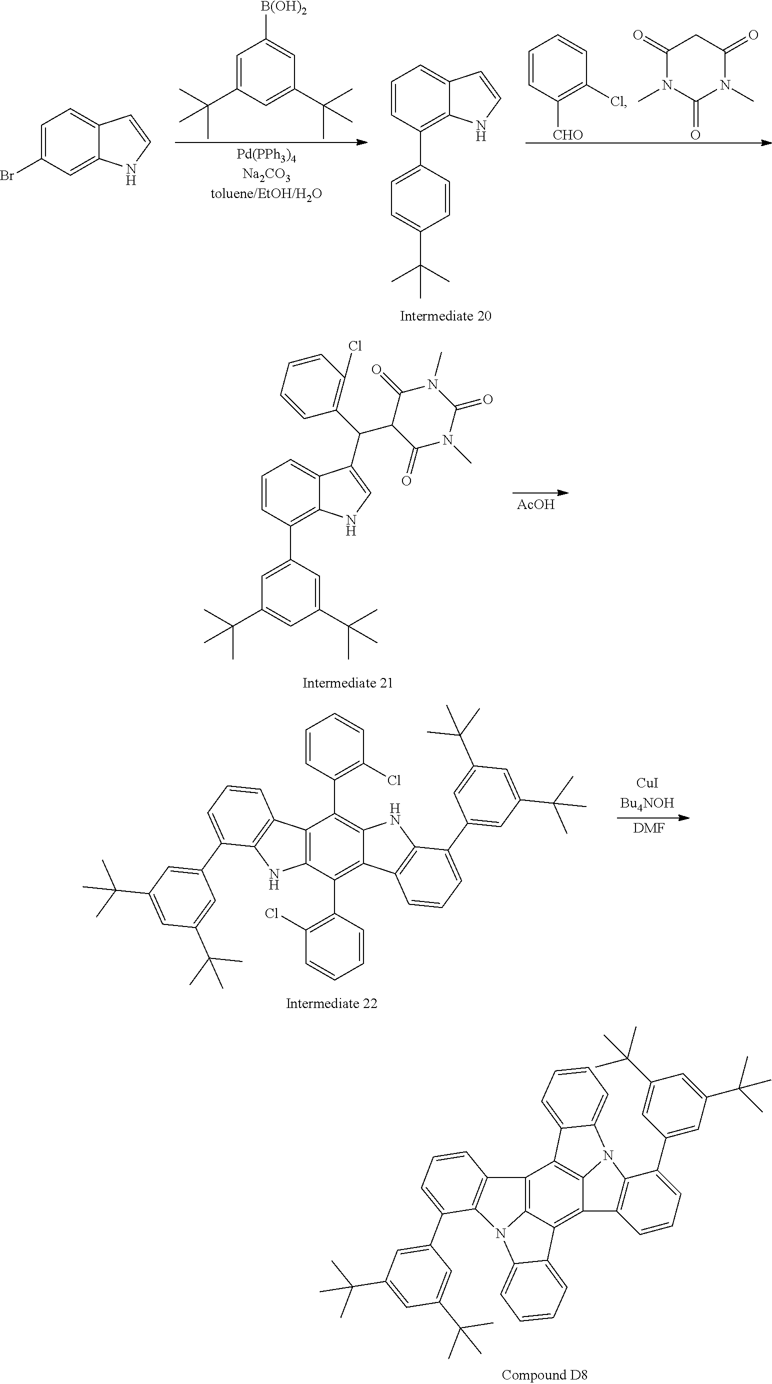

### Synthesis of Intermediate 20

Intermediate 20 (yield of 91%) was obtained in the same manner as in synthesizing Intermediate 1, except for the change in the corresponding reagent.

### Synthesis of Intermediate 21

Intermediate 21 (yield of 14%) was obtained in the same manner as in synthesizing Intermediate 3, except for the change in the corresponding reagent.

### Synthesis of Intermediate 22

Intermediate 22 (yield of 31%) was obtained in the same manner as in synthesizing Intermediate 11, except for the change in the corresponding reagent.

### Synthesis of Compound D8

Compound D8 (yield of 31%) was obtained in the same manner as in synthesizing Compound D1, except for the change in the corresponding reagent.

The structure of the obtained Compound D8 was confirmed by nuclear magnetic resonance (¹H-NMR): ¹H NMR (300 MHz, THF-d8) δ1.48 (s, 36H), 5.52 (d, J=8.4 Hz, 2H), 7.07 (dd, J=7.5, 7.5 Hz, 2H), 7.37 (dd, J=7.5, 7.5 Hz, 2H), 7.55-7.66 (m, 8H), 7.87 (br, 2H), 8.67 (d, J=7.5 Hz, 2H), 8.73-8.77 (m, 2H).

### Synthesis of Compound D9 (Compound 195)

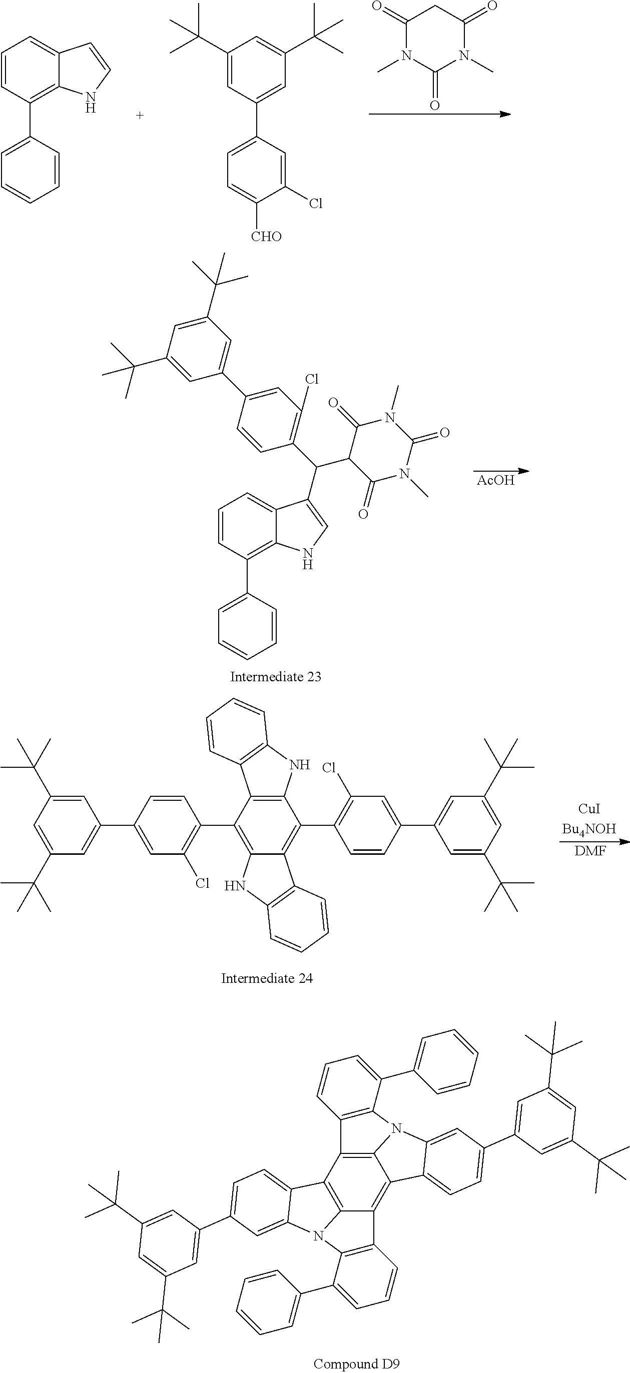

### Synthesis of Intermediate 23

Intermediate 23 (yield of 80%) was obtained in the same manner as in synthesizing Intermediate 3, except for the change in the corresponding reagent.

### Synthesis of Intermediate 24

Intermediate 24 (yield of 49%) was obtained in the same manner as in synthesizing Intermediate 11, except for the change in the corresponding reagent.

### Synthesis of Compound D9

Compound D9 (yield of 20%) was obtained in the same manner as in synthesizing Compound D1, except for the change in the corresponding reagent.

The structure of the obtained Compound D9 was determined by nuclear magnetic resonance (¹H-NMR): ¹H NMR (300 MHz, THF-d8) δ1.49 (s, 36H), 6.34 (d, J=1.5 Hz, 2H), 7.22 (d, J=1.5 Hz, 4H), 7.34-7.43 (m, 2H), 7.51-7.63 (m, 10H), 7.67 (dd, J=7.5, 7.5 Hz, 2H), 7.77-7.84 (m, 4H), 8.75 (d, J=8.1 Hz, 2H), 8.79 (dd, J=6.0, 1.5 Hz, 2H).

PL Peak Wavelength and FWHM of Peak of Emission Spectrum

A toluene solution of the obtained Compounds D1 to D14 and 1×10⁻⁵ M (=mol/dm³, mol/L) of Comparative Compound C1 were prepared. The solutions were measured using a spectro fluorescence photometer F7000 of Hitachi Hightech Inc. at room temperature with an excitation wavelength of 360 nm, and the luminescence peak wavelength (nm) and FWHM of the peak of the emission spectrum were evaluated. The results of the evaluation are shown in Table 11.

TABLE 11
Luminescence peak wavelength of nitrogen-containing condensed
cyclic compound and FWHM of peak of emission spectrum

| | Peak wavelength | FWHM of peak of |
|---|---|---|
| | of luminescence in | luminescence spectrum |
| Compound | PL | in PL |
| No. | [nm] | [nm] |
| D1 | 452 | 12 |
| D2 | 456 | 12 |
| D3 | 457 | 13 |
| D4 | 455 | 13 |
| D5 | 455 | 15 |
| D6 | 459 | 14 |
| D7 | 459 | 16 |
| D8 | 448 | 11 |
| D9 | 456 | 14 |
| C1 | 446 | 13 |

As shown in Table 11, Compound D1 according to the present disclosure showed excellent blue luminescence color in PL, and realized luminescence of high color purity. In addition, as shown in Table 11, Compounds D2 to D9 other than Compound D1 according to the present disclosure show excellent blue luminescence color in PL and realize luminescence of high color purity, just as Compound D1. From the above result, Compounds D2 to D9 according to the present disclosure are assumed to realize excellent blue luminescence color in the organic EL device and luminescence with high color purity, just as Compound D1 according to the present disclosure.

In addition, in Table 11, blue luminescence color is realized in the PL peak wavelength of Comparative Compound C1, and the FWHM of the peak of the emission spectrum in PL of Comparative Compound C1 is narrow and luminescence with high color purity is realized. From the above result, the blue luminescence color and excellent luminescence with high color purity are assumed to be originated from a particular nitrogen-containing condensed cyclic structure that is the parent skeleton.

A graph regarding Compounds D1 to D8 showing the FWHM of a fluorescent luminescence in a measured PL relative to the reorganization energy (eV) calculated according to the DFT is shown in FIG. 6. From the results of FIG. 6, it can be seen that the relocation energy (eV) calculated according to the DFT is related to the FWHM of the fluorescent luminescence, and the smaller the reorganization energy (eV), the smaller the FWHM of the fluorescent luminescence, that is, the spectral width of the fluorescent luminescence is narrowed. Regarding other compounds of which the reorganization energy (eV) is shown in Tables 3 to 9, it may be assumed that the spectral width of the fluorescent luminescence may be narrowed in the same manner as Compounds D1 to D8.

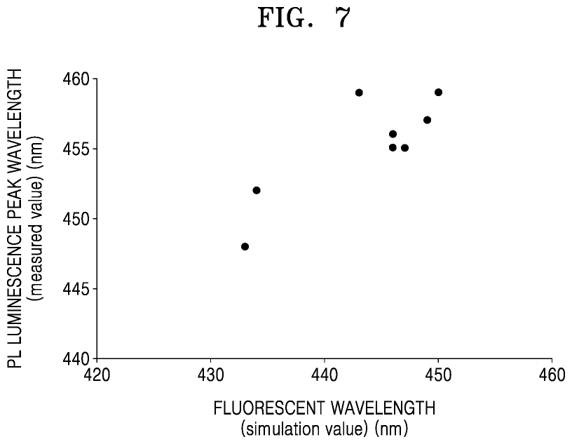

Further, a graph regarding Compounds D1 to D8 showing the fluorescent wavelength in a measured PL relative to the emission wavelength calculated according to the DFT is shown in FIG. 7. From the results of FIG. 7, the emission wavelength calculated according to the DFT was shown to be related to the fluorescent wavelength in a measured PL. Regarding other compounds shown in Tables 3 to 9, it may be assured that blue luminescent color may be realized in the same manner as compounds D1 to D8.

### Evaluation of Solubility in Mesitylene

10 mg of the obtained Compounds D1 to D9 and Comparative Compound C1 were each placed in a test tube, mesitylene was added thereto, and were heated at a temperature of 160° C. Mesitylene was added thereto until the compounds were completely dissolved and the solubility was calculated. The results of evaluation are shown in Table 12.

TABLE 12
Solubility of mesitylene in nitrogen-
containing condensed cyclic compound

| Compound | Solubility in mesitylene |
|---|---|
| No. | [g/L] |
| D1 | 2.0 |
| D2 | 1.3 |
| D3 | 1.0 |
| D4 | 3.2 |
| D5 | 10.1 |
| D6 | 1.0 |
| D7 | 3.1 |
| D8 | 10.3 |
| D9 | 3.0 |
| C1 | 0.75 |

As shown in Table 12, another Compound D1 according to the present disclosure is shown to exhibit a high solubility in mesitylene. As shown in Table 12, other Compounds D2 to D9 according to the present disclosure was shown to have high solubility in mesitylene, just as Compound D1 of the present disclosure. As described above, Compounds D1 to D9 according to the present invention showed a high solubility in mesitylene compared to the Comparative Compound C1. From the above result, it is assumed that the nitrogen-containing condensed cyclic compound according to the present disclosure has a high aggregation inhibiting effect due to a particular substituent. The Comparative Compound C is thought not to have a high aggregation inhibiting effect due to a particular substituent.

### Evaluation of Compounds by Calculation 4

Regarding nitrogen-containing condensed cyclic compounds 1 to 317 described as examples of nitrogen-containing condensed cyclic compounds according to the present disclosure, from the most stable structure calculated according to the calculation method of (I) in the “Calculation through DFT” section, by using the GaussView (Gaussian Inc.), a dihedral angle between a core portion and a group derived from an aromatic ring linked to the core portion by a single bond among substituents including a group derived from an aromatic ring linked to the core portion by a single bond was calculated.

Herein, the dihedral angle between the core portion and the group derived from an aromatic ring linked to the core portion by a single bond refers to, when α1 is a core atom linked to the substituent, α2 is an atom closest to α1 among the cores, β1 is a substituent atom linked to the core, and β2 is an atom closest to β1 among the substituents, an angle between triangle Δα2α1β1 with vertices α2, α1, and β1 and triangle Δβ2β1α1 with vertices β2, β1, and α1.

Particularly, regarding the nitrogen-containing condensed cyclic compound, the dihedral angle between the core portion and the group derived from an aromatic ring linked to the core portion by a single bond was calculated by DFT by using Gaussian 16 (Gaussian Inc.) as a calculation software from the most stable structure calculated by the method of (I):

(I) S₀ Calculation Method: Calculation of Structure Optimization Through DFT Including Functional B3LYP, Basis Function 6-31 G(d, p), and Toluene Solvent Effect (PCM)

The results of the evaluation are shown in Tables 13 to 18. In the Tables, dihedral angles 1 to 4 each indicate a dihedral angle between the core portion and a group derived from an aromatic ring linked to a group derived from another benzene ring of the core portion by a single bond. In the Tables, “-” indicates not having a substituent including a group derived from an aromatic ring linked to the core portion by a single bond to form a corresponding dihedral angle.

TABLE 13
Evaluation result of dihedral angle between core portion of
nitrogen-containing condensed cyclic compound and group derived
from aromatic ring linked to core portion by single bond

| | Dihedral | Dihedral | Dihedral | Dihedral |
|---|---|---|---|---|
| Compound | angle 1 | angle 2 | angle 3 | angle 4 |
| No. | [°] | [°] | [°] | [°] |
| 1 | 54.8 | 54.6 | — | — |
| 2 | 53.9 | 53.7 | — | — |
| 3 | 74.1 | 77.9 | — | — |
| 4 | 49.5 | 49.9 | — | — |
| 7 | 89.7 | 90.0 | — | — |
| 11 | 89.9 | 89.9 | — | — |
| 15 | 65.5 | 65.7 | — | — |
| 16 | 64.2 | 64.2 | — | — |
| 17 | 88.8 | 88.6 | — | — |
| 18 | 67.1 | 67.5 | — | — |
| 21 | 59.5 | 59.0 | — | — |
| 24 | 55.4 | 54.9 | — | — |
| 50 | 89.9 | 89.9 | — | — |
| 51 | 83.3 | 81.0 | — | — |
| 52 | 89.9 | 89.6 | — | — |
| 53 | 75.8 | 75.8 | — | — |
| 54 | 89.6 | 89.6 | — | — |
| 55 | 89.4 | 89.4 | — | — |
| 68 | 52.4 | 52.4 | 51.1 | 51.1 |
| 69 | 89.8 | 89.8 | 89.8 | 89.8 |
| 70 | 90.0 | 89.9 | 89.9 | 90.0 |
| 71 | 89.9 | 89.8 | 89.4 | 89.9 |
| 72 | 57.2 | 57.1 | 57.1 | 57.0 |
| 83 | 51.1 | 57.1 | 52.6 | 52.2 |
| 84 | 89.9 | 89.9 | 89.9 | 89.9 |
| 85 | 89.9 | 89.8 | 89.9 | 89.8 |
| 86 | 51.9 | 52.3 | 39.2 | 39.1 |
| 87 | 53.4 | 52.7 | 39.1 | 38.9 |
| 88 | 59.5 | 59.2 | 39.1 | 39.2 |
| 89 | 55.4 | 55.2 | 38.9 | 38.9 |
| 90 | 52.3 | 53.4 | 39.8 | 40.0 |
| 91 | 53.0 | 53.1 | 38.3 | 39.7 |
| 92 | 55.1 | 55.5 | 40.1 | 40.3 |
| 93 | 57.7 | 57.7 | 39.7 | 38.0 |
| 94 | 58.3 | 58.3 | 58.7 | 58.7 |
| 95 | 55.8 | 54.3 | 54.3 | 54.5 |

TABLE 14
Evaluation result of dihedral angle between core portion of
nitrogen-containing condensed cyclic compound and group derived
from aromatic ring linked to core portion by single bond

| | Dihedral | Dihedral | Dihedral | Dihedral |
|---|---|---|---|---|
| Compound | angle 1 | angle 2 | angle 3 | angle 4 |
| No. | [°] | [°] | [°] | [°] |
| 96 | 54.9 | 54.7 | 51.2 | 52.3 |
| 97 | 54.4 | 55.1 | 52.1 | 50.1 |
| 98 | 52.0 | 50.3 | 38.2 | 38.1 |
| 99 | 51.9 | 50.3 | 37.3 | 37.1 |
| 100 | 51.9 | 50.3 | 39.2 | 38.9 |
| 101 | 51.4 | 50.5 | 38.8 | 39.0 |
| 102 | 50.6 | 53.5 | 38.2 | 38.2 |
| 103 | 50.6 | 51.3 | 40.0 | 40.0 |
| 104 | 53.8 | 53.8 | 51.6 | 51.6 |
| 106 | 53.3 | 51.3 | 51.3 | 53.3 |
| 108 | 50.5 | 51.9 | 52.2 | 54.2 |
| 109 | 55.2 | 55.8 | — | — |
| 110 | 55.2 | 55.7 | — | — |
| 114 | 60.7 | 60.9 | — | — |
| 115 | 60.4 | 60.4 | — | — |
| 116 | 51.4 | 49.8 | — | — |
| 120 | 55.2 | 52.3 | — | — |
| 121 | 51.3 | 53.3 | — | — |
| 122 | 60.6 | 60.3 | 38.0 | 37.9 |
| 123 | 60.3 | 60.2 | 37.9 | 38.1 |
| 124 | 58.7 | 58.7 | — | — |
| 125 | 58.3 | 58.5 | — | — |
| 126 | 61.0 | 60.7 | 60.5 | 60.5 |
| 127 | 61.1 | 60.9 | 60.6 | 60.7 |
| 128 | 58.8 | 58.4 | 58.9 | 58.5 |
| 129 | 60.9 | 60.7 | 38.6 | 38.7 |
| 130 | 56.9 | 56.5 | 38.4 | 38.5 |
| 131 | 56.8 | 56.7 | — | — |
| 132 | 56.6 | 56.6 | — | — |
| 133 | 59.3 | 59.1 | — | — |
| 134 | 60.6 | 60.6 | — | — |
| 135 | 61.1 | 60.9 | — | — |
| 136 | 61.3 | 61.6 | — | — |
| 140 | 57.9 | 57.9 | — | — |
| 141 | 55.6 | 56.5 | — | — |
| 142 | 60.3 | 58.2 | — | — |
| 144 | 54.8 | 54.7 | — | — |
| 145 | 51.3 | 49.3 | — | — |
| 146 | 59.6 | 60.3 | — | — |
| 147 | 51.2 | 51.2 | — | — |
| 148 | 61.4 | 60.8 | — | — |

TABLE 15
Evaluation result of dihedral angle between core portion of
nitrogen-containing condensed cyclic compound and group derived
from aromatic ring linked to core portion by single bond

| | Dihedral | Dihedral | Dihedral | Dihedral |
|---|---|---|---|---|
| Compound | angle 1 | angle 2 | angle 3 | angle 4 |
| No. | [°] | [°] | [°] | [°] |
| 149 | 60.2 | 60.2 | 39.2 | 38.7 |
| 150 | 60.9 | 60.5 | 38.8 | 38.8 |
| 151 | 60.7 | 60.8 | 39.7 | 40.0 |
| 152 | 58.2 | 56.8 | 40.6 | 40.7 |
| 153 | 82.6 | 82.5 | — | — |
| 154 | 49.7 | 51.7 | — | — |
| 155 | 52.0 | 50.7 | — | — |
| 156 | 49.7 | 50.5 | — | — |
| 157 | 60.1 | 60.0 | — | — |
| 158 | 60.1 | 59.4 | — | — |
| 159 | 60.5 | 61.1 | — | — |
| 160 | 61.2 | 60.8 | — | — |
| 194 | 79.0 | 79.3 | — | — |
| 195 | 54.5 | 54.3 | 42.6 | 43.3 |
| 196 | 60.3 | 60.2 | — | — |
| 197 | 59.9 | 59.9 | — | — |
| 198 | 60.0 | 59.9 | — | — |
| 199 | 59.3 | 59.3 | — | — |
| 200 | 60.0 | 59.8 | — | — |
| 201 | 59.3 | 59.4 | — | — |
| 202 | 58.7 | 58.4 | — | — |
| 203 | 59.9 | 59.9 | — | — |
| 204 | 60.4 | 60.3 | — | — |
| 205 | 87.4 | 87.5 | — | — |
| 206 | 58.9 | 58.7 | — | — |
| 207 | 59.7 | 59.5 | — | — |
| 208 | 60.1 | 60.0 | — | — |
| 209 | 85.0 | 60.8 | — | — |
| 210 | 89.6 | 89.8 | — | — |
| 211 | 89.4 | 89.2 | — | — |
| 212 | 67.1 | 67.1 | — | — |
| 213 | 66.8 | 66.9 | — | — |
| 214 | 67.8 | 67.9 | — | — |
| 215 | 67.7 | 67.6 | — | — |
| 216 | 68.1 | 70.0 | — | — |
| 217 | 66.4 | 68.2 | — | — |
| 218 | 50.5 | — | — | — |
| 221 | 64.9 | — | — | — |
| 222 | 54.5 | — | — | — |
| 225 | 63.9 | — | — | — |
| 228 | 56.5 | — | — | — |
| 231 | 64.0 | — | — | — |
| 232 | 60.9 | — | — | — |
| 233 | 60.3 | — | — | — |
| 234 | 58.9 | — | — | — |
| 235 | 60.4 | — | — | — |
| 236 | 60.9 | — | — | — |
| 237 | 61.3 | — | — | — |
| 238 | 89.9 | 89.9 | — | — |
| 239 | 90.0 | 90.0 | — | — |
| 240 | 89.6 | 89.6 | — | — |
| 241 | 86.0 | 86.0 | — | — |
| 242 | 86.8 | 87.8 | — | — |
| 243 | 88.8 | 88.8 | — | — |
| 244 | 85.6 | 85.7 | — | — |
| 245 | 87.0 | 87.1 | — | — |
| 246 | 51.5 | 50.2 | 48.4 | 48.6 |
| 248 | 80.5 | 80.4 | 48.1 | 48.0 |
| 249 | 72.3 | 72.7 | — | — |
| 250 | 70.0 | 70.0 | 44.0 | 44.4 |
| 251 | 64.8 | 64.6 | 37.0 | 37.2 |
| 252 | 55.0 | 54.0 | 39.2 | 39.3 |
| 253 | 64.3 | 62.7 | 38.9 | 38.9 |
| 254 | 79.0 | 79.1 | 38.1 | 38.1 |
| 255 | 64.4 | 63.7 | 35.9 | 36.1 |
| 256 | 53.9 | 55.0 | 38.3 | 38.4 |
| 257 | 64.9 | 62.8 | 37.6 | 37.5 |
| 258 | 79.1 | 79.1 | 37.3 | 37.3 |
| 259 | 62.5 | 62.5 | — | — |
| 260 | 54.7 | 54.7 | — | — |
| 261 | 64.2 | 64.2 | 37.5 | 38.1 |
| 262 | 64.5 | 64.5 | 42.4 | 42.9 |
| 263 | 62.8 | 63.7 | 37.0 | 38.8 |
| 264 | 63.4 | 63.4 | 37.4 | 37.4 |
| 265 | 63.7 | 63.1 | 38.4 | 39.1 |
| 266 | 68.0 | 62.1 | 48.7 | 53.0 |
| 267 | 64.0 | 64.0 | 38.2 | 37.8 |
| 268 | 64.2 | 64.2 | 38.0 | 38.1 |
| 269 | 66.3 | 66.3 | 36.4 | 36.0 |

Table 16: Evaluation result of dihedral angle between core portion of nitrogen-containing condensed cyclic compound and group derived from aromatic ring linked to core portion by single bond

TABLE 17
Evaluation result of dihedral angle between core portion of
nitrogen-containing condensed cyclic compound and group derived
from aromatic ring linked to core portion by single bond

| | Dihedral | Dihedral | Dihedral | Dihedral |
|---|---|---|---|---|
| Compound | angle 1 | angle 2 | angle 3 | angle 4 |
| No. | [°] | [°] | [°] | [°] |
| 270 | 58.8 | 59.9 | 38.1 | 37.8 |
| 271 | 88.7 | 62.9 | 44.8 | 42.1 |
| 272 | 56.7 | 56.7 | 47.2 | 45.0 |
| 273 | 59.3 | 59.3 | 38.3 | 38.3 |
| 274 | 59.8 | 59.8 | 38.6 | 38.5 |
| 275 | 68.4 | 68.2 | 52.6 | 52.5 |
| 276 | 65.7 | 60.1 | 38.7 | 38.4 |
| 277 | 59.9 | 59.8 | 37.8 | 38.3 |
| 278 | 63.4 | 63.4 | 38.0 | 38.6 |
| 279 | 62.7 | 62.7 | 38.1 | 38.0 |
| 280 | 63.3 | 63.3 | 39.7 | 39.7 |
| 281 | 56.3 | 56.4 | 39.3 | 39.2 |
| 282 | 65.0 | 65.6 | 75.0 | 75.1 |
| 283 | 56.9 | 70.4 | 61.3 | 59.4 |
| 284 | 62.3 | 62.7 | 70.6 | 70.3 |
| 285 | 64.4 | 65.6 | 47.3 | 49.8 |
| 286 | 57.0 | 66.7 | 52.2 | 42.5 |
| 287 | 66.6 | 65.1 | 79.1 | 52.4 |
| 289 | 52.5 | 52.5 | — | — |
| 291 | 54.9 | 54.9 | — | — |
| 292 | 61.2 | 61.6 | — | — |
| 293 | 62.2 | 62.2 | — | — |
| 294 | 51.4 | 51.4 | — | — |
| 295 | 61.5 | 61.5 | — | — |
| 296 | 64.4 | 64.4 | — | — |
| 297 | 62.7 | 62.7 | — | — |
| 299 | 69.1 | 69.1 | — | — |
| 300 | 65.2 | 64.6 | — | — |
| 301 | 58.5 | 58.6 | — | — |
| 303 | 56.2 | 56.2 | — | — |
| 304 | 63.6 | 63.6 | — | — |
| 305 | 57.0 | 57.0 | — | — |
| 306 | 62.5 | 62.4 | — | — |
| 308 | 88.5 | 88.7 | — | — |
| 309 | 84.5 | 86.7 | — | — |
| 310 | 86.9 | 86.9 | — | — |
| 311 | 53.1 | 53.2 | — | — |
| 312 | 85.6 | 87.2 | — | — |
| 313 | 84.1 | 84.1 | — | — |
| 314 | 48.9 | 50.6 | — | — |

TABLE 18
Evaluation result of dihedral angle between core portion of
nitrogen-containing condensed cyclic compound and group derived
from aromatic ring linked to core portion by single bond

| | Dihedral | Dihedral | Dihedral | Dihedral |
|---|---|---|---|---|
| Compound | angle 1 | angle 2 | angle 3 | angle 4 |
| No. | [°] | [°] | [°] | [°] |
| 315 | 63.1 | 63.1 | — | — |
| 316 | 67.0 | 67.0 | — | — |
| 317 | 62.6 | 62.7 | — | — |

PL Peak Wavelength and FWHM of Peak of Emission Spectrum in Film State

The obtained Compounds D1 to D8 were co-deposited on a quartz substrate at weight ratio of 1 wt % based on host compound mCBP (3,3′-bis(9H-carbazole-9-yl)-1,1′-biphenyl(3,3′-di(9H-carbazol-9-yl)-1,1′-biphenyl)) (Compound H9 in the description of the host material), at a vacuum degree of 10⁻⁵ Pa to thereby manufacture a thin film having a thickness of 50 nm. Each emission spectrum of the manufactured thin films were measured using a spectro fluorescence photometer F7000 of Hitachi Hightech Inc. at room temperature with an excitation wavelength of 360 nm, and the luminescence peak wavelength (nm) and FWHM of the peak of the emission spectrum were evaluated.

The results of the evaluation are shown in Table 19. Table 19 shows a changed amount of the FWHM of the peak of the emission spectrum from the FWHM of the peak of the emission spectrum measured in the solution state. The “maximum dihedral angle of the substituent substituting the core portion” of Table 19 indicates the maximum dihedral angle among dihedral angles 1 to 4, which are calculated by the “evaluation of compounds by calculation 4.” When only one dihedral angle is calculated in the “evaluation of compounds by calculation 4,” the maximum dihedral angle of the substituent substituting the core portion” indicates the value of the dihedral angle. In addition, because Compound C1 does not have a substituent including a group derived from an aromatic ring linked to the core portion by a single bond, the dihedral angles 1 to 4 cannot be calculated, and thus, “-” was input in the “maximum dihedral angle of substituent substituting core portion” column.

TABLE 19
Luminescence peak wavelength of nitrogen-containing condensed
cyclic compound and FWHM of peak of emission spectrum

| | Result of | | | |
|---|---|---|---|---|
| | evaluation | | | Evaluation |
| | through | | | result in |
| | simulation | Evaluation result in film | | solution |
| | Maximum | state | | state |
| | dihedral angle | Lumines- | FWHM of | FWHM of |
| | of substituent | cence peak | peak of | peak of |
| Com- | substituting | wavelength | emission | emission |
| pound | core portion | in PL | spectrum in PL | spectrum in PL |
| No. | [°] | [nm] | [nm] | [nm] |
| D1 | 90.0 | 458 | 15 | 12 |
| D7 | 60.9 | 462 | 18 | 16 |
| D8 | 67.5 | 454 | 12 | 11 |
| C1 | — | 454 | 22 | 13 |

From the result shown in Table 19, it can be seen that, when the nitrogen-containing condensed cyclic compound according to the present disclosure includes one or two or more substituents including a group derived from an aromatic ring linked to the core portion by a single bond, the dihedral angle between the core portion and the group derived from an aromatic ring linked to the core portion by a single bond may be 50° or more. Thus, regarding the nitrogen-containing condensed cyclic compound, it has been confirmed that the difference between the FWHM of the peak of the luminescence spectrum in PL obtained in a solution state and the FWHM of the peak of the luminescence spectrum in PL obtained in a film state is decreased. From the result, it may be assumed that a small FWHM of the peak of the luminescence spectrum may be obtained in the film state of the nitrogen-containing condensed cyclic compound, and a small FWHM of the peak of the luminescence spectrum may be obtained in an organic EL device using the nitrogen-containing condensed cyclic compound.

### Manufacturing of Organic EL Device

Preparation of Material for Forming Each Layer

For the material for forming each layer of the organic EL device, the materials below were prepared in addition to the obtained Compound D1 and Comparative Compound C1. Here, Compound H-H1, Compound H-E1, and phosphorescent complex Pt1 are respectively the same as described in connection with Compounds H55, H77, and phosphorescent complex P6.

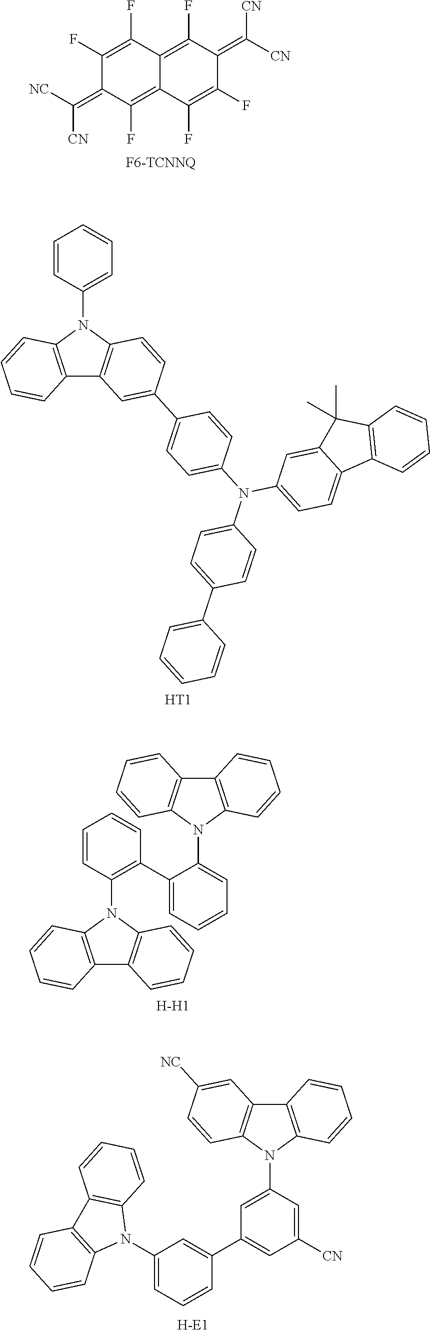

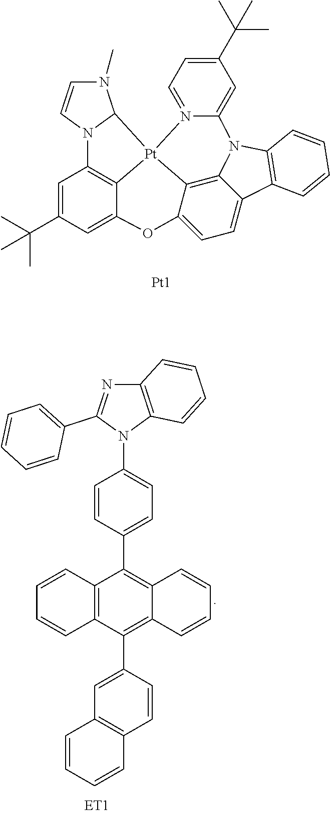

### Example 1

An ITO glass substrate was cut to a size of 50 mm×50 mm×0.5 mm and then, sonicated in acetone, isopropyl, alcohol, and pure water, in this stated order, each for 15 minutes, and then, washed by exposure to UV ozone for 30 minutes. The layers below were deposited on the ITO electrode (anode) of the glass substrate.

F6-TCNNQ was deposited on the ITO electrode to form a hole injection layer having a thickness of 10 nm. Subsequently, Compound HT1 was deposited on the hole injection layer to form a hole transport layer having a thickness of 126 nm. Then, Compound H-H1 was deposited on the hole transport layer to form an electron blocking layer having a thickness of 10 nm. As a result, a hole transport region was formed.

Compounds H-H1, H-E1, phosphorescent complex Pt1, and the obtained Compound D1 were co-deposited on the hole transport region to form an emission layer having a thickness of 40 nm. The emission layerwas formed such that the weight ratio of Compounds H-H1, H-E1, and phosphorescent complex Pt1 in the emission layer is Compound H-H1:Compound H-E1:phosphorescent complex Pt1=60:40:10. The emission layer was formed such that a concentration of Compound D1 in the emission layer is 0.5 wt % based on the total weight of Compounds H-H1 and H-E1, phosphorescent complex Pt1, and Compound D1 (thus, the total weight of the emission layer). Compounds H-H1 and H-E1 are host materials.

Then, Compound H-E1 was vacuum-deposited on the emission layer to form a hole blocking layer having a thickness of 10 nm. Subsequently, Compound ET1 and LiQ were co-deposited to a weight ratio of 5:5 (unit: parts by weight) to form an electron transport layer having a thickness of 36 nm. Then, LiQ was deposited on the electron transport layer to form an electron injection layer having a thickness of 0.5 nm. As a result, a hole transport region was formed.

Al (cathode) having a thickness of 80 nm was deposited on the electron injection layer to thereby manufacture an organic EL device.

In a glove box of a nitrogen atmosphere of water concentration of 1 ppm or less and oxygen concentration of 1 ppm or less, Glass sealing tube with a desiccating agent and an ultraviolet curing resin were (product name WB90US made by MORESCO) used to seal the organic EL device manufactured by the above process. As a result, the manufacturing of the organic EL device was completed.

### Examples 2 and 3

The organic EL devices of Examples 2 and 3 were manufactured in the same manner as in Example 1, except that, when forming the emission layer, the concentration of Compound D1 in the emission layer was changed to 1.5 wt % and 3.0 wt % based on the total weight of Compounds H-H1 and H-E1, phosphorescent complex Pt1, and Compound D1 (thus, the total weight of the emission layer). Then, each of the devices were sealed to complete the manufacturing process.

### Example 4

The organic EL device of Example 4 was manufactured in the same manner as in Example 2, except that, when forming the emission layer, the phosphorescent complex Pt1 was not used. Then, the device was sealed to complete the manufacturing process.

### Comparative Example 1

The organic EL device of Comparative Example 1 was manufactured in the same manner as in Example 2, except that, when forming the emission layer, the nitrogen-containing condensed cyclic compound was changed to Comparative Compound C1. Then, the device was sealed to complete the manufacturing process.

### Organic EL Device Evaluation 1

### Luminance, External Quantum Efficiency, and Lifespan of Device

The results of evaluating luminance, external quantum efficiency, and the lifespan of the device according to the below method are shown in Table 20.

A direct current regulated power supply (Source Meter 2400 made by KEITHLEY) was used to change the applied voltage with respect to the organic EL device while emitting light, and the luminance emission spectrum and the luminescence amount were measured in the luminance measurement apparatus (SR-3 made by Topcon).

Here, the external quantum efficiency was calculated from the luminescence amount of the emission spectrum and the current value at the time of measuring.

In addition, the lifespan (durability) of the device was shown as LT50 by measuring the amount of time taken when the emission luminance, which decays as time lapses, becomes 50% of the initial luminance when the device is continuously driven on a current value having an initial luminance of 1000 cd/m². LT50 in Table 20 indicates a relative value relative to the absolute value (unit: time (hrs)) and LT50 [hr] (LT50 (hrs)) of Example 4. In addition, the device lifespan of Example 4 is 1 hour.

Luminescence Peak Wavelength and FWHM of Peak of Emission Spectrum

The luminescence peak wavelength and the FWHM of the peak of the emission spectrum was read from the result of measuring the emission spectrum.

In this evaluation, the FWHM of the peak of the emission spectrum may be smaller, and when the FWHM is 30 nm or less, the organic EL device is considered to have high color purity, and when the FWHM is 25 nm or less, the color purity is considered as especially high.

In addition, in the present evaluation, the luminescence peak wavelength may be 450 nm or more and 470 nm or less, particularly, 450 nm or more and 465 nm or less.

TABLE 20
Composition of emission layer of each organic EL device and evaluation result

| | Constituents of emission layer | | | | | | | |
|---|---|---|---|---|---|---|---|---|
| | Nitrogen- | | | | | | | |
| | containing | | | Result of evaluation of organic EL device | | | | |
| | heterocyclic | | Phosphorescent | | External | Luminescence | FWHM of | |
| Organic | compound | | complex | | quantum | peak | peak of | |
| EL | | Concentration | Concentration | Luminance | efficiency | wavelength | emission | |
| device | Type | [%] | [%] | [Cd/m²] | [%] | [nm] | spectrum | LT50 |
| Example 1 | D1 | 0.5 | 9 | 1000 | 6.20 | 458 | 19 | 14.0 |
| Example 2 | D1 | 1.5 | 9 | 1000 | 4.42 | 459 | 20 | 10.5 |
| Example 3 | D1 | 3.0 | 9 | 1000 | 3.19 | 459 | 22 | 7.5 |
| Example 4 | D1 | 1.5 | 0 | 1000 | 2.57 | 459 | 19 | 1 |
| Comparative | C1 | 1.5 | 9 | 1000 | 2.45 | 456 | 41 | — |
| Example 1 | | | | | | | | |

As shown in Table 20, for the organic EL devices of Examples 1 to 4 using the nitrogen-containing condensed cyclic compound according to the present disclosure, excellent blue luminescence color was realized, the FWHM of the peak of the emission spectrum from the organic EL device was narrow, and luminescence with high color purity was realized. In addition, the organic EL devices of Examples 1 to 4 using the nitrogen-containing condensed cyclic compound according to the present disclosure were shown to have excellent external quantum efficiency. On the other hand, for the organic EL device of Comparative Example 1 using the nitrogen-containing condensed cyclic compound having a structure outside the scope of the present disclosure, the peak of the emission spectrum was shown to have wide FWHM and less color purity.

As shown in Table 20, in comparing organic EL devices using the same nitrogen-containing condensed cyclic compound, the organic EL device of Examples 1 to 3 using the nitrogen-containing condensed cyclic compound according to the present disclosure in combination with the phosphorescent complex has high external quantum efficiency and long device lifespan compared to the organic EL device of Example 4 not using the phosphorescent complex. In addition, as shown in Tables 10 and 20, in comparing the devices of the tables and the organic EL device of Comparative Example 1, the external quantum efficiency of the organic EL device of Examples 1 to 3 was confirmed to be higher than that of the organic EL device of Comparative Example 1. As shown in Examples 1 to 3, adjusting the concentration of the nitrogen condensed cyclic compound further improved the external quantum efficiency of the organic EL device. Here, the organic EL device of Examples 1 to 3 is an organic EL device that uses a nitrogen-containing condensed cyclic compound satisfying Conditions (i) to (iv) in combination with a phosphorescent complex, according to the present disclosure. The organic EL device of Comparative Example 1 has a structure outside the scope of the present disclosure, does not satisfy Condition (iii), and uses Comparative Compound C1, which is not considered to have TADF characteristics, in combination with a phosphorescent complex.

As shown in Examples 1 to 4 of Table 20, the nitrogen-containing condensed cyclic compound according to the present disclosure was shown to realize excellent blue luminescence color even at a high dopant concentration, the FWHM of the peak of the emission spectrum from the organic EL device was narrow, and luminescence with high color purity was realized. The above result is assumed to be based on the aggregation inhibiting effect of the substituent included in the nitrogen-containing condensed cyclic compound according to the present disclosure.

In addition, as shown in Table 12, Compounds D1 to D14 according to the present disclosure was shown to have high solubility in mesitylene compared to that of Comparative Compound C1. From the above result, it is assumed that the nitrogen-containing condensed cyclic compound according to the present disclosure has a high aggregation inhibiting effect due to a particular substituent.

As shown in Table 11, other Compounds D2 to D14 according to the present disclosure was shown to realize excellent blue luminescence color in PL and luminescence with high color purity, just as Compound D1 according to the present disclosure. As shown in Table 12, other Compounds D2 to D14 according to the present disclosure was shown to have high solubility in mesitylene, just as Compound D1 of the present disclosure. From the above result, Compounds D2 to D14 according to the present disclosure are assumed to realize excellent blue luminescence color in the organic EL device and luminescence with high color purity, just as Compound D1 according to the present disclosure. In addition, it is assumed that the lifespan is lengthened when used in combination with the phosphorescent complex in the organic EL device.

In addition, in Table 11, blue luminescence color is realized in the PL peak wavelength of Comparative Compound C1, and the FWHM of the peak of the emission spectrum in PL of Comparative Compound C1 is narrow and luminescence with high color purity is realized. From the above result, the blue luminescence color and excellent luminescence with high color purity are assumed to be originated from a particular nitrogen-containing condensed cyclic structure that is the parent skeleton. On the other hand, in Table 20, for the organic EL device of Comparative Example 1 using the Comparative Compound C1, the peak of the emission spectrum was shown to have wide FWHM and less color purity. The reason for the above result is assumed to be that Comparative Compound C1 does not have a high aggregation inhibiting effect by a particular substituent, and thus luminescence from the aggregate occurs. From the above result, the nitrogen-containing condensed cyclic compound having the structure represented by Formula (1) according to the present disclosure, the compound having a particular nitrogen-containing condensed cyclic compound structure, which is the parent skeleton, and having a particular substituent for the structure, may exhibit an excellent effect like Compound D1 according to the present disclosure.

### Manufacturing of Organic EL Device

### Example 5

An organic EL device was manufactured by changing various materials used. The manufacturing order is as below.

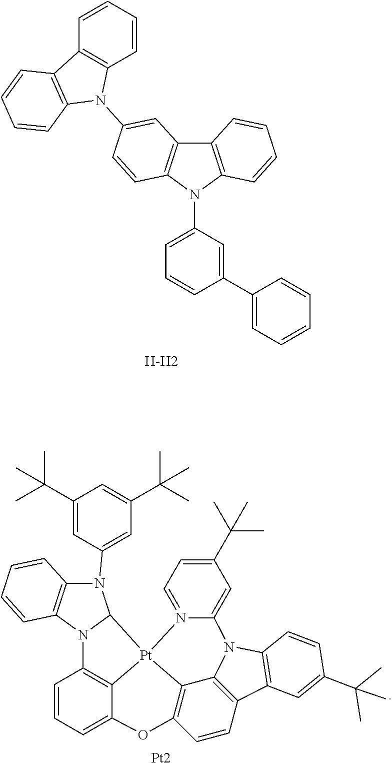

An ITO glass substrate was cut to a size of 50 mm×50 mm×0.5 mm and then, sonicated in acetone, isopropyl, alcohol, and pure water, in this stated order, each for 15 minutes, and then, washed by exposure to UV ozone for 30 minutes. The layers below were deposited on the ITO electrode (anode) of the glass substrate.

First, HAT-CN (manufactured by e-Ray) was deposited on an ITO electrode to form a hole injection layer having a film thickness of 10 nm. Subsequently, Compound HT1 was deposited on the hole transport layer to form a hole transport layer having a film thickness of 125 nm. Then, Compound H-H2 was deposited on the hole transport layer to form an electron blocking layer having a film thickness of 10 nm. As a result, a hole transport region was formed.

Compounds H-H2, H-E1, phosphorescent complex Pt2, and the obtained Compound D3 were co-deposited on the hole transport region to form an emission layer having a film thickness of 40 nm. A film was formed such that the weight ratio of Compounds H-H2, H-E1, and phosphorescent complex Pt2 on the emission layer is Compound H-H2:Compound H-E1:phosphorescent complex Pt2=60:40:10. A film was formed such that a concentration of Compound D3 in the emission layer is 0.5 wt % based on the total weight of Compounds H-H1 and H-E1, phosphorescent complex Pt2, and Compound D3 (thus, the total weight of the emission layer). Compounds H-H2 and H-E1 are host materials.

Then, Compound H-E1 was vacuum-deposited on the emission layer to form a hole blocking layer having a film thickness of 10 nm. Subsequently, Compound ET1 and LiQ were co-deposited to a weight ratio of 5:5 (unit: parts by weight) to form an electron transport layer having a film thickness of 30 nm. Then, LiQ was deposited on the electron transport layer to form an electron injection layer having a film thickness of 1 nm. As a result, a hole transport region was formed. Al (cathode) having a film thickness of 80 nm was deposited on the electron injection layer to thereby manufacture an organic EL device.

In a glove box of a nitrogen atmosphere of water concentration of 1 ppm or less and oxygen concentration of 1 ppm or less, Glass sealing tube with a desiccating agent and an ultraviolet curing resin were (product name WB90US made by MORESCO) used to seal the organic EL device manufactured by the above process. As a result, the manufacturing of the organic EL device was completed.

### Examples 6 to 9

The organic EL device of Examples 6 to 9 were manufactured in the same manner as in Example 5, except that, when forming the emission layer, the nitrogen-containing condensed cyclic compound was changed from D3 to D4, D5, D6, and D7, respectively. Then, the device was sealed to complete the manufacturing process.

### Comparative Example 2

The organic EL device of Comparative Example 2 was manufactured in the same manner as in Example 5, except that, when forming the emission layer, the nitrogen-containing condensed cyclic compound was changed from D3 to Comparative Compound C1. Then, the device was sealed to complete the manufacturing process.

### Organic EL Device Evaluation 2

### Luminance, External Quantum Efficiency, and Lifespan of Device

Regarding the obtained organic EL device of Examples 5 to 9 and Comparative Example 2, the same items as those in Evaluation 1 of the organic EL device were evaluated, and the results thereof are shown in Table 21.

Luminance, external quantum efficiency, luminescence peak wavelength, and FWHM of the peak of the emission spectrum were measured and evaluated in the same manner as in Evaluation 1 of the organic EL device. The device lifespan was evaluated in the same manner as in Evaluation 1 of the organic EL device, except that the amount of time taken for the emission luminance, which decays as time lapses, to become 95% of the initial luminance when the device is continuously driven on a current value having an initial luminance of 1,000 cd/m², was calculated as LT95. LT95 in Table 21 indicates the absolute value (unit: time (hrs)).

TABLE 21
Composition of emission layer of each organic EL device and evaluation result

| | Constituents of emission layer | | | | | | | |
|---|---|---|---|---|---|---|---|---|
| | Nitrogen- | | | Result of evaluation of organic EL device | | | | |
| | containing | | | | | | FWHM of | |
| | heterocyclic | | Phosphorescent | | External | Luminescence | peak of | |
| Organic | compound | | complex | | quantum | peak | emission | |
| EL | | Concentration | Concentration | Luminance | efficiency | wavelength | spectrum | LT95 |
| device | Type | [wt %] | [wt %] | [Cd/m²] | [%] | [nm] | [nm] | [hrs] |
| Example 5 | D3 | 0.5 | 9 | 1000 | 11.8 | 463 | 21 | 14.4 |
| Example 6 | D4 | 0.5 | 9 | 1000 | 13.3 | 466 | 22 | 28.2 |
| Example 7 | D5 | 0.5 | 9 | 1000 | 14.2 | 465 | 24 | 29.4 |
| Example 8 | D6 | 0.5 | 9 | 1000 | 15.3 | 468 | 25 | 34.6 |
| Example 9 | D7 | 0.5 | 9 | 1000 | 13.5 | 462 | 22 | 13.4 |
| Comparative | C1 | 0.5 | 9 | 1000 | 3.6 | 454 | 21 | 1 |
| Example 2 | | | | | | | | |

As shown in Table 21, it can be seen that the organic EL device of Examples 5 to 9 using the nitrogen-containing condensed cyclic compound of the present disclosure have narrow luminescence spectrum peak FWHM, excellent external quantum efficiency due to the realization of luminescence of high color purity, and long device lifespan. On the other hand, it can be seen that the organic EL device of Comparative Example 2, which uses a nitrogen-containing condensed cyclic compound having a structure other than that of the present disclosure, has low external quantum efficiency, and short device lifespan.

In addition, regarding Table 21, the organic EL device of Comparative Example 2 using the Comparative Compound C1 has a narrow luminescence spectrum peak FWHM and a luminescence of high color purity. The reason for such result is assumed to be the difficulty in forming an aggregation when the concentration of the nitrogen-containing condensed cyclic compound is low. However, the organic EL device of Comparative Example 2 shows superior external quantum efficiency and device lifespan. The organic EL device of Examples 5 to 9 using the nitrogen-containing condensed cyclic compound of the present disclosure show great improvement in the external quantum efficiency and device lifespan compared to the organic EL device of Comparative Example 2. The result is assumed to be caused by inhibition of aggregation between molecules and inhibition of intermolecular interaction between other compounds constituting the emission layer, at the same time, by introducing a certain substituent at a certain location, thereby decreasing the non-luminescent component and increasing the external quantum efficiency and device lifespan.

### Organic EL Device Manufacturing 3

### Examples 10 and 11

An organic EL device was manufactured in the same manner as in Example 1, by changing various materials used. The manufacturing order is as below.

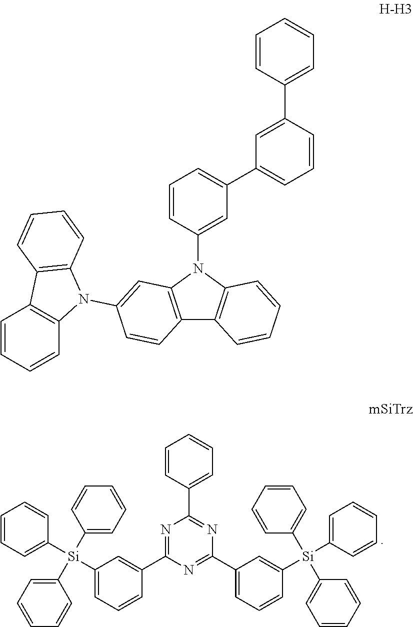 [SMILES: c1ccc(-c2cccc(-c3cccc(-n4c5ccccc5c5ccc(-n6c7ccccc7c7ccccc76)cc54)c3)c2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1]

An ITO glass substrate was cut to a size of 50 mm×50 mm×0.5 mm and then, sonicated in acetone, isopropyl, alcohol, and pure water, in this stated order, each for 15 minutes, and then, washed by exposure to UV ozone for 30 minutes. The layers below were deposited on the ITO electrode (anode) of the glass substrate.

First, HAT-CN (manufactured by e-Ray) was deposited on the ITO electrode to form a hole injection layer having a film thickness of 10 nm. Subsequently, Compound HT1 was deposited on the hole transport layer to form a hole transport layer having a film thickness of 140 nm. Then, Compound H-H3 was deposited on the hole transport layer to form an electron blocking layer having a film thickness of 5 nm. As a result, a hole transport region was formed.

The host material having a hole transport ability (HT-Host compound), Compound H-H3, mSiTrz (manufactured by LUMTEC), which is a host material having an electron transport ability (ET-Host compound), phosphorescent complex Pt2, and the obtained Compound D5 were co-deposited on the hole transport region to form an emission layer having a film thickness of 40 nm. In this state, the weight ratio of Compound H-H3 and mSiTrz of the emission layer was formed as shown in Table 22. The concentration of phosphorescent complex Pt2 of the emission layer was formed to be 7 wt %, based on the total weight of Compound H-H3, mSiTrz, phosphorescent complex Pt2, and Compound D5 (thus, the total weight of the emission layer), and the concentration of Compound D5 of the emission layer was formed to be 0.2 wt %, based on the total weight of Compound H-H3, mSiTrz, phosphorescent complex Pt2, and Compound D5 (thus, the total weight of the emission layer). Compound H-H1 and mSiTrz are host materials.

mSiTrz was vacuum-deposited on the emission layer to form a hole blocking layer having a film thickness of 5 nm. Then, TRE314 (manufactured by Toray Industries, Inc., electron transport material) and LiQ were co-deposited to a weight ratio of TRE314:LiQ=5:5 (unit: parts by weight) to form an electron transport material having a film thickness of 30 nm. Then, LiQ was deposited on the electron transport layer to form an electron injection layer having a film thickness of 1 nm. As a result, a hole transport region was formed.

Al (cathode) having a film thickness of 100 nm was deposited on the electron injection layer to thereby manufacture an organic EL device.

In a glove box of a nitrogen atmosphere of water concentration of 1 ppm or less and oxygen concentration of 1 ppm or less, a glass sealing tube with a desiccating agent and an ultraviolet curing resin were (product name WB90US manufactured by MORESCO) used to seal the organic EL device manufactured by the above process. As a result, the manufacturing of the organic EL device was completed.

### Examples 12 and 13

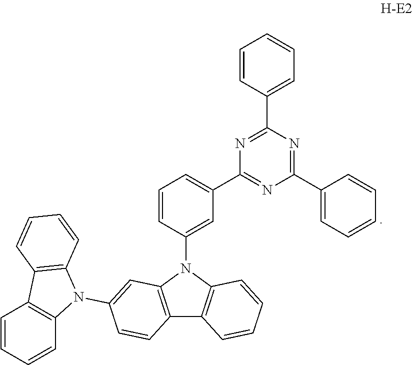 [SMILES: c1ccc(-c2nc(-c3ccccc3)nc(-c3cccc(-n4c5ccccc5c5ccc(-n6c7ccccc7c7ccccc76)cc54)c3)n2)cc1]

An organic EL device was manufactured in the same manner as in Example 10 and sealed, except that ET-Host compound was changed from mSiTrz to H-E2 in the film of the emission layer, the material used in the film of the hole blocking layer was changed from mSiTrz to H-E2, and the weight ratio of Compound H-H3 and mSiTrz of the emission layer was formed to be as shown in Table 22.

### Organic EL Device Evaluation 3: Luminance, External Quantum Efficiency, and Lifespan of Device

Regarding the obtained organic EL device of Examples 10 to 13, the same items as those in Evaluation 1 of the organic EL device were evaluated, and the results thereof are shown in Table 22.

Luminance, external quantum efficiency, luminescence peak wavelength, and FWHM of the peak of the emission spectrum were measured and evaluated in the same manner as in Evaluation 1 of the organic EL device. The device lifespan was evaluated in the same manner as in Evaluation 1 of the organic EL device, except that the amount of time taken for the emission luminance, which decays as time lapses, to become 95% of the initial luminance when the device is continuously driven on a current value having an initial luminance of 1,000 cd/m², was calculated as LT95. LT95 in Table 22 indicates the absolute value (unit: time (hrs)).

TABLE 22
Composition of emission layer of each
organic EL device and evaluation result

| | Constituents of emission layer | | | |
|---|---|---|---|---|
| | Proportion of host | | | Phospho- |
| | material | Nitrogen-containing | | rescent |
| | Compound | heterocyclic compound | | complex Pt2 |
| Organic | H-H3:ET-Host | | Concen- | Concen- |
| EL | compound | | tration | tration |
| device | [weight ratio] | Type | [wt %] | [wt %] |
| Example | 8.5:1.5 | D5 | 0.2 | 7 |
| 10 | | | | |
| Example | 8:2 | D5 | 0.2 | 7 |
| 11 | | | | |
| Example | 7.5:2.5 | D5 | 0.2 | 7 |
| 12 | | | | |
| Example | 7:3 | D5 | 0.2 | 7 |
| 13 | | | | |

| | Result of evaluation of organic EL device | | | | |
|---|---|---|---|---|---|
| | | | | FWHM of peak | |
| | | External | Lumines- | of emission | |
| Organic | Lumi- | quantum | cence peak | spectrum | |
| EL | nance | efficiency | wavelength | (FWHM) | LT95 |
| device | [Cd/m²] | [%] | [nm] | [nm] | [hrs] |
| Example | 1000 | 11.5 | 463 | 18.8 | 10.5 |
| 10 | | | | | |
| Example | 1000 | 11.2 | 463 | 19.6 | 17.0 |
| 11 | | | | | |
| Example | 1000 | 10.3 | 464 | 22.5 | 19.4 |
| 12 | | | | | |
| Example | 1000 | 10.5 | 464 | 22.5 | 17.5 |
| 13 | | | | | |

As shown in Table 22, regarding the organic EL devices of Examples 10 and 11 using, as a host material, the nitrogen-containing condensed cyclic compound of the present disclosure in combination with the compound having a triazine ring structure having a silyl group, the FWHM of the luminescence spectrum is more narrow, a luminescence of high color purity is realized, and the external quantum efficiency is excellent. In addition, the device lifespan is long.

As described above, the nitrogen-containing condensed cyclic compounds according to the present disclosure including Compounds D1 to D7 showed finely adjusted blue luminescence, excellent high color purity, and high emission efficiency in the organic EL device. In particular, the result was shown when the nitrogen-containing condensed cyclic compounds were used in combination with a phosphorescent material, and a significant improvement in device lifespan was shown. The results may sufficiently satisfy the specifications required for future-facing devices having wide color gamut such as BT2020, thereby making it possible to realize high-definition next-generation displays.

According to an embodiment, providing a compound wherein the peak wavelength of the emission spectrum is within the blue wavelength region, thereby having high color purity realizing high emission efficiency may be possible.

According to another embodiment, providing a fluorescence emitter including the compound may be possible.

According to another embodiment, providing a material for an organic EL device including the compound may be possible.

According to another embodiment, providing a method wherein the peak wavelength of the emission spectrum in the organic EL device is within a blue wavelength region, resulting in high color purity and realizing high emission efficiency, may be possible.

While descriptions were made with reference to embodiments and examples, the present disclosure is not limited to certain embodiments and examples, and various changes in form and details may be made therein without departing from the scope as defined by the following claims:

What is claimed is:

1. A material for an organic electroluminescent device, the material comprising a nitrogen-containing condensed cyclic compound having a structure of Formula (1) and a phosphorescent complex: 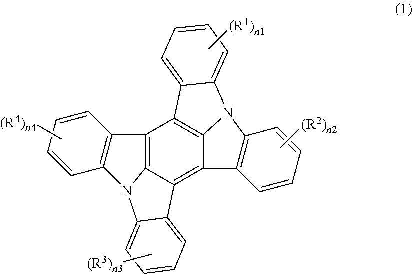
wherein, Formula (1) comprises a core portion, R¹ in the number of n1, R² in the number of n2, R³ in the number of n3, and R⁴ in the number of n4,
wherein, in Formula (1),
R¹ to R⁴ are each independently a group of (a) to (g),
n1 to n4 are each independently 0, 1, 2, 3, or 4, and
not all of n1 to n4 are 0, wherein
(a) is a halogen atom;
(b) is a cyano group;
(c) is a substituted or unsubstituted alkyl group having 1 to 20 carbon atoms;
(d) is a substituted or unsubstituted alkoxy group having 1 to 20 carbon atoms;
(e) is a substituted or unsubstituted aryl amino group having 6 to 20 carbon atoms;
(f) is a substituted or unsubstituted monovalent aromatic hydrocarbon group;
(g) is a substituted or unsubstituted monovalent heterocyclic group,
when one of n1 to n4 is 2 or more, each R¹, each R², each R³, or each R⁴ are identical to or different from each other,
each of R¹ to R⁴ are optionally linked to a ring-forming carbon of the core portion.

2. The material for an organic electroluminescent device of claim 1, wherein, in the nitrogen-containing condensed cyclic compound, the structure represented by Formula (1) is a structure represented by Formula (1A) or (1B): 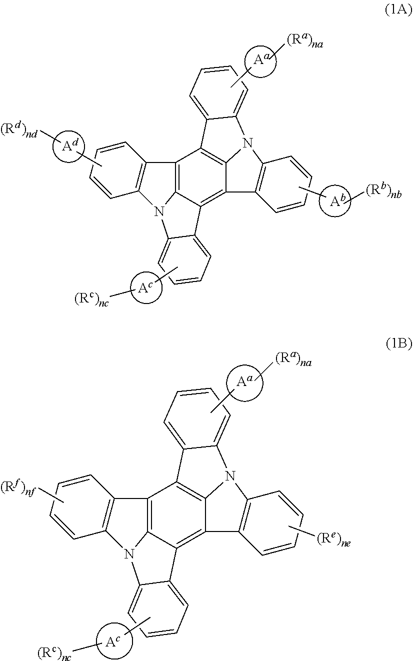
wherein, in Formulae (1A) and (1B),
A^{a} to A^{d} are each independently a group derived from a benzene ring, a group derived from a carbazole ring, or a group represented by Formula (1C), 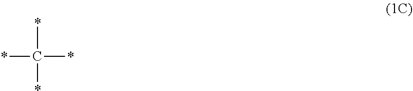 [SMILES: *C(*)(*)*]
R^{a} to R^{d} are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,
R^{e} and R^{f} are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
when each of A^{a} to A^{d} is a group derived from a benzene ring, na to nd are each independently 0, 1, 2, 3, 4, or 5,
when each of A^{a} to A^{d} is a group derived from a carbazole ring, na to nd are each independently 0, 1, 2, 3, 4, 5, 6, 7, or 8,
when each of A^{a} to A^{d} is a group represented by Formula (1C), na to nd are each independently 3,
ne and nf are each independently 0, 1, 2, 3, or 4,
wherein, when na is 2 or more, each R^{a} are identical to or different from each other,
when nb is 2 or more, each R^{b} are identical to or different from each other,
when nc is 2 or more, each R^{c} are identical to or different from each other,
when nd is 2 or more, each R^{d} are identical to or different from each other,
when ne is 2 or more, each R^{e} may be are identical to or different from each other,
when nf is 2 or more, each R^{f} are identical to or different from each other,
each of R^{a} to R^{f} are optionally linked to a ring-forming carbon of the core portion, and
in Formula (1C), * indicates a binding site to a neighboring atom.

3. The material for an organic electroluminescent device of claim 1, wherein
the structure represented by Formula (1) is a structure represented by Formula (2) or (3): 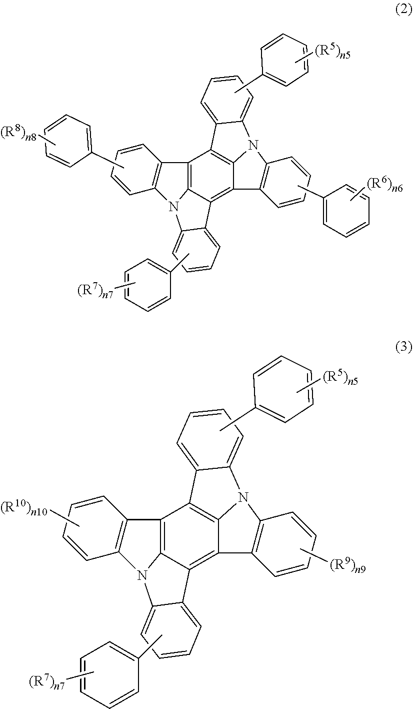
wherein, in Formulae (2) and (3),
R⁵ to R⁸ are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, or an unsubstituted aryl amino group having 6 to 20 carbon atoms,
R⁹ and R¹⁰ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n5 to n8 are each independently 0, 1, 2, 3, 4, or 5,
n9 and n10 are each independently 0, 1, 2, 3, or 4,
when one of n5 to n10 is 2 or more, each R⁵, each R⁶, each R⁷, each R⁸, each R⁹, or each R¹⁰ are identical to or different from each other,
each of R⁵ to R¹⁰ are optionally linked to a ring-forming carbon of the core portion.

4. The material for an organic electroluminescent device of claim 3, wherein
the structure of Formula (1) of the nitrogen-containing condensed cyclic compound is represented by the structure of Formula (2), and
at least one of R⁵ to R⁸ in Formula (2) is an unsubstituted alkyl group having 1 to 20 carbon atoms.

5. The material for an organic electroluminescent device of claim 3, wherein
the structure of Formula (1) of the nitrogen-containing condensed cyclic compound is represented by the structure of Formula (3), and
at least one of R⁵, R⁷, R⁹, and R¹⁰ in Formula (3) is an unsubstituted alkyl group having 1 to 20 carbon atoms.

6. The material for an organic electroluminescent device of claim 3, wherein
the structure of Formula (1) of the nitrogen-containing condensed cyclic compound is represented by the structure of Formula (2), and
in Formula (2), at least one of n5 to n8 is 3 or more or at least one of R⁵ to R⁸ is an unsubstituted branched alkyl group having 4 to 15 carbon atoms.

7. The material for an organic electroluminescent device of claim 3, wherein
the structure of Formula (1) of the nitrogen-containing condensed cyclic compound is represented by the structure of Formula (3), and
in Formula (3), at least one of n5, n7, n9, and n10 is 3 or more, or at least one of R⁵, R⁷, R⁹, and R¹⁰ is an unsubstituted branched alkyl group having 4 to 15 carbon atoms.

8. The material for an organic electroluminescent device of claim 3, wherein
the structure of Formula (2) or (3) of the nitrogen-containing condensed cyclic compound is represented by the structure of one of Formulae (3-1) to (3-9):

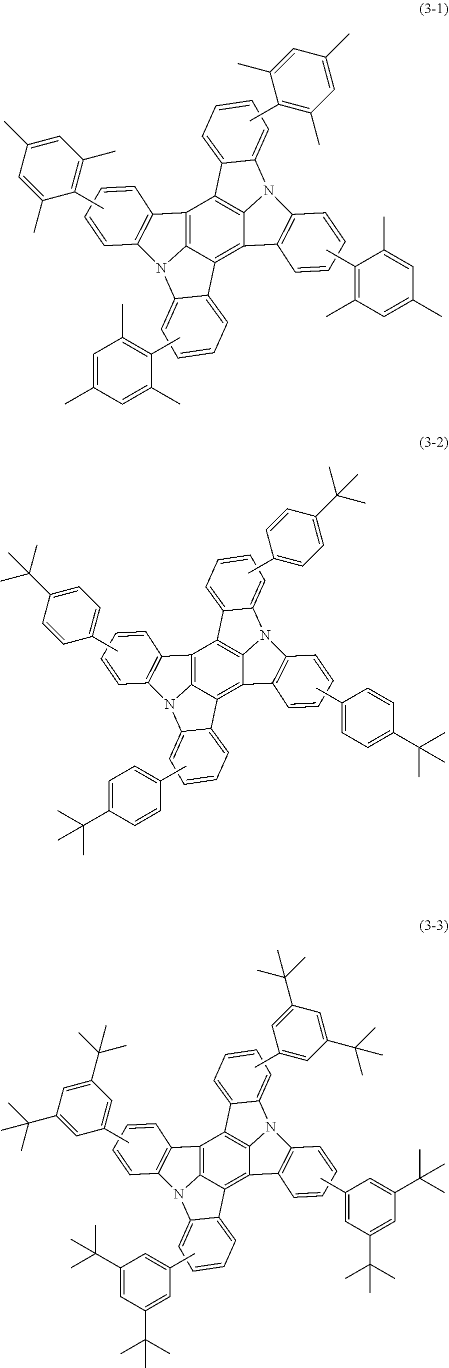

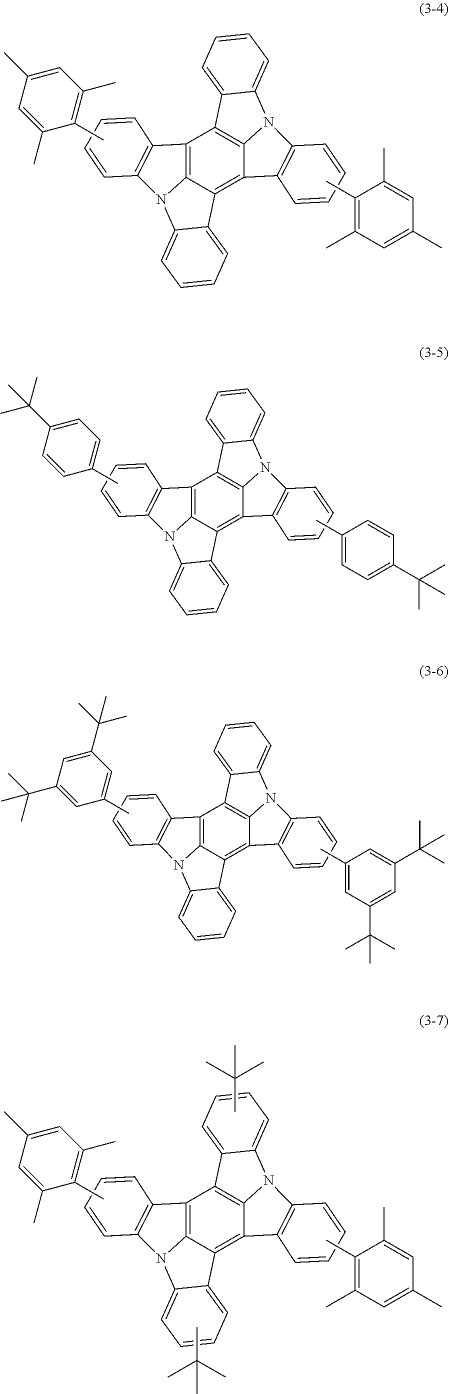

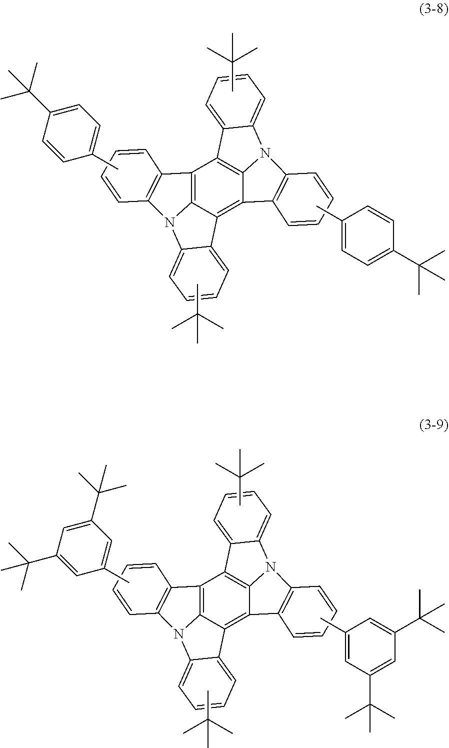

9. The material for an organic electroluminescent device of claim 2, wherein
the structure of Formula (1A), or (1B) of the nitrogen-containing condensed cyclic compound is a structure represented by Formula (7) or (8): 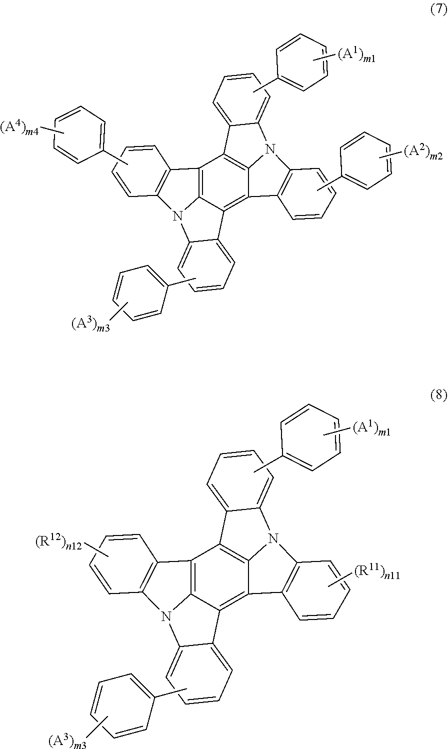
wherein, in Formulae (7) and (8),
A¹ to A⁴ are each independently an unsubstituted alkyl group having 1 to 20 carbon atoms, a substituted or unsubstituted monovalent aromatic hydrocarbon group, or a substituted or unsubstituted monovalent aromatic hetero ring,
R¹¹ and R¹² are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n1 to n4 are each independently 0, 1, 2, 3, 4, or 5,
n11 and n12 are each independently 0, 1, 2, 3, or 4,
wherein, when m1 is 2 or more, each A¹ are identical to or different from each other,
when m2 is 2 or more, each A² are identical to or different from each other,
when m3 is 2 or more, each A³ are identical to or different from each other,
when m4 is 2 or more, each A⁴ are identical to or different from each other,
when n11 is 2 or more, each R¹¹ are identical to or different from each other,
when n12 is 2 or more, each R¹² are identical to or different from each other,
each of A¹ to A⁴ and R¹¹ to R¹² are optionally linked to a ring-forming carbon of the core portion,
wherein, in Formula (7),
not all of A¹ to A⁴ are alkyl groups having 1 to 20 carbon atoms, and not all m1 to m4 are 0,
wherein, in Formula (8),
not all of A¹ to A³ are alkyl groups having 1 to 20 carbon atoms, and not all of m1 and m3 are 0.

10. The material for an organic electroluminescent device of claim 2, wherein
the structure of Formula (1A), or (1B) of the nitrogen-containing condensed cyclic compound is a structure represented by Formulae (12) to (14): 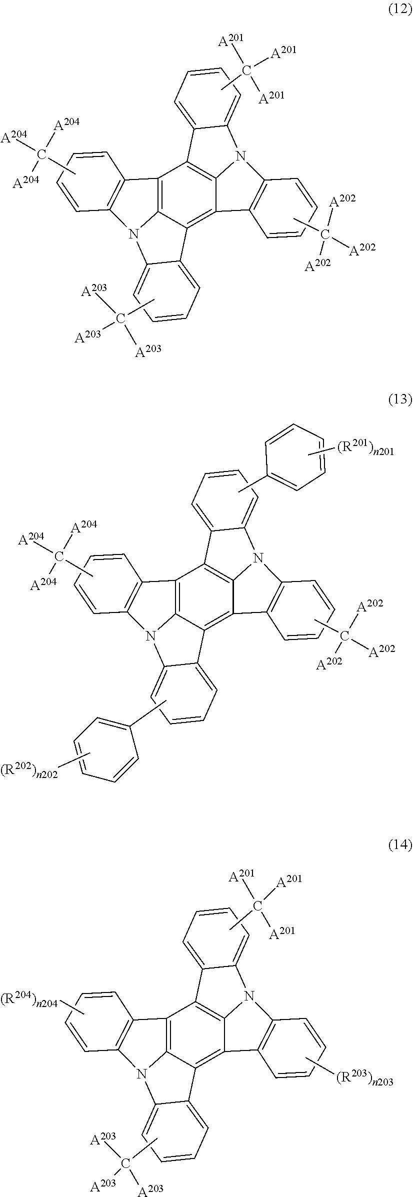
wherein, in Formulae (12) to (14),
A²⁰¹ to A²⁰⁴ are each independently an unsubstituted alkyl group having 1 to 20 carbon atoms, a substituted or unsubstituted monovalent aromatic hydrocarbon group, or a substituted or unsubstituted monovalent aromatic heterocyclic group,
R²⁰¹ to R²⁰² are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,
R²⁰³ and R²⁰⁴ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n201 and n202 are each independently 0, 1, 2, 3, 4, or 5,
n203 and n204 are each independently 0, 1, 2, 3, or 4,
wherein, each A²⁰¹ are identical to or different from each other, each A²⁰² are identical to or different from each other, each A²⁰³ are identical to or different from each other, and each A²⁰⁴ are identical to or different from each other,
optionally, two or more of A²⁰¹, two or more of A²⁰², two or more of A²⁰³, and two or more of A²⁰⁴ each form a ring,
when n201 is 2 or more, each R²⁰¹ are identical to or different from each other, when n202 is 2 or more, each R²⁰² are identical to or different from each other, when n203 is 2 or more, each R²⁰³ are identical to or different from each other, and when n204 is 2 or more, each R²⁰⁴ are identical to or different from each other,
each of A²⁰¹ to A²⁰⁴ are optionally linked to a ring-forming carbon of the core portion of Formula (12),
each of A²⁰², A²⁰⁴, R²⁰¹, and R²⁰² are optionally linked to a ring-forming carbon of the core portion of Formula (13),
each of A²⁰¹, A²⁰³, R²⁰³, and R²⁰⁴ are optionally linked to a ring-forming carbon of the core portion of Formula (14),
in Formula (12), not all of each A²⁰¹, each A²⁰², each A²⁰³, and each A²⁰⁴ are unsubstituted alkyl groups having 1 to 20 carbon atoms,
in Formula (13), not all of each A²⁰² and each A²⁰⁴ are unsubstituted alkyl groups having 1 to 20 carbon atoms, and
in Formula (14), not all of each A²⁰¹ and each A²⁰³ are unsubstituted alkyl groups having 1 to 20 carbon atoms.

11. The material for an organic electroluminescent device of claim 1, the material comprising
at least one of R¹ to R⁴ is an aromatic hydrocarbon group or a heterocyclic group and is linked to the core portion represented by Formula (1-1) by a single bond, wherein
a dihedral angle between the core portion and at least one group derived from an aromatic ring is 50° or more.

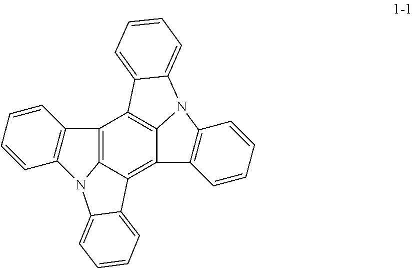 [SMILES: c1ccc2c(c1)c1c3c4ccccc4n4c5ccccc5c(c5c6ccccc6n2c15)c34]

12. The material for an organic electroluminescent device of claim 11, wherein
at least one of R¹ to R⁴ is a group derived from a benzene ring, a group derived from a carbazole ring, a group derived from a dibenzofuran ring, or a combination thereof.

13. The material for an organic electroluminescent device of claim 1, wherein
the nitrogen-containing condensed cyclic compound satisfies Conditions (i) to (iv):
ΔE_{ST}>Δ≤E_{ST2}+ΔE′_{TT} Condition (i)
0 eV<ΔE_{ST2}+ΔE′_{TT}≤1.0 eV Condition (i)
0 eV<ΔE′_{TT}≤0.15 eV Condition (iii)
ΔE_{ST2}>0 eV Condition (iv)
wherein, in Conditions (i) to (iv),
ΔE_{ST}(eV) indicates a difference value obtained by subtracting the lowest triplet excitation energy (eV) calculated from a T₁ equilibrium structure from the lowest singlet excitation energy (eV) calculated from an S₁ equilibrium structure,
ΔE_{ST2}(eV) indicates a difference value obtained by subtracting the second lowest triplet excitation energy (eV) calculated from a T₂ equilibrium structure from the lowest singlet excitation energy (eV) calculated from the S₁ equilibrium structure, and
ΔE′_{TT}(eV) indicates the difference value obtained from subtracting the lowest triplet excitation energy (eV) calculated from the T₂ equilibrium structure from the second lowest triplet excitation energy (eV) calculated from the T₂ equilibrium structure.

14. The material for an organic electroluminescent device of claim 1, wherein
the nitrogen-containing condensed cyclic compound has a mesitylene solubility of 1.0 g/L or more.

15. The material for an organic electroluminescent device of claim 1, wherein
the phosphorescent complex is a platinum complex.

16. The material for an organic electroluminescent device of claim 1, wherein
the material further comprises a host material.

17. The material for an organic electroluminescent device of claim 16, wherein
the host material has a structure represented by Formula (5): 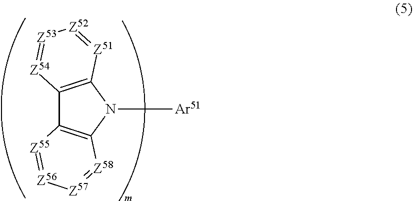
wherein, in Formula (5),
Z⁵¹ is CH, CR⁵¹, or N,
Z⁵² is CH, CR⁵², or N,
Z⁵³ is CH, CR⁵³, or N,
Z⁵⁴ is CH, CR⁵⁴, or N,
Z⁵⁵ is CH, CR⁵⁵, or N,
Z⁵⁶ is CH, CR⁵⁶, or N,
Z⁵⁷ is CH, CR⁵⁷, or N,
Z⁵⁸ is CH, CR⁵⁸, or N,
R⁵¹ to R⁵⁸ are each independently a group of (5a) to (5h), wherein
(5a) is a cyano group,
(5b) is a substituted or unsubstituted alkyl group having 1 to 20 carbon atoms,
(5c) is a substituted or unsubstituted alkoxy group having 1 to 20 carbon atoms,
(5d) is a substituted or unsubstituted aryl amino group having 6 to 20 carbon atoms,
(5e) is a substituted or unsubstituted phosphoryl group (a —POH₂ group),
(5f) is a substituted or unsubstituted silyl group (a —SiH₃ group),
(5g) is a substituted or unsubstituted monovalent aromatic hydrocarbon group,
(5h) is a substituted or unsubstituted monovalent heterocyclic group,
Ar⁵¹ comprises an aromatic hydrocarbon group or a heterocyclic group,
m is 1, 2, 3, 4, 5, or 6,
wherein each of R⁵¹ and R⁵², R⁵² and R⁵³, R⁵³ and R⁵⁴, R⁵⁵ and R⁵⁶, R⁵⁶ and R⁵⁷, or R⁵⁷ and R⁵⁸ are optionally linked to form an aliphatic hydrocarbon ring, an aromatic hydrocarbon ring, or a hetero ring.

18. The material for an organic electroluminescent device of claim 16, wherein
the host material has a structure represented by Formula (6): 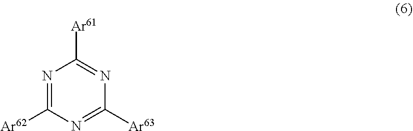
wherein, in Formula (6),
Ar⁶¹ to Ar⁶³ are each independently a substituted or unsubstituted monovalent aromatic hydrocarbon group or a substituted or unsubstituted monovalent heterocyclic group.

19. The material for an organic electroluminescent device of claim 1, wherein the material is a fluorescence emitter.

20. A nitrogen-containing condensed cyclic compound having the structure represented by Formula (1) and satisfying Conditions (i) to (iv): 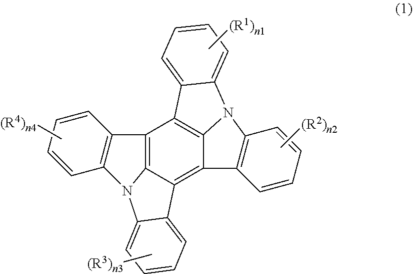
Wherein Formula (1) comprises a core portion, R¹ in the number of n1, R² in the number of n2, R³ in the number of n3, and R⁴ in the number of n4,
wherein, in Formula (1),
R¹ to R⁴ are each independently a group of (a) to (g),
n1 to n4 are each independently 0, 1, 2, 3, or 4, and
not all of n1 to n4 are 0, wherein
(a) is a halogen atom;
(b) is a cyano group;
(c) is a substituted or unsubstituted alkyl group having 1 to 20 carbon atoms;
(d) is a substituted or unsubstituted alkoxy group having 1 to 20 carbon atoms;
(e) is a substituted or unsubstituted aryl amino group having 6 to 20 carbon atoms;
(f) is a substituted or unsubstituted monovalent aromatic hydrocarbon group;
(g) is a substituted or unsubstituted monovalent heterocyclic group,
wherein, when one of n1 to n4 is 2 or more, each R¹, each R², each R³, or each R⁴ are identical to or different from each other,
each of R¹ to R⁴ are optionally linked to a ring-forming carbon of the core portion,
ΔE_{ST}>ΔE_{ST2}+ΔE′_{TT} Condition (i)
0 eV<ΔE_{ST2}+ΔE′_{TT}≤Δ1.0 eV Condition (ii)
0 eV<ΔE′_{TT}≤0.15 eV Condition (iii)
ΔE_{ST2}>0 eV Condition (iv)
wherein, in Conditions (i) to (iv),
ΔE_{ST}(eV) indicates a difference value obtained by subtracting the lowest triplet excitation energy (eV) calculated from a T₁ equilibrium structure from the lowest singlet excitation energy (eV) calculated from an S₁ equilibrium structure,
ΔE_{ST2}(eV) indicates a difference value obtained by subtracting the second lowest triplet excitation energy (eV) calculated from a T₂ equilibrium structure from the lowest singlet excitation energy (eV) calculated from the S₁ equilibrium structure, and
ΔE′_{TT}(eV) indicates a difference value obtained by subtracting the lowest triplet excitation energy (eV) calculated from the T₂ equilibrium structure from the second lowest triplet excitation energy (eV) calculated from the T₂ equilibrium structure.

21. The nitrogen-containing condensed cyclic compound of claim 20, wherein
the structure represented by Formula (1) is represented by a structure represented by Formula (1A) or (1B): 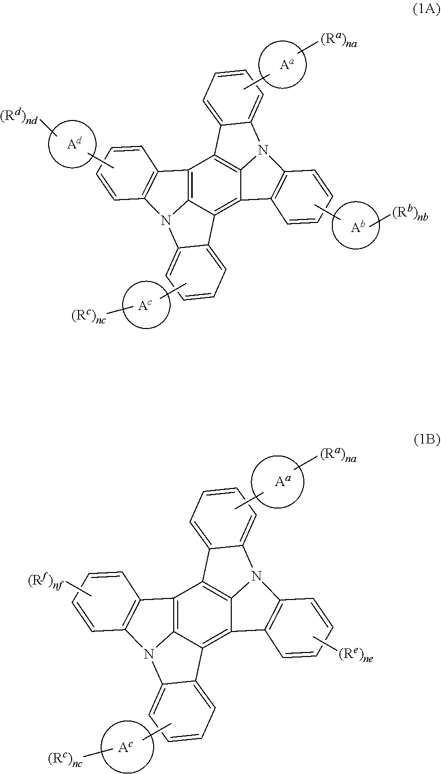
wherein, in Formulae (1A) and (1B),
A^{a} to A^{d} are each independently a group derived from a benzene ring, a group derived from a carbazole ring, or a group represented by Formula (1C), 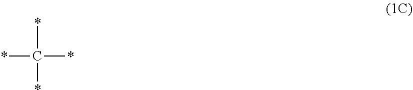 [SMILES: *C(*)(*)*]
R^{a} to R^{d} are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,
R^{e} and R^{f} are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
when each of A^{a} to A^{d} is a group derived from a benzene ring, na to nd are each independently 0, 1, 2, 3, 4, or 5,
when each of A^{a} to A^{d} is a group derived from a carbazole ring, na to nd are each independently 0, 1, 2, 3, 4, 5, 6, 7, or 8,
when each of A^{a} to A^{d} is a group represented by Formula (1C), na to nd are each independently 3,
ne and nf are each independently 0, 1, 2, 3, or 4,
wherein, when na is 2 or more, each R^{a} are identical to or different from each other, when nb is 2 or more, each R^{b} are identical to or different from each other, when nc is 2 or more, each R^{c} are identical to or different from each other, when nd is 2 or more, each R^{d} are identical to or different from each other, when ne is 2 or more, each R^{e} are identical to or different from each other, and when nf is 2 or more, each R^{f} are identical to or different from each other,
each of R^{a} to R^{f} are optionally linked to a ring-forming carbon of the core portion, and
in Formula (1C), * indicates a binding site to a neighboring atom.

22. The nitrogen-containing condensed cyclic compound of claim 21, wherein
in Formula (1A), when each of A^{a} to A^{d} is a group derived from a benzene ring, at least one of na to nd is 3 or more, or at least one of R^{a} to R^{d} is an unsubstituted branched alkyl group having 4 to 15 carbon atoms, and
in Formula (1B), when each of A^{a} and A^{d} is a group derived from a benzene ring, at least one of na, nc, ne, and nf is 3 or more, or at least one of R^{a}, R^{c}, R^{e}, and R^{f} is an unsubstituted branched alkyl group having 4 to 15 carbon atoms.

23. The nitrogen-containing condensed cyclic compound of claim 20, wherein
the structure represented by Formula (1) has a structure represented by Formula (2) or (3): 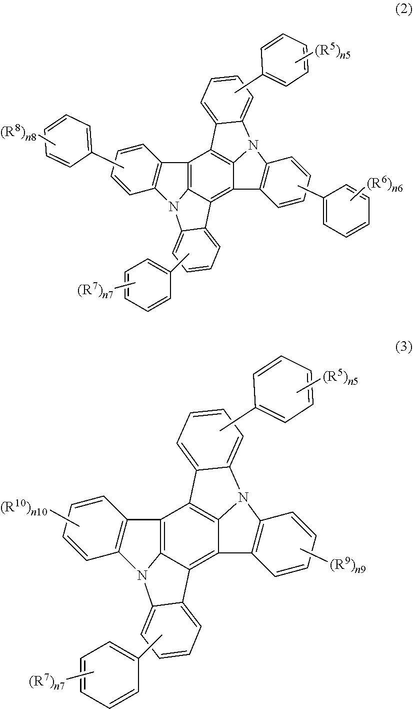
wherein, in Formulae (2) and (3),
R⁵ to R⁸ are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, or an unsubstituted aryl amino group having 6 to 20 carbon atoms,
R⁹ and R¹⁰ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n5 to n8 are each independently 0, 1, 2, 3, 4, or 5,
n9 and n10 are each independently 0, 1, 2, 3, or 4, and
when one of n5 to n10 is 2 or more, each R⁵, each R⁶, each R⁷, each R⁸, each R⁹, or each R¹⁰ are identical to or different from each other,
each of R⁵ to R¹⁰ are optionally linked to a ring-forming carbon of the core portion.

24. The nitrogen-containing condensed cyclic compound of claim 23, wherein
in Formula (2), at least one of n5 to n8 is 3 or more or at least one of R⁵ to R⁸ is an unsubstituted branched alkyl group having 4 to 15 carbon atoms, and
in Formula (3), at least one of n5, n7, n9, and n10 is 3 or more, or at least one of R⁵, R⁷, R⁹, and R¹⁰ is an unsubstituted branched alkyl group having 4 to 15 carbon atoms.

25. The nitrogen-containing condensed cyclic compound of claim 20, wherein
the structure represented by Formula (1) has a structure represented by Formula (7) or (8): 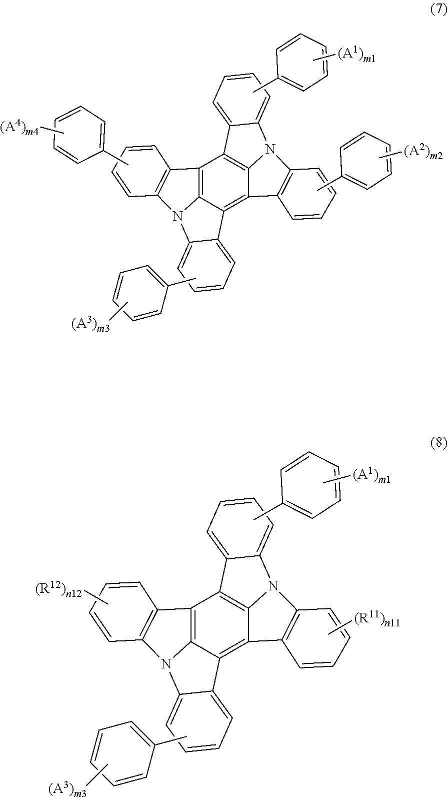
wherein, in Formulae (7) and (8),
A¹ to A⁴ are each independently an unsubstituted alkyl group having 1 to 20 carbon atoms, a substituted or unsubstituted monovalent aromatic hydrocarbon group, or a substituted or unsubstituted monovalent aromatic hetero ring,
R¹¹ and R¹² are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n1 to n4 are each independently 0, 1, 2, 3, 4, or 5,
n11 and n12 are each independently 0, 1, 2, 3, or 4,
wherein, when m1 is 2 or more, each A¹ are identical to or different from each other,
when m2 is 2 or more, each A² are identical to or different from each other,
when m3 is 2 or more, each A³ are identical to or different from each other,
when m4 is 2 or more, each A⁴ are identical to or different from each other,
when n11 is 2 or more, each R¹¹ are identical to or different from each other,
when n12 is 2 or more, each R¹² are identical to or different from each other,
each of A¹ to A⁴ and R¹¹ to R¹² are optionally linked to a ring-forming carbon of the core portion,
wherein, in Formula (7),
not all of A¹ to A⁴ are alkyl groups having 1 to 20 carbon atoms, and not all m1 to m4 are 0,
wherein, in Formula (8),
not all of A¹ to A³ are alkyl groups having 1 to 20 carbon atoms, and not all of m1 and m3 are 0.

26. The nitrogen-containing condensed cyclic compound of claim 20, wherein
the structure represented by Formula (1) is a structure selected from a group represented by one of Formulae (12) to (14): 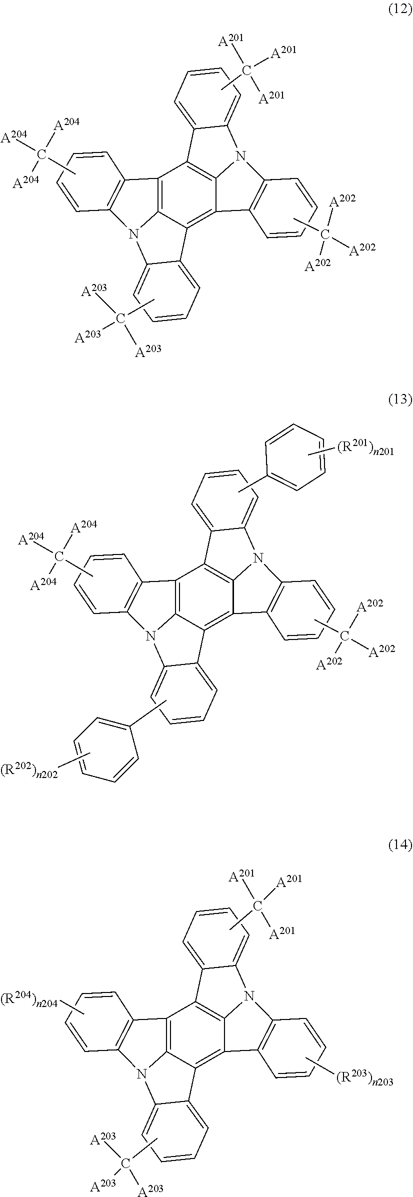
wherein, in Formulae (12) to (14),
A²⁰¹ to A²⁰⁴ are each independently an unsubstituted alkyl group having 1 to 20 carbon atoms, a substituted or unsubstituted monovalent aromatic hydrocarbon group, or a substituted or unsubstituted monovalent aromatic heterocyclic group,
R²⁰¹ to R²⁰² are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,
R²⁰³ and R²⁰⁴ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n201 and n202 are each independently 0, 1, 2, 3, 4, or 5,
n203 and n204 are each independently 0, 1, 2, 3, or 4,
wherein, each A²⁰¹ are identical to or different from each other, each A²⁰² are identical to or different from each other, each A²⁰³ are identical to or different from each other, and each A²⁰⁴ are identical to or different from each other,
two or more of A²⁰¹, two or more of A²⁰², two or more of A²⁰³, and two or more of A²⁰⁴ are optionally linked to form a ring,
when n201 is 2 or more, each R²⁰¹ are identical to or different from each other, when n202 is 2 or more, each R²⁰² are identical to or different from each other, when n203 is 2 or more, each R²⁰³ are identical to or different from each other, and when n204 is 2 or more, each R²⁰⁴ are identical to or different from each other,
each of A²⁰¹ to A²⁰⁴ are optionally linked to a ring-forming carbon of the core portion of Formula (12),
each of A²⁰², A²⁰⁴, R²⁰¹, and R²⁰² are optionally linked to a ring-forming carbon of the core portion of Formula (13),
each of A²⁰¹, A²⁰³, R²⁰³, and R²⁰⁴ are optionally linked to a ring-forming carbon of the core portion of Formula (14),
in Formula (12), not all of each A²⁰¹, each A²⁰², each A²⁰³, and each A²⁰⁴ are unsubstituted alkyl groups having 1 to 20 carbon atoms,
in Formula (13), not all of each A²⁰² and each A²⁰⁴ are unsubstituted alkyl groups having 1 to 20 carbon atoms, and
in Formula (14), not all of each A²⁰¹ and each A²⁰³ are unsubstituted alkyl groups having 1 to 20 carbon atoms.

27. A nitrogen-containing condensed cyclic compound having a structure selected from a group represented by Formulae (2), (3), (7), (8), and (12) to (14): 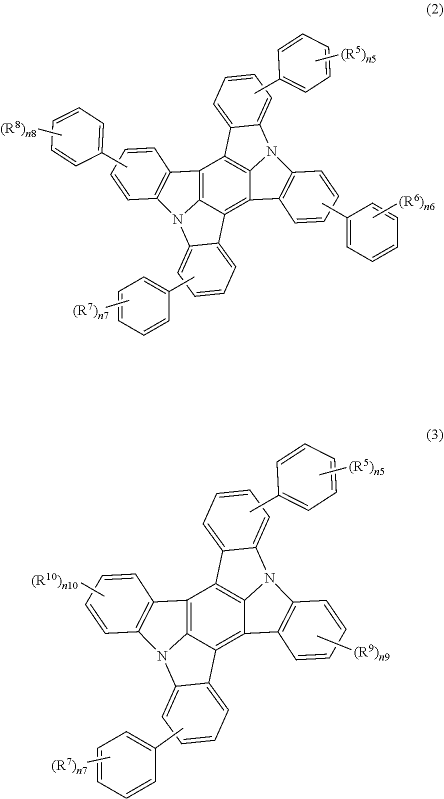
wherein, in Formulae (2) and (3),
R⁵ to R⁸ are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, or an unsubstituted aryl amino group having 6 to 20 carbon atoms,
R⁹ and R¹⁰ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n5 to n8 are each independently 0, 1, 2, 3, 4, or 5,
n9 and n10 are each independently 0, 1, 2, 3, or 4,
when one of n5 to n10 is 2 or more, each R⁵, each R⁶, each R⁷, each R⁸, each R⁹, or each R¹⁰ are identical to or different from each other,
each of R⁵ to R¹⁰ are optionally linked to a ring-forming carbon of the core portion,
in Formula (2), at least one of n5 to n8 is 3 or more or at least one of R⁵ to R⁸ is an unsubstituted branched alkyl group having 4 to 15 carbon atoms, and
in Formula (3), at least one of n5, n7, n9, and n10 is 3 or more, or at least one of R⁵, R⁷, R⁹, and R¹⁰ is an unsubstituted branched alkyl group having 4 to 15 carbon atoms, 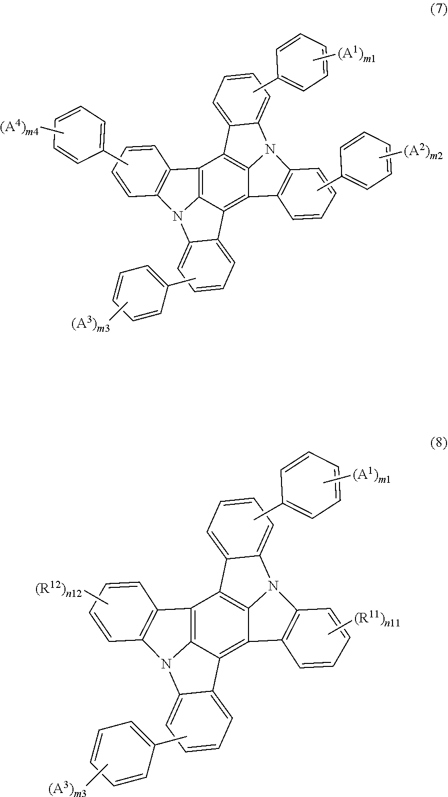
wherein, in Formulae (7) and (8),
A¹ to A⁴ are each independently an unsubstituted alkyl group having 1 to 20 carbon atoms, a substituted or unsubstituted monovalent aromatic hydrocarbon group, or a substituted or unsubstituted monovalent aromatic hetero ring,
R¹¹ and R¹² are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n1 to n4 are each independently 0, 1, 2, 3, 4, or 5,
n11 and n12 are each independently 0, 1, 2, 3, or 4,
wherein, when m1 is 2 or more, each A¹ are identical to or different from each other,
when m2 is 2 or more, each A² are identical to or different from each other,
when m3 is 2 or more, each A³ are identical to or different from each other,
when m4 is 2 or more, each A⁴ are identical to or different from each other,
when n11 is 2 or more, each R¹¹ are identical to or different from each other,
when n12 is 2 or more, each R¹² are identical to or different from each other, and
each of A¹ to A⁴ and R¹¹ to R¹² are optionally linked to a ring-forming carbon of the core portion,
wherein, in Formula (7),
not all of A¹ to A⁴ are alkyl groups having 1 to 20 carbon atoms, and not all m1 to m4 are 0,
wherein, in Formula (8),
not all of A¹ to A³ are alkyl groups having 1 to 20 carbon atoms, and not all of m1 and m3 are 0. 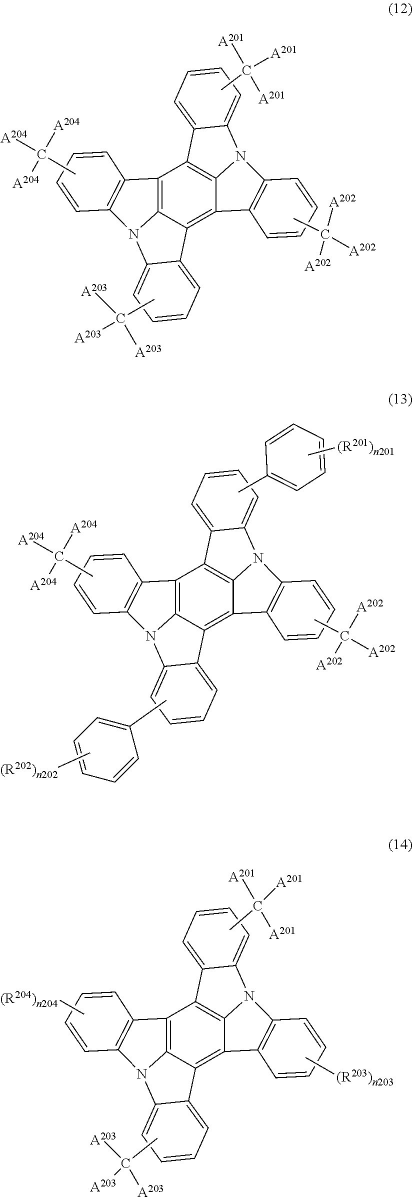
wherein, in Formulae (12) to (14),
A²⁰¹ to A²⁰⁴ are each independently an unsubstituted alkyl group having 1 to 20 carbon atoms, a substituted or unsubstituted monovalent aromatic hydrocarbon group, or a substituted or unsubstituted monovalent aromatic heterocyclic group,
R²⁰¹ to R²⁰² are each independently a halogen atom, a cyano group, an unsubstituted alkyl group having 1 to 20 carbon atoms, an unsubstituted halo alkyl group having 1 to 20 carbon atoms, an unsubstituted alkoxy group having 1 to 20 carbon atoms, an unsubstituted alkyl amino group having 1 to 20 carbon atoms, an unsubstituted aryl amino group having 6 to 20 carbon atoms, or a substituted or unsubstituted monovalent aromatic heterocyclic group,
R²⁰³ and R²⁰⁴ are each independently a halogen atom, a cyano group, an unsubstituted alkyl group having 1 to 20 carbon atoms, an unsubstituted alkyl group having 1 to 20 carbon atoms, an unsubstituted alkoxy group having 1 to 20 carbon atoms, an unsubstituted halo alkoxy group having 1 to 20 carbon atoms, or an unsubstituted aryl amino group having 6 to 20 carbon atoms,
n201 and n202 are each independently 0, 1, 2, 3, 4, or 5,
n203 and n204 are each independently 0, 1, 2, 3, or 4,
wherein, each A²⁰¹ are identical to or different from each other, each A²⁰² are identical to or different from each other, each A²⁰³ are identical to or different from each other, and each A²⁰⁴ are identical to or different from each other,
two or more of A²⁰¹, two or more of A²⁰², two or more of A²⁰³, and two or more of A²⁰⁴ are optionally linked to form a ring,
when n201 is 2 or more, each R²⁰¹ are identical to or different from each other, when n202 is 2 or more, each R²⁰² are identical to or different from each other, when n203 is 2 or more, each R²⁰³ are identical to or different from each other, and when n204 is 2 or more, each R²⁰⁴ are identical to or different from each other,
each of A²⁰¹ to A²⁰⁴ are optionally linked to a ring-forming carbon of the core portion of Formula (12),
each of A²⁰², A²⁰⁴, R²⁰¹, and R²⁰² are optionally linked to a ring-forming carbon of the core portion of Formula (13),
each of A²⁰¹, A²⁰³, R²⁰³, and R²⁰⁴ are optionally linked to a ring-forming carbon of the core portion of Formula (14),
in Formula (12), not all of each A²⁰¹, each A²⁰², each A²⁰³, and each A²⁰⁴ are unsubstituted alkyl groups having 1 to 20 carbon atoms,
in Formula (13), not all of each A²⁰² and each A²⁰⁴ are unsubstituted alkyl groups having 1 to 20 carbon atoms, and
in Formula (14), not all of each A²⁰¹ and each A²⁰³ are unsubstituted alkyl groups having 1 to 20 carbon atoms.

28. The nitrogen-containing condensed cyclic compound of claim 23, wherein
the structure represented by Formula (2), or (3) is represented by one of Formulae (3-1) to (3-9):

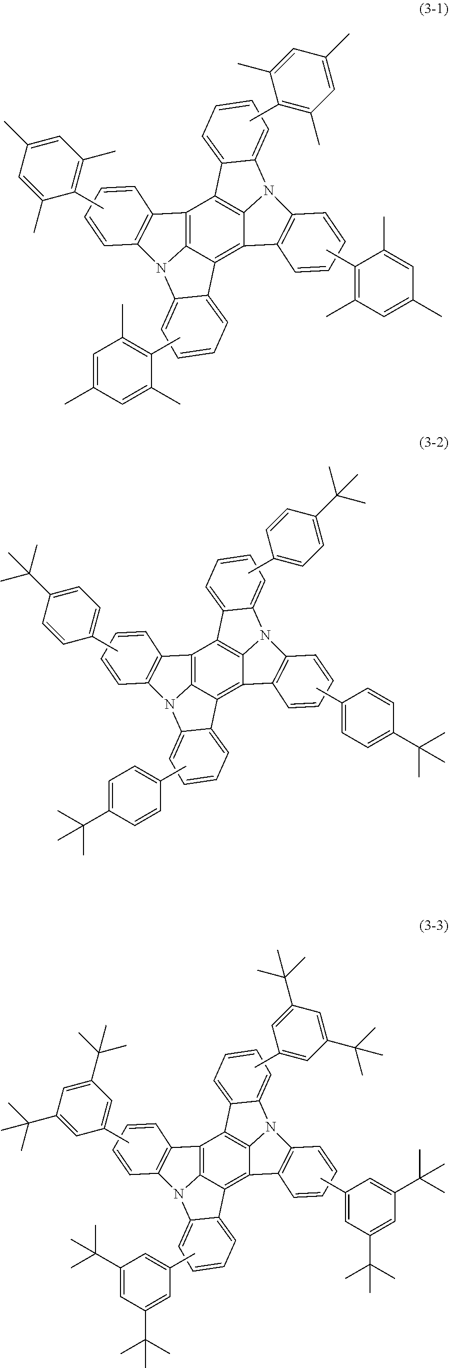

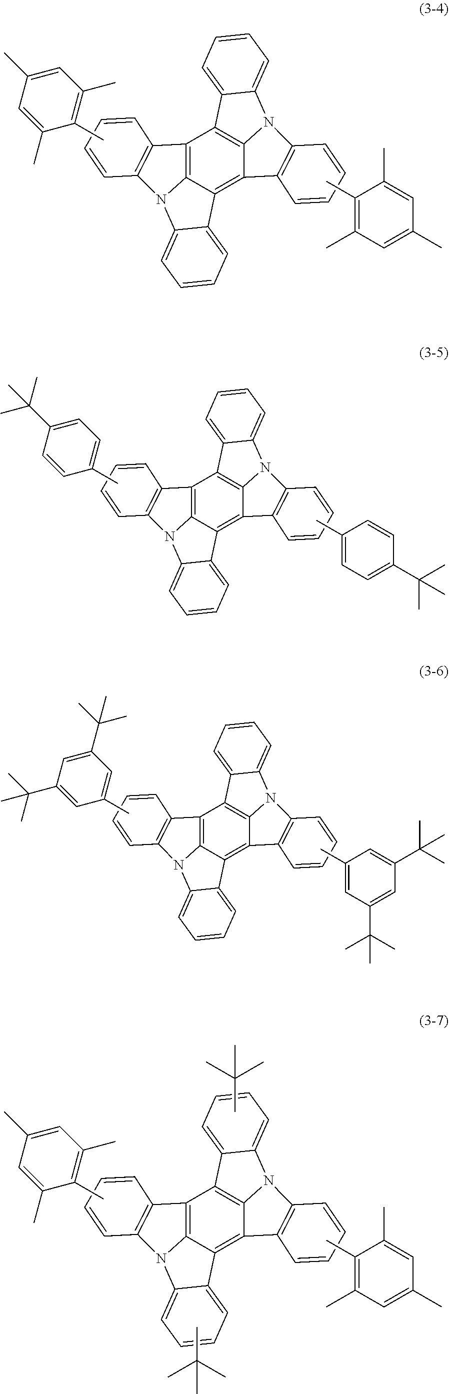

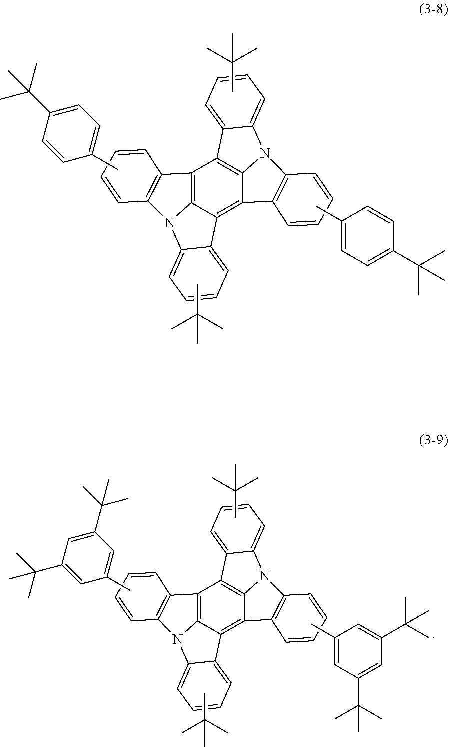

29. The nitrogen-containing condensed cyclic compound of claim 20, the compound comprising
at least one of R¹ to R⁴ is an aromatic hydrocarbon group or a heterocyclic group and is linked to the core portion represented by Formula (1-1) by a single bond, wherein
a dihedral angle between the core portion and the at least one of R¹ to R⁴ is 50° or more.

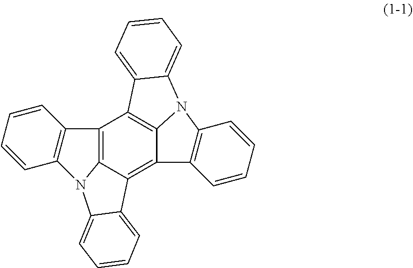 [SMILES: c1ccc2c(c1)c1c3c4ccccc4n4c5ccccc5c(c5c6ccccc6n2c15)c34]

30. The nitrogen-containing condensed cyclic compound of claim 29, wherein
the at least one of R¹ to R⁴ is a group derived from a benzene ring, a group derived from a carbazole ring, a group derived from a dibenzofuran ring, or a combination thereof.

31. The nitrogen-containing condensed cyclic compound of claim 20, wherein
the nitrogen-containing condensed cyclic compound has a mesitylene solubility of 1.0 g/L or more.

32. An organic electroluminescent device comprising the material for an organic electroluminescent device of claim 1.

33. An organic electroluminescent device comprising the nitrogen-containing condensed cyclic compound of claim 20.

34. The organic electroluminescent device of claim 33, wherein
the organic electroluminescent device comprises: the fluorescence emitter or the nitrogen-containing condensed cyclic compound; and a host material.

35. The organic electroluminescent device of claim 34, wherein
the host material has a structure represented by Formula (5): 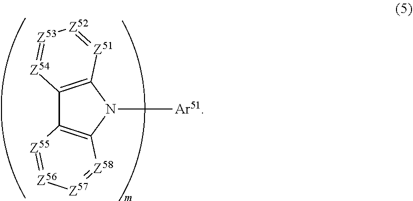
wherein, in Formula (5),
Z⁵¹ is CH, CR⁵¹, or N,
Z⁵² is CH, CR⁵², or N,
Z⁵³ is CH, CR⁵³, or N,
Z⁵⁴ is CH, CR⁵⁴, or N,
Z⁵⁵ is CH, CR⁵⁵, or N,
Z⁵⁶ is CH, CR⁵⁶, or N,
Z⁵⁷ is CH, CR⁵⁷, or N,
Z⁵⁸ is CH, CR⁵⁸, or N,
R⁵¹ to R⁵⁸ are each independently one group of (5a) to (5h),
(5a) is a cyano group,
(5b) is a substituted or unsubstituted alkyl group having 1 to 20 carbon atoms,
(5c) is a substituted or unsubstituted alkoxy group having 1 to 20 carbon atoms,
(5d) is a substituted or unsubstituted aryl amino group having 6 to 20 carbon atoms,
(5e) is a substituted or unsubstituted phosphoryl group (a —POH₂ group),
(5f) is a substituted or unsubstituted silyl group (a —SiH₃ group),
(5g) is a substituted or unsubstituted monovalent aromatic hydrocarbon group,
(5h) is a substituted or unsubstituted monovalent heterocyclic group,
Ar⁵¹ comprises an aromatic hydrocarbon group or a heterocyclic group,
m is 1, 2, 3, 4, 5, or 6,
wherein R⁵¹ and R⁵², R⁵² and R⁵³, R⁵³ and R⁵⁴, R⁵⁵ and R⁵⁶, R⁵⁶ and R⁵⁷, or R⁵⁷ and R⁵⁸ can form an aliphatic hydrocarbon ring, an aromatic hydrocarbon ring, or a hetero ring.

36. The organic electroluminescent device of claim 34, wherein
the host material has a structure represented by Formula (6): 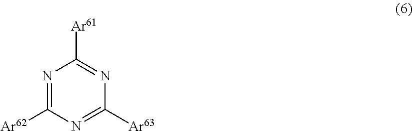
wherein, in Formula (6),
Ar⁶¹ to Ar⁶³ are each independently a substituted or unsubstituted monovalent aromatic hydrocarbon group or a substituted or unsubstituted monovalent heterocyclic group.

37. The organic electroluminescent device of any one of claim 32, wherein
the organic electroluminescent device comprises an emission layer, wherein the emission layer comprises the material for an organic electroluminescent device, the fluorescence emitter, or the nitrogen-containing condensed cyclic compound.
